SEQUENCE LISTING (169 sequences)

SEQ ID NO: 1 - DNA, 43 bp, Artificial
  PCR primer
aataatggatccgtatccggatttaaaaggaaaagtcgtcgct

SEQ ID NO: 2 - DNA, 42 bp, Artificial
  PCR primer
aataataagcttaccgcggcctgcctggaatgaaggatattg

SEQ ID NO: 3 - DNA, 42 bp, Artificial
  PCR primer
aataatcatatgcttgtgaatgtactgatcgtccacgctcac

SEQ ID NO: 4 - DNA, 42 bp, Artificial
  PCR primer
aataatctcgagcgtcagcgggtacaacgcttcatcgaactg

SEQ ID NO: 5 - DNA, 154 bp, Artificial
  GGGSGGGS-Fos direct, HindIII/AflII
aagcttggcggcggcagcggcggcggcagcctgaccgataccctgcaggcggaaaccgatcagctggaagataaaaaaagcgcgctgcagaccgaaattgcgaacctgctgaaagaaaaagaaaaactggaatttattctggcggcgtattaac

SEQ ID NO: 6 - DNA, 153 bp, Artificial
  GGGSGGGS-Fos complement, HindIII/AflII
ttaagttaatacgccgccagaataaattccagtttttctttttctttcagcaggttcgcaatttcggtctgcagcgcgctttttttatcttccagctgatcggtttccgcctgcagggtatcggtcaggctgccgccgccgctgccgccgcca

SEQ ID NO: 7 - protein, 48 aa, Artificial
  GGGSGGGS-Fos
GGGSGGGSLTDTLQAETDQLEDKKSALQTEIANLLKEKEKLEFILAAY

SEQ ID NO: 8 - DNA, 153 bp, Artificial
  GGGSGGGS-Jun direct, XhoI/AvrII
tcgagggcggcggcagcggcggcggcagccgtattgcgcgtctggaagaaaaagtgaaaaccctgaaagcgcagaacagcgaactggcgagcaccgcgaacatgctgcgtgaacaggtggcgcagctgaaacagaaagtgatgaactattaac

SEQ ID NO: 9 - DNA, 153 bp, Artificial
  GGGSGGGS-Jun complement, XhoI/AvrII
ctaggttaatagttcatcactttctgtttcagctgcgccacctgttcacgcagcatgttcgcggtgctcgccagttcgctgttctgcgctttcagggttttcactttttcttccagacgcgcaatacggctgccgccgccgctgccgccgccc

SEQ ID NO: 10 - protein, 48 aa, Artificial
  GGGSGGGS-Jun
GGGSGGGSRIARLEEKVKTLKAQNSELASTANMLREQVAQLKQKVMNY

SEQ ID NO: 11 - DNA, 7028 bp, Artificial
  plasmid, pETDuet-busGDH-Fos/ppuDp-Jun
ggggaattgtgagcggataacaattcccctctagaaataattttgtttaactttaagaaggagatataccatgggcagcagccatcaccatcatcaccacagccaggatccgtatccggatttaaaaggaaaagtcgtcgctattacaggagctgcttcagggctcggaaaggcgatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaggagttcggcacactcgatattatgattaataatgccggtcttgaaaatcctgtgccatctcacgaaatgccgctcaaggattgggataaagtcatcggcacgaacttaacgggtgcctttttaggaagccgtgaagcgattaaatatttcgtagaaaacgatatcaagggaaatgtcattaacatgtccagtgtgcacgaagtgattccttggccgttatttgtccactatgcggcaagtaaaggcgggataaagctgatgacagaaacattagcgttggaatacgcgccgaagggcattcgcgtcaataatattgggccaggtgcgatcaacacgccaatcaatgctgaaaaattcgctgaccctaaacagaaagctgatgtagaaagcatgattccaatgggatatatcggcgaaccggaggagatcgccgcagtagcagcctggcttgcttcgaaggaagccagctacgtcacaggcatcacgttattcgcggacggcggtatgacacaatatccttcattccaggcaggccgcggtaagcttggcggcggcagcggcggcggcagcctgaccgataccctgcaggcggaaaccgatcagctggaagataaaaaaagcgcgctgcagaccgaaattgcgaacctgctgaaagaaaaagaaaaactggaatttattctggcggcgtattaacttaagtcgaacagaaagtaatcgtattgtacacggccgcataatcgaaattaatacgactcactataggggaattgtgagcggataacaattccccatcttagtatattagttaagtataagaaggagatatacatatgcttgtgaatgtactgatcgtccacgctcaccccgaaccgcaatcgttcacggcagccctgcgtgaccaggctgtggaaaccttccgtgcccaaggtcatcaggtgcaggtcagtgacttgtacgccatgggctggaacccggtggccagtgccgatgactttacccagcgcgagaaccccgagtacctcgtgtatgcgctggagcagcgtcagggcgtcaagcgcggtgccattgccgcagatatccagcaggagctggacaagctgttgtgggccgatctgctggtgctgaacttcccgatcttctggttctcggccccggccatgctcaagggctggatcgaccgggtgctggtgtcgggggtgtgctatggcggcaagcgcttctacgaccagggcgggttggccggcaagagggcgctggtgaccgtgaccctgggcgggcgcgagcacatgtttggcgacggcgcgattcacgggccgctggaagacatgctgcggccgattctgcggggcacgctggcctatgtcgggttcgaggtgctgccgccgttcgtggcgtggcatgtgccgtacatcagtgctgatgcgcggcaggactttctgcagcaataccggcagcgtctggagcagctctcggacgaccagccactggtgttcccgcgcctggcgcagttcgatgaagcgttgtacccgctgacgctcgagggcggcggcagcggcggcggcagccgtattgcgcgtctggaagaaaaagtgaaaaccctgaaagcgcagaacagcgaactggcgagcaccgcgaacatgctgcgtgaacaggtggcgcagctgaaacagaaagtgatgaactattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 12 - DNA, 42 bp, Artificial
  PCR primer
aataatggatcctatccggatttaaaaggaaaagtcgtcgct

SEQ ID NO: 13 - DNA, 42 bp, Artificial
  PCR primer
aataatctcgagtcacgtcagcgggtacaacgcttcatcgaa

SEQ ID NO: 14 - DNA, 5146 bp, Artificial
  plasmid, pCDFDuet-busGDH-ppuDp
ggggaattgtgagcggataacaattcccctgtagaaataattttgtttaactttaataaggagatataccatggcgccggatttaaaaggaaaagtcgtcgctattacaggagctgcttcagggctcggaaaggcgatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaggagttcggcacactcgatattatgattaataatgccggtcttgaaaatcctgtgccatctcacgaaatgccgctcaaggattgggataaagtcatcggcacgaacttaacgggtgcctttttaggaagccgtgaagcgattaaatatttcgtagaaaacgatatcaagggaaatgtcattaacatgtccagtgtgcacgaagtgattccttggccgttatttgtccactatgcggcaagtaaaggcgggataaagctgatgacagaaacattagcgttggaatacgcgccgaagggcattcgcgtcaataatattgggccaggtgcgatcaacacgccaatcaatgctgaaaaattcgctgaccctaaacagaaagctgatgtagaaagcatgattccaatgggatatatcggcgaaccggaggagatcgccgcagtagcagcctggcttgcttcgaaggaagccagctacgtcacaggcatcacgttattcgcggacggcggtatgacacaatatccttcattccaggcaggccgcggtaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgcttgtgaatgtactgatcgtccacgctcaccccgaaccgcaatcgttcacggcagccctgcgtgaccaggctgtggaaaccttccgtgcccaaggtcatcaggtgcaggtcagtgacttgtacgccatgggctggaacccggtggccagtgccgatgactttacccagcgcgagaaccccgagtacctcgtgtatgcgctggagcagcgtcagggcgtcaagcgcggtgccattgccgcagatatccagcaggagctggacaagctgttgtgggccgatctgctggtgctgaacttcccgatcttctggttctcggccccggccatgctcaagggctggatcgaccgggtgctggtgtcgggggtgtgctatggcggcaagcgcttctacgaccagggcgggttggccggcaagagggcgctggtgaccgtgaccctgggcgggcgcgagcacatgtttggcgacggcgcgattcacgggccgctggaagacatgctgcggccgattctgcggggcacgctggcctatgtcgggttcgaggtgctgccgccgttcgtggcgtggcatgtgccgtacatcagtgctgatgcgcggcaggactttctgcagcaataccggcagcgtctggagcagctctcggacgaccagccactggtgttcccgcgcctggcgcagttcgatgaagcgttgtacccgctgacgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 15 - protein, 324 aa, Artificial
  His-bsuGDH-Fos
MGSSHHHHHHSQDPYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLENPVPSHEMPLKDWDKVIGTNLTGAFLGSREAIKYFVENDIKGNVINMSSVHEVIPWPLFVHYAASKGGIKLMTETLALEYAPKGIRVNNIGPGAINTPINAEKFADPKQKADVESMIPMGYIGEPEEIAAVAAWLASKEASYVTGITLFADGGMTQYPSFQAGRGKLGGGSGGGSLTDTLQAETDQLEDKKSALQTEIANLLKEKEKLEFILAAY

SEQ ID NO: 16 - protein, 285 aa, Artificial
  ppuDp-Jun
MLVNVLIVHAHPEPQSFTAALRDQAVETFRAQGHQVQVSDLYAMGWNPVASADDFTQRENPEYLVYALEQRQGVKRGAIAADIQQELDKLLWADLLVLNFPIFWFSAPAMLKGWIDRVLVSGVCYGGKRFYDQGGLAGKRALVTVTLGGREHMFGDGAIHGPLEDMLRPILRGTLAYVGFEVLPPFVAWHVPYISADARQDFLQQYRQRLEQLSDDQPLVFPRLAQFDEALYPLTLEGGGSGGGSRIARLEEKVKTLKAQNSELASTANMLREQVAQLKQKVMNY

SEQ ID NO: 17 - DNA, 42 bp, Artificial
  PCR primer
aataatcatatgtatccggatttaaaaggaaaagtcgtcgct

SEQ ID NO: 18 - DNA, 42 bp, Artificial
  PCR primer
aataatctcgagaccgcggcctgcctggaatgaaggatattg

SEQ ID NO: 19 - DNA, 43 bp, Artificial
  PCR primer
aataatggatccgaacattttaataacagcttcttcaagagga

SEQ ID NO: 20 - DNA, 42 bp, Artificial
  PCR primer
aataataagcttaagaagaacgctcctagtcattgctccatc

SEQ ID NO: 21 - DNA, 42 bp, Artificial
  PCR primer
aataatcatatgaagatagtagttataggatctggaactgct

SEQ ID NO: 22 - DNA, 39 bp, Artificial
  PCR primer
aataatctcgagtgacttaaattttctcatggccatttc

SEQ ID NO: 23 - DNA, 7637 bp, Artificial
  plasmid, pETDuet-pfuGDH-Fos/phoDp-Jun
ggggaattgtgagcggataacaattcccctctagaaataattttgtttaactttaagaaggagatataccatgggcagcagccatcaccatcatcaccacagccaggatccgaacattttaataacagcttcttcaagaggaataggcttcaatgtggccaaggaactcctcaaaaaaggacacagagtagtaatatcgtctagcaatgaaaaaaatttagaaaaagccctagaagagctaaaagaatatgaaaaagtctatccgattaaagcagatctctcaaaaaaagaagaaattaaaaacatggtagaaaaggcctgggaaattttaggaaaaatagatgtccttgtttggaatgcccctaatgtaaagtgtgaaccctgcatgattcacgaattagattatgaagattgggtagaaggagccttgcttcacataattgccccaggctacctaacaactcttctccttaataaatggttgaaaaataacacaagagggataataatctacttgagttctgcttcagttttagagccaatgcctcccctccttctcgcagacagtactagggcaggattaattcagcttgctaaaggaatatctagggcctatggaaagagaggaataagggcatacacagtactacttgggagctttgataccccaggagctagagaaaacctaagaagactagcagaaaaaaggggtgttgatccagaagagctatggaaaaaggaagttattgaaagaactcctcttcaaagaactggaaaatgggaagaactaggagaattaatagaattcttacttagcgataacgccgaatatatgctaggctctacaattctctttgatggagcaatgactaggagcgttcttctgaagcttggcggcggcagcggcggcggcagcctgaccgataccctgcaggcggaaaccgatcagctggaagataaaaaaagcgcgctgcagaccgaaattgcgaacctgctgaaagaaaaagaaaaactggaatttattctggcggcgtattaacttaagtcgaacagaaagtaatcgtattgtacacggccgcataatcgaaattaatacgactcactataggggaattgtgagcggataacaattccccatcttagtatattagttaagtataagaaggagatatacatatgaagatagtagttataggatctggaactgctggaagcaattttgctctcttcatgaggaagctcgataggaaggccgagataatagttataggaaaggaacctacaatgcagtactccccctgtgcccttccacacgttattagcggaaccatagagaagcctgaagacgttatagtgtttccgaatgagttctatgagaagcagagaataaagatgatgcttggagtcgaagctaaaaagatagatagggagagaaaggttataattaccgataagggggaggttccctacgataagctggttcttgctactggatcaagggcttttattcccccgataaagggtgtcgaaaacgagggagtgttcacgctaaagagcctcgatgacgtaaggaggataaaggaatacataagcaagaggaaacctaaaaaggttgtcgttattggcgcaggtttaataggtctagaaggagcagaagcctttgcaaagattggaatggatgttctagttgttgaattactggagcatctattaccgactatgctcgacaaagacatggccaagatcgttcaagaggagatggagaaacatggagttaagttcaagtttggagttggagtcagcgagataataggaaatcctgtcaaggaagttaaaattggagaagagagtgtagaagcagatctagtcctagtggcaacgggagttagggctaatgttgacctagcgaaagatgctgggctggaagtgagtagggggatagttgttaacgagtatcttcagaccagtgatcctgatatatatgctataggagactgtgcagaggtattcgatgccgtaactggaaagagaaccctaagccaattgggaacttctgctgtcagaatggccaaagttgccgctgagaacatagctggaaagaaggtgaaatttaggccagtcttcaacacagctataactgaattattcgacctggaaataggaacatttggaataacagaagagagggctaaaaaggagggaattgaagtcgtaattggaaagtttaaaggttcaactaagccagagtattatcctggaggaaaaccaattacggttaagttaatatttagaaaggaagataagagactaataggggcccaaatagttggaggagagagagtttgggggaggataatgactttatcggcactcgcccagaaaggagctaccgttgaagatgttgcctatctagaaacgagttatgcaccacccataagcccaacgatagacccaatcacggtagccgctgaaatggccatgagaaaatttaagtcactcgagggcggcggcagcggcggcggcagccgtattgcgcgtctggaagaaaaagtgaaaaccctgaaagcgcagaacagcgaactggcgagcaccgcgaacatgctgcgtgaacaggtggcgcagctgaaacagaaagtgatgaactattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 24 - DNA, 50 bp, Artificial
  PCR primer
aataatccatggcgattttaataacagcttcttcaagaggaataggcttc

SEQ ID NO: 25 - DNA, 42 bp, Artificial
  PCR primer
aataatctcgagtcatgacttaaattttctcatggccatttc

SEQ ID NO: 26 - DNA, 5755 bp, Artificial
  plasmid, pCDFDuet-pfuGDH-phoDp
taatacgactcactataggggaattgtgagcggataacaattcccctgtagaaataattttgtttaactttaataaggagatataccatggcgattttaataacagcttcttcaagaggaataggcttcaatgtggccaaggaactcctcaaaaaaggacacagagtagtaatatcgtctagcaatgaaaaaaatttagaaaaagccctagaagagctaaaagaatatgaaaaagtctatccgattaaagcagatctctcaaaaaaagaagaaattaaaaacatggtagaaaaggcctgggaaattttaggaaaaatagatgtccttgtttggaatgcccctaatgtaaagtgtgaaccctgcatgattcacgaattagattatgaagattgggtagaaggagccttgcttcacataattgccccaggctacctaacaactcttctccttaataaatggttgaaaaataacacaagagggataataatctacttaagttctgcttcagttttagagccaatgcctcccctccttctcgcagacagtactagggcaggattaattcagcttgctaaaggaatatctagggcctatggaaagagaggaataagggcatacacagtactacttgggagctttgataccccaggagctagagaaaacctaagaagactagcagaaaaaaggggtgttgatccagaagagctatggaaaaaggaagttattgaaagaactcctcttcaaagaactggaaaatgggaagaactaggagaattaatagaattcttacttagcgataacgccgaatatatgctaggctctacaattctctttgatggagcaatgactaggagcgttcttcttaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgaagatagtagttataggatctggaactgctggaagcaattttgctctcttcatgaggaagctcgataggaaggccgagataatagttataggaaaggaacctacaatgcagtactccccctgtgcccttccacacgttattagcggaaccatagagaagcctgaagacgttatagtgtttccgaatgagttctatgagaagcagagaataaagatgatgcttggagtcgaagctaaaaagatagatagggagagaaaggttataattaccgataagggggaggttccctacgataagctggttcttgctactggatcaagggcttttattcccccgataaagggtgtcgaaaacgagggagtgttcacgctaaagagcctcgatgacgtaaggaggataaaggaatacataagcaagaggaaacctaaaaaggttgtcgttattggcgcaggtttaataggtctagaaggagcagaagcctttgcaaagattggaatggatgttctagttgttgaattactggagcatctattaccgactatgctcgacaaagacatggccaagatcgttcaagaggagatggagaaacatggagttaagttcaagtttggagttggagtcagcgagataataggaaatcctgtcaaggaagttaaaattggagaagagagtgtagaagcagatctagtcctagtggcaacgggagttagggctaatgttgacctagcgaaagatgctgggctggaagtgagtagggggatagttgttaacgagtatcttcagaccagtgatcctgatatatatgctataggagactgtgcagaggtattcgatgccgtaactggaaagagaaccctaagccaattgggaacttctgctgtcagaatggccaaagttgccgctgagaacatagctggaaagaaggtgaaatttaggccagtcttcaacacagctataactgaattattcgacctggaaataggaacatttggaataacagaagagagggctaaaaaggagggaattgaagtcgtaattggaaagtttaaaggttcaactaagccagagtattatcctggaggaaaaccaattacggttaagttaatatttagaaaggaagataagagactaataggggcccaaatagttggaggagagagagtttgggggaggataatgactttatcggcactcgcccagaaaggagctaccgttgaagatgttgcctatctagaaacgagttatgcaccacccataagcccaacgatagacccaatcacggtagccgctgaaatggccatgagaaaatttaagtcatgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 27 - protein, 322 aa, Artificial
  pfuGDH-Fos
MGSSHHHHHHSQDPNILITASSRGIGFNVAKELLKKGHRVVISSSNEKNLEKALEELKEYEKVYPIKADLSKKEEIKNMVEKAWEILGKIDVLVWNAPNVKCEPCMIHELDYEDWVEGALLHIIAPGYLTTLLLNKWLKNNTRGIIIYLSSASVLEPMPPLLLADSTRAGLIQLAKGISRAYGKRGIRAYTVLLGSFDTPGARENLRRLAEKRGVDPEELWKKEVIERTPLQRTGKWEELGELIEFLLSDNAEYMLGSTILFDGAMTRSVLLKLGGGSGGGSLTDTLQAETDQLEDKKSALQTEIANLLKEKEKLEFILAAY

SEQ ID NO: 28 - protein, 490 aa, Artificial
  phoDp-Jun
MKIVVIGSGTAGSNFALFMRKLDRKAEIIVIGKEPTMQYSPCALPHVISGTIEKPEDVIVFPNEFYEKQRIKMMLGVEAKKIDRERKVIITDKGEVPYDKLVLATGSRAFIPPIKGVENEGVFTLKSLDDVRRIKEYISKRKPKKVVVIGAGLIGLEGAEAFAKIGMDVLVVELLEHLLPTMLDKDMAKIVQEEMEKHGVKFKFGVGVSEIIGNPVKEVKIGEESVEADLVLVATGVRANVDLAKDAGLEVSRGIVVNEYLQTSDPDIYAIGDCAEVFDAVTGKRTLSQLGTSAVRMAKVAAENIAGKKVKFRPVFNTAITELFDLEIGTFGITEERAKKEGIEVVIGKFKGSTKPEYYPGGKPITVKLIFRKEDKRLIGAQIVGGERVWGRIMTLSALAQKGATVEDVAYLETSYAPPISPTIDPITVAAEMAMRKFKSLEGGGSGGGSRIARLEEKVKTLKAQNSELASTANMLREQVAQLKQKVMNY

SEQ ID NO: 29 - DNA, 42 bp, Artificial
  PCR primer
aataatcatatgaacattttaataacagcttcttcaagagga

SEQ ID NO: 30 - DNA, 42 bp, Artificial
  PCR primer
aataatctcgagaagaagaacgctcctagtcattgctccatc

SEQ ID NO: 31 - DNA, 43 bp, Artificial
  PCR primer
aataatggatccgccttcgcaagtcattcctgaaaaacaaaag

SEQ ID NO: 32 - DNA, 42 bp, Artificial
  PCR primer
aataataagctttttagaagtctcaacaacatatctaccaac

SEQ ID NO: 33 - DNA, 7298 bp, Artificial
  plasmid, pETDuet-sceADH-Fos/ppuDp-Jun
ggggaattgtgagcggataacaattcccctctagaaataattttgtttaactttaagaaggagatataccatgggcagcagccatcaccatcatcaccacagccaggatccgccttcgcaagtcattcctgaaaaacaaaaggctattgtcttttatgagacagatggaaaattggaatataaagacgtcacagttccggaacctaagcctaacgaaattttagtccacgttaaatattctggtgtttgtcatagtgacttgcacgcgtggcacggtgattggccatttcaattgaaatttccattaatcggtggtcacgaaggtgctggtgttgttgttaagttgggatctaacgttaagggctggaaagtcggtgattttgcaggtataaaatggttgaatgggacttgcatgtcctgtgaatattgtgaagtaggtaatgaatctcaatgtccttatttggatggtactggcttcacacatgatggtacttttcaagaatacgcaactgccgatgccgttcaagctgcccatattccaccaaacgtcaatcttgctgaagttgccccaatcttgtgtgcaggtatcactgtttataaggcgttgaaaagagccaatgtgataccaggccaatgggtcactatatccggtgcatgcggtggcttgggttctctggcaatccaatacgcccttgctatgggttacagggtcattggtatcgatggtggtaatgccaagcgaaagttatttgaacaattaggcggagaaatattcatcgatttcacggaagaaaaagacattgttggtgctataataaaggccactaatggcggttctcatggagttattaatgtgtctgtttctgaagcagctatcgaggcttctacgaggtattgtaggcccaatggtactgtcgtcctggttggtatgccagctcatgcttactgcaattccgatgttttcaatcaagttgtaaaatcaatctccatcgttggatcttgtgttggaaatagagctgatacaagggaggctttagatttcttcgccagaggtttgatcaaatctccgatccacttagctggcctatcggatgttcctgaaatttttgcaaagatggagaagggtgaaattgttggtagatatgttgttgagacttctaaaaagcttggcggcggcagcggcggcggcagcctgaccgataccctgcaggcggaaaccgatcagctggaagataaaaaaagcgcgctgcagaccgaaattgcgaacctgctgaaagaaaaagaaaaactggaatttattctggcggcgtattaacttaagtcgaacagaaagtaatcgtattgtacacggccgcataatcgaaattaatacgactcactataggggaattgtgagcggataacaattccccatcttagtatattagttaagtataagaaggagatatacatatgcttgtgaatgtactgatcgtccacgctcaccccgaaccgcaatcgttcacggcagccctgcgtgaccaggctgtggaaaccttccgtgcccaaggtcatcaggtgcaggtcagtgacttgtacgccatgggctggaacccggtggccagtgccgatgactttacccagcgcgagaaccccgagtacctcgtgtatgcgctggagcagcgtcagggcgtcaagcgcggtgccattgccgcagatatccagcaggagctggacaagctgttgtgggccgatctgctggtgctgaacttcccgatcttctggttctcggccccggccatgctcaagggctggatcgaccgggtgctggtgtcgggggtgtgctatggcggcaagcgcttctacgaccagggcgggttggccggcaagagggcgctggtgaccgtgaccctgggcgggcgcgagcacatgtttggcgacggcgcgattcacgggccgctggaagacatgctgcggccgattctgcggggcacgctggcctatgtcgggttcgaggtgctgccgccgttcgtggcgtggcatgtgccgtacatcagtgctgatgcgcggcaggactttctgcagcaataccggcagcgtctggagcagctctcggacgaccagccactggtgttcccgcgcctggcgcagttcgatgaagcgttgtacccgctgacgctcgagggcggcggcagcggcggcggcagccgtattgcgcgtctggaagaaaaagtgaaaaccctgaaagcgcagaacagcgaactggcgagcaccgcgaacatgctgcgtgaacaggtggcgcagctgaaacagaaagtgatgaactattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 34 - DNA, 44 bp, Artificial
  PCR primer
aataatccatggcgtcgcaagtcattcctgaaaaacaaaaggct

SEQ ID NO: 35 - DNA, 5416 bp, Artificial
  plasmid, pCDFDuet-sceADH-ppuDp
taatacgactcactataggggaattgtgagcggataacaattcccctgtagaaataattttgtttaactttaataaggagatataccatggcgtcgcaagtcattcctgaaaaacaaaaggctattgtcttttatgagacagatggaaaattggaatataaagacgtcacagttccggaacctaagcctaacgaaattttagtccacgttaaatattctggtgtttgtcatagtgacttgcacgcgtggcacggtgattggccatttcaattgaaatttccattaatcggtggtcacgaaggtgctggtgttgttgttaagttgggatctaacgttaagggctggaaagtcggtgattttgcaggtataaaatggttgaatgggacttgcatgtcctgtgaatattgtgaagtaggtaatgaatctcaatgtccttatttggatggtactggcttcacacatgatggtacttttcaagaatacgcaactgccgatgccgttcaagctgcccatattccaccaaacgtcaatcttgctgaagttgccccaatcttgtgtgcaggtatcactgtttataaggcgttgaaaagagccaatgtgataccaggccaatgggtcactatatccggtgcatgcggtggcttgggttctctggcaatccaatacgcccttgctatgggttacagggtcattggtatcgatggtggtaatgccaagcgaaagttatttgaacaattaggcggagaaatattcatcgatttcacggaagaaaaagacattgttggtgctataataaaggccactaatggcggttctcatggagttattaatgtgtctgtttctgaagcagctatcgaggcttctacgaggtattgtaggcccaatggtactgtcgtcctggttggtatgccagctcatgcttactgcaattccgatgttttcaatcaagttgtaaaatcaatctccatcgttggatcttgtgttggaaatagagctgatacaagggaggctttagatttcttcgccagaggtttgatcaaatctccgatccacttagctggcctatcggatgttcctgaaatttttgcaaagatggagaagggtgaaattgttggtagatatgttgttgagacttctaaaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgcttgtgaatgtactgatcgtccacgctcaccccgaaccgcaatcgttcacggcagccctgcgtgaccaggctgtggaaaccttccgtgcccaaggtcatcaggtgcaggtcagtgacttgtacgccatgggctggaacccggtggccagtgccgatgactttacccagcgcgagaaccccgagtacctcgtgtatgcgctggagcagcgtcagggcgtcaagcgcggtgccattgccgcagatatccagcaggagctggacaagctgttgtgggccgatctgctggtgctgaacttcccgatcttctggttctcggccccggccatgctcaagggctggatcgaccgggtgctggtgtcgggggtgtgctatggcggcaagcgcttctacgaccagggcgggttggccggcaagagggcgctggtgaccgtgaccctgggcgggcgcgagcacatgtttggcgacggcgcgattcacgggccgctggaagacatgctgcggccgattctgcggggcacgctggcctatgtcgggttcgaggtgctgccgccgttcgtggcgtggcatgtgccgtacatcagtgctgatgcgcggcaggactttctgcagcaataccggcagcgtctggagcagctctcggacgaccagccactggtgttcccgcgcctggcgcagttcgatgaagcgttgtacccgctgacgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 36 - protein, 414 aa, Artificial
  His-sceADH-Fos
MGSSHHHHHHSQDPPSQVIPEKQKAIVFYETDGKLEYKDVTVPEPKPNEILVHVKYSGVCHSDLHAWHGDWPFQLKFPLIGGHEGAGVVVKLGSNVKGWKVGDFAGIKWLNGTCMSCEYCEVGNESQCPYLDGTGFTHDGTFQEYATADAVQAAHIPPNVNLAEVAPILCAGITVYKALKRANVIPGQWVTISGACGGLGSLAIQYALAMGYRVIGIDGGNAKRKLFEQLGGEIFIDFTEEKDIVGAIIKATNGGSHGVINVSVSEAAIEASTRYCRPNGTVVLVGMPAHAYCNSDVFNQVVKSISIVGSCVGNRADTREALDFFARGLIKSPIHLAGLSDVPEIFAKMEKGEIVGRYVVETSKKLGGGSGGGSLTDTLQAETDQLEDKKSALQTEIANLLKEKEKLEFILAAY

SEQ ID NO: 37 - DNA, 42 bp, Artificial
  PCR primer
aataatcatatgccttcgcaagtcattcctgaaaaacaaaag

SEQ ID NO: 38 - DNA, 42 bp, Artificial
  PCR primer
aataatctcgagtttagaagtctcaacaacatatctaccaac

SEQ ID NO: 39 - DNA, 43 bp, Artificial
  PCR primer
aataatggatccgtttttcctaaagactaagattatcgaggga

SEQ ID NO: 40 - DNA, 42 bp, Artificial
  PCR primer
aataataagcttatctccatagaaggccctctcataaattct

SEQ ID NO: 41 - DNA, 7988 bp, Artificial
  plasmid, pETDuet-pfuADH-Fos/phoDp-Jun
ggggaattgtgagcggataacaattcccctctagaaataattttgtttaactttaagaaggagatataccatgggcagcagccatcaccatcatcaccacagccaggatccgtttttcctaaagactaagattatcgagggaaggggaagtttggagtatcttcaaaaggaagctattgggttcgagagggctttaatcttagcgtcaagctctatgaaaaggttagggtttctgaaggaagctgaagactatctaagggaaggtggagttgaggtaatttcaatagcaggccttcctcctgagcccacgaaagacgttgtcgacgagttcctcccaaaagttagggagttttctccagagatcctcattgcaattggtggaggaagtgttatcgatatggccaaagctctaaaggtattttacgatgtcccaggaatttcatttgaggaagttgcaatcaagagtagattttcgaagccgagacccgttccaaagctgaaaactccccttatagctattccctcaacgagcggtgctggaagtgaggtttcagcagcgagcgtgattaaggtaggggacgttaagtacacgattgtaactcccgagatagctcctgaagtggctatacttgaccctagattacccgaaggaatgcccccagaagttgccaggaattctggaatggatgttttagttcatgcgatagaggcttatgtttcaaaggcttcctcaccctttacagatgcaatggcagttaaagcggcaaaagctgtgttggagcatttagaggagagcattagaggagacaaaaatgccagagagaaaatccactatgcagctacaatggctggcatagctttcttaaacgcaaggcttggactcgttcatgcgatgagtcataaggccgcatggataggaccgcatggtctcgtaaatgccattctacttccctatgtgatggagtataacatggcacgcaggagagagaaatatgatgcattggcgagggaattgggatttgacaatgctgaagagctccttgaagagataaaagctctgaacgaaaggcttggaataggacccatctcaagttacgtcagcgaggaggagttcttagcgaagctggatgaaatggtagagagagcctatgaagatcccctcgttaacttcaacccagttgagccgagaaaagaggacattagaagaatttatgagagggccttctatggagataagcttggcggcggcagcggcggcggcagcctgaccgataccctgcaggcggaaaccgatcagctggaagataaaaaaagcgcgctgcagaccgaaattgcgaacctgctgaaagaaaaagaaaaactggaatttattctggcggcgtattaacttaagtcgaacagaaagtaatcgtattgtacacggccgcataatcgaaattaatacgactcactataggggaattgtgagcggataacaattccccatcttagtatattagttaagtataagaaggagatatacatatgaagatagtagttataggatctggaactgctggaagcaattttgctctcttcatgaggaagctcgataggaaggccgagataatagttataggaaaggaacctacaatgcagtactccccctgtgcccttccacacgttattagcggaaccatagagaagcctgaagacgttatagtgtttccgaatgagttctatgagaagcagagaataaagatgatgcttggagtcgaagctaaaaagatagatagggagagaaaggttataattaccgataagggggaggttccctacgataagctggttcttgctactggatcaagggcttttattcccccgataaagggtgtcgaaaacgagggagtgttcacgctaaagagcctcgatgacgtaaggaggataaaggaatacataagcaagaggaaacctaaaaaggttgtcgttattggcgcaggtttaataggtctagaaggagcagaagcctttgcaaagattggaatggatgttctagttgttgaattactggagcatctattaccgactatgctcgacaaagacatggccaagatcgttcaagaggagatggagaaacatggagttaagttcaagtttggagttggagtcagcgagataataggaaatcctgtcaaggaagttaaaattggagaagagagtgtagaagcagatctagtcctagtggcaacgggagttagggctaatgttgacctagcgaaagatgctgggctggaagtgagtagggggatagttgttaacgagtatcttcagaccagtgatcctgatatatatgctataggagactgtgcagaggtattcgatgccgtaactggaaagagaaccctaagccaattgggaacttctgctgtcagaatggccaaagttgccgctgagaacatagctggaaagaaggtgaaatttaggccagtcttcaacacagctataactgaattattcgacctggaaataggaacatttggaataacagaagagagggctaaaaaggagggaattgaagtcgtaattggaaagtttaaaggttcaactaagccagagtattatcctggaggaaaaccaattacggttaagttaatatttagaaaggaagataagagactaataggggcccaaatagttggaggagagagagtttgggggaggataatgactttatcggcactcgcccagaaaggagctaccgttgaagatgttgcctatctagaaacgagttatgcaccacccataagcccaacgatagacccaatcacggtagccgctgaaatggccatgagaaaatttaagtcatgactcgagggcggcggcagcggcggcggcagccgtattgcgcgtctggaagaaaaagtgaaaaccctgaaagcgcagaacagcgaactggcgagcaccgcgaacatgctgcgtgaacaggtggcgcagctgaaacagaaagtgatgaactattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 42 - DNA, 44 bp, Artificial
  PCR primer
aataatccatggcgttcctaaagactaagattatcgagggaagg

SEQ ID NO: 43 - DNA, 6103 bp, Artificial
  plasmid, pCDFDuet-pfuADH-phoDp
taatacgactcactataggggaattgtgagcggataacaattcccctgtagaaataattttgtttaactttaataaggagatataccatggcgttcctaaagactaagattatcgagggaaggggaagtttggagtatcttcaaaaggaagctattgggttcgagagggctttaatcttagcgtcaagctctatgaaaaggttagggtttctgaaggaagctgaagactatctaagggaaggtggagttgaggtaatttcaatagcaggccttcctcctgagcccacgaaagacgttgtcgacgagttcctcccaaaagttagggagttttctccagagatcctcattgcaattggtggaggaagtgttatcgatatggccaaagctctaaaggtattttacgatgtcccaggaatttcatttgaggaagttgcaatcaagagtagattttcgaagccgagacccgttccaaagctgaaaactccccttatagctattccctcaacgagcggtgctggaagtgaggtttcagcagcgagcgtgattaaggtaggggacgttaagtacacgattgtaactcccgagatagctcctgaagtggctatacttgaccctagattacccgaaggaatgcccccagaagttgccaggaattctggaatggatgttttagttcatgcgatagaggcttatgtttcaaaggcttcctcaccctttacagatgcaatggcagttaaagcggcaaaagctgtgttggagcatttagaggagagcattagaggagacaaaaatgccagagagaaaatccactatgcagctacaatggctggcatagctttcttaaacgcaaggcttggactcgttcatgcgatgagtcataaggccgcatggataggaccgcatggtctcgtaaatgccattctacttccctatgtgatggagtataacatggcacgcaggagagagaaatatgatgcattggcgagggaattgggatttgacaatgctgaagagctccttgaagagataaaagctctgaacgaaaggcttggaataggacccatctcaagttacgtcagcgaggaggagttcttagcgaagctggatgaaatggtagagagagcctatgaagatcccctcgttaacttcaacccagttgagccgagaaaagaggacattagaagaatttatgagagggccttctatggagataagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgaagatagtagttataggatctggaactgctggaagcaattttgctctcttcatgaggaagctcgataggaaggccgagataatagttataggaaaggaacctacaatgcagtactccccctgtgcccttccacacgttattagcggaaccatagagaagcctgaagacgttatagtgtttccgaatgagttctatgagaagcagagaataaagatgatgcttggagtcgaagctaaaaagatagatagggagagaaaggttataattaccgataagggggaggttccctacgataagctggttcttgctactggatcaagggcttttattcccccgataaagggtgtcgaaaacgagggagtgttcacgctaaagagcctcgatgacgtaaggaggataaaggaatacataagcaagaggaaacctaaaaaggttgtcgttattggcgcaggtttaataggtctagaaggagcagaagcctttgcaaagattggaatggatgttctagttgttgaattactggagcatctattaccgactatgctcgacaaagacatggccaagatcgttcaagaggagatggagaaacatggagttaagttcaagtttggagttggagtcagcgagataataggaaatcctgtcaaggaagttaaaattggagaagagagtgtagaagcagatctagtcctagtggcaacgggagttagggctaatgttgacctagcgaaagatgctgggctggaagtgagtagggggatagttgttaacgagtatcttcagaccagtgatcctgatatatatgctataggagactgtgcagaggtattcgatgccgtaactggaaagagaaccctaagccaattgggaacttctgctgtcagaatggccaaagttgccgctgagaacatagctggaaagaaggtgaaatttaggccagtcttcaacacagctataactgaattattcgacctggaaataggaacatttggaataacagaagagagggctaaaaaggagggaattgaagtcgtaattggaaagtttaaaggttcaactaagccagagtattatcctggaggaaaaccaattacggttaagttaatatttagaaaggaagataagagactaataggggcccaaatagttggaggagagagagtttgggggaggataatgactttatcggcactcgcccagaaaggagctaccgttgaagatgttgcctatctagaaacgagttatgcaccacccataagcccaacgatagacccaatcacggtagccgctgaaatggccatgagaaaatttaagtcatgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 44 - protein, 438 aa, Artificial
  His-pfuADH-Fos
MGSSHHHHHHSQDPFFLKTKIIEGRGSLEYLQKEAIGFERALILASSSMKRLGFLKEAEDYLREGGVEVISIAGLPPEPTKDVVDEFLPKVREFSPEILIAIGGGSVIDMAKALKVFYDVPGISFEEVAIKSRFSKPRPVPKLKTPLIAIPSTSGAGSEVSAASVIKVGDVKYTIVTPEIAPEVAILDPRLPEGMPPEVARNSGMDVLVHAIEAYVSKASSPFTDAMAVKAAKAVLEHLEESIRGDKNAREKIHYAATMAGIAFLNARLGLVHAMSHKAAWIGPHGLVNAILLPYVMEYNMARRREKYDALARELGFDNAEELLEEIKALNERLGIGPISSYVSEEEFLAKLDEMVERAYEDPLVNFNPVEPRKEDIRRIYERAFYGDKLGGGSGGGSLTDTLQAETDQLEDKKSALQTEIANLLKEKEKLEFILAAY

SEQ ID NO: 45 - DNA, 42 bp, Artificial
  PCR primer
aataatcatatgtttttcctaaagactaagattatcgaggga

SEQ ID NO: 46 - DNA, 42 bp, Artificial
  PCR primer
aataatctcgagatctccatagaaggccctctcataaattct

SEQ ID NO: 47 - DNA, 43 bp, Artificial
  PCR primer
aataatggatccgtcaagcatgccaaatcatcaaaaagttgtg

SEQ ID NO: 48 - DNA, 42 bp, Artificial
  PCR primer
aataataagctttttattttctaattcagctaaaccgtcgtt

SEQ ID NO: 49 - DNA, 42 bp, Artificial
  PCR primer
aataatcatatgtgcgatacgttcgatctgattgttgttggt

SEQ ID NO: 50 - DNA, 42 bp, Artificial
  PCR primer
aataatctcgaggatatgcagcgggccgtcgaaggccgccag

SEQ ID NO: 51 - DNA, 147 bp, Artificial
  GGGSGGGSGGGS-Dzip1 direct, HindIII/AflII
agcttggcggcggcagcggcggcggcagcggcggcggcagcaccgtggcgcagctggaagaaaaagtgaaaaccctgcgtgcgcagaactatgaactgaaaagccgtgtgcagcgtctgcgtgaacaggtggcgcagctggcgtaac

SEQ ID NO: 52 - DNA, 147 bp, Artificial
  GGGSGGGSGGGS-Dzip1 complement, HindIII/AflII
ttaagttacgccagctgcgccacctgttcacgcagacgctgcacacggcttttcagttcatagttctgcgcacgcagggttttcactttttcttccagctgcgccacggtgctgccgccgccgctgccgccgccgctgccgccgcca

SEQ ID NO: 53 - protein, 46 aa, Artificial
  GGGSGGGSGGGS-Dzip1
GGGSGGGSGGGSTVAQLEEKVKTLRAQNYELKSRVQRLREQVAQLA

SEQ ID NO: 54 - DNA, 144 bp, Artificial
  GGGSGGGSGGGS-Dzip2 direct, XhoI/AvrII
tcgagggcggcggcagcggcggcggcagcggcggcggcagcagcgtggatgaactgcaggcggaagtggatcagctgcaggatgaaaactatgcgctgaaaaccaaagtggcgcagctgcgtaaaaaagtggaaaaactgtaac

SEQ ID NO: 55 - DNA, 144 bp, Artificial
  GGGSGGGSGGGS-Dzip2 complement, XhoI/AvrII
ctaggttacagtttttccacttttttacgcagctgcgccactttggttttcagcgcatagttttcatcctgcagctgatccacttccgcctgcagttcatccacgctgctgccgccgccgctgccgccgccgctgccgccgccc

SEQ ID NO: 56 - protein, 45 aa, Artificial
  GGGSGGGSGGGS-Dzip2
GGGSGGGSGGGSSVDELQAEVDQLQDENYALKTKVAQLRKKVEKL

SEQ ID NO: 57 - DNA, 7889 bp, Artificial
  plasmid, pETDuet-lplLDH-Dzip1/goxDp-Dzip2
ggggaattgtgagcggataacaattcccctctagaaataattttgtttaactttaagaaggagatataccatgggcagcagccatcaccatcatcaccacagccaggatccgtcaagcatgccaaatcatcaaaaagttgtgttagtcggcgacggcgctgttggttctagttacgcttttgcgatggcacaacaaggaattgctgaagaatttgtaattgtcgatgttgttaaagatcggacaaagggtgacgcccttgatcttgaagacgcccaagcattcaccgctcccaagaagatttactcaggcgaatattcagattgtaaggacgctgacttagttgttattacagccggtgcgcctcaaaagcctggtgaatcacgtttagacttagttaacaagaatttaaatatcctatcatccattgtcaaaccagttgttgactccggctttgacggcatcttcttagttgctgctaaccctgttgacatcttaacttacgctacttggaaattctcaggtttcccaaaggatcgtgtcattggttcagggacttccttagactcttcacgtttacgcgttgcgttaggcaaacaattcaatgttgatcctcgttccgttgatgcttacatcatgggtgaacacggtgattctgaatttgctgcttactcaactgcaaccatcgggacacgtccagttcgcgatgtcgctaaggaacaaggcgtttctgacgaagatttagccaagttagaagacggtgttcgtaacaaagcgtacgacatcatcaacttgaagggtgccacgttctacggtatcgggactgctttaatgcggatttccaaagccattttacgtgatgaaaatgccgttttaccagtaggtgcctacatggacggccaatacggcttaaacgacatttatatcgggactccggctgtgattggtggaactggtttgaaacaaatcatcgaatcaccactttcagctgacgaactcaagaagatgcaagattccgccgcaactttgaaaaaagtgcttaacgacggtttagctgaattagaaaataaaaagcttggcggcggcagcggcggcggcagcggcggcggcagcaccgtggcgcagctggaagaaaaagtgaaaaccctgcgtgcgcagaactatgaactgaaaagccgtgtgcagcgtctgcgtgaacaggtggcgcagctggcgtaacttaagtcgaacagaaagtaatcgtattgtacacggccgcataatcgaaattaatacgactcactataggggaattgtgagcggataacaattccccatcttagtatattagttaagtataagaaggagatatacatatgtgcgatacgttcgatctgattgttgttggtggcggccccggcggctatgtggcggcgctgcgggccagccagcttggcatgagcgtggcgctcgtggaaagcacgcatttcggcggcgtgtgcctcaactggggctgcattccgaccaaggcgcttctacggtcttcggaaattcaccacctgcttcacgaactggggacgttcggactttccgccgacaatatctcgttcgacctgtccaagatcgttggccgttcacgcagcatcgcacgccggatgggtggcggcattgcacatctgctgaagaagaccaaggtcacgacctttgacggtcgtgcaaagctggccggccgttcgggtgaggcgcatcaggttgccatcaccaaggatggcgcagcagttgccaccatcaaggcgccgcatgtcatccttgcaacgggtgcgcgcggtcgtcagcttccgggtcttgagacagacggcaccctgatctggggcgcacgcgaggccatgacaccgaaggaactccccaagcggctcctcgtcatcggttcgggcgctattggcatcgagttcgcatccttctaccgaaacatgggcagcgaggtcacgattgccgaagtggcggaccggatcctgattgccgaagatccggaaatcagcgccgctgcccgcaaggcttttgagaagcaggggatgaagatcatcaccagcgcgaaggtcggaccgctgaacaagggtgagaacgaggtctccacgacgatcgagagcccgaccgggaaggtcgatctgacggtggatcgcgttatctgcgcggttggcattgtcggcaatgtcgaggatctggggcttgagggcaccaaggttcaggtcgagcggacccatatcgtgacggacggcttctgccgcaccggcgaaccgggcatctatgcgatcggcgacgttgcgggtgctccgtggctggcacacaaggcgagccatgaaggcatcctgtgcgtcgagaagattgctggccgttcgccgcagccgctccatccgctgaatatcccgggctgtacctattcgcgtccgcagatcgcctctgtcgggcttagcgaagagaaggccattgctgccggccacaaggtcaaggtcggccgcttcccgttcatcgccaacggcaaggccgtcgcgatgggtgagacggacggcatggtcaaaaccgtgttcgatgccacgagcggcgaacttcttggcgcccacatgatcggcgcggaagtcacggaaatgatccaaggctacgtcatcacccgcacaggcgaactgaccgaagcggagctggtcgagacggttttcccgcatccgaccatttccgaaaccatgcatgaggcaactctggcggccttcgacggcccgctgcatatcctcgagggcggcggcagcggcggcggcagcggcggcggcagcagcgtggatgaactgcaggcggaagtggatcagctgcaggatgaaaactatgcgctgaaaaccaaagtggcgcagctgcgtaaaaaagtggaaaaactgtaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 58 - DNA, 44 bp, Artificial
  PCR primer
aataatccatggcgagcatgccaaatcatcaaaaagttgtgtta

SEQ ID NO: 59 - DNA, 42 bp, Artificial
  PCR primer
aataatctcgagtcagatatgcagcgggccgtcgaaggccgc

SEQ ID NO: 60 - DNA, 6022 bp, Artificial
  plasmid, pCDFDuet-lplLDH-goxDp
taatacgactcactataggggaattgtgagcggataacaattcccctgtagaaataattttgtttaactttaataaggagatataccatggcgagcatgccaaatcatcaaaaagttgtgttagtcggcgacggcgctgttggttctagttacgcttttgcgatggcacaacaaggaattgctgaagaatttgtaattgtcgatgttgttaaagatcggacaaagggtgacgcccttgatcttgaagacgcccaagcattcaccgctcccaagaagatttactcaggcgaatattcagattgtaaggacgctgacttagttgttattacagccggtgcgcctcaaaagcctggtgaatcacgtttagacttagttaacaagaatttaaatatcctatcatccattgtcaaaccagttgttgactccggctttgacggcatcttcttagttgctgctaaccctgttgacatcttaacttacgctacttggaaattctcaggtttcccaaaggatcgtgtcattggttcagggacttccttagactcttcacgtttacgcgttgcgttaggcaaacaattcaatgttgatcctcgttccgttgatgcttacatcatgggtgaacacggtgattctgaatttgctgcttactcaactgcaaccatcgggacacgtccagttcgcgatgtcgctaaggaacaaggcgtttctgacgaagatttagccaagttagaagacggtgttcgtaacaaagcgtacgacatcatcaacttgaagggtgccacgttctacggtatcgggactgctttaatgcggatttccaaagccattttacgtgatgaaaatgccgttttaccagtaggtgcctacatggacggccaatacggcttaaacgacatttatatcgggactccggctgtgattggtggaactggtttgaaacaaatcatcgaatcaccactttcagctgacgaactcaagaagatgcaagattccgccgcaactttgaaaaaagtgcttaacgacggtttagctgaattagaaaataaaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgtgcgatacgttcgatctgattgttgttggtggcggccccggcggctatgtggcggcgctgcgggccagccagcttggcatgagcgtggcgctcgtggaaagcacgcatttcggcggcgtgtgcctcaactggggctgcattccgaccaaggcgcttctacggtcttcggaaattcaccacctgcttcacgaactggggacgttcggactttccgccgacaatatctcgttcgacctgtccaagatcgttggccgttcacgcagcatcgcacgccggatgggtggcggcattgcacatctgctgaagaagaccaaggtcacgacctttgacggtcgtgcaaagctggccggccgttcgggtgaggcgcatcaggttgccatcaccaaggatggcgcagcagttgccaccatcaaggcgccgcatgtcatccttgcaacgggtgcgcgcggtcgtcagcttccgggtcttgagacagacggcaccctgatctggggcgcacgcgaggccatgacaccgaaggaactccccaagcggctcctcgtcatcggttcgggcgctattggcatcgagttcgcatccttctaccgaaacatgggcagcgaggtcacgattgccgaagtggcggaccggatcctgattgccgaagatccggaaatcagcgccgctgcccgcaaggcttttgagaagcaggggatgaagatcatcaccagcgcgaaggtcggaccgctgaacaagggtgagaacgaggtctccacgacgatcgagagcccgaccgggaaggtcgatctgacggtggatcgcgttatctgcgcggttggcattgtcggcaatgtcgaggatctggggcttgagggcaccaaggttcaggtcgagcggacccatatcgtgacggacggcttctgccgcaccggcgaaccgggcatctatgcgatcggcgacgttgcgggtgctccgtggctggcacacaaggcgagccatgaaggcatcctgtgcgtcgagaagattgctggccgttcgccgcagccgctccatccgctgaatatcccgggctgtacctattcgcgtccgcagatcgcctctgtcgggcttagcgaagagaaggccattgctgccggccacaaggtcaaggtcggccgcttcccgttcatcgccaacggcaaggccgtcgcgatgggtgagacggacggcatggtcaaaaccgtgttcgatgccacgagcggcgaacttcttggcgcccacatgatcggcgcggaagtcacggaaatgatccaaggctacgtcatcacccgcacaggcgaactgaccgaagcggagctggtcgagacggttttcccgcatccgaccatttccgaaaccatgcatgaggcaactctggcggccttcgacggcccgctgcatatctgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 61 - protein, 381 aa, Artificial
  His-lplLDH-Dzip1
MGSSHHHHHHSQDPSSMPNHQKVVLVGDGAVGSSYAFAMAQQGIAEEFVIVDVVKDRTKGDALDLEDAQAFTAPKKIYSGEYSDCKDADLVVITAGAPQKPGESRLDLVNKNLNILSSIVKPVVDSGFDGIFLVAANPVDILTYATWKFSGFPKDRVIGSGTSLDSSRLRVALGKQFNVDPRSVDAYIMGEHGDSEFAAYSTATIGTRPVRDVAKEQGVSDEDLAKLEDGVRNKAYDIINLKGATFYGIGTALMRISKAILRDENAVLPVGAYMDGQYGLNDIYIGTPAVIGGTGLKQIIESPLSADELKKMQDSAATLKKVLNDGLAELENKKLGGGSGGGSGGGSTVAQLEEKVKTLRAQNYELKSRVQRLREQVAQLA

SEQ ID NO: 62 - protein, 515 aa, Artificial
  goxDp-Dzip2
MCDTFDLIVVGGGPGGYVAALRASQLGMSVALVESTHFGGVCLNWGCIPTKALLRSSEIHHLLHELGTFGLSADNISFDLSKIVGRSRSIARRMGGGIAHLLKKTKVTTFDGRAKLAGRSGEAHQVAITKDGAAVATIKAPHVILATGARGRQLPGLETDGTLIWGAREAMTPKELPKRLLVIGSGAIGIEFASFYRNMGSEVTIAEVADRILIAEDPEISAAARKAFEKQGMKIITSAKVGPLNKGENEVSTTIESPTGKVDLTVDRVICAVGIVGNVEDLGLEGTKVQVERTHIVTDGFCRTGEPGIYAIGDVAGAPWLAHKASHEGILCVEKIAGRSPQPLHPLNIPGCTYSRPQIASVGLSEEKAIAAGHKVKVGRFPFIANGKAVAMGETDGMVKTVFDATSGELLGAHMIGAEVTEMIQGYVITRTGELTEAELVETVFPHPTISETMHEATLAAFDGPLHILEGGGSGGGSGGGSSVDELQAEVDQLQDENYALKTKVAQLRKKVEKL

SEQ ID NO: 63 - DNA, 42 bp, Artificial
  PCR primer
aataatcatatgtcaagcatgccaaatcatcaaaaagttgtg

SEQ ID NO: 64 - DNA, 42 bp, Artificial
  PCR primer
aataatctcgagtttattttctaattcagctaaaccgtcgtt

SEQ ID NO: 65 - DNA, 43 bp, Artificial
  PCR primer
aataatggatccgaaaataggtatcgtaggactcggaagggtt

SEQ ID NO: 66 - DNA, 42 bp, Artificial
  PCR primer
aataataagcttaccgctggtgttctggtgcttgttctcttc

SEQ ID NO: 67 - DNA, 42 bp, Artificial
  PCR primer
aataatcatatgtacgatgctgtgatcataggaggaggaccc

SEQ ID NO: 68 - DNA, 42 bp, Artificial
  PCR primer
aataatctcgagcagatgaatgggttttcctgagaccccttc

SEQ ID NO: 69 - DNA, 7829 bp, Artificial
  plasmid, pETDuet-tmaLDH-Dzip1/tmaDp-Dzip2
ggggaattgtgagcggataacaattcccctctagaaataattttgtttaactttaagaaggagatataccatgggcagcagccatcaccatcatcaccacagccaggatccgaaaataggtatcgtaggactcggaagggttggttccagcacggcttttgcactcctgatgaaaggttttgcgagggaaatggttctgatagacgtcgataagaaaagagcagaaggagacgctctcgatctcattcacggaacacctttcacgagaagagcgaacatctacgctggagactatgcggatctgaaaggatcagatgtggtgatcgtcgctgcgggagtacctcagaaaccaggagagacgaggcttcagcttcttgggagaaatgcaagagtgatgaaagaaatagcgcgaaacgtctccaaatacgctcctgattcgattgtcatcgtggtcacgaatcccgtcgatgtcctcacgtatttcttcctcaaagagtccggaatggaccccaggaaggtgttcggttccggaacggttctcgacacagcaaggctcagaactttgatagcgcaacactgcggcttttctccaagaagtgtgcacgtgtacgtgataggagaacatggagattcagaggttcctgtgtggagcggtgccatgataggaggtatccccctacagaacatgtgtcagatttgtcagaagtgcgattcgaagatactggaaaacttcgctgaaaaaaccaaaagagccgcgtacgagatcatagagagaaagggagccacccattacgccatagctctcgccgtggccgacatagtggagagcatcttcttcgacgaaaagagagtgcttacgctttccgtttatctcgaagattacctcggtgtaaaagatctctgtatcagtgttcctgttacccttggaaagcacggagtggaaagaatcctcgaactgaacctgaacgaagaagaactggaagccttcagaaagtcagcttccatattgaaaaacgccatcaacgaaataacggccgaagagaacaagcaccagaacaccagcggtaagcttggcggcggcagcggcggcggcagcggcggcggcagcaccgtggcgcagctggaagaaaaagtgaaaaccctgcgtgcgcagaactatgaactgaaaagccgtgtgcagcgtctgcgtgaacaggtggcgcagctggcgtaacttaagtcgaacagaaagtaatcgtattgtacacggccgcataatcgaaattaatacgactcactataggggaattgtgagcggataacaattccccatcttagtatattagttaagtataagaaggagatatacatatgtacgatgctgtgatcataggaggaggacccggaggttacgtctgcgctataaaactcgcacagcttggaaagaaggtcgcacttgtcgaaaaagatgccctcgggggaacctgtaccaacaggggatgcattccaacgaaggcgatgctcacagtctcccatctcatggatgaaatgaaagagaaagcgtcaaagtacggcttgaaagtctcaggagtcgaatacgacgtcgccgccataatgaaacacgttcaaaagagtgtgatgatgtcaagaaagggaatagagtacctgctgaaaaagaatggggttgaagtcttcaaaggaacggccgtcgtggaaaacaagaacacggttgttgtccaggaaacgggagagaaactggaagcaaagaatcttgttctcgcacacggatccgttccctccgtgttttctcccttcgatatcgacggagtctggacgagtgatgacgtgttcaatttgaaagaatttccaaagtcgcttgtgatagtgggcggtggtgtcatcggagttgaattcgcgacgtttttcggttctttcggtgtggatgtgacgatcgttgagatcgcagaacacatcctcccctatgaagactcagatgtggcagaagaagtgaaaaaggccctcaaaaggaagggtgttaagatcctggaaaaaacaaaaatttcctccctgagcaaagtcgatgacggctttgaagtcgcactcgaaaatggagaaaccctgaaggcagaaaaggtcctgctcgccgcaggaagaaagcccaacataccggaggacgtgaaagctctcggagtgaagatagaaaaaggagtcgtcactgatagcaggatgagaacgaacgtggaaaacgtttacgcgataggagatatcagaagtggaatcatgctcgctcatgtggccatgtacgagggaatagtcgctgcaaagaacatcgcgggagaagaggaagaaatggattactcggccgttccgagtatcatcttctcctctccggaagtggcatctgtcggtgtgagagaaaaagacgtgaaccccgaagaagttgtgatctccaagttcccagtctccgcaaacggaagggcaagaacgatgcttgaaaacatagggtttgccaaggtcatagcggacaagaaagacggaacggttctcggcatgagcatcgtttcaccatcggctaccgatatgatcatggaaggtgtcatagccgtgaagttcaggatgaaagcggaagatctggagaaagccattcacccgcatccgactttgacagaaaccatactcggggctcttgaaggggtctcaggaaaacccattcatctgctcgagggcggcggcagcggcggcggcagcggcggcggcagcagcgtggatgaactgcaggcggaagtggatcagctgcaggatgaaaactatgcgctgaaaaccaaagtggcgcagctgcgtaaaaaagtggaaaaactgtaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 70 - DNA, 44 bp, Artificial
  PCR fragment
aataatccatggcgataggtatcgtaggactcggaagggttggt

SEQ ID NO: 71 - DNA, 42 bp, Artificial
  PCR primer
aataatctcgagtcacagatgaatgggttttcctgagacccc

SEQ ID NO: 72 - DNA, 5962 bp, Artificial
  plasmid, pCDFDuet-tmaLDH-tmaDp
taatacgactcactataggggaattgtgagcggataacaattcccctgtagaaataattttgtttaactttaataaggagatataccatggcgataggtatcgtaggactcggaagggttggttccagcacggcttttgcactcctgatgaaaggttttgcgagggaaatggttctgatagacgtcgataagaaaagagcagaaggagacgctctcgatctcattcacggaacacctttcacgagaagagcgaacatctacgctggagactatgcggatctgaaaggatcagatgtggtgatcgtcgctgcgggagtacctcagaaaccaggagagacgaggcttcagcttcttgggagaaatgcaagagtgatgaaagaaatagcgcgaaacgtctccaaatacgctcctgattcgattgtcatcgtggtcacgaatcccgtcgatgtcctcacgtatttcttcctcaaagagtccggaatggaccccaggaaggtgttcggttccggaacggttctcgacacagcaaggctcagaactttgatagcgcaacactgcggcttttctccaagaagtgtgcacgtgtacgtgataggagaacatggagattcagaggttcctgtgtggagcggtgccatgataggaggtatccccctacagaacatgtgtcagatttgtcagaagtgcgattcgaagatactggaaaacttcgctgaaaaaaccaaaagagccgcgtacgagatcatagagagaaagggagccacccattacgccatagctctcgccgtggccgacatagtggagagcatcttcttcgacgaaaagagagtgcttacgctttccgtttatctcgaagattacctcggtgtaaaagatctctgtatcagtgttcctgttacccttggaaagcacggagtggaaagaatcctcgaactgaacctgaacgaagaagaactggaagccttcagaaagtcagcttccatattgaaaaacgccatcaacgaaataacggccgaagagaacaagcaccagaacaccagcggtaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgtacgatgctgtgatcataggaggaggacccggaggttacgtctgcgctataaaactcgcacagcttggaaagaaggtcgcacttgtcgaaaaagatgccctcgggggaacctgtaccaacaggggatgcattccaacgaaggcgatgctcacagtctcccatctcatggatgaaatgaaagagaaagcgtcaaagtacggcttgaaagtctcaggagtcgaatacgacgtcgccgccataatgaaacacgttcaaaagagtgtgatgatgtcaagaaagggaatagagtacctgctgaaaaagaatggggttgaagtcttcaaaggaacggccgtcgtggaaaacaagaacacggttgttgtccaggaaacgggagagaaactggaagcaaagaatcttgttctcgcacacggatccgttccctccgtgttttctcccttcgatatcgacggagtctggacgagtgatgacgtgttcaatttgaaagaatttccaaagtcgcttgtgatagtgggcggtggtgtcatcggagttgaattcgcgacgtttttcggttctttcggtgtggatgtgacgatcgttgagatcgcagaacacatcctcccctatgaagactcagatgtggcagaagaagtgaaaaaggccctcaaaaggaagggtgttaagatcctggaaaaaacaaaaatttcctccctgagcaaagtcgatgacggctttgaagtcgcactcgaaaatggagaaacccttaaggcagaaaaggtcctgctcgccgcaggaagaaagcccaacataccggaggacgtgaaagctctcggagtgaagatagaaaaaggagtcgtcactgatagcaggatgagaacgaacgtggaaaacgtttacgcgataggagatatcagaagtggaatcatgctcgctcatgtggccatgtacgagggaatagtcgctgcaaagaacatcgcgggagaagaggaagaaatggattactcggccgttccgagtatcatcttctcctctccggaagtggcatctgtcggtgtgagagaaaaagacgtgaaccccgaagaagttgtgatctccaagttcccagtctccgcaaacggaagggcaagaacgatgcttgaaaacatagggtttgccaaggtcatagcggacaagaaagacggaacggttctcggcatgagcatcgtttcaccatcggctaccgatatgatcatggaaggtgtcatagccgtgaagttcaggatgaaagcggaagatctggagaaagccattcacccgcatccgactttgacagaaaccatactcggggctcttgaaggggtctcaggaaaacccattcatctgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 73 - protein, 380 aa, Artificial
  His-tmaLDH-Dzip1
MGSSHHHHHHSQDPKIGIVGLGRVGSSTAFALLMKGFAREMVLIDVDKKRAEGDALDLIHGTPFTRRANIYAGDYADLKGSDVVIVAAGVPQKPGETRLQLLGRNARVMKEIARNVSKYAPDSIVIVVTNPVDVLTYFFLKESGMDPRKVFGSGTVLDTARLRTLIAQHCGFSPRSVHVYVIGEHGDSEVPVWSGAMIGGIPLQNMCQICQKCDSKILENFAEKTKRAAYEIIERKGATHYAIALAVADIVESIFFDEKRVLTLSVYLEDYLGVKDLCISVPVTLGKHGVERILELNLNEEELEAFRKSASILKNAINEITAEENKHQNTSGKLGGGSGGGSGGGSTVAQLEEKVKTLRAQNYELKSRVQRLREQVAQLA

SEQ ID NO: 74 - protein, 496 aa, Artificial
  tmaDp-Dzip2
MYDAVIIGGGPGGYVCAIKLAQLGKKVALVEKDALGGTCTNRGCIPTKAMLTVSHLMDEMKEKASKYGLKVSGVEYDVAAIMKHVQKSVMMSRKGIEYLLKKNGVEVFKGTAVVENKNTVVVQETGEKLEAKNLVLAHGSVPSVFSPFDIDGVWTSDDVFNLKEFPKSLVIVGGGVIGVEFATFFGSFGVDVTIVEIAEHILPYEDSDVAEEVKKALKRKGVKILEKTKISSLSKVDDGFEVALENGETLKAEKVLLAAGRKPNIPEDVKALGVKIEKGVVTDSRMRTNVENVYAIGDIRSGIMLAHVAMYEGIVAAKNIAGEEEEMDYSAVPSIIFSSPEVASVGVREKDVNPEEVVISKFPVSANGRARTMLENIGFAKVIADKKDGTVLGMSIVSPSATDMIMEGVIAVKFRMKAEDLEKAIHPHPTLTETILGALEGVSGKPIHLLEGGGSGGGSGGGSSVDELQAEVDQLQDENYALKTKVAQLRKKVEKL

SEQ ID NO: 75 - DNA, 42 bp, Artificial
  PCR primer
aataatcatatgaaaataggtatcgtaggactcggaagggtt

SEQ ID NO: 76 - DNA, 42 bp, Artificial
  PCR primer
aataatctcgagaccgctggtgttctggtgcttgttctcttc

SEQ ID NO: 77 - DNA, 43 bp, Artificial
  PCR primer
aataatggatccgtcagacctgaaaaccgccgctctcgaatat

SEQ ID NO: 78 - DNA, 42 bp, Artificial
  PCR primer
aataataagcttgccgttgaacacttcatccacgctggtcag

SEQ ID NO: 79 - DNA, 8195 bp, Artificial
  plasmid, pETDuet-ppuMDH-Dzip1/goxDp-Dzip2
ggggaattgtgagcggataacaattcccctctagaaataattttgtttaactttaagaaggagatataccatgggcagcagccatcaccatcatcaccacagccaggatccgtcagacctgaaaaccgccgctctcgaatatcacgctcaacctcgtccggggaaactgagcgttgaactctccaagcccactgccaccgcccgtgacctcgccctggcctacagcccaggtgttgcagagccggtgcgcgaaattggccgtgatccagagctggcttacaaatacaccggcaaaggcaacctggttgcggtgatttccgatggcactgccatcctcggtctgggtgacctcggcccactggcttccaagccggtcatggaaggcaagggtgttctgttcaagcgtttcgccggtatcgatgtgttcgacatcgaagtcgagtcggaaagcccgcaagcgttcatcgacaccgttcgccgcatttcgatcaccttcggtggcatcaaccttgaagacatcaaggcacctgagtgctttgaaatcgaacgtaccctgatcgaacagtgcgacatcccggtgttccacgatgaccagcacggtactgccatcgtgactgccgctggcatgatcaacgccctggaaatcgccggcaagaaactggaagaagccaagatcgtctgcctgggcgccggcgctgctgccatttcctgcatgaagctgctggtaagcatgggtgcgaaggtcgagaacatcttcatgatcgaccgtagtggcgtgatccacgctggccgtgacgacctgaaccagtacaaggcgcagttcgcccacgcgaccgacaagcgcaccctggccgacgcccttgacggtgccgacgtgttcgtaggtctgtctggcccgaacctgctgagcccggaaggcctgaagtcgatggctgccaacccgatcgtgttcgcctgctcgaacccggacccggaaatctcgccagagctggcgcatgccactcgcaacgacgtgatcatggctaccggtcgttccgactacccgaaccaggtcaacaacgtactgggcttcccgttcatcttccgtggtgctctggacgttcgcgccaagcgtatcaacgaagaaatgaagatcgctgccgctatcgccctgaaagacctggccaagctgccagtgccgaaggaagtttgcgaagcctatggcgttgaaggcctggagttcggtcgtgagtacatcattccgaagccgctggatgcacgcttgatcaccgtcgtttccgatgctgtggccaaggctgccatcgaatccggcgtggctaccctgccttatccgaagcattacccgctgaccagcgtggatgaagtgttcaacggcaagcttggcggcggcagcggcggcggcagcggcggcggcagcaccgtggcgcagctggaagaaaaagtgaaaaccctgcgtgcgcagaactatgaactgaaaagccgtgtgcagcgtctgcgtgaacaggtggcgcagctggcgtaacttaagtcgaacagaaagtaatcgtattgtacacggccgcataatcgaaattaatacgactcactataggggaattgtgagcggataacaattccccatcttagtatattagttaagtataagaaggagatatacatatgtgcgatacgttcgatctgattgttgttggtggcggccccggcggctatgtggcggcgctgcgggccagccagcttggcatgagcgtggcgctcgtggaaagcacgcatttcggcggcgtgtgcctcaactggggctgcattccgaccaaggcgcttctacggtcttcggaaattcaccacctgcttcacgaactggggacgttcggactttccgccgacaatatctcgttcgacctgtccaagatcgttggccgttcacgcagcatcgcacgccggatgggtggcggcattgcacatctgctgaagaagaccaaggtcacgacctttgacggtcgtgcaaagctggccggccgttcgggtgaggcgcatcaggttgccatcaccaaggatggcgcagcagttgccaccatcaaggcgccgcatgtcatccttgcaacgggtgcgcgcggtcgtcagcttccgggtcttgagacagacggcaccctgatctggggcgcacgcgaggccatgacaccgaaggaactccccaagcggctcctcgtcatcggttcgggcgctattggcatcgagttcgcatccttctaccgaaacatgggcagcgaggtcacgattgccgaagtggcggaccggatcctgattgccgaagatccggaaatcagcgccgctgcccgcaaggcttttgagaagcaggggatgaagatcatcaccagcgcgaaggtcggaccgctgaacaagggtgagaacgaggtctccacgacgatcgagagcccgaccgggaaggtcgatctgacggtggatcgcgttatctgcgcggttggcattgtcggcaatgtcgaggatctggggcttgagggcaccaaggttcaggtcgagcggacccatatcgtgacggacggcttctgccgcaccggcgaaccgggcatctatgcgatcggcgacgttgcgggtgctccgtggctggcacacaaggcgagccatgaaggcatcctgtgcgtcgagaagattgctggccgttcgccgcagccgctccatccgctgaatatcccgggctgtacctattcgcgtccgcagatcgcctctgtcgggcttagcgaagagaaggccattgctgccggccacaaggtcaaggtcggccgcttcccgttcatcgccaacggcaaggccgtcgcgatgggtgagacggacggcatggtcaaaaccgtgttcgatgccacgagcggcgaacttcttggcgcccacatgatcggcgcggaagtcacggaaatgatccaaggctacgtcatcacccgcacaggcgaactgaccgaagcggagctggtcgagacggttttcccgcatccgaccatttccgaaaccatgcatgaggcaactctggcggccttcgacggcccgctgcatatcctcgagggcggcggcagcggcggcggcagcggcggcggcagcagcgtggatgaactgcaggcggaagtggatcagctgcaggatgaaaactatgcgctgaaaaccaaagtggcgcagctgcgtaaaaaagtggaaaaactgtaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 80 - DNA, 44 bp, Artificial
  PCR primer
aataatccatggcggacctgaaaaccgccgctctcgaatatcac

SEQ ID NO: 81 - DNA, 6328 bp, Artificial
  plasmid, pCDFDuet-ppuMDH-goxDp
taatacgactcactataggggaattgtgagcggataacaattcccctgtagaaataattttgtttaactttaataaggagatataccatggcggacctgaaaaccgccgctctcgaatatcacgctcaacctcgtccggggaaactgagcgttgaactctccaagcccactgccaccgcccgtgacctcgccctggcctacagcccaggtgttgcagagccggtgcgcgaaattggccgtgatccagagctggcttacaaatacaccggcaaaggcaacctggttgcggtgatttccgatggcactgccatcctcggtctgggtgacctcggcccactggcttccaagccggtcatggaaggcaagggtgttctgttcaagcgtttcgccggtatcgatgtgttcgacatcgaagtcgagtcggaaagcccgcaagcgttcatcgacaccgttcgccgcatttcgatcaccttcggtggcatcaaccttgaagacatcaaggcacctgagtgctttgaaatcgaacgtaccctgatcgaacagtgcgacatcccggtgttccacgatgaccagcacggtactgccatcgtgactgccgctggcatgatcaacgccctggaaatcgccggcaagaaactggaagaagccaagatcgtctgcctgggcgccggcgctgctgccatttcctgcatgaagctgctggtaagcatgggtgcgaaggtcgagaacatcttcatgatcgaccgtagtggcgtgatccacgctggccgtgacgacctgaaccagtacaaggcgcagttcgcccacgcgaccgacaagcgcaccctggccgacgcccttgacggtgccgacgtgttcgtaggtctgtctggcccgaacctgctgagcccggaaggcctgaagtcgatggctgccaacccgatcgtgttcgcctgctcgaacccggacccggaaatctcgccagagctggcgcatgccactcgcaacgacgtgatcatggctaccggtcgttccgactacccgaaccaggtcaacaacgtactgggcttcccgttcatcttccgtggtgctctggacgttcgcgccaagcgtatcaacgaagaaatgaagatcgctgccgctatcgccctgaaagacctggccaagctgccagtgccgaaggaagtttgcgaagcctatggcgttgaaggcctggagttcggtcgtgagtacatcattccgaagccgctggatgcacgcttgatcaccgtcgtttccgatgctgtggccaaggctgccatcgaatccggcgtggctaccctgccttatccgaagcattacccgctgaccagcgtggatgaagtgttcaacggcaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgtgcgatacgttcgatctgattgttgttggtggcggccccggcggctatgtggcggcgctgcgggccagccagcttggcatgagcgtggcgctcgtggaaagcacgcatttcggcggcgtgtgcctcaactggggctgcattccgaccaaggcgcttctacggtcttcggaaattcaccacctgcttcacgaactggggacgttcggactttccgccgacaatatctcgttcgacctgtccaagatcgttggccgttcacgcagcatcgcacgccggatgggtggcggcattgcacatctgctgaagaagaccaaggtcacgacctttgacggtcgtgcaaagctggccggccgttcgggtgaggcgcatcaggttgccatcaccaaggatggcgcagcagttgccaccatcaaggcgccgcatgtcatccttgcaacgggtgcgcgcggtcgtcagcttccgggtcttgagacagacggcaccctgatctggggcgcacgcgaggccatgacaccgaaggaactccccaagcggctcctcgtcatcggttcgggcgctattggcatcgagttcgcatccttctaccgaaacatgggcagcgaggtcacgattgccgaagtggcggaccggatcctgattgccgaagatccggaaatcagcgccgctgcccgcaaggcttttgagaagcaggggatgaagatcatcaccagcgcgaaggtcggaccgctgaacaagggtgagaacgaggtctccacgacgatcgagagcccgaccgggaaggtcgatctgacggtggatcgcgttatctgcgcggttggcattgtcggcaatgtcgaggatctggggcttgagggcaccaaggttcaggtcgagcggacccatatcgtgacggacggcttctgccgcaccggcgaaccgggcatctatgcgatcggcgacgttgcgggtgctccgtggctggcacacaaggcgagccatgaaggcatcctgtgcgtcgagaagattgctggccgttcgccgcagccgctccatccgctgaatatcccgggctgtacctattcgcgtccgcagatcgcctctgtcgggcttagcgaagagaaggccattgctgccggccacaaggtcaaggtcggccgcttcccgttcatcgccaacggcaaggccgtcgcgatgggtgagacggacggcatggtcaaaaccgtgttcgatgccacgagcggcgaacttcttggcgcccacatgatcggcgcggaagtcacggaaatgatccaaggctacgtcatcacccgcacaggcgaactgaccgaagcggagctggtcgagacggttttcccgcatccgaccatttccgaaaccatgcatgaggcaactctggcggccttcgacggcccgctgcatatctgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 82 - protein, 483 aa, Artificial
  His-ppuMDH-Dzip1
MGSSHHHHHHSQDPSDLKTAALEYHAQPRPGKLSVELSKPTATARDLALAYSPGVAEPVREIGRDPELAYKYTGKGNLVAVISDGTAILGLGDLGPLASKPVMEGKGVLFKRFAGIDVFDIEVESESPQAFIDTVRRISITFGGINLEDIKAPECFEIERTLIEQCDIPVFHDDQHGTAIVTAAGMINALEIAGKKLEEAKIVCLGAGAAAISCMKLLVSMGAKVENIFMIDRSGVIHAGRDDLNQYKAQFAHATDKRTLADALDGADVFVGLSGPNLLSPEGLKSMAANPIVFACSNPDPEISPELAHATRNDVIMATGRSDYPNQVNNVLGFPFIFRGALDVRAKRINEEMKIAAAIALKDLAKLPVPKEVCEAYGVEGLEFGREYIIPKPLDARLITVVSDAVAKAAIESGVATLPYPKHYPLTSVDEVFNGKLGGGSGGGSGGGSTVAQLEEKVKTLRAQNYELKSRVQRLREQVAQLA

SEQ ID NO: 83 - DNA, 42 bp, Artificial
  PCR primer
aataatcatatgtcagacctgaaaaccgccgctctcgaatat

SEQ ID NO: 84 - DNA, 42 bp, Artificial
  PCR primer
aataatctcgaggccgttgaacacttcatccacgctggtcag

SEQ ID NO: 85 - DNA, 43 bp, Artificial
  PCR primer
aataatggatccggctaggatcactgaggaacaaagaaggaaa

SEQ ID NO: 86 - DNA, 42 bp, Artificial
  PCR primer
aataataagcttagtaatgtactgtttccttctttcatttaa

SEQ ID NO: 87 - DNA, 8177 bp, Artificial
  plasmid, pETDuet-pfuMDH-Dzip1/tmaDp-Dzip2
taatacgactcactatacgggaattgtgagcggataacaattcccctctagaaataattttgtttaactttaagaaggagatataccatgggcagcagccatcaccatcatcaccacagccaggatccggctaggatcactgaggaacaaagaaggaaacttgctcaagatgcgttggagtatcacagaaataatttcccagggaatggaaaaattgaagtgataccaaaggttcctcttagagatttctatgatttgagcttagcatataccccaggagtagcggagccatgtaaagtaattgcaaagaagggaaactatgaagattacactataattccaaatacagttgccgtcgttacagacggatcggcaatcttgggattaggaaacataggtgttctggcaggaatgccagttatggaaggtaaatgtgttctctttaaagctctagctggaatagatgcgtttcctatcttaattgacacacaggatgtcgagaaaatcgtggaaacaataaaacttatatcaaagggatttggcgggataaacctagaagatatctccgctccaagatgcttcgaaattgaaatgaggttgaagagagagcttgatattcccgtatttcacgatgatcaacatggaactgctgtagtaactttagcgggtctaattaacgcattaaagttagtgggcaaaaaattcaacgagataaaagttgctataagtggagctggagctgctggaatcgcaatagcaaagttgctccatcatgttggggttaaagaaatattgcttgttgatagagctggaataatctatgaagggagaaaagaaaacatgaatccttacaaagaggaagtcgcaaggttcaacattcatggggttcaaggagatttggcaaaagctatggagggtgctgatgtttttattggagtaagcgtaggaggaatagtaaaaccctggatgataaagaaaatggccgatgatcccatagtgtttgcaatggcgaatccaattccagaaattatgccagaagatgcgaaaaaggctggggcaaaaatagtggccacggggaggagtgactatccaaaccaaataaacaatgtcttgggctttcctgggatatttaggggggcactagatgttagggctaccgatataactttaaatatgaacattgcagcggctgaggcaatagcatctgttgtgagcgatgaagaacttagtgaggactacataataccctctccactacatcccgacgtttatccaaaagaagccagagcagttgcagaacaagcaattagggatggtgtagctagaagaaagctatctggggagtgggtagaagagcatactaggaagctccgtgaattttatgccgcactcattgagcccttaaatgaaagaaggaaacagtacattactaagcttggcggcggcagcggcggcggcagcggcggcggcagcaccgtggcgcagctggaagaaaaagtgaaaaccctgcgtgcgcagaactatgaactgaaaagccgtgtgcagcgtctgcgtgaacaggtggcgcagctggcgtaacttaagtcgaacagaaagtaatcgtattgtacacggccgcataatcgaaattaatacgactcactataggggaattgtgagcggataacaattccccatcttagtatattagttaagtataagaaggagatatacatatgtacgatgctgtgatcataggaggaggacccggaggttacgtctgcgctataaaactcgcacagcttggaaagaaggtcgcacttgtcgaaaaagatgccctcgggggaacctgtaccaacaggggatgcattccaacgaaggcgatgctcacagtctcccatctcatggatgaaatgaaagagaaagcgtcaaagtacggcttgaaagtctcaggagtcgaatacgacgtcgccgccataatgaaacacgttcaaaagagtgtgatgatgtcaagaaagggaatagagtacctgctgaaaaagaatggggttgaagtcttcaaaggaacggccgtcgtggaaaacaagaacacggttgttgtccaggaaacgggagagaaactggaagcaaagaatcttgttctcgcacacggatccgttccctccgtgttttctcccttcgatatcgacggagtctggacgagtgatgacgtgttcaatttgaaagaatttccaaagtcgcttgtgatagtgggcggtggtgtcatcggagttgaattcgcgacgtttttcggttctttcggtgtggatgtgacgatcgttgagatcgcagaacacatcctcccctatgaagactcagatgtggcagaagaagtgaaaaaggccctcaaaaggaagggtgttaagatcctggaaaaaacaaaaatttcctccctgagcaaagtcgatgacggctttgaagtcgcactcgaaaatggagaaaccctgaaggcagaaaaggtcctgctcgccgcaggaagaaagcccaacataccggaggacgtgaaagctctcggagtgaagatagaaaaaggagtcgtcactgatagcaggatgagaacgaacgtggaaaacgtttacgcgataggagatatcagaagtggaatcatgctcgctcatgtggccatgtacgagggaatagtcgctgcaaagaacatcgcgggagaagaggaagaaatggattactcggccgttccgagtatcatcttctcctctccggaagtggcatctgtcggtgtgagagaaaaagacgtgaaccccgaagaagttgtgatctccaagttcccagtctccgcaaacggaagggcaagaacgatgcttgaaaacatagggtttgccaaggtcatagcggacaagaaagacggaacggttctcggcatgagcatcgtttcaccatcggctaccgatatgatcatggaaggtgtcatagccgtgaagttcaggatgaaagcggaagatctggagaaagccattcacccgcatccgactttgacagaaaccatactcggggctcttgaaggggtctcaggaaaacccattcatctgctcgagggcggcggcagcggcggcggcagcggcggcggcagcagcgtggatgaactgcaggcggaagtggatcagctgcaggatgaaaactatgcgctgaaaaccaaagtggcgcagctgcgtaaaaaagtggaaaaactgtaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaat

SEQ ID NO: 88 - DNA, 44 bp, Artificial
  PCR primer
aataatccatggcgaggatcactgaggaacaaagaaggaaactt

SEQ ID NO: 89 - DNA, 6310 bp, Artificial
  plasmid, pCDFDuet-pfuMDH-tmaDp
taatacgactcactataggggaattgtgagcggataacaattcccctgtagaaataattttgtttaactttaataaggagatataccatggcgaggatcactgaggaacaaagaaggaaacttgctcaagatgcgttggagtatcacagaaataatttcccagggaatggaaaaattgaagtgataccaaaggttcctcttagagatttctatgatttgagcttagcatataccccaggagtagcggagccatgtaaagtaattgcaaagaagggaaactatgaagattacactataattccaaatacagttgccgtcgttacagacggatcggcaatcttgggattaggaaacataggtgttctggcaggaatgccagttatggaaggtaaatgtgttctctttaaagctctagctggaatagatgcgtttcctatcttaattgacacacaggatgtcgagaaaatcgtggaaacaataaaacttatatcaaagggatttggcgggataaacctagaagatatctccgctccaagatgcttcgaaattgaaatgaggttgaagagagagcttgatattcccgtatttcacgatgatcaacatggaactgctgtagtaactttagcgggtctaattaacgcattaaagttagtgggcaaaaaattcaacgagataaaagttgctataagtggagctggagctgctggaatcgcaatagcaaagttgctccatcatgttggggttaaagaaatattgcttgttgatagagctggaataatctatgaagggagaaaagaaaacatgaatccttacaaagaggaagtcgcaaggttcaacattcatggggttcaaggagatttggcaaaagctatggagggtgctgatgtttttattggagtaagcgtaggaggaatagtaaaaccctggatgataaagaaaatggccgatgatcccatagtgtttgcaatggcgaatccaattccagaaattatgccagaagatgcgaaaaaggctggggcaaaaatagtggccacggggaggagtgactatccaaaccaaataaacaatgtcttgggctttcctgggatatttaggggggcactagatgttagggctaccgatataactttaaatatgaacattgcagcggctgaggcaatagcatctgttgtgagcgatgaagaacttagtgaggactacataataccctctccactacatcccgacgtttatccaaaagaagccagagcagttgcagaacaagcaattagggatggtgtagctagaagaaagctatctggggagtgggtagaagagcatactaggaagctccgtgaattttatgccgcactcattgagcccttaaatgaaagaaggaaacagtacattactaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgtacgatgctgtgatcataggaggaggacccggaggttacgtctgcgctataaaactcgcacagcttggaaagaaggtcgcacttgtcgaaaaagatgccctcgggggaacctgtaccaacaggggatgcattccaacgaaggcgatgctcacagtctcccatctcatggatgaaatgaaagagaaagcgtcaaagtacggcttgaaagtctcaggagtcgaatacgacgtcgccgccataatgaaacacgttcaaaagagtgtgatgatgtcaagaaagggaatagagtacctgctgaaaaagaatggggttgaagtcttcaaaggaacggccgtcgtggaaaacaagaacacggttgttgtccaggaaacgggagagaaactggaagcaaagaatcttgttctcgcacacggatccgttccctccgtgttttctcccttcgatatcgacggagtctggacgagtgatgacgtgttcaatttgaaagaatttccaaagtcgcttgtgatagtgggcggtggtgtcatcggagttgaattcgcgacgtttttcggttctttcggtgtggatgtgacgatcgttgagatcgcagaacacatcctcccctatgaagactcagatgtggcagaagaagtgaaaaaggccctcaaaaggaagggtgttaagatcctggaaaaaacaaaaatttcctccctgagcaaagtcgatgacggctttgaagtcgcactcgaaaatggagaaacccttaaggcagaaaaggtcctgctcgccgcaggaagaaagcccaacataccggaggacgtgaaagctctcggagtgaagatagaaaaaggagtcgtcactgatagcaggatgagaacgaacgtggaaaacgtttacgcgataggagatatcagaagtggaatcatgctcgctcatgtggccatgtacgagggaatagtcgctgcaaagaacatcgcgggagaagaggaagaaatggattactcggccgttccgagtatcatcttctcctctccggaagtggcatctgtcggtgtgagagaaaaagacgtgaaccccgaagaagttgtgatctccaagttcccagtctccgcaaacggaagggcaagaacgatgcttgaaaacatagggtttgccaaggtcatagcggacaagaaagacggaacggttctcggcatgagcatcgtttcaccatcggctaccgatatgatcatggaaggtgtcatagccgtgaagttcaggatgaaagcggaagatctggagaaagccattcacccgcatccgactttgacagaaaccatactcggggctcttgaaggggtctcaggaaaacccattcatctgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 90 - protein, 496 aa, Artificial
  His-pfuMDH-Dzip1
MGSSHHHHHHSQDPARITEEQRRKLAQDALEYHRNNFPGNGKIEVIPKVPLRDFYDLSLAYTPGVAEPCKVIAKKGNYEDYTIIPNTVAVVTDGSAILGLGNIGVLAGMPVMEGKCVLFKALAGIDAFPILIDTQDVEKIVETIKLISKGFGGINLEDISAPRCFEIEMRLKRELDIPVFHDDQHGTAVVTLAGLINALKLVGKKFNEIKVAISGAGAAGIAIAKLLHHVGVKEILLVDRAGIIYEGRKENMNPYKEEVARFNIHGVQGDLAKAMEGADVFIGVSVGGIVKPWMIKKMADDPIVFAMANPIPEIMPEDAKKAGAKIVATGRSDYPNQINNVLGFPGIFRGALDVRATDITLNMNIAAAEAIASVVSDEELSEDYIIPSPLHPDVYPKEARAVAEQAIRDGVARRKLSGEWVEEHTRKLREFYAALIEPLNERRKQYITKLGGGSGGGSGGGSTVAQLEEKVKTLRAQNYELKSRVQRLREQVAQLA

SEQ ID NO: 91 - DNA, 43 bp, Artificial
  PCR primer
aataatcatatggctaggatcactgaggaacaaagaaggaaat

SEQ ID NO: 92 - DNA, 42 bp, Artificial
  PCR primer
aataatctcgagagtaatgtactgtttccttctttcatttaa

SEQ ID NO: 93 - DNA, 43 bp, Artificial
  PCR primer
aataatggatccgtcttcgcaatcgcagtccccgtccgtcgcg

SEQ ID NO: 94 - DNA, 42 bp, Artificial
  PCR primer
aataataagcttggggtacagaccgcgcatttcgcgcgccat

SEQ ID NO: 95 - DNA, 8231 bp, Artificial
  plasmid, pETDuet-bmaEDH-Dzip1/goxDp-Dzip2
ggggaattgtgagcggataacaattcccctctagaaataattttgtttaactttaagaaggagatataccatgggcagcagccatcaccatcatcaccacagccaggatccgtcttcgcaatcgcagtccccgtccgtcgcgcagtccattccgtcgtacctgcacgcggacgatctcggcccctggggcaactatttgcagcaagtcgatcgcgtcgcgccgtatctcggctcgctgtcgcgctggatcgagacgctcaagcgcccgaaacgcatcctgatcgtcgacgtgccgatcgagctggacaacggcaccgtcgcgcacttcgagggctaccgcgtgcagcacaacgtgtcgcgcggcccgggcaagggcggcgtgcgctaccaccaggacgtgacgctgtccgaggtgatggcgctgtccgcatggatgtcggtgaagaacgcggccgtgaacgtgccgtacggcggcgcgaagggcggtatccgcgtcgatccgcgcaagctctcgcgcggcgagctggagcgcgtgacgcgccgctacacgagcgaaatcggcatcatcatcggcccgaacaccgatatccccgcgcctgacgtcaacacgaacgagcagatcatggcgtggatgatggacacgtactcgatgaaccagggccagacggccacgggcgtcgtgaccggcaagccgatctcgctcggtggctcgctcggccgcaaggaagcgacgggccgcggcgtgttcgtcgtcggctgcgaggcggcgaagaagaaggggctggagatcgaaggcgcgcgcatcgcggtccagggcttcggcaatgtcggcgggatcgccgcgaagctgttccaggaagcgggcgcgaaggtgatcgcggtgcaggatcacacgggcacgattcaccagccggcgggcgtcgatacggtcaagctgctggagcacgtcggccgcacgggcggcgtcgcgggcttcgaaggcgcggaaccgatgccgaacgacgagttctggaccgtcgaaaccgatatcctgattccggccgcgctggaaaaccagatcaccgagaagaacgcgtcgaagatccgcacgaagatcatcgtcgagggcgcgaacggcccgacgacgacggccgcggacgatatcctgagcgcgaacggcgtgctcgtgatccccgacgtgatcgcgaacgcgggcggcgtgaccgtgtcgtatttcgaatgggtgcaggatttctggagcttcttctggacggaggacgagatcaatcaccgtctggagcgcgtgatgcgcgaggcgttcgccggcgtatgggcggtggccgaagagcacaacgtgtcggtgcgcacggcggcgttcatcgtcgcgtgcaagcgcatcctgatggcgcgcgaaatgcgcggtctgtaccccaagcttggcggcggcagcggcggcggcagcggcggcggcagcaccgtggcgcagctggaagaaaaagtgaaaaccctgcgtgcgcagaactatgaactgaaaagccgtgtgcagcgtctgcgtgaacaggtggcgcagctggcgtaacttaagtcgaacagaaagtaatcgtattgtacacggccgcataatcgaaattaatacgactcactataggggaattgtgagcggataacaattccccatcttagtatattagttaagtataagaaggagatatacatatgtgcgatacgttcgatctgattgttgttggtggcggccccggcggctatgtggcggcgctgcgggccagccagcttggcatgagcgtggcgctcgtggaaagcacgcatttcggcggcgtgtgcctcaactggggctgcattccgaccaaggcgcttctacggtcttcggaaattcaccacctgcttcacgaactggggacgttcggactttccgccgacaatatctcgttcgacctgtccaagatcgttggccgttcacgcagcatcgcacgccggatgggtggcggcattgcacatctgctgaagaagaccaaggtcacgacctttgacggtcgtgcaaagctggccggccgttcgggtgaggcgcatcaggttgccatcaccaaggatggcgcagcagttgccaccatcaaggcgccgcatgtcatccttgcaacgggtgcgcgcggtcgtcagcttccgggtcttgagacagacggcaccctgatctggggcgcacgcgaggccatgacaccgaaggaactccccaagcggctcctcgtcatcggttcgggcgctattggcatcgagttcgcatccttctaccgaaacatgggcagcgaggtcacgattgccgaagtggcggaccggatcctgattgccgaagatccggaaatcagcgccgctgcccgcaaggcttttgagaagcaggggatgaagatcatcaccagcgcgaaggtcggaccgctgaacaagggtgagaacgaggtctccacgacgatcgagagcccgaccgggaaggtcgatctgacggtggatcgcgttatctgcgcggttggcattgtcggcaatgtcgaggatctggggcttgagggcaccaaggttcaggtcgagcggacccatatcgtgacggacggcttctgccgcaccggcgaaccgggcatctatgcgatcggcgacgttgcgggtgctccgtggctggcacacaaggcgagccatgaaggcatcctgtgcgtcgagaagattgctggccgttcgccgcagccgctccatccgctgaatatcccgggctgtacctattcgcgtccgcagatcgcctctgtcgggcttagcgaagagaaggccattgctgccggccacaaggtcaaggtcggccgcttcccgttcatcgccaacggcaaggccgtcgcgatgggtgagacggacggcatggtcaaaaccgtgttcgatgccacgagcggcgaacttcttggcgcccacatgatcggcgcggaagtcacggaaatgatccaaggctacgtcatcacccgcacaggcgaactgaccgaagcggagctggtcgagacggttttcccgcatccgaccatttccgaaaccatgcatgaggcaactctggcggccttcgacggcccgctgcatatcctcgagggcggcggcagcggcggcggcagcggcggcggcagcagcgtggatgaactgcaggcggaagtggatcagctgcaggatgaaaactatgcgctgaaaaccaaagtggcgcagctgcgtaaaaaagtggaaaaactgtaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 96 - DNA, 44 bp, Artificial
  PCR primer
aataatccatggcgtcgcaatcgcagtccccgtccgtcgcgcag

SEQ ID NO: 97 - DNA, 6364 bp, Artificial
  plasmid, pCDFDuet-bmaEDH-goxDp
taatacgactcactataggggaattgtgagcggataacaattcccctgtagaaataattttgtttaactttaataaggagatataccatggcgtcgcaatcgcagtccccgtccgtcgcgcagtccattccgtcgtacctgcacgcggacgatctcggcccctggggcaactatttgcagcaagtcgatcgcgtcgcgccgtatctcggctcgctgtcgcgctggatcgagacgctcaagcgcccgaaacgcatcctgatcgtcgacgtgccgatcgagctggacaacggcaccgtcgcgcacttcgagggctaccgcgtgcagcacaacgtgtcgcgcggcccgggcaagggcggcgtgcgctaccaccaggacgtgacgctgtccgaggtgatggcgctgtccgcatggatgtcggtgaagaacgcggccgtgaacgtgccgtacggcggcgcgaagggcggtatccgcgtcgatccgcgcaagctctcgcgcggcgagctggagcgcgtgacgcgccgctacacgagcgaaatcggcatcatcatcggcccgaacaccgatatccccgcgcctgacgtcaacacgaacgagcagatcatggcgtggatgatggacacgtactcgatgaaccagggccagacggccacgggcgtcgtgaccggcaagccgatctcgctcggtggctcgctcggccgcaaggaagcgacgggccgcggcgtgttcgtcgtcggctgcgaggcggcgaagaagaaggggctggagatcgaaggcgcgcgcatcgcggtccagggcttcggcaatgtcggcgggatcgccgcgaagctgttccaggaagcgggcgcgaaggtgatcgcggtgcaggatcacacgggcacgattcaccagccggcgggcgtcgatacggtcaagctgctggagcacgtcggccgcacgggcggcgtcgcgggcttcgaaggcgcggaaccgatgccgaacgacgagttctggaccgtcgaaaccgatatcctgattccggccgcgctggaaaaccagatcaccgagaagaacgcgtcgaagatccgcacgaagatcatcgtcgagggcgcgaacggcccgacgacgacggccgcggacgatatcctgagcgcgaacggcgtgctcgtgatccccgacgtgatcgcgaacgcgggcggcgtgaccgtgtcgtatttcgaatgggtgcaggatttctggagcttcttctggacggaggacgagatcaatcaccgtctggagcgcgtgatgcgcgaggcgttcgccggcgtatgggcggtggccgaagagcacaacgtgtcggtgcgcacggcggcgttcatcgtcgcgtgcaagcgcatcctgatggcgcgcgaaatgcgcggtctgtaccccaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgtgcgatacgttcgatctgattgttgttggtggcggccccggcggctatgtggcggcgctgcgggccagccagcttggcatgagcgtggcgctcgtggaaagcacgcatttcggcggcgtgtgcctcaactggggctgcattccgaccaaggcgcttctacggtcttcggaaattcaccacctgcttcacgaactggggacgttcggactttccgccgacaatatctcgttcgacctgtccaagatcgttggccgttcacgcagcatcgcacgccggatgggtggcggcattgcacatctgctgaagaagaccaaggtcacgacctttgacggtcgtgcaaagctggccggccgttcgggtgaggcgcatcaggttgccatcaccaaggatggcgcagcagttgccaccatcaaggcgccgcatgtcatccttgcaacgggtgcgcgcggtcgtcagcttccgggtcttgagacagacggcaccctgatctggggcgcacgcgaggccatgacaccgaaggaactccccaagcggctcctcgtcatcggttcgggcgctattggcatcgagttcgcatccttctaccgaaacatgggcagcgaggtcacgattgccgaagtggcggaccggatcctgattgccgaagatccggaaatcagcgccgctgcccgcaaggcttttgagaagcaggggatgaagatcatcaccagcgcgaaggtcggaccgctgaacaagggtgagaacgaggtctccacgacgatcgagagcccgaccgggaaggtcgatctgacggtggatcgcgttatctgcgcggttggcattgtcggcaatgtcgaggatctggggcttgagggcaccaaggttcaggtcgagcggacccatatcgtgacggacggcttctgccgcaccggcgaaccgggcatctatgcgatcggcgacgttgcgggtgctccgtggctggcacacaaggcgagccatgaaggcatcctgtgcgtcgagaagattgctggccgttcgccgcagccgctccatccgctgaatatcccgggctgtacctattcgcgtccgcagatcgcctctgtcgggcttagcgaagagaaggccattgctgccggccacaaggtcaaggtcggccgcttcccgttcatcgccaacggcaaggccgtcgcgatgggtgagacggacggcatggtcaaaaccgtgttcgatgccacgagcggcgaacttcttggcgcccacatgatcggcgcggaagtcacggaaatgatccaaggctacgtcatcacccgcacaggcgaactgaccgaagcggagctggtcgagacggttttcccgcatccgaccatttccgaaaccatgcatgaggcaactctggcggccttcgacggcccgctgcatatctgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 98 - protein, 495 aa, Artificial
  His-bmaEDH-Dzip1
MGSSHHHHHHSQDPSSQSQSPSVAQSIPSYLHADDLGPWGNYLQQVDRVAPYLGSLSRWIETLKRPKRILIVDVPIELDNGTVAHFEGYRVQHNVSRGPGKGGVRYHQDVTLSEVMALSAWMSVKNAAVNVPYGGAKGGIRVDPRKLSRGELERVTRRYTSEIGIIIGPNTDIPAPDVNTNEQIMAWMMDTYSMNQGQTATGVVTGKPISLGGSLGRKEATGRGVFVVGCEAAKKKGLEIEGARIAVQGFGNVGGIAAKLFQEAGAKVIAVQDHTGTIHQPAGVDTVKLLEHVGRTGGVAGFEGAEPMPNDEFWTVETDILIPAALENQITEKNASKIRTKIIVEGANGPTTTAADDILSANGVLVIPDVIANAGGVTVSYFEWVQDFWSFFWTEDEINHRLERVMREAFAGVWAVAEEHNVSVRTAAFIVACKRILMAREMRGLYPKLGGGSGGGSGGGSTVAQLEEKVKTLRAQNYELKSRVQRLREQVAQLA

SEQ ID NO: 99 - DNA, 42 bp, Artificial
  PCR primer
aataatcatatgtcttcgcaatcgcagtccccgtccgtcgcg

SEQ ID NO: 100 - DNA, 42 bp, Artificial
  PCR primer
aataatctcgagggggtacagaccgcgcatttcgcgcgccat

SEQ ID NO: 101 - DNA, 43 bp, Artificial
  PCR primer
aataatggatccggttgagcaagacccctatgaaattgttatt

SEQ ID NO: 102 - DNA, 42 bp, Artificial
  PCR primer
aataataagcttgtgcttgacccatccacggtcaagcattgc

SEQ ID NO: 103 - DNA, 8132 bp, Artificial
  plasmid, pETDuet-pfuEDH-Dzip1/tmaDp-Dzip2
ggggaattgtgagcggataacaattcccctctagaaataattttgtttaactttaagaaggagatataccatgggcagcagccatcaccatcatcaccacagccaggatccggttgagcaagacccctatgaaattgttattaagcaacttgaaagagctgcccaatatatggagataagtgaagaagctcttgagttcttaaagagacctcaaagaattgttgaggtcacaattccagtagaaatggatgacggttctgtaaaagttttcactggatttagagtacaacacaactgggctagaggtccaactaagggtggaattagatggcatccagaagaaacccttagcactgttaaagctcttgcagcttggatgacatggaagactgctgtaatggatctcccgtatggtggaggtaagggtggaataattgtagatccaaagaagctctccgacagagagaaggagaggcttgcaagaggttacattagagcaatttatgatgttattagcccgtatgaagacattccagcacccgatgtttatacaaacccacaaataatggcatggatgatggatgagtacgagacaataagcaggagaaagacaccggcctttggaattatcactggaaagcctcttagcattggtggatcacttggaagaattgaggcaactgcaagaggtgcaagttacacaattagagaggctgcaaaggttcttggatgggacaccctcaagggcaagacaatagcaatccagggttacggtaacgcgggttattatcttgcaaagatcatgagtgaagactttggaatgaaggttgtagctgtgagcgacagcaagggtggaatatacaaccccgatggtcttaatgctgacgaggttctcaagtggaagaatgagcatggaagcgttaaagacttcccaggagcaaccaacataacgaatgaggagctacttgagcttgaggttgatgttctcgctccggcagctatagaagaagtgataactaagaagaacgcagacaacattaaggctaagatcgttgcagaagtagcaaacggtccagttactccagaagctgatgagatactattcgagaaaggaatccttcagatcccagacttcctatgtaatgctggtggagttacagtcagctacttcgagtgggtacagaacataactggatactactggacaattgaggaggttagagagagactcgacaagaagatgacaaaagcattctacgacgtctacaacatagcaaaggagaagaacatacacatgagagatgcagcttacgtagttgcagtccagagagtttatcaagcaatgcttgaccgtggatgggtcaagcacaagcttggcggcggcagcggcggcggcagcggcggcggcagcaccgtggcgcagctggaagaaaaagtgaaaaccctgcgtgcgcagaactatgaactgaaaagccgtgtgcagcgtctgcgtgaacaggtggcgcagctggcgtaacttaagtcgaacagaaagtaatcgtattgtacacggccgcataatcgaaattaatacgactcactataggggaattgtgagcggataacaattccccatcttagtatattagttaagtataagaaggagatatacatatgtacgatgctgtgatcataggaggaggacccggaggttacgtctgcgctataaaactcgcacagcttggaaagaaggtcgcacttgtcgaaaaagatgccctcgggggaacctgtaccaacaggggatgcattccaacgaaggcgatgctcacagtctcccatctcatggatgaaatgaaagagaaagcgtcaaagtacggcttgaaagtctcaggagtcgaatacgacgtcgccgccataatgaaacacgttcaaaagagtgtgatgatgtcaagaaagggaatagagtacctgctgaaaaagaatggggttgaagtcttcaaaggaacggccgtcgtggaaaacaagaacacggttgttgtccaggaaacgggagagaaactggaagcaaagaatcttgttctcgcacacggatccgttccctccgtgttttctcccttcgatatcgacggagtctggacgagtgatgacgtgttcaatttgaaagaatttccaaagtcgcttgtgatagtgggcggtggtgtcatcggagttgaattcgcgacgtttttcggttctttcggtgtggatgtgacgatcgttgagatcgcagaacacatcctcccctatgaagactcagatgtggcagaagaagtgaaaaaggccctcaaaaggaagggtgttaagatcctggaaaaaacaaaaatttcctccctgagcaaagtcgatgacggctttgaagtcgcactcgaaaatggagaaaccctgaaggcagaaaaggtcctgctcgccgcaggaagaaagcccaacataccggaggacgtgaaagctctcggagtgaagatagaaaaaggagtcgtcactgatagcaggatgagaacgaacgtggaaaacgtttacgcgataggagatatcagaagtggaatcatgctcgctcatgtggccatgtacgagggaatagtcgctgcaaagaacatcgcgggagaagaggaagaaatggattactcggccgttccgagtatcatcttctcctctccggaagtggcatctgtcggtgtgagagaaaaagacgtgaaccccgaagaagttgtgatctccaagttcccagtctccgcaaacggaagggcaagaacgatgcttgaaaacatagggtttgccaaggtcatagcggacaagaaagacggaacggttctcggcatgagcatcgtttcaccatcggctaccgatatgatcatggaaggtgtcatagccgtgaagttcaggatgaaagcggaagatctggagaaagccattcacccgcatccgactttgacagaaaccatactcggggctcttgaaggggtctcaggaaaacccattcatctgctcgagggcggcggcagcggcggcggcagcggcggcggcagcagcgtggatgaactgcaggcggaagtggatcagctgcaggatgaaaactatgcgctgaaaaccaaagtggcgcagctgcgtaaaaaagtggaaaaactgtaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 104 - DNA, 44 bp, Artificial
  PCR primer
aataatccatggcggagcaagacccctatgaaattgttattaag

SEQ ID NO: 105 - DNA, 6265 bp, Artificial
  plasmid, pCDFDuet-pfuEDH-tmaDp
taatacgactcactataggggaattgtgagcggataacaattcccctgtagaaataattttgtttaactttaataaggagatataccatggcggagcaagacccctatgaaattgttattaagcaacttgaaagagctgcccaatatatggagataagtgaagaagctcttgagttcttaaagagacctcaaagaattgttgaggtcacaattccagtagaaatggatgacggttctgtaaaagttttcactggatttagagtacaacacaactgggctagaggtccaactaagggtggaattagatggcatccagaagaaacccttagcactgttaaagctcttgcagcttggatgacatggaagactgctgtaatggatctcccgtatggtggaggtaagggtggaataattgtagatccaaagaagctctccgacagagagaaggagaggcttgcaagaggttacattagagcaatttatgatgttattagcccgtatgaagacattccagcacccgatgtttatacaaacccacaaataatggcatggatgatggatgagtacgagacaataagcaggagaaagacaccggcctttggaattatcactggaaagcctcttagcattggtggatcacttggaagaattgaggcaactgcaagaggtgcaagttacacaattagagaggctgcaaaggttcttggatgggacaccctcaagggcaagacaatagcaatccagggttacggtaacgcgggttattatcttgcaaagatcatgagtgaagactttggaatgaaggttgtagctgtgagcgacagcaagggtggaatatacaaccccgatggtcttaatgctgacgaggttctcaagtggaagaatgagcatggaagcgttaaagacttcccaggagcaaccaacataacgaatgaggagctacttgagcttgaggttgatgttctcgctccggcagctatagaagaagtgataactaagaagaacgcagacaacattaaggctaagatcgttgcagaagtagcaaacggtccagttactccagaagctgatgagatactattcgagaaaggaatccttcagatcccagacttcctatgtaatgctggtggagttacagtcagctacttcgagtgggtacagaacataactggatactactggacaattgaggaggttagagagagactcgacaagaagatgacaaaagcattctacgacgtctacaacatagcaaaggagaagaacatacacatgagagatgcagcttacgtagttgcagtccagagagtttatcaagcaatgcttgaccgtggatgggtcaagcacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgtacgatgctgtgatcataggaggaggacccggaggttacgtctgcgctataaaactcgcacagcttggaaagaaggtcgcacttgtcgaaaaagatgccctcgggggaacctgtaccaacaggggatgcattccaacgaaggcgatgctcacagtctcccatctcatggatgaaatgaaagagaaagcgtcaaagtacggcttgaaagtctcaggagtcgaatacgacgtcgccgccataatgaaacacgttcaaaagagtgtgatgatgtcaagaaagggaatagagtacctgctgaaaaagaatggggttgaagtcttcaaaggaacggccgtcgtggaaaacaagaacacggttgttgtccaggaaacgggagagaaactggaagcaaagaatcttgttctcgcacacggatccgttccctccgtgttttctcccttcgatatcgacggagtctggacgagtgatgacgtgttcaatttgaaagaatttccaaagtcgcttgtgatagtgggcggtggtgtcatcggagttgaattcgcgacgtttttcggttctttcggtgtggatgtgacgatcgttgagatcgcagaacacatcctcccctatgaagactcagatgtggcagaagaagtgaaaaaggccctcaaaaggaagggtgttaagatcctggaaaaaacaaaaatttcctccctgagcaaagtcgatgacggctttgaagtcgcactcgaaaatggagaaacccttaaggcagaaaaggtcctgctcgccgcaggaagaaagcccaacataccggaggacgtgaaagctctcggagtgaagatagaaaaaggagtcgtcactgatagcaggatgagaacgaacgtggaaaacgtttacgcgataggagatatcagaagtggaatcatgctcgctcatgtggccatgtacgagggaatagtcgctgcaaagaacatcgcgggagaagaggaagaaatggattactcggccgttccgagtatcatcttctcctctccggaagtggcatctgtcggtgtgagagaaaaagacgtgaaccccgaagaagttgtgatctccaagttcccagtctccgcaaacggaagggcaagaacgatgcttgaaaacatagggtttgccaaggtcatagcggacaagaaagacggaacggttctcggcatgagcatcgtttcaccatcggctaccgatatgatcatggaaggtgtcatagccgtgaagttcaggatgaaagcggaagatctggagaaagccattcacccgcatccgactttgacagaaaccatactcggggctcttgaaggggtctcaggaaaacccattcatctgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 106 - protein, 481 aa, Artificial
  His-pfuEDH-Dzip1
MGSSHHHHHHSQDPVEQDPYEIVIKQLERAAQYMEISEEALEFLKRPQRIVEVTIPVEMDDGSVKVFTGFRVQHNWARGPTKGGIRWHPEETLSTVKALAAWMTWKTAVMDLPYGGGKGGIIVDPKKLSDREKERLARGYIRAIYDVISPYEDIPAPDVYTNPQIMAWMMDEYETISRRKTPAFGIITGKPLSIGGSLGRIEATARGASYTIREAAKVLGWDTLKGKTIAIQGYGNAGYYLAKIMSEDFGMKVVAVSDSKGGIYNPDGLNADEVLKWKNEHGSVKDFPGATNITNEELLELEVDVLAPAAIEEVITKKNADNIKAKIVAEVANGPVTPEADEILFEKGILQIPDFLCNAGGVTVSYFEWVQNITGYYWTIEEVRERLDKKMTKAFYDVYNIAKEKNIHMRDAAYVVAVQRVYQAMLDRGWVKHKLGGGSGGGSGGGSTVAQLEEKVKTLRAQNYELKSRVQRLREQVAQLA

SEQ ID NO: 107 - DNA, 42 bp, Artificial
  PCR primer
aataatcatatggttgagcaagacccctatgaaattgttatt

SEQ ID NO: 108 - DNA, 42 bp, Artificial
  PCR primer
aataatctcgaggtgcttgacccatccacggtcaagcattgc

SEQ ID NO: 109 - DNA, 42 bp, Artificial
  PCR primer
aataatcatatgacggcatctctccgttgcgaagtggcagcg

SEQ ID NO: 110 - DNA, 42 bp, Artificial
  PCR primer
aataatctcgagggcgtccgccgaaccgccatacacatcgga

SEQ ID NO: 111 - DNA, 8114 bp, Artificial
  plasmid, pETDuet-sceADH-Dzip1/goxALDH-Dzip2
ggggaattgtgagcggataacaattcccctctagaaataattttgtttaactttaagaaggagatataccatgggcagcagccatcaccatcatcaccacagccaggatccgccttcgcaagtcattcctgaaaaacaaaaggctattgtcttttatgagacagatggaaaattggaatataaagacgtcacagttccggaacctaagcctaacgaaattttagtccacgttaaatattctggtgtttgtcatagtgacttgcacgcgtggcacggtgattggccatttcaattgaaatttccattaatcggtggtcacgaaggtgctggtgttgttgttaagttgggatctaacgttaagggctggaaagtcggtgattttgcaggtataaaatggttgaatgggacttgcatgtcctgtgaatattgtgaagtaggtaatgaatctcaatgtccttatttggatggtactggcttcacacatgatggtacttttcaagaatacgcaactgccgatgccgttcaagctgcccatattccaccaaacgtcaatcttgctgaagttgccccaatcttgtgtgcaggtatcactgtttataaggcgttgaaaagagccaatgtgataccaggccaatgggtcactatatccggtgcatgcggtggcttgggttctctggcaatccaatacgcccttgctatgggttacagggtcattggtatcgatggtggtaatgccaagcgaaagttatttgaacaattaggcggagaaatattcatcgatttcacggaagaaaaagacattgttggtgctataataaaggccactaatggcggttctcatggagttattaatgtgtctgtttctgaagcagctatcgaggcttctacgaggtattgtaggcccaatggtactgtcgtcctggttggtatgccagctcatgcttactgcaattccgatgttttcaatcaagttgtaaaatcaatctccatcgttggatcttgtgttggaaatagagctgatacaagggaggctttagatttcttcgccagaggtttgatcaaatctccgatccacttagctggcctatcggatgttcctgaaatttttgcaaagatggagaagggtgaaattgttggtagatatgttgttgagacttctaaaaagcttggcggcggcagcggcggcggcagcggcggcggcagcaccgtggcgcagctggaagaaaaagtgaaaaccctgcgtgcgcagaactatgaactgaaaagccgtgtgcagcgtctgcgtgaacaggtggcgcagctggcgtaacttaagtcgaacagaaagtaatcgtattgtacacggccgcataatcgaaattaatacgactcactataggggaattgtgagcggataacaattccccatcttagtatattagttaagtataagaaggagatatacatatgacggcatctctccgttgcgaagtggcagcgctcctcgacagccttggcgtgtcacctgatctgtatacggggggcagtctggcggtgaagtctccgctgacgggagaggtgattgccgaggtctcggaagtcagcgcggagcaggcgaaggagacgatcgccagttcactcgacgcgttcaaagcctggcgcagggttccggctccgcgtcgcggtgagcttgtccgcctgctgggtgaggagcttcgggccagcaaggaggcgctcggacggctcgtgacgctggaagtgggcaaggtgccgtccgaaggtctgggcgaagtgcaggaaatgatcgatatctgcgacttcgcggtcggtctgtcccgtcagctttatggcctgacgatccagtccgaacgtccggatcatcgcctgacagagcagtggcatcccgccgggccggtgggcatcatttcggcattcaatttccccgtggccgtctggtcctggaatgctgcgctggcactggtttgcggtgacagcgtgatctggaaaccttcggaaaagaccccgctgacagcgctggccacgcaggccctgttcctgaaagccgctgcccgtttcggcagtgacgcaccggtcgccctcagcacgcttctggtcggtggccgcgagatcggcgaactgatggtgaaagaccgtcgtgtccctgtcatctcggcgacgggttcgacgcggatgggccgcgatgtcggtgagcgtgtggcgcggcgtttcgggcgctccattctggaacttggcgggaacaatgcgtccatcgtcaccccctccgccgatctggatctaacgctgcgggctgttgctttcggtgctatgggaaccgccggccagcgctgcacgaccctgcgtcgcctgttcgtgcattccagcgtctatgacacgctcgtgccaaagctggtcaatgtctacaagacgatttcggtcggtaacccggttgagggggacgcgctcgttggtccgctgatcgatgtggcgtccttcaaaatgatgcaggacagcctgaaagccgccgcggacgcaggtggcacggttcatggcggaacgcgtgtggatgtgaacggcgaggcgtcgttctatgtccgtccggcaatggtcgagatgccgtcgcagacggggccggtgctggaagaaaccttcgcgccgatcctttacgtcatgaaatatgacacgctcgatgaagccatcgcgcttcagaacgacgtggtgcagggcctgtcgtcttcgatctttgcgaccgatatccgcgaggtcgagcagttcctgtctgcgcagggttccgactgcggtattgcgaacgtcaatatggggccgtccggcgcagaaatcgggggagccttcgggggcgagaaggaaactggcggcgggcgggagtccggttccgatgcgtggaaagcctatatgcgccggcagaccaatgccatcaattacgggcggaccctgccgcttgcgcagggtgtgaagttcgatgtgtatggcggttcggcggacgccctcgagggcggcggcagcggcggcggcagcggcggcggcagcagcgtggatgaactgcaggcggaagtggatcagctgcaggatgaaaactatgcgctgaaaaccaaagtggcgcagctgcgtaaaaaagtggaaaaactgtaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 112 - DNA, 42 bp, Artificial
  PCR primer
aataatctcgagtcaggcgtccgccgaaccgccatacacatc

SEQ ID NO: 113 - DNA, 6247 bp, Artificial
  plasmid, pCDFDuet-sceADH-goxALDH
taatacgactcactataggggaattgtgagcggataacaattcccctgtagaaataattttgtttaactttaataaggagatataccatggcgtcgcaagtcattcctgaaaaacaaaaggctattgtcttttatgagacagatggaaaattggaatataaagacgtcacagttccggaacctaagcctaacgaaattttagtccacgttaaatattctggtgtttgtcatagtgacttgcacgcgtggcacggtgattggccatttcaattgaaatttccattaatcggtggtcacgaaggtgctggtgttgttgttaagttgggatctaacgttaagggctggaaagtcggtgattttgcaggtataaaatggttgaatgggacttgcatgtcctgtgaatattgtgaagtaggtaatgaatctcaatgtccttatttggatggtactggcttcacacatgatggtacttttcaagaatacgcaactgccgatgccgttcaagctgcccatattccaccaaacgtcaatcttgctgaagttgccccaatcttgtgtgcaggtatcactgtttataaggcgttgaaaagagccaatgtgataccaggccaatgggtcactatatccggtgcatgcggtggcttgggttctctggcaatccaatacgcccttgctatgggttacagggtcattggtatcgatggtggtaatgccaagcgaaagttatttgaacaattaggcggagaaatattcatcgatttcacggaagaaaaagacattgttggtgctataataaaggccactaatggcggttctcatggagttattaatgtgtctgtttctgaagcagctatcgaggcttctacgaggtattgtaggcccaatggtactgtcgtcctggttggtatgccagctcatgcttactgcaattccgatgttttcaatcaagttgtaaaatcaatctccatcgttggatcttgtgttggaaatagagctgatacaagggaggctttagatttcttcgccagaggtttgatcaaatctccgatccacttagctggcctatcggatgttcctgaaatttttgcaaagatggagaagggtgaaattgttggtagatatgttgttgagacttctaaaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgacggcatctctccgttgcgaagtggcagcgctcctcgacagccttggcgtgtcacctgatctgtatacggggggcagtctggcggtgaagtctccgctgacgggagaggtgattgccgaggtctcggaagtcagcgcggagcaggcgaaggagacgatcgccagttcactcgacgcgttcaaagcctggcgcagggttccggctccgcgtcgcggtgagcttgtccgcctgctgggtgaggagcttcgggccagcaaggaggcgctcggacggctcgtgacgctggaagtgggcaaggtgccgtccgaaggtctgggcgaagtgcaggaaatgatcgatatctgcgacttcgcggtcggtctgtcccgtcagctttatggcctgacgatccagtccgaacgtccggatcatcgcctgacagagcagtggcatcccgccgggccggtgggcatcatttcggcattcaatttccccgtggccgtctggtcctggaatgctgcgctggcactggtttgcggtgacagcgtgatctggaaaccttcggaaaagaccccgctgacagcgctggccacgcaggccctgttcctgaaagccgctgcccgtttcggcagtgacgcaccggtcgccctcagcacgcttctggtcggtggccgcgagatcggcgaactgatggtgaaagaccgtcgtgtccctgtcatctcggcgacgggttcgacgcggatgggccgcgatgtcggtgagcgtgtggcgcggcgtttcgggcgctccattctggaacttggcgggaacaatgcgtccatcgtcaccccctccgccgatctggatctaacgctgcgggctgttgctttcggtgctatgggaaccgccggccagcgctgcacgaccctgcgtcgcctgttcgtgcattccagcgtctatgacacgctcgtgccaaagctggtcaatgtctacaagacgatttcggtcggtaacccggttgagggggacgcgctcgttggtccgctgatcgatgtggcgtccttcaaaatgatgcaggacagcctgaaagccgccgcggacgcaggtggcacggttcatggcggaacgcgtgtggatgtgaacggcgaggcgtcgttctatgtccgtccggcaatggtcgagatgccgtcgcagacggggccggtgctggaagaaaccttcgcgccgatcctttacgtcatgaaatatgacacgctcgatgaagccatcgcgcttcagaacgacgtggtgcagggcctgtcgtcttcgatctttgcgaccgatatccgcgaggtcgagcagttcctgtctgcgcagggttccgactgcggtattgcgaacgtcaatatggggccgtccggcgcagaaatcgggggagccttcgggggcgagaaggaaactggcggcgggcgggagtccggttccgatgcgtggaaagcctatatgcgccggcagaccaatgccatcaattacgggcggaccctgccgcttgcgcagggtgtgaagttcgatgtgtatggcggttcggcggacgcctgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 114 - protein, 412 aa, Artificial
  His-sceADH-Dzip1
MGSSHHHHHHSQDPPSQVIPEKQKAIVFYETDGKLEYKDVTVPEPKPNEILVHVKYSGVCHSDLHAWHGDWPFQLKFPLIGGHEGAGVVVKLGSNVKGWKVGDFAGIKWLNGTCMSCEYCEVGNESQCPYLDGTGFTHDGTFQEYATADAVQAAHIPPNVNLAEVAPILCAGITVYKALKRANVIPGQWVTISGACGGLGSLAIQYALAMGYRVIGIDGGNAKRKLFEQLGGEIFIDFTEEKDIVGAIIKATNGGSHGVINVSVSEAAIEASTRYCRPNGTVVLVGMPAHAYCNSDVFNQVVKSISIVGSCVGNRADTREALDFFARGLIKSPIHLAGLSDVPEIFAKMEKGEIVGRYVVETSKKLGGGSGGGSGGGSTVAQLEEKVKTLRAQNYELKSRVQRLREQVAQLA

SEQ ID NO: 115 - protein, 559 aa, Artificial
  goxALDH-Dzip2
MTASLRCEVAALLDSLGVSPDLYTGGSLAVKSPLTGEVIAEVSEVSAEQAKETIASSLDAFKAWRRVPAPRRGELVRLLGEELRASKEALGRLVTLEVGKVPSEGLGEVQEMIDICDFAVGLSRQLYGLTIQSERPDHRLTEQWHPAGPVGIISAFNFPVAVWSWNAALALVCGDSVIWKPSEKTPLTALATQALFLKAAARFGSDAPVALSTLLVGGREIGELMVKDRRVPVISATGSTRMGRDVGERVARRFGRSILELGGNNASIVTPSADLDLTLRAVAFGAMGTAGQRCTTLRRLFVHSSVYDTLVPKLVNVYKTISVGNPVEGDALVGPLIDVASFKMMQDSLKAAADAGGTVHGGTRVDVNGEASFYVRPAMVEMPSQTGPVLEETFAPILYVMKYDTLDEAIALQNDVVQGLSSSIFATDIREVEQFLSAQGSDCGIANVNMGPSGAEIGGAFGGEKETGGGRESGSDAWKAYMRRQTNAINYGRTLPLAQGVKFDVYGGSADALEGGGSGGGSGGGSSVDELQAEVDQLQDENYALKTKVAQLRKKVEKL

SEQ ID NO: 116 - DNA, 42 bp, Artificial
  PCR primer
aataatcatatgcgcaaggcggcaggcaagtacgggaacacc

SEQ ID NO: 117 - DNA, 42 bp, Artificial
  PCR primer
aataatctcgagaagccccagcacctccccccagggcgtggg

SEQ ID NO: 118 - DNA, 8240 bp, Artificial
  plasmid, pETDuet-pfuADH-Dzip1/tthALDH-Dzip2
ggggaattgtgagcggataacaattcccctctagaaataattttgtttaactttaagaaggagatataccatgggcagcagccatcaccatcatcaccacagccaggatccgtttttcctaaagactaagattatcgagggaaggggaagtttggagtatcttcaaaaggaagctattgggttcgagagggctttaatcttagcgtcaagctctatgaaaaggttagggtttctgaaggaagctgaagactatctaagggaaggtggagttgaggtaatttcaatagcaggccttcctcctgagcccacgaaagacgttgtcgacgagttcctcccaaaagttagggagttttctccagagatcctcattgcaattggtggaggaagtgttatcgatatggccaaagctctaaaggtattttacgatgtcccaggaatttcatttgaggaagttgcaatcaagagtagattttcgaagccgagacccgttccaaagctgaaaactccccttatagctattccctcaacgagcggtgctggaagtgaggtttcagcagcgagcgtgattaaggtaggggacgttaagtacacgattgtaactcccgagatagctcctgaagtggctatacttgaccctagattacccgaaggaatgcccccagaagttgccaggaattctggaatggatgttttagttcatgcgatagaggcttatgtttcaaaggcttcctcaccctttacagatgcaatggcagttaaagcggcaaaagctgtgttggagcatttagaggagagcattagaggagacaaaaatgccagagagaaaatccactatgcagctacaatggctggcatagctttcttaaacgcaaggcttggactcgttcatgcgatgagtcataaggccgcatggataggaccgcatggtctcgtaaatgccattctacttccctatgtgatggagtataacatggcacgcaggagagagaaatatgatgcattggcgagggaattgggatttgacaatgctgaagagctccttgaagagataaaagctctgaacgaaaggcttggaataggacccatctcaagttacgtcagcgaggaggagttcttagcgaaacttgatgaaatggtagagagagcctatgaagatcccctcgttaacttcaacccagttgagccgagaaaagaggacattagaagaatttatgagagggccttctatggagataagcttggcggcggcagcggcggcggcagcggcggcggcagcaccgtggcgcagctggaagaaaaagtgaaaaccctgcgtgcgcagaactatgaactgaaaagccgtgtgcagcgtctgcgtgaacaggtggcgcagctggcgtaacttaagtcgaacagaaagtaatcgtattgtacacggccgcataatcgaaattaatacgactcactataggggaattgtgagcggataacaattccccatcttagtatattagttaagtataagaaggagatatacatatgcgcaaggcggcaggcaagtacgggaacaccttggagtttggccacctcgtgggcggggaggaggtcctggagggcccccttctggagcgccgcaacccctcggaccgggaggacgtggttgcccgctttcccgaggcggacaaggacctggtgcgcaaggccgccttgaaggcccgggaggccttcgccgagtggagccggacccccgcccccatccggggccaggtcctcttcaacctggtgaagatcctggagcgggagaagcccaccctcacccggctcatggtgcgggaggtgggcaagacccccaaggaggcggcgggggacgtgcaggaggccatagacaccgccctcttcttcgcctcggagggccgcaggctttacggccagaccgtccccagcgagatgcgggacaaggagcttttcaccttccgcaggcccttgggcgtggtggggatcatcaccgccgggaacttccccatcgccgtccccagctggaagctcatccccgccgtcctcacggggaacaccgtcgtgtggaagccttccgaggacgcccccaccctctccttcgtcttcgccaagctctttgaggaggcgggcctgcccccgggcgtcctcaacgtcgtcttcggcggcgggaagggctccacgggccagtggatggtggagctcatggacgagggcctcttccagaagttcgccttcaccggctccacccaggtggggcgctggatcggggaggtggcggggcggaacctgatcaggcccaccctggagcttgggggcaagaaccccttggtggtcatgcgggacgccgacctggacctcgccgtggagggggcctggtggagcgccttcgccacgggagggcagcggtgcacctccgcggggaacatcctggtggatgcccccatctacgaggagttcaagcgccgcttcctggagcgggtggaggccaccctggtgggcaaccccctcctccaccccgaggtcacctacgggcccttcatcaacgagcgcttcttcgcccgctggcaggagcactaccgggtgggggaggcggagggggcgaggctcctcttcggccggggccggatcacgcgggaaaacccctacccccgcttcctgggggacccggaggccgggctttacggctggcccacggtctgggaggtgaggccggggacgcgcctcttcacggaggaggtcttcgggcccacgatcaacctcgtcaaggtggacgggattgaggaggccatagcggtggccaacagcaccccctacggcctttctagcgccatctacaccaaccaccgccactgggcctacctcttcaaggtgggcatccgggccggcatgaccagcatcaacaacgccaccgtgggcgccgaggcccacctccccttcggcggggtgaaggcgagcggcaacggggggagggagtcggggatctgggtcttggaggagtacacctactggcacgccgtgaacgaggagtactcggggaggctgcagctcgcccagatggacacgggctacgtgagccccaaggcgcccacgccctggggggaggtgctggggcttctcgagggcggcggcagcggcggcggcagcggcggcggcagcagcgtggatgaactgcaggcggaagtggatcagctgcaggatgaaaactatgcgctgaaaaccaaagtggcgcagctgcgtaaaaaagtggaaaaactgtaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 119 - DNA, 42 bp, Artificial
  PCR primer
aataatctcgagttaaagccccagcacctccccccagggcgt

SEQ ID NO: 120 - DNA, 6373 bp, Artificial
  plasmid, pCDFDuet-pfuADH-tthALDH
taatacgactcactataggggaattgtgagcggataacaattcccctgtagaaataattttgtttaactttaataaggagatataccatggcgttcctaaagactaagattatcgagggaaggggaagtttggagtatcttcaaaaggaagctattgggttcgagagggctttaatcttagcgtcaagctctatgaaaaggttagggtttctgaaggaagctgaagactatctaagggaaggtggagttgaggtaatttcaatagcaggccttcctcctgagcccacgaaagacgttgtcgacgagttcctcccaaaagttagggagttttctccagagatcctcattgcaattggtggaggaagtgttatcgatatggccaaagctctaaaggtattttacgatgtcccaggaatttcatttgaggaagttgcaatcaagagtagattttcgaagccgagacccgttccaaagctgaaaactccccttatagctattccctcaacgagcggtgctggaagtgaggtttcagcagcgagcgtgattaaggtaggggacgttaagtacacgattgtaactcccgagatagctcctgaagtggctatacttgaccctagattacccgaaggaatgcccccagaagttgccaggaattctggaatggatgttttagttcatgcgatagaggcttatgtttcaaaggcttcctcaccctttacagatgcaatggcagttaaagcggcaaaagctgtgttggagcatttagaggagagcattagaggagacaaaaatgccagagagaaaatccactatgcagctacaatggctggcatagctttcttaaacgcaaggcttggactcgttcatgcgatgagtcataaggccgcatggataggaccgcatggtctcgtaaatgccattctacttccctatgtgatggagtataacatggcacgcaggagagagaaatatgatgcattggcgagggaattgggatttgacaatgctgaagagctccttgaagagataaaagctctgaacgaaaggcttggaataggacccatctcaagttacgtcagcgaggaggagttcttagcgaaacttgatgaaatggtagagagagcctatgaagatcccctcgttaacttcaacccagttgagccgagaaaagaggacattagaagaatttatgagagggccttctatggagataagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgcgcaaggcggcaggcaagtacgggaacaccttggagtttggccacctcgtgggcggggaggaggtcctggagggcccccttctggagcgccgcaacccctcggaccgggaggacgtggttgcccgctttcccgaggcggacaaggacctggtgcgcaaggccgccttgaaggcccgggaggccttcgccgagtggagccggacccccgcccccatccggggccaggtcctcttcaacctggtgaagatcctggagcgggagaagcccaccctcacccggctcatggtgcgggaggtgggcaagacccccaaggaggcggcgggggacgtgcaggaggccatagacaccgccctcttcttcgcctcggagggccgcaggctttacggccagaccgtccccagcgagatgcgggacaaggagcttttcaccttccgcaggcccttgggcgtggtggggatcatcaccgccgggaacttccccatcgccgtccccagctggaagctcatccccgccgtcctcacggggaacaccgtcgtgtggaagccttccgaggacgcccccaccctctccttcgtcttcgccaagctctttgaggaggcgggcctgcccccgggcgtcctcaacgtcgtcttcggcggcgggaagggctccacgggccagtggatggtggagctcatggacgagggcctcttccagaagttcgccttcaccggctccacccaggtggggcgctggatcggggaggtggcggggcggaacctgatcaggcccaccctggagcttgggggcaagaaccccttggtggtcatgcgggacgccgacctggacctcgccgtggagggggcctggtggagcgccttcgccacgggagggcagcggtgcacctccgcggggaacatcctggtggatgcccccatctacgaggagttcaagcgccgcttcctggagcgggtggaggccaccctggtgggcaaccccctcctccaccccgaggtcacctacgggcccttcatcaacgagcgcttcttcgcccgctggcaggagcactaccgggtgggggaggcggagggggcgaggctcctcttcggccggggccggatcacgcgggaaaacccctacccccgcttcctgggggacccggaggccgggctttacggctggcccacggtctgggaggtgaggccggggacgcgcctcttcacggaggaggtcttcgggcccacgatcaacctcgtcaaggtggacgggattgaggaggccatagcggtggccaacagcaccccctacggcctttctagcgccatctacaccaaccaccgccactgggcctacctcttcaaggtgggcatccgggccggcatgaccagcatcaacaacgccaccgtgggcgccgaggcccacctccccttcggcggggtgaaggcgagcggcaacggggggagggagtcggggatctgggtcttggaggagtacacctactggcacgccgtgaacgaggagtactcggggaggctgcagctcgcccagatggacacgggctacgtgagccccaaggcgcccacgccctggggggaggtgctggggctttaactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 121 - protein, 436 aa, Artificial
  His-pfuADH-Dzip1
MGSSHHHHHHSQDPFFLKTKIIEGRGSLEYLQKEAIGFERALILASSSMKRLGFLKEAEDYLREGGVEVISIAGLPPEPTKDVVDEFLPKVREFSPEILIAIGGGSVIDMAKALKVFYDVPGISFEEVAIKSRFSKPRPVPKLKTPLIAIPSTSGAGSEVSAASVIKVGDVKYTIVTPEIAPEVAILDPRLPEGMPPEVARNSGMDVLVHAIEAYVSKASSPFTDAMAVKAAKAVLEHLEESIRGDKNAREKIHYAATMAGIAFLNARLGLVHAMSHKAAWIGPHGLVNAILLPYVMEYNMARRREKYDALARELGFDNAEELLEEIKALNERLGIGPISSYVSEEEFLAKLDEMVERAYEDPLVNFNPVEPRKEDIRRIYERAFYGDKLGGGSGGGSGGGSTVAQLEEKVKTLRAQNYELKSRVQRLREQVAQLA

SEQ ID NO: 122 - protein, 577 aa, Artificial
  tthALDH-Dzip2
MRKAAGKYGNTLEFGHLVGGEEVLEGPLLERRNPSDREDVVARFPEADKDLVRKAALKAREAFAEWSRTPAPIRGQVLFNLVKILEREKPTLTRLMVREVGKTPKEAAGDVQEAIDTALFFASEGRRLYGQTVPSEMRDKELFTFRRPLGVVGIITAGNFPIAVPSWKLIPAVLTGNTVVWKPSEDAPTLSFVFAKLFEEAGLPPGVLNVVFGGGKGSTGQWMVELMDEGLFQKFAFTGSTQVGRWIGEVAGRNLIRPTLELGGKNPLVVMRDADLDLAVEGAWWSAFATGGQRCTSAGNILVDAPIYEEFKRRFLERVEATLVGNPLLHPEVTYGPFINERFFARWQEHYRVGEAEGARLLFGRGRITRENPYPRFLGDPEAGLYGWPTVWEVRPGTRLFTEEVFGPTINLVKVDGIEEAIAVANSTPYGLSSAIYTNHRHWAYLFKVGIRAGMTSINNATVGAEAHLPFGGVKASGNGGRESGIWVLEEYTYWHAVNEEYSGRLQLAQMDTGYVSPKAPTPWGEVLGLLEGGGSGGGSGGGSSVDELQAEVDQLQDENYALKTKVAQLRKKVEKL

SEQ ID NO: 123 - DNA, 42 bp, Artificial
  PCR primer
aataatcatatgcaagcctattttgaccagctcgatcgcgtt

SEQ ID NO: 124 - DNA, 42 bp, Artificial
  PCR primer
aataatctcgagtttgtcgaacagataatggtttaccagatt

SEQ ID NO: 125 - DNA, 7628 bp, Artificial
  plasmid, pETDuet-busGDH-Dzip1/ecoISO-Dzip2
ggggaattgtgagcggataacaattcccctctagaaataattttgtttaactttaagaaggagatataccatgggcagcagccatcaccatcatcaccacagccaggatccgtatccggatttaaaaggaaaagtcgtcgctattacaggagctgcttcagggctcggaaaggcgatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaggagttcggcacactcgatattatgattaataatgccggtcttgaaaatcctgtgccatctcacgaaatgccgctcaaggattgggataaagtcatcggcacgaacttaacgggtgcctttttaggaagccgtgaagcgattaaatatttcgtagaaaacgatatcaagggaaatgtcattaacatgtccagtgtgcacgaagtgattccttggccgttatttgtccactatgcggcaagtaaaggcgggataaagctgatgacagaaacattagcgttggaatacgcgccgaagggcattcgcgtcaataatattgggccaggtgcgatcaacacgccaatcaatgctgaaaaattcgctgaccctaaacagaaagctgatgtagaaagcatgattccaatgggatatatcggcgaaccggaggagatcgccgcagtagcagcctggcttgcttcgaaggaagccagctacgtcacaggcatcacgttattcgcggacggcggtatgacacaatatccttcattccaggcaggccgcggtaagcttggcggcggcagcggcggcggcagcggcggcggcagcaccgtggcgcagctggaagaaaaagtgaaaaccctgcgtgcgcagaactatgaactgaaaagccgtgtgcagcgtctgcgtgaacaggtggcgcagctggcgtaacttaagtcgaacagaaagtaatcgtattgtacacggccgcataatcgaaattaatacgactcactataggggaattgtgagcggataacaattccccatcttagtatattagttaagtataagaaggagatatacatatgcaagcctattttgaccagctcgatcgcgttcgttatgaaggctcaaaatcctcaaacccgttagcattccgtcactacaatcccgacgaactggtgttgggtaagcgtatggaagagcacttgcgttttgccgcctgctactggcacaccttctgctggaacggggcggatatgtttggtgtgggggcgtttaatcgtccgtggcagcagcctggtgaggcactggcgttggcgaagcgtaaagcagatgtcgcatttgagtttttccacaagttacatgtgccattttattgcttccacgatgtggatgtttcccctgagggcgcgtcgttaaaagagtacatcaataattttgcgcaaatggttgatgtcctggcaggcaagcaagaagagagcggcgtgaagctgctgtggggaacggccaactgctttacaaaccctcgctacggcgcgggtgcggcgacgaacccagatcctgaagtcttcagctgggcggcaacgcaagttgttacagcgatggaagcaacccataaattgggcggtgaaaactatgtcctgtggggcggtcgtgaaggttacgaaacgctgttaaataccgacttgcgtcaggagcgtgaacaactgggccgctttatgcagatggtggttgagcataaacataaaatcggtttccagggcacgttgcttatcgaaccgaaaccgcaagaaccgaccaaacatcaatatgattacgatgccgcgacggtctatggcttcctgaaacagtttggtctggaaaaagagattaaactgaacattgaagctaaccacgcgacgctggcaggtcactctttccatcatgaaatagccaccgccattgcgcttggcctgttcggttctgtcgacgccaaccgtggcgatgcgcaactgggctgggacaccgaccagttcccgaacagtgtggaagagaatgcgctggtgatgtatgaaattctcaaagcaggcggtttcaccaccggtggtctgaacttcgatgccaaagtacgtcgtcaaagtactgataaatatgatctgttttacggtcatatcggcgcgatggatacgatggcactggcgctgaaaattgcagcgcgcatgattgaagatggcgagctggataaacgcatcgcgcagcgttattccggctggaatagcgaattgggccagcaaatcctgaaaggccaaatgtcactggcagatttagccaaatatgctcaggaacatcatttgtctccggtgcatcagagtggtcgccaggaacaactggaaaatctggtaaaccattatctgttcgacaaactcgagggcggcggcagcggcggcggcagcggcggcggcagcagcgtggatgaactgcaggcggaagtggatcagctgcaggatgaaaactatgcgctgaaaaccaaagtggcgcagctgcgtaaaaaagtggaaaaactgtaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 126 - DNA, 42 bp, Artificial
  PCR primer
aataatctcgagttatttgtcgaacagataatggtttaccag

SEQ ID NO: 127 - DNA, 5761 bp, Artificial
  plasmid, pCDFDuet-busGDH-ecoISO
taatacgactcactataggggaattgtgagcggataacaattcccctgtagaaataattttgtttaactttaataaggagatataccatggcgccggatttaaaaggaaaagtcgtcgctattacaggagctgcttcagggctcggaaaggcgatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaggagttcggcacactcgatattatgattaataatgccggtcttgaaaatcctgtgccatctcacgaaatgccgctcaaggattgggataaagtcatcggcacgaacttaacgggtgcctttttaggaagccgtgaagcgattaaatatttcgtagaaaacgatatcaagggaaatgtcattaacatgtccagtgtgcacgaagtgattccttggccgttatttgtccactatgcggcaagtaaaggcgggataaagctgatgacagaaacattagcgttggaatacgcgccgaagggcattcgcgtcaataatattgggccaggtgcgatcaacacgccaatcaatgctgaaaaattcgctgaccctaaacagaaagctgatgtagaaagcatgattccaatgggatatatcggcgaaccggaggagatcgccgcagtagcagcctggcttgcttcgaaggaagccagctacgtcacaggcatcacgttattcgcggacggcggtatgacacaatatccttcattccaggcaggccgcggtaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgcaagcctattttgaccagctcgatcgcgttcgttatgaaggctcaaaatcctcaaacccgttagcattccgtcactacaatcccgacgaactggtgttgggtaagcgtatggaagagcacttgcgttttgccgcctgctactggcacaccttctgctggaacggggcggatatgtttggtgtgggggcgtttaatcgtccgtggcagcagcctggtgaggcactggcgttggcgaagcgtaaagcagatgtcgcatttgagtttttccacaagttacatgtgccattttattgcttccacgatgtggatgtttcccctgagggcgcgtcgttaaaagagtacatcaataattttgcgcaaatggttgatgtcctggcaggcaagcaagaagagagcggcgtgaagctgctgtggggaacggccaactgctttacaaaccctcgctacggcgcgggtgcggcgacgaacccagatcctgaagtcttcagctgggcggcaacgcaagttgttacagcgatggaagcaacccataaattgggcggtgaaaactatgtcctgtggggcggtcgtgaaggttacgaaacgctgttaaataccgacttgcgtcaggagcgtgaacaactgggccgctttatgcagatggtggttgagcataaacataaaatcggtttccagggcacgttgcttatcgaaccgaaaccgcaagaaccgaccaaacatcaatatgattacgatgccgcgacggtctatggcttcctgaaacagtttggtctggaaaaagagattaaactgaacattgaagctaaccacgcgacgctggcaggtcactctttccatcatgaaatagccaccgccattgcgcttggcctgttcggttctgtcgacgccaaccgtggcgatgcgcaactgggctgggacaccgaccagttcccgaacagtgtggaagagaatgcgctggtgatgtatgaaattctcaaagcaggcggtttcaccaccggtggtctgaacttcgatgccaaagtacgtcgtcaaagtactgataaatatgatctgttttacggtcatatcggcgcgatggatacgatggcactggcgctgaaaattgcagcgcgcatgattgaagatggcgagctggataaacgcatcgcgcagcgttattccggctggaatagcgaattgggccagcaaatcctgaaaggccaaatgtcactggcagatttagccaaatatgctcaggaacatcatttgtctccggtgcatcagagtggtcgccaggaacaactggaaaatctggtaaaccattatctgttcgacaaataactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 128 - protein, 322 aa, Artificial
  His-busGDH-Dzip1
MGSSHHHHHHSQDPYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLENPVPSHEMPLKDWDKVIGTNLTGAFLGSREAIKYFVENDIKGNVINMSSVHEVIPWPLFVHYAASKGGIKLMTETLALEYAPKGIRVNNIGPGAINTPINAEKFADPKQKADVESMIPMGYIGEPEEIAAVAAWLASKEASYVTGITLFADGGMTQYPSFQAGRGKLGGGSGGGSGGGSTVAQLEEKVKTLRAQNYELKSRVQRLREQVAQLA

SEQ ID NO: 129 - protein, 487 aa, Artificial
  ecoISO-Dzip2
MQAYFDQLDRVRYEGSKSSNPLAFRHYNPDELVLGKRMEEHLRFAACYWHTFCWNGADMFGVGAFNRPWQQPGEALALAKRKADVAFEFFHKLHVPFYCFHDVDVSPEGASLKEYINNFAQMVDVLAGKQEESGVKLLWGTANCFTNPRYGAGAATNPDPEVFSWAATQVVTAMEATHKLGGENYVLWGGREGYETLLNTDLRQEREQLGRFMQMVVEHKHKIGFQGTLLIEPKPQEPTKHQYDYDAATVYGFLKQFGLEKEIKLNIEANHATLAGHSFHHEIATAIALGLFGSVDANRGDAQLGWDTDQFPNSVEENALVMYEILKAGGFTTGGLNFDAKVRRQSTDKYDLFYGHIGAMDTMALALKIAARMIEDGELDKRIAQRYSGWNSELGQQILKGQMSLADLAKYAQEHHLSPVHQSGRQEQLENLVNHYLFDKLEGGGSGGGSGGGSSVDELQAEVDQLQDENYALKTKVAQLRKKVEKL

SEQ ID NO: 130 - DNA, 42 bp, Artificial
  PCR primer
aataatcatatggcagaatttttcccagaaatcccaaagatt

SEQ ID NO: 131 - DNA, 42 bp, Artificial
  PCR primer
aataatctcgagcctcagttctgctattgtcttcactatgta

SEQ ID NO: 132 - DNA, 7634 bp, Artificial
  plasmid, pETDuet-pfuGDH-Dzip1/tmaISO-Dzip2
ggggaattgtgagcggataacaattcccctctagaaataattttgtttaactttaagaaggagatataccatgggcagcagccatcaccatcatcaccacagccaggatccgaacattttaataacagcttcttcaagaggaataggcttcaatgtggccaaggaactcctcaaaaaaggacacagagtagtaatatcgtctagcaatgaaaaaaatttagaaaaagccctagaagagctaaaagaatatgaaaaagtctatccgattaaagcagatctctcaaaaaaagaagaaattaaaaacatggtagaaaaggcctgggaaattttaggaaaaatagatgtccttgtttggaatgcccctaatgtaaagtgtgaaccctgcatgattcacgaattagattatgaagattgggtagaaggagccttgcttcacataattgccccaggctacctaacaactcttctccttaataaatggttgaaaaataacacaagagggataataatctacttgagttctgcttcagttttagagccaatgcctcccctccttctcgcagacagtactagggcaggattaattcagcttgctaaaggaatatctagggcctatggaaagagaggaataagggcatacacagtactacttgggagctttgataccccaggagctagagaaaacctaagaagactagcagaaaaaaggggtgttgatccagaagagctatggaaaaaggaagttattgaaagaactcctcttcaaagaactggaaaatgggaagaactaggagaattaatagaattcttacttagcgataacgccgaatatatgctaggctctacaattctctttgatggagcaatgactaggagcgttcttctgaagcttggcggcggcagcggcggcggcagcggcggcggcagcaccgtggcgcagctggaagaaaaagtgaaaaccctgcgtgcgcagaactatgaactgaaaagccgtgtgcagcgtctgcgtgaacaggtggcgcagctggcgtaacttaagtcgaacagaaagtaatcgtattgtacacggccgcataatcgaaattaatacgactcactataggggaattgtgagcggataacaattccccatcttagtatattagttaagtataagaaggagatatacatatggcagaatttttcccagaaatcccaaagattcagtttgaaggtaaggaaagcacgaacccgctggcgttcaggttctacgatccgaacgaggtgatcgatgggaagcccctcaaggatcatctgaagttttccgtcgccttctggcacactttcgtgaacgagggaagagatccgtttggagatccaaccgctgaacgcccatggaacaggttctccgatcccatggacaaagcgtttgcaagggtggacgctctcttcgagttctgcgaaaagctcaacatcgagtacttctgtttccacgacagagacatcgctcccgaaggaaagacgctcagagagacaaacaagatactggacaaagtggtggaaagaattaaggaaagaatgaaggacagcaacgtgaaactcctctggggaacggcgaatctgttctcccacccgaggtacatgcacggcgccgcgacgacatgcagtgcggatgtctttgcctacgcggcggcccaggtgaagaaggccctggagatcacaaaagaactaggaggagaaggctacgtcttctggggtggaagagaaggctatgaaacactcctcaacacagatctcgggctggaactcgaaaatctcgcgagatttctcagaatggcagtggagtacgcaaagaagatcggcttcaccggacagttcctcatagaacccaagccgaaagaacccaccaagcaccagtacgatttcgatgtggcaacggcctacgctttcctgaagaaccacggccttgatgagtacttcaagttcaacattgaagcgaaccacgcgacacttgccggtcacacattccagcacgaactgaggatggcaaggatcctcggaaaacttggaagcatcgacgccaaccagggagatctcctgctcggctgggacaccgaccagttcccaacgaacatctacgacacaacacttgccatgtacgaagtgataaaagcaggaggattcacaaagggtggcctcaacttcgacgcgaaggtgaggagggcttcctacaaggtagaagatctcttcatcggtcacatcgcaggaatggacaccttcgccctcggcttcaagatagcgtacaaactcgcgaaagacggagtgttcgacaagttcatcgaggaaaaataccgtagcttcaaagaaggtatcggaaaagagatcgtagaaggaaaaaccgatttcgaaaaactcgaagagtatataatagacaaagaagatatcgaacttccatctggaaagcaggagtacctcgaaagcctgctcaacagctacatagtgaagacaatagcagaactgaggctcgagggcggcggcagcggcggcggcagcggcggcggcagcagcgtggatgaactgcaggcggaagtggatcagctgcaggatgaaaactatgcgctgaaaaccaaagtggcgcagctgcgtaaaaaagtggaaaaactgtaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 133 - DNA, 42 bp, Artificial
  PCR primer
aataatctcgagtcacctcagttctgctattgtcttcactat

SEQ ID NO: 134 - DNA, 5767 bp, Artificial
  plasmid, pCDFDuet-pfuGDH-tmaISO
taatacgactcactataggggaattgtgagcggataacaattcccctgtagaaataattttgtttaactttaataaggagatataccatggcgattttaataacagcttcttcaagaggaataggcttcaatgtggccaaggaactcctcaaaaaaggacacagagtagtaatatcgtctagcaatgaaaaaaatttagaaaaagccctagaagagctaaaagaatatgaaaaagtctatccgattaaagcagatctctcaaaaaaagaagaaattaaaaacatggtagaaaaggcctgggaaattttaggaaaaatagatgtccttgtttggaatgcccctaatgtaaagtgtgaaccctgcatgattcacgaattagattatgaagattgggtagaaggagccttgcttcacataattgccccaggctacctaacaactcttctccttaataaatggttgaaaaataacacaagagggataataatctacttaagttctgcttcagttttagagccaatgcctcccctccttctcgcagacagtactagggcaggattaattcagcttgctaaaggaatatctagggcctatggaaagagaggaataagggcatacacagtactacttgggagctttgataccccaggagctagagaaaacctaagaagactagcagaaaaaaggggtgttgatccagaagagctatggaaaaaggaagttattgaaagaactcctcttcaaagaactggaaaatgggaagaactaggagaattaatagaattcttacttagcgataacgccgaatatatgctaggctctacaattctctttgatggagcaatgactaggagcgttcttcttaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagaatttttcccagaaatcccaaagattcagtttgaaggtaaggaaagcacgaacccgctggcgttcaggttctacgatccgaacgaggtgatcgatgggaagcccctcaaggatcatctgaagttttccgtcgccttctggcacactttcgtgaacgagggaagagatccgtttggagatccaaccgctgaacgcccatggaacaggttctccgatcccatggacaaagcgtttgcaagggtggacgctctcttcgagttctgcgaaaagctcaacatcgagtacttctgtttccacgacagagacatcgctcccgaaggaaagacgctcagagagacaaacaagatactggacaaagtggtggaaagaattaaggaaagaatgaaggacagcaacgtgaaactcctctggggaacggcgaatctgttctcccacccgaggtacatgcacggcgccgcgacgacatgcagtgcggatgtctttgcctacgcggcggcccaggtgaagaaggccctggagatcacaaaagaactaggaggagaaggctacgtcttctggggtggaagagaaggctatgaaacactcctcaacacagatctcgggctggaactcgaaaatctcgcgagatttctcagaatggcagtggagtacgcaaagaagatcggcttcaccggacagttcctcatagaacccaagccgaaagaacccaccaagcaccagtacgatttcgatgtggcaacggcctacgctttcctgaagaaccacggccttgatgagtacttcaagttcaacattgaagcgaaccacgcgacacttgccggtcacacattccagcacgaactgaggatggcaaggatcctcggaaaacttggaagcatcgacgccaaccagggagatctcctgctcggctgggacaccgaccagttcccaacgaacatctacgacacaacacttgccatgtacgaagtgataaaagcaggaggattcacaaagggtggcctcaacttcgacgcgaaggtgaggagggcttcctacaaggtagaagatctcttcatcggtcacatcgcaggaatggacaccttcgccctcggcttcaagatagcgtacaaactcgcgaaagacggagtgttcgacaagttcatcgaggaaaaataccgtagcttcaaagaaggtatcggaaaagagatcgtagaaggaaaaaccgatttcgaaaaactcgaagagtatataatagacaaagaagatatcgaacttccatctggaaagcaggagtacctcgaaagcctgctcaacagctacatagtgaagacaatagcagaactgaggtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 135 - protein, 320 aa, Artificial
  His-pfuGDH-Dzip1
MGSSHHHHHHSQDPNILITASSRGIGFNVAKELLKKGHRVVISSSNEKNLEKALEELKEYEKVYPIKADLSKKEEIKNMVEKAWEILGKIDVLVWNAPNVKCEPCMIHELDYEDWVEGALLHIIAPGYLTTLLLNKWLKNNTRGIIIYLSSASVLEPMPPLLLADSTRAGLIQLAKGISRAYGKRGIRAYTVLLGSFDTPGARENLRRLAEKRGVDPEELWKKEVIERTPLQRTGKWEELGELIEFLLSDNAEYMLGSTILFDGAMTRSVLLKLGGGSGGGSGGGSTVAQLEEKVKTLRAQNYELKSRVQRLREQVAQLA

SEQ ID NO: 136 - protein, 491 aa, Artificial
  tmaISO-Dzip2
MAEFFPEIPKIQFEGKESTNPLAFRFYDPNEVIDGKPLKDHLKFSVAFWHTFVNEGRDPFGDPTAERPWNRFSDPMDKAFARVDALFEFCEKLNIEYFCFHDRDIAPEGKTLRETNKILDKVVERIKERMKDSNVKLLWGTANLFSHPRYMHGAATTCSADVFAYAAAQVKKALEITKELGGEGYVFWGGREGYETLLNTDLGLELENLARFLRMAVEYAKKIGFTGQFLIEPKPKEPTKHQYDFDVATAYAFLKNHGLDEYFKFNIEANHATLAGHTFQHELRMARILGKLGSIDANQGDLLLGWDTDQFPTNIYDTTLAMYEVIKAGGFTKGGLNFDAKVRRASYKVEDLFIGHIAGMDTFALGFKIAYKLAKDGVFDKFIEEKYRSFKEGIGKEIVEGKTDFEKLEEYIIDKEDIELPSGKQEYLESLLNSYIVKTIAELRLEGGGSGGGSGGGSSVDELQAEVDQLQDENYALKTKVAQLRKKVEKL

SEQ ID NO: 137 - DNA, 147 bp, Artificial
  GGGSGGGSGGGS-Dzip3 direct, HindIII/AflII
agcttggcggcggcagcggcggcggcagcggcggcggcagcgcggaaattgcggcgattgaatatgaacaggcggcgattaaagaagaaattgcggcgattaaagataaaattgcggcgattaaagaatatattgcggcgatttaac

SEQ ID NO: 138 - DNA, 147 bp, Artificial
  GGGSGGGSGGGS-Dzip3 complement, HindIII/AflII
ttaagttaaatcgccgcaatatattctttaatcgccgcaattttatctttaatcgccgcaatttcttctttaatcgccgcctgttcatattcaatcgccgcaatttccgcgctgccgccgccgctgccgccgccgctgccgccgcca

SEQ ID NO: 139 - protein, 46 aa, Artificial
  GGGSGGGSGGGS-Dzip3
GGGSGGGSGGGSAEIAAIEYEQAAIKEEIAAIKDKIAAIKEYIAAI

SEQ ID NO: 140 - DNA, 147 bp, Artificial
  GGGSGGGSGGGS-Dzip4 direct, XhoI/AvrII
tcgagggcggcggcagcggcggcggcagcggcggcggcagcgaaaaaattgcggcgattaaagaagaacaggcggcgattgaagaagaaattcaggcgattaaagaagaaattgcggcgattaaatatctgattgcggcgatttaac

SEQ ID NO: 141 - DNA, 147 bp, Artificial
  GGGSGGGSGGGS-Dzip4 complement, XhoI/AvrII
ctaggttaaatcgccgcaatcagatatttaatcgccgcaatttcttctttaatcgcctgaatttcttcttcaatcgccgcctgttcttctttaatcgccgcaattttttcgctgccgccgccgctgccgccgccgctgccgccgccc

SEQ ID NO: 142 - protein, 46 aa, Artificial
  GGGSGGGSGGGS-Dzip4
GGGSGGGSGGGSEKIAAIKEEQAAIEEEIQAIKEEIAAIKYLIAAI

SEQ ID NO: 143 - DNA, 8117 bp, Artificial
  plasmid, pETDuet-sceADH-Dzip3/goxALDH-Dzip4
ggggaattgtgagcggataacaattcccctctagaaataattttgtttaactttaagaaggagatataccatgggcagcagccatcaccatcatcaccacagccaggatccgccttcgcaagtcattcctgaaaaacaaaaggctattgtcttttatgagacagatggaaaattggaatataaagacgtcacagttccggaacctaagcctaacgaaattttagtccacgttaaatattctggtgtttgtcatagtgacttgcacgcgtggcacggtgattggccatttcaattgaaatttccattaatcggtggtcacgaaggtgctggtgttgttgttaagttgggatctaacgttaagggctggaaagtcggtgattttgcaggtataaaatggttgaatgggacttgcatgtcctgtgaatattgtgaagtaggtaatgaatctcaatgtccttatttggatggtactggcttcacacatgatggtacttttcaagaatacgcaactgccgatgccgttcaagctgcccatattccaccaaacgtcaatcttgctgaagttgccccaatcttgtgtgcaggtatcactgtttataaggcgttgaaaagagccaatgtgataccaggccaatgggtcactatatccggtgcatgcggtggcttgggttctctggcaatccaatacgcccttgctatgggttacagggtcattggtatcgatggtggtaatgccaagcgaaagttatttgaacaattaggcggagaaatattcatcgatttcacggaagaaaaagacattgttggtgctataataaaggccactaatggcggttctcatggagttattaatgtgtctgtttctgaagcagctatcgaggcttctacgaggtattgtaggcccaatggtactgtcgtcctggttggtatgccagctcatgcttactgcaattccgatgttttcaatcaagttgtaaaatcaatctccatcgttggatcttgtgttggaaatagagctgatacaagggaggctttagatttcttcgccagaggtttgatcaaatctccgatccacttagctggcctatcggatgttcctgaaatttttgcaaagatggagaagggtgaaattgttggtagatatgttgttgagacttctaaaaagcttggcggcggcagcggcggcggcagcggcggcggcagcgcggaaattgcggcgattgaatatgaacaggcggcgattaaagaagaaattgcggcgattaaagataaaattgcggcgattaaagaatatattgcggcgatttaacttaagtcgaacagaaagtaatcgtattgtacacggccgcataatcgaaattaatacgactcactataggggaattgtgagcggataacaattccccatcttagtatattagttaagtataagaaggagatatacatatgacggcatctctccgttgcgaagtggcagcgctcctcgacagccttggcgtgtcacctgatctgtatacggggggcagtctggcggtgaagtctccgctgacgggagaggtgattgccgaggtctcggaagtcagcgcggagcaggcgaaggagacgatcgccagttcactcgacgcgttcaaagcctggcgcagggttccggctccgcgtcgcggtgagcttgtccgcctgctgggtgaggagcttcgggccagcaaggaggcgctcggacggctcgtgacgctggaagtgggcaaggtgccgtccgaaggtctgggcgaagtgcaggaaatgatcgatatctgcgacttcgcggtcggtctgtcccgtcagctttatggcctgacgatccagtccgaacgtccggatcatcgcctgacagagcagtggcatcccgccgggccggtgggcatcatttcggcattcaatttccccgtggccgtctggtcctggaatgctgcgctggcactggtttgcggtgacagcgtgatctggaaaccttcggaaaagaccccgctgacagcgctggccacgcaggccctgttcctgaaagccgctgcccgtttcggcagtgacgcaccggtcgccctcagcacgcttctggtcggtggccgcgagatcggcgaactgatggtgaaagaccgtcgtgtccctgtcatctcggcgacgggttcgacgcggatgggccgcgatgtcggtgagcgtgtggcgcggcgtttcgggcgctccattctggaacttggcgggaacaatgcgtccatcgtcaccccctccgccgatctggatctaacgctgcgggctgttgctttcggtgctatgggaaccgccggccagcgctgcacgaccctgcgtcgcctgttcgtgcattccagcgtctatgacacgctcgtgccaaagctggtcaatgtctacaagacgatttcggtcggtaacccggttgagggggacgcgctcgttggtccgctgatcgatgtggcgtccttcaaaatgatgcaggacagcctgaaagccgccgcggacgcaggtggcacggttcatggcggaacgcgtgtggatgtgaacggcgaggcgtcgttctatgtccgtccggcaatggtcgagatgccgtcgcagacggggccggtgctggaagaaaccttcgcgccgatcctttacgtcatgaaatatgacacgctcgatgaagccatcgcgcttcagaacgacgtggtgcagggcctgtcgtcttcgatctttgcgaccgatatccgcgaggtcgagcagttcctgtctgcgcagggttccgactgcggtattgcgaacgtcaatatggggccgtccggcgcagaaatcgggggagccttcgggggcgagaaggaaactggcggcgggcgggagtccggttccgatgcgtggaaagcctatatgcgccggcagaccaatgccatcaattacgggcggaccctgccgcttgcgcagggtgtgaagttcgatgtgtatggcggttcggcggacgccctcgagggcggcggcagcggcggcggcagcggcggcggcagcgaaaaaattgcggcgattaaagaagaacaggcggcgattgaagaagaaattcaggcgattaaagaagaaattgcggcgattaaatatctgattgcggcgatttaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 144 - DNA, 42 bp, Artificial
  PCR primer
aataatccatggttgtgaatgtactgatcgtccacgctcacc

SEQ ID NO: 145 - DNA, 42 bp, Artificial
  PCR primer
aataataagcttcgtcagcgggtacaacgcttcatcgaactg

SEQ ID NO: 146 - DNA, 147 bp, Artificial
  GGGSGGGSGGGS-Dzip5, direct HindIII/AflII
agcttggcggcggcagcggcggcggcagcggcggcggcagcgcggaaattgcggcgattaaatataaacaggcggcgattaaaaacgaaattgcggcgattaaacaggaaattgcggcgattgaacagatgattgcggcgatttaac

SEQ ID NO: 147 - DNA, 147 bp, Artificial
  GGGSGGGSGGGS-Dzip5 complement HindIII/AflII
ttaagttaaatcgccgcaatcatctgttcaatcgccgcaatttcctgtttaatcgccgcaatttcgtttttaatcgccgcctgtttatatttaatcgccgcaatttccgcgctgccgccgccgctgccgccgccgctgccgccgcca

SEQ ID NO: 148 - protein, 46 aa, Artificial
  GGGSGGGSGGGS-Dzip5
GGGSGGGSGGGSAEIAAIKYKQAAIKNEIAAIKQEIAAIEQMIAAI

SEQ ID NO: 149 - DNA, 5133 bp, Artificial
  plasmid, pCOLADuet-ppuDp-Dzip5/ppuDp
ggggaattgtgagcggataacaattcccctgtagaaataattttgtttaactttaataaggagatataccatggttgtgaatgtactgatcgtccacgctcaccccgaaccgcaatcgttcacggcagccctgcgtgaccaggctgtggaaaccttccgtgcccaaggtcatcaggtgcaggtcagtgacttgtacgcgatgggctggaacccggtggccagtgccgatgactttacccagcgcgagaaccccgagtacctcgtgtatgcgctggagcagcgtcagggcgtcaagcgcggtgccattgccgcagatatccagcaggagctggacaagctgttgtgggccgatctgctggtgctgaacttcccgatcttctggttctcggccccggccatgctcaagggctggatcgaccgggtgctggtgtcgggggtgtgctatggcggcaagcgcttctacgaccagggcgggttggccggcaagagggcgctggtgaccgtgaccctgggcgggcgcgagcacatgtttggcgacggcgcgattcacgggccgctggaagacatgctgcggccgattctgcggggcacgctggcctatgtcgggttcgaggtgctgccgccgttcgtggcgtggcatgtgccgtacatcagtgctgatgcgcggcaggactttctgcagcaataccggcagcgtctggagcagctctcggacgaccagccactggtgttcccgcgcctggcgcagttcgatgaagcgttgtacccgctgacgaagcttggcggcggcagcggcggcggcagcggcggcggcagcgcggaaattgcggcgattaaatataaacaggcggcgattaaaaacgaaattgcggcgattaaacaggaaattgcggcgattgaacagatgattgcggcgatttaacttaagtcgaacagaaagtaatcgtattgtacacggccgcataatcgaaattaatacgactcactataggggaattgtgagcggataacaattccccatcttagtatattagttaagtataagaaggagatatacatatgcttgtgaatgtactgatcgtccacgctcaccccgaaccgcaatcgttcacggcagccctgcgtgaccaggctgtggaaaccttccgtgcccaaggtcatcaggtgcaggtcagtgacttgtacgcgatgggctggaacccggtggccagtgccgatgactttacccagcgcgagaaccccgagtacctcgtgtatgcgctggagcagcgtcagggcgtcaagcgcggtgccattgccgcagatatccagcaggagctggacaagctgttgtgggccgatctgctggtgctgaacttcccgatcttctggttctcggccccggccatgctcaagggctggatcgaccgggtgctggtgtcgggggtgtgctatggcggcaagcgcttctacgaccagggcgggttggccggcaagagggcgctggtgaccgtgaccctgggcgggcgcgagcacatgtttggcgacggcgcgattcacgggccgctggaagacatgctgcggccgattctgcggggcacgctggcctatgtcgggttcgaggtgctgccgccgttcgtggcgtggcatgtgccgtacatcagtgctgatgcgcggcaggactttctgcagcaataccggcagcgtctggagcagctctcggacgaccagccactggtgttcccgcgcctggcgcagttcgatgaagcgttgtacccgctgacgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtatttggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 150 - protein, 412 aa, Artificial
  His-sceADH-Dzip3
MGSSHHHHHHSQDPPSQVIPEKQKAIVFYETDGKLEYKDVTVPEPKPNEILVHVKYSGVCHSDLHAWHGDWPFQLKFPLIGGHEGAGVVVKLGSNVKGWKVGDFAGIKWLNGTCMSCEYCEVGNESQCPYLDGTGFTHDGTFQEYATADAVQAAHIPPNVNLAEVAPILCAGITVYKALKRANVIPGQWVTISGACGGLGSLAIQYALAMGYRVIGIDGGNAKRKLFEQLGGEIFIDFTEEKDIVGAIIKATNGGSHGVINVSVSEAAIEASTRYCRPNGTVVLVGMPAHAYCNSDVFNQVVKSISIVGSCVGNRADTREALDFFARGLIKSPIHLAGLSDVPEIFAKMEKGEIVGRYVVETSKKLGGGSGGGSGGGSAEIAAIEYEQAAIKEEIAAIKDKIAAIKEYIAAI

SEQ ID NO: 151 - protein, 560 aa, Artificial
  goxALDH-Dzip4
MTASLRCEVAALLDSLGVSPDLYTGGSLAVKSPLTGEVIAEVSEVSAEQAKETIASSLDAFKAWRRVPAPRRGELVRLLGEELRASKEALGRLVTLEVGKVPSEGLGEVQEMIDICDFAVGLSRQLYGLTIQSERPDHRLTEQWHPAGPVGIISAFNFPVAVWSWNAALALVCGDSVIWKPSEKTPLTALATQALFLKAAARFGSDAPVALSTLLVGGREIGELMVKDRRVPVISATGSTRMGRDVGERVARRFGRSILELGGNNASIVTPSADLDLTLRAVAFGAMGTAGQRCTTLRRLFVHSSVYDTLVPKLVNVYKTISVGNPVEGDALVGPLIDVASFKMMQDSLKAAADAGGTVHGGTRVDVNGEASFYVRPAMVEMPSQTGPVLEETFAPILYVMKYDTLDEAIALQNDVVQGLSSSIFATDIREVEQFLSAQGSDCGIANVNMGPSGAEIGGAFGGEKETGGGRESGSDAWKAYMRRQTNAINYGRTLPLAQGVKFDVYGGSADALEGGGSGGGSGGGSEKIAAIKEEQAAIEEEIQAIKEEIAAIKYLIAAI

SEQ ID NO: 152 - protein, 283 aa, Artificial
  ppuDp-Dzip5
MVVNVLIVHAHPEPQSFTAALRDQAVETFRAQGHQVQVSDLYAMGWNPVASADDFTQRENPEYLVYALEQRQGVKRGAIAADIQQELDKLLWADLLVLNFPIFWFSAPAMLKGWIDRVLVSGVCYGGKRFYDQGGLAGKRALVTVTLGGREHMFGDGAIHGPLEDMLRPILRGTLAYVGFEVLPPFVAWHVPYISADARQDFLQQYRQRLEQLSDDQPLVFPRLAQFDEALYPLTKLGGGSGGGSGGGSAEIAAIKYKQAAIKNEIAAIKQEIAAIEQMIAAI

SEQ ID NO: 153 - DNA, 8243 bp, Artificial
  plasmid, pETDuet-pfuADH-Dzip3/tthALDH-Dzip4
ggggaattgtgagcggataacaattcccctctagaaataattttgtttaactttaagaaggagatataccatgggcagcagccatcaccatcatcaccacagccaggatccgtttttcctaaagactaagattatcgagggaaggggaagtttggagtatcttcaaaaggaagctattgggttcgagagggctttaatcttagcgtcaagctctatgaaaaggttagggtttctgaaggaagctgaagactatctaagggaaggtggagttgaggtaatttcaatagcaggccttcctcctgagcccacgaaagacgttgtcgacgagttcctcccaaaagttagggagttttctccagagatcctcattgcaattggtggaggaagtgttatcgatatggccaaagctctaaaggtattttacgatgtcccaggaatttcatttgaggaagttgcaatcaagagtagattttcgaagccgagacccgttccaaagctgaaaactccccttatagctattccctcaacgagcggtgctggaagtgaggtttcagcagcgagcgtgattaaggtaggggacgttaagtacacgattgtaactcccgagatagctcctgaagtggctatacttgaccctagattacccgaaggaatgcccccagaagttgccaggaattctggaatggatgttttagttcatgcgatagaggcttatgtttcaaaggcttcctcaccctttacagatgcaatggcagttaaagcggcaaaagctgtgttggagcatttagaggagagcattagaggagacaaaaatgccagagagaaaatccactatgcagctacaatggctggcatagctttcttaaacgcaaggcttggactcgttcatgcgatgagtcataaggccgcatggataggaccgcatggtctcgtaaatgccattctacttccctatgtgatggagtataacatggcacgcaggagagagaaatatgatgcattggcgagggaattgggatttgacaatgctgaagagctccttgaagagataaaagctctgaacgaaaggcttggaataggacccatctcaagttacgtcagcgaggaggagttcttagcgaaacttgatgaaatggtagagagagcctatgaagatcccctcgttaacttcaacccagttgagccgagaaaagaggacattagaagaatttatgagagggccttctatggagataagcttggcggcggcagcggcggcggcagcggcggcggcagcgcggaaattgcggcgattgaatatgaacaggcggcgattaaagaagaaattgcggcgattaaagataaaattgcggcgattaaagaatatattgcggcgatttaacttaagtcgaacagaaagtaatcgtattgtacacggccgcataatcgaaattaatacgactcactataggggaattgtgagcggataacaattccccatcttagtatattagttaagtataagaaggagatatacatatgcgcaaggcggcaggcaagtacgggaacaccttggagtttggccacctcgtgggcggggaggaggtcctggagggcccccttctggagcgccgcaacccctcggaccgggaggacgtggttgcccgctttcccgaggcggacaaggacctggtgcgcaaggccgccttgaaggcccgggaggccttcgccgagtggagccggacccccgcccccatccggggccaggtcctcttcaacctggtgaagatcctggagcgggagaagcccaccctcacccggctcatggtgcgggaggtgggcaagacccccaaggaggcggcgggggacgtgcaggaggccatagacaccgccctcttcttcgcctcggagggccgcaggctttacggccagaccgtccccagcgagatgcgggacaaggagcttttcaccttccgcaggcccttgggcgtggtggggatcatcaccgccgggaacttccccatcgccgtccccagctggaagctcatccccgccgtcctcacggggaacaccgtcgtgtggaagccttccgaggacgcccccaccctctccttcgtcttcgccaagctctttgaggaggcgggcctgcccccgggcgtcctcaacgtcgtcttcggcggcgggaagggctccacgggccagtggatggtggagctcatggacgagggcctcttccagaagttcgccttcaccggctccacccaggtggggcgctggatcggggaggtggcggggcggaacctgatcaggcccaccctggagcttgggggcaagaaccccttggtggtcatgcgggacgccgacctggacctcgccgtggagggggcctggtggagcgccttcgccacgggagggcagcggtgcacctccgcggggaacatcctggtggatgcccccatctacgaggagttcaagcgccgcttcctggagcgggtggaggccaccctggtgggcaaccccctcctccaccccgaggtcacctacgggcccttcatcaacgagcgcttcttcgcccgctggcaggagcactaccgggtgggggaggcggagggggcgaggctcctcttcggccggggccggatcacgcgggaaaacccctacccccgcttcctgggggacccggaggccgggctttacggctggcccacggtctgggaggtgaggccggggacgcgcctcttcacggaggaggtcttcgggcccacgatcaacctcgtcaaggtggacgggattgaggaggccatagcggtggccaacagcaccccctacggcctttctagcgccatctacaccaaccaccgccactgggcctacctcttcaaggtgggcatccgggccggcatgaccagcatcaacaacgccaccgtgggcgccgaggcccacctccccttcggcggggtgaaggcgagcggcaacggggggagggagtcggggatctgggtcttggaggagtacacctactggcacgccgtgaacgaggagtactcggggaggctgcagctcgcccagatggacacgggctacgtgagccccaaggcgcccacgccctggggggaggtgctggggcttctcgagggcggcggcagcggcggcggcagcggcggcggcagcgaaaaaattgcggcgattaaagaagaacaggcggcgattgaagaagaaattcaggcgattaaagaagaaattgcggcgattaaatatctgattgcggcgatttaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 154 - DNA, 39 bp, Artificial
  PCR primer
aataatccatggagatagtagttataggatctggaactg

SEQ ID NO: 155 - DNA, 42 bp, Artificial
  PCR primer
aataataagctttgacttaaattttctcatggccatttcagc

SEQ ID NO: 156 - DNA, 6363 bp, Artificial
  plasmid, pCOLADuet-phoDp-Dzip5/phoDp
ggggaattgtgagcggataacaattcccctgtagaaataattttgtttaactttaataaggagatataccatggagatagtagttataggatctggaactgctggaagcaattttgctctcttcatgaggaagctcgataggaaggccgagataatagttataggaaaggaacctacaatgcagtactccccctgtgcccttccacacgttattagcggaaccatagagaagcctgaagacgttatagtgtttccgaatgagttctatgagaagcagagaataaagatgatgcttggagtcgaagctaaaaagatagatagggagagaaaggttataattaccgataagggggaggttccctacgataagctggttcttgctactggatcaagggcttttattcccccgataaagggtgtcgaaaacgagggagtgttcacgctaaagagcctcgatgacgtaaggaggataaaggaatacataagcaagaggaaacctaaaaaggttgtcgttattggcgcaggtttaataggtctagaaggagcagaagcctttgcaaagattggaatggatgttctagttgttgaattactggagcatctattaccgactatgctcgacaaagacatggccaagatcgttcaagaggagatggagaaacatggagttaagttcaagtttggagttggagtcagcgagataataggaaatcctgtcaaggaagttaaaattggagaagagagtgtagaagcagatctagtcctagtggcaacgggagttagggctaatgttgacctagcgaaagatgctgggctggaagtgagtagggggatagttgttaacgagtatcttcagaccagtgatcctgatatatatgctataggagactgtgcagaggtattcgatgccgtaactggaaagagaaccctaagccaattgggaacttctgctgtcagaatggccaaagttgccgctgagaacatagctggaaagaaggtgaaatttaggccagtcttcaacacagctataactgaattattcgacctggaaataggaacatttggaataacagaagagagggctaaaaaggagggaattgaagtcgtaattggaaagtttaaaggttcaactaagccagagtattatcctggaggaaaaccaattacggttaagttaatatttagaaaggaagataagagactaataggggcccaaatagttggaggagagagagtttgggggaggataatgactttatcggcactcgcccagaaaggagctaccgttgaagatgttgcctatctagaaacgagttatgcaccacccataagcccaacgatagacccaatcacggtagccgctgaaatggccatgagaaaatttaagtcaaagcttggcggcggcagcggcggcggcagcggcggcggcagcgcggaaattgcggcgattaaatataaacaggcggcgattaaaaacgaaattgcggcgattaaacaggaaattgcggcgattgaacagatgattgcggcgatttaacttaagtcgaacagaaagtaatcgtattgtacacggccgcataatcgaaattaatacgactcactataggggaattgtgagcggataacaattccccatcttagtatattagttaagtataagaaggagatatacatatgaagatagtagttataggatctggaactgctggaagcaattttgctctcttcatgaggaagctcgataggaaggccgagataatagttataggaaaggaacctacaatgcagtactccccctgtgcccttccacacgttattagcggaaccatagagaagcctgaagacgttatagtgtttccgaatgagttctatgagaagcagagaataaagatgatgcttggagtcgaagctaaaaagatagatagggagagaaaggttataattaccgataagggggaggttccctacgataagctggttcttgctactggatcaagggcttttattcccccgataaagggtgtcgaaaacgagggagtgttcacgctaaagagcctcgatgacgtaaggaggataaaggaatacataagcaagaggaaacctaaaaaggttgtcgttattggcgcaggtttaataggtctagaaggagcagaagcctttgcaaagattggaatggatgttctagttgttgaattactggagcatctattaccgactatgctcgacaaagacatggccaagatcgttcaagaggagatggagaaacatggagttaagttcaagtttggagttggagtcagcgagataataggaaatcctgtcaaggaagttaaaattggagaagagagtgtagaagcagatctagtcctagtggcaacgggagttagggctaatgttgacctagcgaaagatgctgggctggaagtgagtagggggatagttgttaacgagtatcttcagaccagtgatcctgatatatatgctataggagactgtgcagaggtattcgatgccgtaactggaaagagaaccctaagccaattgggaacttctgctgtcagaatggccaaagttgccgctgagaacatagctggaaagaaggtgaaatttaggccagtcttcaacacagctataactgaattattcgacctggaaataggaacatttggaataacagaagagagggctaaaaaggagggaattgaagtcgtaattggaaagtttaaaggttcaactaagccagagtattatcctggaggaaaaccaattacggttaagttaatatttagaaaggaagataagagactaataggggcccaaatagttggaggagagagagtttgggggaggataatgactttatcggcactcgcccagaaaggagctaccgttgaagatgttgcctatctagaaacgagttatgcaccacccataagcccaacgatagacccaatcacggtagccgctgaaatggccatgagaaaatttaagtcatgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtatttggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 157 - protein, 436 aa, Artificial
  His-pfuADH-Dzip3
MGSSHHHHHHSQDPFFLKTKIIEGRGSLEYLQKEAIGFERALILASSSMKRLGFLKEAEDYLREGGVEVISIAGLPPEPTKDVVDEFLPKVREFSPEILIAIGGGSVIDMAKALKVFYDVPGISFEEVAIKSRFSKPRPVPKLKTPLIAIPSTSGAGSEVSAASVIKVGDVKYTIVTPEIAPEVAILDPRLPEGMPPEVARNSGMDVLVHAIEAYVSKASSPFTDAMAVKAAKAVLEHLEESIRGDKNAREKIHYAATMAGIAFLNARLGLVHAMSHKAAWIGPHGLVNAILLPYVMEYNMARRREKYDALARELGFDNAEELLEEIKALNERLGIGPISSYVSEEEFLAKLDEMVERAYEDPLVNFNPVEPRKEDIRRIYERAFYGDKLGGGSGGGSGGGSAEIAAIEYEQAAIKEEIAAIKDKIAAIKEYIAAI

SEQ ID NO: 158 - protein, 578 aa, Artificial
  tthALDH-Dzip4
MRKAAGKYGNTLEFGHLVGGEEVLEGPLLERRNPSDREDVVARFPEADKDLVRKAALKAREAFAEWSRTPAPIRGQVLFNLVKILEREKPTLTRLMVREVGKTPKEAAGDVQEAIDTALFFASEGRRLYGQTVPSEMRDKELFTFRRPLGVVGIITAGNFPIAVPSWKLIPAVLTGNTVVWKPSEDAPTLSFVFAKLFEEAGLPPGVLNVVFGGGKGSTGQWMVELMDEGLFQKFAFTGSTQVGRWIGEVAGRNLIRPTLELGGKNPLVVMRDADLDLAVEGAWWSAFATGGQRCTSAGNILVDAPIYEEFKRRFLERVEATLVGNPLLHPEVTYGPFINERFFARWQEHYRVGEAEGARLLFGRGRITRENPYPRFLGDPEAGLYGWPTVWEVRPGTRLFTEEVFGPTINLVKVDGIEEAIAVANSTPYGLSSAIYTNHRHWAYLFKVGIRAGMTSINNATVGAEAHLPFGGVKASGNGGRESGIWVLEEYTYWHAVNEEYSGRLQLAQMDTGYVSPKAPTPWGEVLGLLEGGGSGGGSGGGSEKIAAIKEEQAAIEEEIQAIKEEIAAIKYLIAAI

SEQ ID NO: 159 - protein, 488 aa, Artificial
  phoDp-Dzip5
MEIVVIGSGTAGSNFALFMRKLDRKAEIIVIGKEPTMQYSPCALPHVISGTIEKPEDVIVFPNEFYEKQRIKMMLGVEAKKIDRERKVIITDKGEVPYDKLVLATGSRAFIPPIKGVENEGVFTLKSLDDVRRIKEYISKRKPKKVVVIGAGLIGLEGAEAFAKIGMDVLVVELLEHLLPTMLDKDMAKIVQEEMEKHGVKFKFGVGVSEIIGNPVKEVKIGEESVEADLVLVATGVRANVDLAKDAGLEVSRGIVVNEYLQTSDPDIYAIGDCAEVFDAVTGKRTLSQLGTSAVRMAKVAAENIAGKKVKFRPVFNTAITELFDLEIGTFGITEERAKKEGIEVVIGKFKGSTKPEYYPGGKPITVKLIFRKEDKRLIGAQIVGGERVWGRIMTLSALAQKGATVEDVAYLETSYAPPISPTIDPITVAAEMAMRKFKSKLGGGSGGGSGGGSAEIAAIKYKQAAIKNEIAAIKQEIAAIEQMIAAI

SEQ ID NO: 160 - protein, 5 aa, Artificial
  spacer sequence
GGGGS

SEQ ID NO: 161 - protein, 8 aa, Artificial
  spacer sequence
STGDKQNA

SEQ ID NO: 162 - protein, 5 aa, Artificial
  spacer sequence
GSGSG

SEQ ID NO: 163 - protein, 5 aa, Artificial
  spacer sequence
SGGSG

SEQ ID NO: 164 - protein, 5 aa, Artificial
  spacer sequence
NGGGG

SEQ ID NO: 165 - protein, 14 aa, Artificial
  spacer sequence
EGKSSGSGSESKST

SEQ ID NO: 166 - protein, 5 aa, Artificial
  spacer sequence
SKSTS

SEQ ID NO: 167 - protein, 19 aa, Artificial
  spacer sequence
PVPSTPPTPSPSTPPTPSM

SEQ ID NO: 168 - protein, 8 aa, Artificial
  portion of association site sequence
GGGSGGGS

SEQ ID NO: 169 - protein, 12 aa, Artificial
  portion of association site sequence
GGGSGGGSGGGS

(19) United States
(12) Patent Application Publication
(10) Pub. No.: US 2007/0131547 A1
(43) Pub. Date: Jun. 14, 2007

(54) Enzyme electrode

(75) Inventors: Tsuyoshi Nomoto, Tokyo (JP); Wataru Kubo, Tokyo (JP); Tetsuya Yano, Tokyo (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/637,782

(22) Filed: Dec. 13, 2006

(30) Foreign Application Priority Data
Dec. 13, 2005 (JP) 2005-359172

(51) Int. Cl. G01N 33/487; H01M 4/90

(52) U.S. Cl. 204/403.01; 429/43

(57) ABSTRACT

The invention provides an enzyme electrode usable as a highly sensitive sensor, a high-output bio fuel cell or an electrochemical reaction device of a high reaction efficiency. The enzyme electrode includes an electroconductive base member and an enzyme, wherein the enzyme is formed by an associated protein in which two or more different enzyme proteins are associated.

### BACKGROUND OF THE INVENTION

1. Field of the Invention

The present invention relates to an enzyme electrode, and more particularly to an enzyme electrode adapted for use in a biosensor, a biofuel cell or an electrochemical reaction device.

2. Description of the Related Art

An enzyme, which is a biocatalyst of protein type formed in living cells, has a stronger function in milder conditions, in comparison with ordinary catalyst. Also a substrate, that is a substance causing a chemical reaction by the function of enzyme, has a high specificity, and each enzyme in general catalyzes a given reaction of a given substrate. If such characteristics of the enzyme, particularly an oxyreductase, can be ideally applied to a redox reaction in an electrode, an electrode of a low overvoltage and a high selectivity will be obtained.

As a technology for realizing an electron transferring reaction of a low overvoltage in the enzyme electrode, there is proposed a structure of utilizing two enzymes involved in the related reactions. More specifically, there is proposed an enzyme electrode simultaneously utilizing an enzyme 1 catalyzing a reaction which generates a reaction product 1 from a reaction substrate 1, and an enzyme 2 catalyzing a reaction which generates a reaction product 2 from a reaction substrate 2, wherein a part of the reaction product 1 includes a part of the reaction substrate 2.

An example of such electrode is shown in the following.

Ikura et al. (Patent Reference: Japanese Patent Application Laid-open No. 2003-279525), discloses an enzyme electrode which includes a reaction layer formed on the electrode and containing diaphorase, dehydrogenase and nicotinamide-adenine dinucleotide synthetase, wherein at least a part of the reaction layer is positioned on an active electrode area, and diaphorase, dehydrogenase and nicotinamide-adenine dinucleotide synthetase contained in such part of the reaction layer are immobilized on the surface of the active electrode area.

### SUMMARY OF THE INVENTION

As described above, an enzyme electrode utilizing, in a same reaction layer, an enzyme 1 catalyzing a chemical reaction which generates a reaction product 1 from a reaction substrate 1, and an enzyme 2 catalyzing a chemical reaction which generates a reaction product 2 from a reaction substrate 2 is already known. However, the enzyme 1 and the enzyme 2 are random in a relative distance and orientations thereof, so that a diffusion process in which a part of the reaction product 1, generated by the enzyme 1, becomes available as a part of the reaction substrate 2 for the enzyme 2, becomes a rate governing step. Therefore, the entire electron transferring reaction from the reaction substrate 1 to the electrode cannot be considered as optimized in the reaction rate and in the reaction efficiency in the presence of substrates of low concentrations. More specifically, such prior electrode, when utilized as a sensor, involves a drawback that a current measured on the electrode as a result of an oxidation reaction of the reaction substrate 1 is small, thus resulting in a low sensitivity with respect to the reaction substrate 1. Therefore, an object of the present invention is to provide an enzyme electrode utilizing novel enzymes.

The aforementioned object can be accomplished by the present invention in the following manner.

An enzyme electrode of the present invention, includes a conductive substrate and an enzyme, wherein the enzyme is an associated protein, formed by an association of two or more different enzyme proteins.

A sensor of the present invention includes the enzyme electrode of the above-described structure as a detecting portion for detecting a substance. A fuel cell of the present invention includes the enzyme electrode of the above-described structure as an anode or a cathode. An electrochemical reaction device of the present invention includes the enzyme electrode of the above-described structure as a reaction electrode.

According to the present invention, a novel enzyme electrode utilizing an associated protein is provided. Particularly in the case that two enzymes, where a product of a reaction involving an enzyme 1 (first enzyme) is utilized as a substrate for an enzyme 2 (second enzyme), the enzyme 1 and the enzyme 2 have a small relative distance, whereby an electron transferring reaction from the reaction by the enzyme 1 to the electrode proceeds efficiently.

Therefore, the sensor utilizing the enzyme electrode of the present invention shows a large current by an oxidation of the substrate, thus providing a high sensitivity. Also the fuel cell utilizing the enzyme electrode of the present invention allows to take out a large current. Also the electrochemical reaction device of the present invention utilizing the enzyme electrode of the present invention shows a high reaction efficiency. Also in case of utilizing an enzyme derived from thermophilic bacteria, the enzyme electrode shows a better storage stability in comparison with prior ones. Also the associated protein formed by associating different enzymes, for example an oxyreductase, to be employed in the enzyme electrode of the present invention, can be purified to a high purity by a simple heating operation, without requiring cumbersome operations such as a chromatography.

Further features of the present invention will become apparent from the following description of exemplary embodiments, with reference to the attached drawings.

### BRIEF DESCRIPTION OF THE DRAWINGS

FIG. 1 is a conceptual view of an enzyme electrode utilizing, as an embodiment of the enzyme electrode of the present invention, an associated protein of glucose dehydrogenase (GDH) and diaphorase (Dp) as a constituent element.

FIG. 2 is a conceptual view of an enzyme electrode utilizing, as an embodiment of the enzyme electrode of the present invention, an associated protein of alcohol dehydrogenase (ADH) and diaphorase (Dp) as a constituent element.

FIG. 3 is a conceptual view of an enzyme electrode utilizing, as an embodiment of the enzyme electrode of the present invention, an associated protein of lactic acid dehydrogenase (LDH) and diaphorase (Dp) as a constituent element.

FIG. 4 is a conceptual view of an enzyme electrode utilizing, as an embodiment of the enzyme electrode of the present invention, an associated protein of malic acid dehydrogenase (MDH) and diaphorase (Dp) as a constituent element.

FIG. 5 is a conceptual view of an enzyme electrode utilizing, as an embodiment of the enzyme electrode of the present invention, an associated protein of glutamic acid dehydrogenase (EDH) and diaphorase (Dp) as a constituent element.

FIG. 6 is a conceptual view of an enzyme electrode utilizing, as an embodiment of the enzyme electrode of the present invention, an associated protein of alcohol dehydrogenase (ADH) and aldehyde dehydrogenase (ALDH) as a constituent element.

FIG. 7 is a conceptual view of an enzyme electrode utilizing, as an embodiment of the enzyme electrode of the present invention, an associated protein of isomerase (ISO) and glucode dehydrogenase (GDH) as a constituent element.

FIG. 8 is a conceptual view of an enzyme electrode utilizing, as an embodiment of the enzyme electrode of the present invention, an associated protein of alcohol dehydrogenase (ADH), aldehyde dehydrogenase (ALDH) and diaphorase (Dp) as a constituent element.

FIG. 9 is a schematic view showing a polypeptide association in an embodiment of the present invention.

FIG. 10 is a conceptual view of a sensor, constructed with an enzyme electrode of the present invention.

FIG. 11 is a conceptual view of a fuel cell, constructed with an enzyme electrode of the present invention.

FIG. 12 is a conceptual view of an enzyme electrode, utilizing an associated protein formed by glucose dehydrogenase and diaphorase in an embodiment of the present invention.

FIG. 13 is a chart showing a relationship between a substrate concentration of a current density, measured with a glucose sensor of Example 2.

FIG. 14 is a conceptual view of an enzyme electrode constituted by glucose dehydrogenase and diaphorase, in a comparative example of the invention.

### DESCRIPTION OF THE EMBODIMENTS

The enzyme electrode of the present invention has a structure in which an enzyme, capable of being involved in an electrochemical reaction, is immobilized on an electroconductive base member, and the enzyme is constituted of at least an associated protein formed by associating two or more enzyme proteins. The immobilization of the enzyme is executed when necessitated, and is not essential in the present invention. The enzyme is preferably immobilized in an enzyme immobilizing layer, provided on the conductive base member. Also the enzyme immobilizing layer preferably further contains an electron transfer mediator, which electrically connects the enzyme and the conductive base member. The associated protein can be formed by combining two or more enzyme proteins, which provide the effects of the present invention. A preferred example of the combination of the two enzyme proteins constituting the associated protein is a combination of an enzyme 1 (first enzyme) and an enzyme 2 (second enzyme) shown below:

Enzyme 1, catalyzing a chemical reaction which generates a reaction product 1 (first reaction product) from a reaction substrate 1 (first reaction substrate); and

Enzyme 2, catalyzing a chemical reaction which generates a reaction product 2 (second reaction product) from a reaction substrate 2 (second reaction substrate). At least a chemical substance of the reaction product 1 of the reaction involving the enzyme 1 is same as at least a chemical substance of the reaction substrate 2.

Specific examples of the preferable combination of enzymes include the following ones:

(1) Enzyme 1 is dehydrogenase, and enzyme 2 is diaphorase;

(2) Enzyme 1 is alcohol dehydrogenase and enzyme 2 is aldehyde dehydrogenase. In this case, diaphorase is preferably immobilized further in the enzyme immobilizing layer;

(3) Enzyme 1 is isomerase and enzyme 2 is glucose dehydrogenase. In this case, diaphorase is preferably immobilized further in the enzyme immobilizing layer

In the enzyme electrode of the present invention, the conductive base member is in contact with the enzyme immobilizing layer, and is electrically connected with the external circuit when the enzyme electrode is in use. The conductive base member may be formed by a material which has, in an interface with the enzyme immobilizing layer, at least a conductive portion that can be electrically connected with the external circuit, and which has a rigidity sufficient for storage and measurement and a sufficient electrical and chemical stability under the condition of use of the electrode. Examples of the material to be employed in the conductive portion include a metal, a conductive polymer, a metal oxide and a carbon material.

Examples of the metal include Au, Pt, Ag and substances containing at least one of these elements. Such substances may be an alloy. The metal material may also be utilized as a plated film or layer.

Examples of the conductive polymer to be employed in the conductive base member include those containing at least a compound among polyacetylenes, polyarylenes and polyarylenevinylenes. Examples of the metal oxide to be employed in forming the conductive base member include those containing at least an element of In, Sn, Zn and Ti. In the case that the metal oxide does not have an electric conductivity or is insufficient in the electric conductivity, a material for the conductive base member having a desired conductivity, by mixing the metal oxide and another conductive material. Examples of the conductive material that can be used by mixing with the metal oxide include metals, conductive polymers and carbon materials. Examples of the carbon material include graphite, carbon black, carbon nanotube, carbon nanohorn, fullerene compounds and derivatives thereof. In the case that the portion formed by the carbon material is not conductive, a conductivity may be provided in such portion by another conductive material. Also even when the portion formed by the carbon material is has a conductivity, the conductivity in such portion may be improved by another conductive material.

The conductive base member may include a cavity at least in a part thereof, and such cavity may be continuous one-dimensionally, two-dimensionally or three-dimensionally. An example of the one-dimensionally continuous cavity is a columnar cavity, and an example of the two-dimensionally continuous cavity is a network-like cavity. Also an example of the three-dimensionally continuous cavity is a sponge-like cavity, a cavity formed after adhering fine particles, or a cavity of a structural material prepared utilizing the aforementioned cavity as a template. Such cavity is preferably large enough allowing to introduce enzyme and/or allowing sufficient flow and diffusion of the substrate and small enough allowing to obtain a sufficient ratio of an effective surface area to a projected area. An example of an average size of the cavity is within a range of from 5 nm to 500 μm, more preferably from 10 nm to 10 μm. Also the conductive base member is required to have a thickness so large enough that the enzyme can be uniformly introduced into a deep part of the conductive base member and/or as to obtain sufficient flow and diffusion of the substrate and so small enough as to obtain a sufficient ratio of an effective surface area to a projected area. An example of the thickness of the conductive base member is within a range of from 100 nm to 1 cm, more preferably from 1 μm to 5 mm. As to the ratio of the effective surface area to the projected area, the conductive base member having cavity is required to have a sufficient large ratio of the effective surface area to the projected area, for example 10 times or larger and more preferably 100 times or larger.

In the conductive base member having cavity, a pore rate is preferably so selected as to satisfy at least one of the following requirements (1) to (3) and a requirement (4):

(1) so large enough as to obtain a sufficient large ratio of the effective surface area to the projected area;

(2) so large enough that the enzyme or carrier can be introduced in a sufficient amount;

(3) so large enough that the substrate can flow and diffuse sufficiently;

(4) so small enough as to obtain a sufficient mechanical strength.

An example of the pore rate is from 20 to 99%, preferably from 30 to 98%.

Examples of the metallic conductive base member having plural cavities include a foamed metal, an electrodeposited metal, an electrolytic metal, a sintered metal, a fibrous metal and a material corresponding to one or more thereof.

Producing methods for a conductive polymer containing plural cavities include those utilized for producing a porous resin, including examples shown below:

(1) A method of placing, in a conductive polymer, a material serving as a mold for a cavity portion, then molding the conductive polymer into a predetermined shape, and removing the mold material;

(2) A method of including, in a precursor of a conductive polymer, a material serving as a mold for a cavity portion, then polymerizing the precursor to obtain a polymer, and removing the mold material;

(3) A method of forming a layer of particles serving as a mold for a cavity portion, then filling a polymer in the space between the particles thereby forming a layer, and removing the particles from the layer;

(4) A method of forming a layer of particles serving as a mold for a cavity portion, then filling a precursor of a polymer in the space between the particles thereby forming a layer, polymerizing the precursor to obtain a polymer layer and removing the particles from the layer.

Examples of the producing method for the metal oxide having plural cavities include an electrodeposition, a sputtering, a sintering, a chemical vapor deposition (CVD), an electrolysis and a combination thereof.

Examples of the producing method for the carbon material having plural cavities include a method of molding fibers or particles formed by graphite, carbon black, carbon nanotube, carbon nanohorn, a fullerene compound or a derivative thereof into a predetermined shape, followed by a sintering.

In the case that the enzyme electrode of the present invention includes an enzyme immobilizing layer, such enzyme immobilizing layer is positioned in contact with the conductive base member. More specifically, the enzyme immobilizing layer is laminated directly on an electroconductive surface of the conductive base member. By forming the enzyme immobilizing layer directly on the conductive base member, the enzyme and the electron transfer mediator, which is added when necessitated, can be captured in a physical proximity of the conductive base member. As a result, a prompt electron transferring reaction between the enzyme and the conductive base member, via the electron transfer mediator when present, can be promoted, and the enzyme and the electron transfer mediator are prevented from being lost from the proximity of the conductive base member, whereby the enzyme electrode may be rendered repeatedly usable and may be improved in the durability.

The enzyme immobilizing layer, to be used for capturing at least an enzyme (enzyme formed by an associated protein) in the physical proximity of the conductive base member, can be prepared by a method already known to those skilled in the art. Examples of the specific method for preparing the enzyme immobilizing layer include those (1) to (6) shown below:

(1) Covalent Bond Method:

A functional group is directly introduced onto the surface of the conductive base member, and the functional group and the enzyme are covalent bonded to immobilize the enzyme. Otherwise, a functional group is introduced into a carrier provided in contact with the conductive base member, and the functional group and the enzyme are covalent bonded to immobilize the enzyme.

Examples of the functional group usable for such covalent bonding include a hydroxyl group, a carboxyl group and an amino group.

Otherwise, utilizing a fact that a thiol group of an alkylthiol reacts and is bonded with a metal such as gold to easily form a monomolecular layer (self-assembling monolayer), the enzyme is immobilized via a functional group introduced in advance into the alkyl group of alkylthiol. A covalent bonding between the functional group introduced in advance into the alkyl group of alkylthiol and the enzyme may be formed for example with a bifunctional reagent. Representative examples of the bifunctional reagent include glutaraldehyde, periodic acid and N,N′-o-phenylene dimaleimide.

(2) Crosslinking Method:

A crosslinking agent such as glutaraldehyde is used to form a crosslink between enzymes, thereby bonding and immobilizing the enzymes. Otherwise, a matrix substance such as gelatin or albumin is added to an enzyme to form a crosslink between the matrix substance and the enzyme, thereby immobilizing the enzyme. At the immobilization, it is possible to also add a synthetic polymer such as polyallylamine or polylysine, thereby controlling the characteristics of the enzyme immobilizing layer such as a layer strength or a substrate transmission property.

(3) Enclosing Method:

An enzyme is immobilized by enclosing in a polymer matrix, such as agarose, a decomposition product of agarose, K-carrageenan, agar, alginic acid, polyacrylamide, polyisopropylacrylamide, polyvinyl alcohol or a copolymer thereof.

(4) Adsorption Method (No. 1):

An enzyme is immobilized by a physical adsorption utilizing a hydrophobic interaction of a carrier and an enzyme. The carrier may be formed by a polyanion or a polycation, such as polyallylamine, polylysine, polyvinylpyridine, amino group-denatured dextran (for example DEAE-dextran), chitosan, polyglutamate, polystyrenesulfonic acid, or dextran sulfate. The enzyme is immobilized on the carrier by an ionic bonding utilizing an electrostatic interaction between the carrier and the enzyme, and such carrier, with immobilized enzyme, is positioned in contact with the conductive base member.

(5) Membrane Method:

A transmission restricting film such as a polyimide film, a cellulose acetate film, a polysulfone film, or a perfluorosulfonic acid polymer film (for example Nafion (trade name) of DuPont) is used as a membrane to cover and immobilize an enzyme on the conductive base member.

(6) Adsorption Method (No. 2):

An enzyme is immobilized utilizing various affinity tags used for facilitating purification of recombinant DNA proteins. For example, the enzyme is immobilized by an epitope tage such as HA (hemaglutinine), FLAG or Myc; GST, a maltose-bonded protein, a biotinized peptide, or an oligohistidine tag.

The associated protein to be employed in the enzyme electrode of the present invention is formed by an association of two or more different enzyme proteins. More specifically, the associated protein employed in the present invention is formed by preparing plural proteins and placing them under a condition capable of an association. Therefore, the associated protein employed in the present invention is structurally different from a fused member of two or more proteins produced by a recombinant DNA technology.

The associated protein can be obtained by combining plural enzyme proteins capable of providing the effects of the present invention. Preferred examples of the combination of two proteins include a combination of enzyme 1 and enzyme 2 as described above. The associated protein in such case is a polyfunctional enzyme, having an enzyme activity of the enzyme 1 and that of the enzyme 2. At least a chemical substance of the reaction product 1, generated by the enzyme activity of the enzyme 1, is promptly utilized as a reaction substrate for the enzyme 2, present in the physical proximity of the enzyme 1. As a result, an enzyme activity (reaction speed) of the associated protein for generating the reaction product 2 from the reaction substrate 1 becomes higher than in a case where the enzyme 1 and the enzyme 2 are separated from each other.

The specific combination of the enzyme 1 and the enzyme 2 is not particularly restricted, as long as at least a chemical substance in the reaction product 1 of the enzyme 1 is same as at least a chemical substance in the reaction substrate 2 of the enzyme 2. A case where the enzyme 1 is dehydrogenase and the enzyme 2 is a diaphorase is preferable as it can reduce the influence of oxygen on an electrochemical response, observed on the enzyme electrode by an oxidation reaction of the substrate. Also many dehydrogenases utilize nicotinamide adenine nucleotide or nicotinamide adenine nucleotidephosphoric acid as receptors for an electron and a hydrogen atom. It is therefore possible to construct a sensor having a wide applicability to the objects of detection, by selecting the type of dehydrogenase to be associated with diaphorase. Examples of such dehydrogenase include glucose dehydrogenase, alcohol dehydrogenase, glutamic acid dehydrogenase, cholesterol dehydrogenase, aldehyde dehydrogenase, fructose dehydrogenase, sorbitol dehydrogenase, lactic acid dehydrogenase, malic acid dehydrogenase, glycerol dehydrogenase, 17B hydroxysteroid dehydrogenase, estradiol 17B dehydrogenase, amino acid dehydrogenase, glyceraldehyde 3-phosphate dehydrogenase, and 3-hydroxysteroid dehydrogenase.

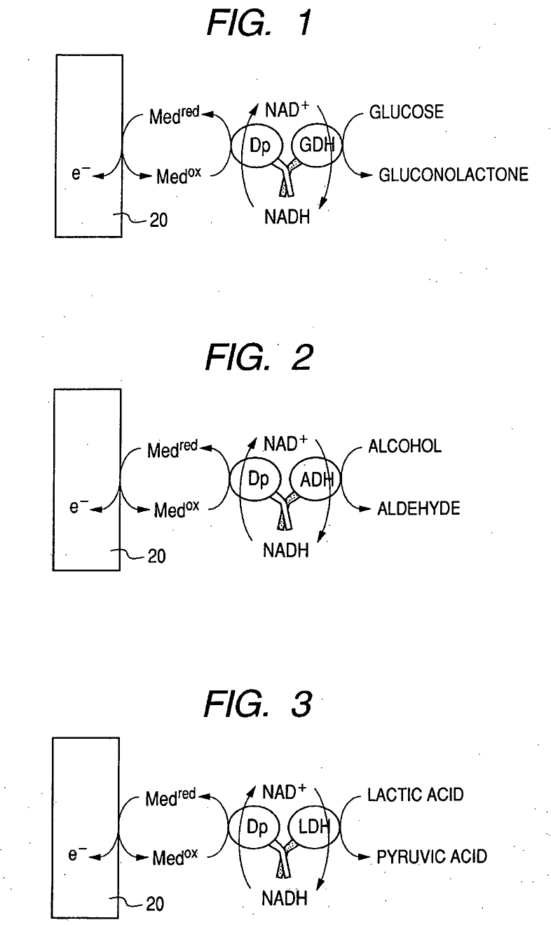

As an example, in case of constructing an enzyme electrode by employing glucose dehydrogenase as the enzyme 1 (first enzyme) and diaphorase (Dp) as the enzyme 2 (second enzyme) and utilizing an associated protein thereof, there can be obtained a state shown in FIG. 1. An enzyme immobilizing layer, containing an associated protein of glucose dehydrogenase (GDH) and diaphorase, and an electron transfer mediator, is provided on and in contact with the conductive base member. When glucose (first reaction substrate) is made to act on the enzyme electrode, glucose is oxidized by the catalytic action of glucose dehydrogenase in the presence of nicotinamide adenine dinucleotide (NAD⁺). As a result, gluconolactone (first reaction product) and nicotinamide adenine dinucleotide (NADH) in a reduced form are generated. The reduced nicotinamide adenine dinucleotide is immediately oxidized, in the presence of the oxidizing electron transfer mediator, by the catalytic action of diaphorase, present in the physical proximity of glucose dehydrogenase. As a result, nicotinamide adenine dinucleotide and the electron transfer mediator in a reduced form are generated. The thus obtained electron transfer mediator in the reduced form can transport an electron to the conductive base member. In comparison with the prior structure in which glucose dehydrogenase and diaphorase are individually immobilized on the electrode, the enzyme electrode of the present invention can achieve a more efficient electron transport from glucose to the electrode. Therefore, the enzyme electrode as shown in FIG. 1 may be utilized as a glucose sensor of a higher detection sensitivity, or as a glucose fuel cell of a higher output, or as an electrochemical reaction device for glucose, having a higher reaction efficiency.

In particular, by employing an associated protein of glucose dehydrogenase and diaphorase derived from thermophilic bacteria, there can be obtained an enzyme electrode improved in heat resistance, durability, and response at a high temperature state.

In the aforementioned example, the first reaction substrate is glucose and NAD⁺, and the first reaction product is gluconolactone and NADH. Also the second reaction substrate is NADH and Med^{ox}, and the second reaction product is NAD⁺ and Med^{red}. Thus NADH is a first reaction product, and also is a second reaction substrate.

In case of constructing an enzyme electrode by employing alcohol dehydrogenase as the enzyme 1 and diaphorase as the enzyme 2, there can be obtained a state shown in FIG. 2. An enzyme immobilizing layer, containing an associated protein of alcohol dehydrogenase and diaphorase, and an electron transfer mediator, is provided on and in contact with the conductive base member. When alcohol is made to act on the enzyme electrode, alcohol is oxidized by the catalytic action of alcohol dehydrogenase in the presence of nicotinamide adenine dinucleotide. As a result, aldehyde and nicotinamide adenine dinucleotide in a reduced form are generated. The reduced nicotinamide adenine dinucleotide is immediately oxidized, in the presence of the oxidizing electron transfer mediator, by the catalytic action of diaphorase present in the physical proximity of alcohol dehydrogenase. As a result, nicotinamide adenine dinucleotide and the electron transfer mediator in a reduced form are generated.

In case of constructing an enzyme electrode by employing lactic acid dehydrogenase as the enzyme 1 and diaphorase as the enzyme 2, there can be obtained a state shown in FIG. 3. An enzyme immobilizing layer, containing an associated protein of lactic acid dehydrogenase and diaphorase, and an electron transfer mediator, is provided on and in contact with the conductive base member. When lactic acid is made to act on the enzyme electrode, lactic acid is oxidized by the catalytic action of lactic acid dehydrogenase in the presence of nicotinamide adenine dinucleotide. As a result, pyruvic acid and nicotinamide adenine dinucleotide in a reduced form are generated. The reduced nicotinamide adenine dinucleotide is immediately oxidized, in the presence of the oxidizing electron transfer mediator, by the catalytic action of diaphorase present in the physical proximity of lactic acid dehydrogenase. As a result, nicotinamide adenine dinucleotide and the electron transfer mediator in a reduced form are generated.

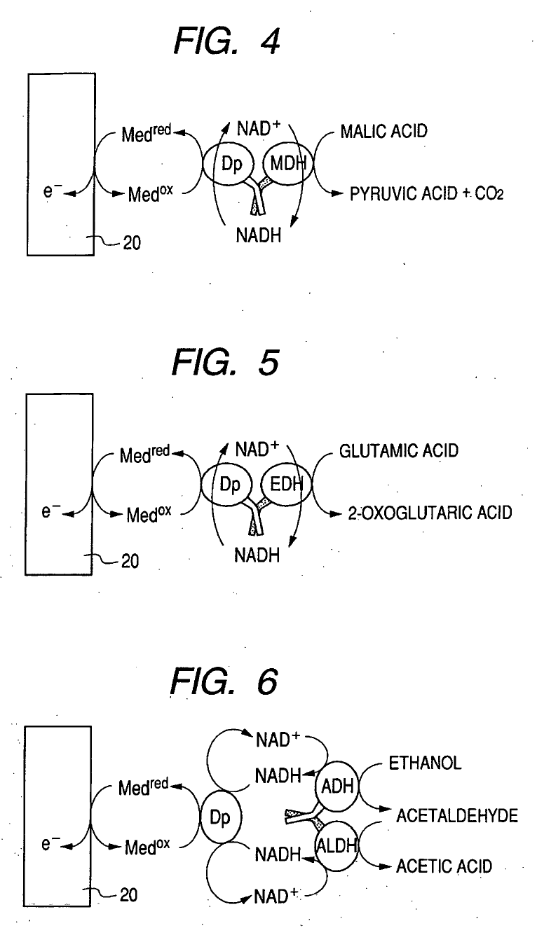

In case of constructing an enzyme electrode by employing malic acid dehydrogenase as the enzyme 1 and diaphorase as the enzyme 2, there can be obtained a state shown in FIG. 4. An enzyme immobilizing layer, containing an associated protein of malic acid dehydrogenase and diaphorase, and an electron transfer mediator, is provided on and in contact with the conductive base member. When malic acid is made to act on the enzyme electrode, malic acid is oxidized by the catalytic action of malic acid dehydrogenase in the presence of nicotinamide adenine dinucleotide. As a result, pyruvic acid and nicotinamide adenine dinucleotide in a reduced form are generated. The reduced nicotinamide adenine dinucleotide is immediately oxidized, in the presence of the oxidizing electron transfer mediator, by the catalytic action of diaphorase present in the physical proximity of malic acid dehydrogenase. As a result, nicotinamide adenine dinucleotide and the electron transfer mediator in a reduced form are generated.

In case of constructing an enzyme electrode by employing glutamic acid dehydrogenase as the enzyme 1 and diaphorase as the enzyme 2, there can be obtained a state shown in FIG. 5. An enzyme immobilizing layer, containing an associated protein of glutamic acid dehydrogenase and diaphorase, and an electron transfer mediator, is provided on and in contact with the conductive base member. When glutamic acid is made to act on the enzyme electrode, glutamic acid is oxidized by the catalytic action of glutamic acid dehydrogenase in the presence of nicotinamide adenine dinucleotide. As a result, 2-oxoglutaric acid and nicotinamide adenine dinucleotide in a reduced form are generated. The reduced nicotinamide adenine dinucleotide is immediately oxidized, in the presence of the oxidizing electron transfer mediator, by the catalytic action of diaphorase present in the physical proximity of glutamic acid dehydrogenase. As a result, nicotinamide adenine dinucleotide and the electron transfer mediator in a reduced form are generated.

In case of constructing an enzyme electrode by employing alcohol dehydrogenase as the enzyme 1 and aldehyde dehydrogenase as the enzyme 2, there can be obtained a state shown in FIG. 6. An enzyme immobilizing layer, containing an associated protein of alcohol dehydrogenase and aldehyde dehydrogenase, diaphorase and an electron transfer mediator, is provided on and in contact with the conductive base member. When alcohol is made to act on the enzyme electrode, alcohol is oxidized by the catalytic action of alcohol dehydrogenase in the presence of nicotinamide adenine dinucleotide. As a result, an aldehyde and nicotinamide adenine dinucleotide in a reduced form are generated. Thus generated aldehyde is immediately oxidized, in the presence of nicotinamide adenine dinucleotide, by the catalytic action of aldehyde dehydrogenase present in the physical proximity of alcohol dehydrogenase. As a result, a carboxylic acid and nicotinamide adenine dinucleotide in a reduced form are generated. The reduced nicotinamide adenine dinucleotide, thus generated, is oxidized by the catalytic action of diaphorase in the presence of the oxidizing electron transfer mediator, thereby generating nicotinamide adenine dinucleotide and the electron transfer mediator in a reduced form. The reduced electron transfer mediator, thus generated, can transport an electron to the conductive base member. Also such enzyme electrode can prevent an accumulation of the aldehyde in the proximity of the enzyme electrode, thereby alleviating a deactivation reaction of enzyme protein by aldehyde and suppressing a loss in the activity of the enzyme electrode. In comparison with the prior structure in which alcohol dehydrogenase and aldehyde dehydrogenase are individually immobilized on the electrode, the enzyme electrode of the present invention can achieve a higher efficiency in the oxidation reaction from alcohol to carboxylic acid, and in the electron transport to the electrode. Therefore, the enzyme electrode as shown in FIG. 6 may be utilized as an alcohol sensor of a high detection sensitivity, or as an alcohol fuel cell of a high output, or as an electrochemical reaction device for alcohol, having a high reaction efficiency.

In particular, by employing an associated protein of alcohol dehydrogenase and aldehyde dehydrogenase derived from thermophilic bacteria, there can be obtained an enzyme electrode excellent in heat resistance, durability, and response at a high temperature state.

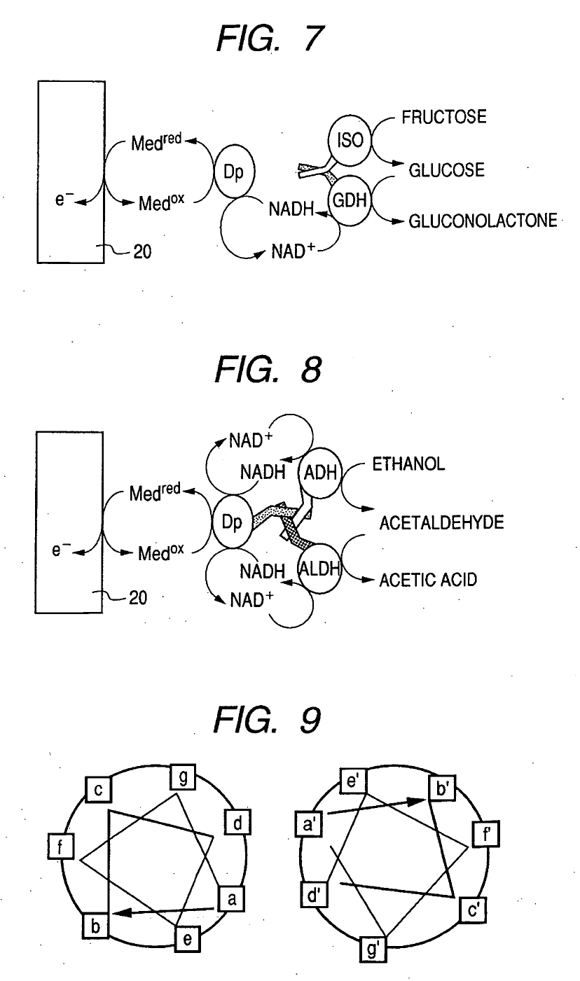

In case of constructing an enzyme electrode by employing isomerase as the enzyme 1 and glucose dehydrogenase as the enzyme 2, there can be obtained a state shown in FIG. 7. An enzyme immobilizing layer, containing an associated protein of isomerase and glucose dehydrogenase, diaphorase and an electron transfer mediator, is provided on and in contact with the conductive base member. When fructose is made to act on the enzyme electrode, fructose is isomerized by the catalytic action of isomerase, thereby generating glucose. Thus generated glucose is immediately oxidized, in the presence of nicotinamide adenine dinucleotide, by the catalytic action of glucose dehydrogenase present in the physical proximity of isomerase, thereby generating gluconolactone and nicotinamide adenine dinucleotide in a reduced form. Thus generated nicotinamide adenine dinucleotide in a reduced form is oxidized, in the presence of the oxidizing electron transfer mediator, by the catalytic action of diaphorase. As a result, nicotinamide adenine dinucleotide and the electron transfer mediator in a reduced form. The reduced electron transfer mediator, thus generated, can transport an electron to the conductive base member.

The associated protein, constituting the enzyme electrode of the present invention, may also be formed by an association of three or more proteins. For example, in case of constructing an enzyme electrode by employing an associated protein of three enzymes, namely alcohol dehydrogenase, aldehyde dehydrogenase and diaphorase, there can be obtained a state shown in FIG. 8. An enzyme immobilizing layer, containing an associated protein of alcohol dehydrogenase, aldehyde dehydrogenase and diaphorase, and an electron transfer mediator, is provided on and in contact with the conductive base member. When alcohol is made to act on the enzyme electrode, alcohol is oxidized by the catalytic action of alcohol dehydrogenase in the presence of nicotinamide adenine dinucleotide, thereby generating an aldehyde and nicotinamide adenine dinucleotide in a reduced form. Thus generated aldehyde is immediately oxidized, in the presence of nicotinamide adenine dinucleotide, by the catalytic action of aldehyde dehydrogenase present in the physical proximity of alcohol dehydrogenase. As a result, a carboxylic acid and nicotinamide adenine dinucleotide in a reduced form are generated. The reduced nicotinamide adenine dinucleotide, thus generated, is oxidized by the catalytic action of diaphorase, which is present in physical proximity of alcohol dehydrogenase and aldehyde dehydrogenase, in the presence of the oxidizing electron transfer mediator. As a result, nicotinamide adenine dinucleotide and the electron transfer mediator in a reduced form are generated.

(Association Site in Polypeptide)

The associated protein, to be employed in the enzyme electrode of the present invention, preferably has a structure in which plural enzyme proteins are associated, utilizing a part of the polypeptide chain in each of the plural enzyme proteins as an association site. Such association site, formed by peptide, may be in any position as long as not hindering the function (enzyme activity) of each protein.

The association formed by the association sites of the proteins may be based on a covalent bond or a non-covalent bond. Examples of the non-covalent bond include a van der Waals force, a hydrogen bond, an ionic bond and a hydrophobic interaction, which are induced by the associating site, particularly an amino group contained in the associating site.

The association sites of two enzyme proteins are preferably formed by two peptide chains having a complementary interaction. Thus, in case of associating the enzyme 1 and the enzyme 2, a peptide chain, having a complementary interaction with the peptide chain in the association site of the enzyme 1, is provided in the association site of the enzyme 2. An association of three enzyme proteins may be realized by applying such combination of the association sites of two enzyme proteins to two combinations selected from three or more enzyme proteins. The association site may be formed by an oligopeptide, a polypeptide or a protein constituted of plural domains, as long as the complementary interaction is available. As a specific example, an alpha-domain or an omega-domain of β-galactosidase may be utilized. Also antibody segments VH and VL may be used as the polypeptide association site. It is also possible to utilize an α-helical coiled coil structure often found in natural protein structures. The α-helical coiled coil structure is a structure in which several α-helixes are wound with each other under an interaction (association). An amino acid of 7 residues corresponds to 2 rotations of the helix, and 7 positions are often represented, as shown in FIG. 9, by a, b, c, d, e, f and g. Examples of a and d each may be a hydrophobic amino acid (Val, Ile) important for the association between the helixes, or Glu, Lys, Gln or Arg. Val or Ils is particularly preferable. A coiled coil formed by plural α-helixes may be formed by suitably selecting amino acids on such associating faces. It is also possible, by introducing His in the positions a and d, to derive a coiled coil structure formation utilizing a co-existing metal ion Co(II) or Ni(II).

Examples of a model structure of such polypeptide association site include a transcription factor GCN4 formed by a peptide having some repeating amino acids, and a leucine zipper such as a cancer gene Fos or Jun.

Since the associated protein in the enzyme electrode of the present invention is a polyfunctional enzyme having plural different enzyme activities, it is preferable, in associating two enzymes, that the α-helical-coiled coils to be fused are less liable to form a homodimer. In this sense, it is desirable to utilize an α-helical-coiled coil structure for forming a heterodimer such as Jun/Fos, or a heteromacromer. It is also known that a stable hetero-coiled coil structure can be formed by adopting Glu in the positions e and g in one of the two α-helical-coiled coils and adopting Lys in these positions in the other. Such hetero-coiled coil structure may be utilized in the association of two enzyme proteins.

For forming an α-helical-coiled coil structure in a part of the polypeptide chain of the enzyme protein, a method of fusing an α-helical-coiled coil to the polypeptide chain, utilizing the recombinant DNA technique, may be advantageously used.

It is furthermore possible to utilize two polypeptide chains, respectively having mutually opposite charges, as respective association sites of two enzyme proteins.

The peptide association site is selected in such a size that does not hinder a structure formation necessary for productivity or activity of each enzyme protein. Such size is preferably 50 amino acids or less, and more preferably from 15 to 35 amino acids.

Also it is effective, in the peptide association site or in the vicinity thereof, to form a covalent bond between a pair of the association sites associated with each other, in view of stabilization of the associated protein. More specifically, it is possible, in the two polypeptide association sites in the model shown in FIG. 9, to form an intermolecular disulfide bond by introducing Cys in a position a and/or d or a position g and/or e of either peptide chain and, in the other, in at least a position corresponding to these positions and not hindering the enzyme activities of both enzymes. It is known, for example in an α-helical-coiled coil structure formed by two chains, that a disulfide bond in the position a-a is more stable in energy than a disulfide bond in the position d-d. Such introduction is also possible, in case of associating three or more enzyme proteins, under suitable selection. Also under a similar principle, the association site formed of peptide may be subjected to a modification by a photo-crosslinkable group or an introduction of a non-natural amino acid in which a photo-crosslinkable group is introduced. In such case, a photo-functional group is not introduced into all the polypeptides, but is preferably introduced into the association site or the vicinity thereof of at least two polypeptide chains. In case of forming a macromer by three or more enzyme proteins, the introducing position is preferably determined suitably in consideration of the arrangement thereof.

The peptide association site to be provided in each enzyme is preferably designed in such an arrangement as not to hinder the enzyme activity, and, as an example, is preferably expressed as a fusion protein, generally fused at a C-terminal or an N-terminal of the enzyme polypeptide chain. Such fusion protein can be prepared with a recombinant DNA, in which a DNA sequence encoding the amino acid sequence of the association site is connected or inserted at an upstream or downstream side of or in the interior of a gene encoding the amino acid sequence of the enzyme protein, with mutually matching reading frames. In case of inserting the DNA sequence encoding the amino acid sequence of the association site into the interior of the gene encoding the amino acid sequence of the enzyme protein, the inserting position is so selected as not to hinder the function of the enzyme protein after the association. The associated protein can be prepared by expressing such recombinant DNA as a recombinant protein utilizing a suitable host-vector system, then purifying the fusion protein thus expressed, and mixing the proteins respectively fused with the polypeptide association parts thereby reconstructing them as an associated protein. It is also possible to co-express the respective recombinant DNAs in a host cell, thereby constructing the associated protein simultaneously with the expression in the cell.

(Spacer)

In case of fusing an association site, to an amino acid sequence, of an enzyme protein for association, a spacer sequence may be inserted between the amino acid sequence of the enzyme protein and the amino acid sequence of the association site. The spacer sequence preferably satisfies the following requirements:

(1) To enable that each enzyme to be associated assumes a specific folding, and to ensure that each enzyme maintains its enzyme activity; and

(2) In case of employing a combination of two enzymes in which a reaction product of either enzyme is utilized as a substrate of the other, the spacer satisfies the requirement (1) and has such a sequence and a length that the reaction product of either enzyme can reach the other enzyme as promptly as possible by diffusion.

In the requirement (2), the spacer sequence preferably has a length of about from 3 to 400 amino acids. The spacer sequence is not particularly restricted as long as it has the aforementioned properties, and examples thereof include SEQ ID NO:160 (G: glycine, S: serine) and a sequence in which such unit sequence is repeated by about 2 to 5 times. Also the following sequences, described by Patrick (J. Mol. Boil. (1990) 211, 943-958) may also be utilized:

(1) a sequence formed solely of serine (S), threonine (T) and/or glycine (G) (type I (STG type));

(2) a peptide formed, in addition to serine, threonine and/or glycine, and one of aspartic acid (D), lysine (K), glutamine (Q), asparagine (N), alanine (A) and proline (P) (type II);

(3) a combination sequence formed by amino acid residues of the STG types I and II (type III (SEQ ID NO:161 type));

(4) a sequence of type I or II having plural prolines (type IV (Pro type));

(5) a sequence formed by an arbitrary combination of amino acid residues contained in type III and having at least 8 amino acid residues (type V); and SEQ ID NO:162, SEQ ID NO:163, SEQ ID NO:164, SEQ ID NO:165, SEQ ID NO:166, and SEQ ID NO:167.

The DNA sequence encoding the spacer sequence may be inserted between a gene encoding the amino acid sequence of the enzyme protein for association and a DNA sequence encoding the association site, in such a manner that the reading frames thereof mutually match, and may thus be inserted by the technology already known in the art. In case of bonding the association site to an N-terminal or a C-terminal of the enzyme protein for association, a spacer is preferably provided between the association site and the N-terminal or C-terminal of the enzyme protein for association. Also in case of inserting the association site into a polypeptide chain of the enzyme protein for association, a spacer may be inserted at the side of an N-terminal and/or a C-terminal of the association site.

The gene DNA sequence encoding the amino acid sequence of the enzyme constituting the enzyme protein for association is not particularly restricted in the source thereof, as long as the function thereof is identified.

As glucose dehydrogenase (EC 1.1.1.47) for association, there may be utilized those of which a function of catalyzing the following chemical reaction is identified and a base sequence of the gene DNA or an amino acid sequence of the enzyme is already known:
beta-D-glucose+NAD(P)⁺=D-glucono-1,5-lactone+NAD(P)H+H⁺
Examples of such glucose dehydrogenase gene include those derived from Bacilluus genus, such as Bacillus subtilis 168 [Nature 390:249-56(1997)], Gloeobacter genus, such as Gloeobacter violaceus PCC7421 [DNA Res 10:137-45 (2003)], Thermoplasma genus such as Thermoplasma acidophilus DSM 1728 [Nature 407:508-13 (2000)] and Thermoplasma volcanium GSS1 [Proc Natl Acad Sci, USA 97:14257-62 (2000)], Picrophilus genus such as Picrophilus torridus DSM 9790 [Proc Natl Acad Sci, USA 101:9091-6 (2004)], Pyrococcus genus such as Pyrococcus furiosus DSM 3638 [Genetics 152:1299-305 (1999)], and Sulfolobus genus such as Sulfolobus solfataricus [Proc Natl Acad Sci, USA 98:7835-40 (2001)] and Sulfolobus tokodaii strain 7 [DNA Res 8:123-40 (2001)], any of which is usable as a constituent of the associated protein in the present invention. Particularly the enzymes derived from Pyrococcus furiosus and Sulfolobus solfataricus have heat resistance, and are usable advantageously in the present invention.

As alcohol dehydrogenase (EC 1.1.1.1) for association, there may be utilized those of which a function of catalyzing the following chemical reaction is identified and a base sequence of the gene DNA or an amino acid sequence of the enzyme is already known:
alcohol+NAD⁺=aldehyde or ketone+NADH+H⁺
Examples of such alcohol dehydrogenase gene include those derived from Saccharomyces genus, such as Saccharomyces cerevisiae S288C [Science 274:546-67 (1996)], Pseudomonas genus, such as Pseudomonas aeruginosa PA01 [Nature 406:959-64 (2000)] or Pseudomonas putida KT2440 [Environ Microbiol 4:799-808 (2002)], Acinetobacter genus, such as Acinetobacter sp. ADP1 [Nucleic Acids Res 32:5766-79 (2004)], Bacillus genus, such as Bacillus subtilis 168 [Nature 390:249-56 (1997)], Lactococcus genus, such as Lactococcus lactis subsp. lactis IL1403 [Genome Res 11:731-53 (2001)], Lactobacillus genus, such as Lactobacillus plantarum WCFS1 [Proc Natl Acad Sci USA 100:1990-5 (2003)], Thermus genus, such as Thermus thermophilus HB27[Nat Biotechnol 22:547-53 (2004)], Aquifex genus, such as Aquifex aeolicus VF5[Nature 392:353-8 (1998)], Thermotoga genus, such as Thermotoga maritima MSB8[Nature 399:323-9 (1999)], Methanococcus genus, such as Methanococcus maripaludis S2[J Bacteriol 186:6956-69 (2004)], Methanosarcina genus, such as Methanosarcina acetivorans C2A[Genome Res 12:532-42 (2002)] or Methanosarcina mazei Goel [J Mol Microbiol Biotechnol 4:453-61 (2002)], Thermoplasma genus such as Thermoplasma acidophilum DSM 1728 [Nature 407:508-13 (2000)] or Thermoplasma volcanium GSS1 [Proc Natl Acad Sci USA 97:14257-62 (2000)], Pyrococcus genus such as Pyrococcus horikoshii OT3 [DNA Res 5:55-76 (1998)], Pyrococcus abyssi GE5 or Pyrococcus furiosus DSM 3638 [Genetics 152:1299-305 (1999), Mol Microbiol 38:684-93 (2000), Methods Enzymol 330:134-57 (2001)], Aeropyrum genus, such as Aeropyrum pernix K1 [DNA Res 6:83-101, 145-52 (1999)], Sulfolobus genus, such as Sulfolobus solfataricus [Proc Natl Acad Sci USA 98:7835-40 (2001)] or Sulfolobus tokodaii strain7 [DNA Res 8:123-40 (2001)], and Pyrobaculum genus, such as Pyrobaculum aerophilum IM2[Proc Natl Acad Sci USA 99:984-9 (2002)], any of which is usable as a constituent of the associated protein in the present invention. Particularly the enzymes derived from Corynebacterium efficiens, Thermus thermophilus Aquifex aeolicus, Thermotoga maritima, Archaeoglobus fulgidus, Pyrococcus horikoshii, Pyrococcus abyssi, Pyrococcus furiosus, Aeropyrum pernix, and Pyrobaculum aerophilum have heat resistance, and are usable advantageously in the present invention.

As aldehyde dehydrogenase for association, there may be utilized those of which a function of catalyzing the following chemical reaction (1) or (2) is identified and a base sequence of the gene DNA or an amino acid sequence of the enzyme is already known:

(1) enzyme (EC 1.2.1.3) requiring NAD as an electron acceptor:
aldehyde+NAD⁺+H₂O=acid+NADH+H⁺

(2) enzyme (EC 1.2.1.5) requiring NAD or NADP as an electron acceptor:
aldehyde+NAD(P)+H₂O=acid+NAD(P)H+H⁺
In particular, as aldehyde dehydrogenase for oxidizing acetaldehyde, there may be utilized those of which a function of catalyzing the following chemical reaction is identified (EC 1.2.1.10) and a base sequence of the gene DNA or an amino acid sequence of the enzyme is already known:
acetaldehyde+CoA+NAD⁺=acetyl-CoA+NADH+H⁺
Examples of such glucose dehydrogenase gene, in case of aldehyde dehydrogenase requiring NAD as an electron acceptor (EC 1.2.1.3), include those derived from Acinetobacter genus, such as Acinetobacter sp. ADP1 [Nucleic Acids Res 32:5766-79 (2004)], Bacillus genus, such as Bacillus subtilis 168 [Nature 390:249-56 (1997)], Thermus genus, such as Thermus thermophilus HB27 [Nat Biotechnol 22:547-53 (2004)], Pyrococcus genus, such as Pyrococcus furiosus DSM 3638 [Genetics 152:1299-305 (1999), Mol Microbiol 38:684-93 (2000), Methods Enzymol 330:134-57 (2001)], and Aquifex genus such as Aquifex aeolicus VF5 [Nature 392:353-8 (1998)], any of which is usable as a constituent of the associated protein in the present invention.

Also in case of aldehyde dehydrogenase requiring NAD or NADP as an electron acceptor (EC 1.2.1.5), there are included for example those derived from Caenorhabditis genus, such as Caenorhabditis elegans [Science 282:2012-8 (1998)], and Bacillus genus, such as Bacillus thuringiensis 97-27 (serovar konkukian), any of which is usable as a constituent of the associated protein in the present invention.

Also in case of acetaldehydro dehydrogenase (EC 1.2.1.10), there are included for example those derived from Bacillus genus, such as Bacillus cereus ATCC 14579[Nature 423:87-91 (2003)], and Bifidobacterium genus, such as Bifidobacterium longum NCC2705 [Proc Natl Acad Sci USA 99:14422-7 (2002)], any of which is usable as a constituent of the associated protein in the present invention.

In particular, enzymes derived for example from Thermus thermophilus, Pyrococcus furiosus, and Aquifex aeolicus have heat resistance, and can be utilized advantageously in the present invention.

As lactic acid dehydrogenase (EC 1.1.1.27) for association, there may be utilized those of which a function of catalyzing the following chemical reaction is identified and a base sequence of the gene DNA or an amino acid sequence of the enzyme is already known:
(S)-lactate +NAD⁺=pyruvate+NADH+H⁺
Examples of such lactic acid dehydrogenase gene include those derived from Bacillus genus, such as Bacillus subtilis 168 [Nature 390:249-56 (1997)], Lactococcus genus, such as Lactococcus lactis subsp. lactis IL1403 [Genome Res 11:731-53 (2001)], Lactobacillus genus, such as Lactobacillus plantarum WCFS1[Proc Natl Acad Sci USA 100:1990-5 (2003)) or Lactobacillus johnsonii NCC 533 [Proc Natl Acad Sci USA : (2004)], Deinococcus genus, such as Deinococcus radiodurans R1 [Science 286:1571-7 (1999)], Thermus genus, such as Thermus thermophilus HB27 [Nat Biotechnol 22:547-53 (2004)], and Thermotoga genus, such as Thermotoga maritima MSB8 [Nature 399:323-9 (1999)], any of which is usable as a constituent of the associated protein in the present invention. In particular, enzymes derived for example from Thermus thermophilus and Thermotoga maritima have heat resistance, and can be utilized advantageously in the present invention.

As diaphorase for association, there may be utilized those of which a diaphorase activity is confirmed and a base sequence of the gene DNA or an amino acid sequence of the enzyme is already known , any of which is usable as a constituent of the associated protein in the present invention. The diaphorase activity means a catalytic activity for a reaction of oxidizing NADH or NADPH in the presence of an artificial electron acceptor such as methylene blue, or 2,6-dichlorophenol-indophenol. The enzymes having such diaphorase activity are classified, depending on specificity on either of NADH and NADPH or both thereof, as follows:

EC 1.6.99.1; NADPH:(acceptor) oxidoreductase

EC 1.6.99.2; NAD(P)H:(quinone-acceptor) oxidoreductase

EC 1.6.99.3; NADH:(acceptor) oxidoreductase

EC 1.6.99.5; NADH:(quinone-acceptor) oxidoreductase

EC 1.8.1.4; protein-N6-(dihydrolipoyl)lysine:NAD+oxidoreductase

EC 1.14.13.39; L-arginine, NADPH:oxygen oxidoreductase

(nitric-oxide-forming)

In the enzyme electrode of the present invention, depending on whether dehydrogenase, to be associated or to be co-existing with diaphorase, has a substrate specificity whether on NADPH or NADH, it is desirable to select a diaphorase having a substrate specificity same as that of dehydrogenase.

In case of employing diaphorase (1) above, there may be utilized those of which a function of catalyzing the following chemical reaction is identified and a base sequence of the gene DNA or an amino acid sequence of the enzyme is already known:
NADPH+H⁺+acceptor=NADP⁺+reduced acceptor
Examples of such diaphorase gene include those derived from Saccharomyces genus, such as Saccharomyces cerevisiae S288C (Science 274:546-67 (1996), Proc Natl Acad Sci USA 92:3809-13 (1995), EMBO J 13:5795-809 (1994), Nature 357:38-46 (1992), Nature 387:75-8 (1997), Nature 387:78-81 (1997), Nat Genet 10:261-8 (1995), Nature 387:81-4 (1997), Science 265:2077-82 (1994), Nature 387:84-7 (1997), EMBO J 15:2031-49 (1996), Nature 369:371-8 (1994), Nature 387:87-90 (1997), Nature 387:90-3 (1997), Nature 387:93-8 (1997), Nature 387:98-102 (1997), Nature 387:103-5 (1997)], and Candida genus, such as Candida albicans SC5314 [Proc Natl Acad Sci USA 101:7329-34 (2004)].

In case of employing diaphorase (2) above, there may be utilized those of which a function of catalyzing the following chemical reaction is identified and a base sequence of the gene DNA or an amino acid sequence of the enzyme is already known:
NAD(P)H+H⁺+acceptor=NAD(P)+reduced acceptor
Examples of such diaphorase gene include those derived from Pseudomonas genus, such as Pseudomonas aeruginosa PA01 [Nature 406:959-64 (2000)] or Pseudomonas putida KT2440 [Environ Microbiol 4:799-808 (2002)], and Bacillus genus, such as Bacillus cereus ATCC 14579 [Nature 423:87-91 (2003)].

In case of employing diaphorase (3) above, there may be utilized those of which a function of catalyzing the following chemical reaction is identified and a base sequence of the gene DNA or an amino acid sequence of the enzyme is already known:
NADH+H⁺+acceptor=NAD⁺+reduced acceptor
Examples of such diaphorase gene include those derived from Saccharomyces genus, such as Saccharomyces cerevisiae S288C [Science 274:546-67 (1996), Proc Natl Acad Sci USA 92:3809-13 (1995), EMBO J 13:5795-809 (1994), Nature 357:38-46 (1992), Nature 387:75-8 (1997), Nature 387:78-81 (1997), Nat Genet 10:261-8 (1995), Nature 387:81-4 (1997), Science 265:2077-82 (1994), Nature 387:84-7 (1997), EMBO J 15:2031-49 (1996), Nature 369:371-8 (1994), Nature 387:87-90 (1997), Nature 387:90-3 (1997), Nature 387:93-8 (1997), Nature 387:98-102 (1997), Nature 387:103-5 (1997)], Pseudomonas genus, such as Pseudomonas aeruginosa PA01 [Nature 406:959-64 (2000)] or Pseudomonas putida KT2440 [Environ Microbiol 4:799-808 (2002)], Acinetobacter genus, such as Acinetobacter sp. ADP1 [Nucleic Acids Res 32:5766-79 (2004)], Bacillus genus, such as Bacillus subtilis 168 [Nature 390:249-56 (1997)], Lactobacillus genus, such as Lactobacillus plantarum WCFS1 [Proc Natl Acad Sci USA 100:1990-5 (2003)], Deinococcus genus, such as Deinococcus radiodurans R1 [Science 286:1571-7 (1999)], Thermus genus, such as Thermus thermophilus HB27 [Nat Biotechnol 22:547-53 (2004)), Aquifex genus, such as Aquifex aeolicus VF5 [Nature 392:353-8 (1998)], and Pyrococcus genus, such as Pyrococcus abyssi GE5, or Pyrococcus furiosus DSM 3638 [Genetics 152:1299-305 (1999), Mol Microbiol 38:684-93 (2000), Methods Enzymol 330:134-57 (2001)].

In case of employing diaphorase (4) above, there may be utilized those of which a function of catalyzing the following chemical reaction is identified and a base sequence of the gene DNA or an amino acid sequence of the enzyme is already known:
NADH+H⁺+acceptor=NAD⁺+reduced acceptor
Examples of such diaphorase gene include those derived from Burkholderia genus, such as Burkholderia mallei ATCC 23344 [Proc Natl Acad Sci USA 101:14246-51 (2004)], and Haloarcula genus, such as Haloarcula marismortui ATCC 43049 [Genome Res 14:2221-34 (2004)].

In case of employing diaphorase (5) above, there may be utilized those of which a function of catalyzing the following chemical reaction is identified and a base sequence of the gene DNA or an amino acid sequence of the enzyme is already known:
protein N6-(dihydrolipoyl)lysine+NAD⁺=protein N6-(lipoyl)lysine+NADH+H⁺
Examples of such diaphorase gene include those derived from Saccharomyces genus, such as Saccharomyces cerevisiae S288C [Science 274:546-67 (1996), Proc Natl Acad Sci USA 92:3809-13 (1995), EMBO J 13:5795-809 (1994), Nature 357:38-46 (1992), Nature 387:75-8 (1997), Nature 387:78-81 (1997), Nat Genet 10:261-8 (1995), Nature 387:81-4 (1997), Science 265:2077-82 (1994), Nature 387:84-7 (1997), EMBO J 15:2031-49 (1996), Nature 369:371-8 (1994),Nature 387:87-90 (1997), Nature 387:90-3 (1997), Nature 387:93-8 (1997), Nature 387:98-102 (1997), Nature 387:103-5 (1997)], Lactobacillus genus, such as Lactobacillus plantarum WCFS1 [Proc Natl Acad Sci USA 100:1990-5 (2003)], Deinococcus genus, such as Deinococcus radiodurans R1 [Science 286:1571-7 (1999)], Thermus genus, such as Thermus thermophilus HB27 [Nat Biotechnol 22:547-53 (2004)], Aquifex genus, such as Aquifex aeolicus VF5 [Nature 392:353-8 (1998)], Thermotoga genus, such as Thermotoga maritima MSB8 [Nature 399:323-9 (1999)], Sulfolobus genus, such as Sulfolobus solfataricus [Proc Natl Acad Sci USA 98:7835-40 (2001)], or Sulfolobus tokodaii strain7 [DNA Res 8:123-40 (2001)], and Pyrobaculum genus, such as Pyrobaculum aerophilum IM2 [Proc Natl Acad Sci USA 99:984-9 (2002)].

In case of employing diaphorase (6) above, there may be utilized those of which a function of catalyzing the following chemical reaction is identified and a base sequence of the gene DNA or an amino acid sequence of the enzyme is already known:
L-arginine+nNADPH+nH⁺+mO₂=citrulline+nitric oxide+nNADP⁺
Examples of such diaphorase gene include those derived from Bacillus genus, such as Bacillus cereus ATCC14579 [Nature 423:87-91 (2003)]. In particular, enzymes derived from Thermus thermophilus, Thermotoga maritima, Sulfolobus tokodaii, Pyrobaculum aerophilum have heat resistance and may be utilized advantageously in the present invention.

As malic acid dehydrogenase for association, there may be utilized those of which a malic acid dehydrogenase activity is confirmed and a base sequence of the gene DNA or an amino acid sequence of the enzyme is already known. The malic acid dehydrogenases are classified as follows:

(A) EC 1.1.1.37: (S)-malate:NAD+oxidoreductase

(B) EC 1.1.1.38: (S)-malate:NAD+oxidoreductase (oxaloacetate-decarboxylating)

(C) EC 1.1.1.39: (S)-malate:NAD+oxidoreductase (decarboxylating)

(D) EC 1.1.1.40: (S)-malate:NADP+oxidoreductase (oxaloacetate-decarboxylating)

among which enzymes classified as EC 1.1.1.38, EC 1.1.1.39 and EC 1.1.1.40 are preferable, since, in the enzyme of EC 1.1.1.37, the equilibrium of chemical reaction is deviated to the malic acid side. Examples of such malic acid dehydrogenase gene include those derived from Bacillus genus, such as Bacillus cereus ATCC10987 [Nucleic Acids Res 32:977-88 (2004)] or Bacillus subtilis 168 [Nature 390:249-56 (1997)], Deinococcus genus, such as Deinococcus radiodurans R1 [Science 286:1571-7 (1999)], Lactobacillus genus, such as Lactobacillus plantarum WCFS1 [Proc Natl Acad Sci USA 100:1990-5 (2003)], Pseudomonas genus, such as Pseudomonas aeruginosa PA01 [Nature 406:959-64 (2000)], Pseudomonas putida KT2440 [Environ Microbiol 4:799-808 (2002)] or Pyrococcus furiosus DSM 3638 [Genetics 152:1299-305 (1999), Mol Microbiol 38:684-93 (2000), Methods Enzymol 330:134-57 (2001)], Saccharomyces genus, such as Saccharomyces cerevisiae S288C [Science 274:546-67 (1996), Proc Natl Acad Sci USA 92:3809-13 (1995), EMBO J 13:5795-809 (1994), Nature 357:38-46 (1992), Nature 387:75-8 (1997), Nature 387:78-81 (1997), Nat Genet 10:261-8 (1995), Nature 387:81-4 (1997), Science 265:2077-82 (1994), Nature 387:84-7 (1997), EMBO J 15:2031-49 (1996), Nature 369:371-8 (1994), Nature 387:87-90 (1997), Nature 387:90-3 (1997), Nature 387:93-8 (1997), Nature 387:98-102 (1997), Nature 387:103-5 (1997)], Sulfolobus genus, such as Sulfolobus solfataricus [Proc Natl Acad Sci USA 98:7835-40 (2001)] or Sulfolobus tokodaii strain7 [DNA Res 8:123-40 (2001)], Thermotoga genus, such as Thermotoga maritima MSB8 [Nature 399:323-9 (1999)], and Thermus genus, such as Thermus thermophilus HB27 [Nat Biotechnol 22:547-53 (2004)]. In particularly enzymes derived from Thermus thermophilus, Thermotoga maritima, Pyrococcus furiosus and Sulfolobus tokodaii have heat resistance, and may be utilized advantageously in the present invention.

As glutamic acid dehydrogenase (EC 1.4.1.2, EC 1.4.1.3 and EC 1.4.1.4) for association, there may be utilized those of which a function of catalyzing the following chemical reaction is identified and a base sequence of the gene DNA or an amino acid sequence of the enzyme is already known:
L-glutamate+H₂O+NAD(P)+=2-oxoglutarate+NH₃+NAD(P)H+ H⁺
Examples of such glutamic acid dehydrogenase gene include those derived from Bacillus genus, such as Bacillus subtilis 168 [Nature 390:249-56 (1997)], Deinococcus genus, such as Deinococcus radiodurans R1 [Science 286:1571-7 (1999)], Geobacillus genus, such as Geobacillus kaustophilus HTA426 [Nucleic Acids Res 32:6292-303 (2004)], Lactobacillus genus, such as Lactobacillus plantarum WCFS1 [Proc Natl Acad Sci USA 100:1990-5 (2003)], Pyrococcus genus, such as Pyrococcus horikoshii OT3 [DNA Res 5:55-76 (1998)], Pseudomonas genus, such as Pseudomonas aeruginosa PA01 [Nature 406:959-64 (2000)] or Pseudomonas putida KT2440 [Environ Microbiol 4:799-808 (2002)], Pyrococcus genus, such as Pyrococcus abyssi GE5 or Pyrococcus furiosus DSM 3638[Genetics 152:1299-305 (1999), Mol Microbiol 38:684-93 (2000), Methods Enzymol 330:134-57 (2001)], Sulfolobus genus, such as Sulfolobus solfataricus [Proc Natl Acad Sci USA 98:7835-40 (2001)] or Sulfolobus tokodaii strain7[DNA Res 8:123-40 (2001)], Thermococcus genus, such as Thermococcus kodakaraensis KOD1 [Genome Res 15:352-63 (2005)], and Thermus genus, such as Thermus thermophilus HB27 [Nat Biotechnol 22:547-53 (2004)] or Thermus thermophilus HB8. In particular, enzymes derived from Thermus thermophilus, Thermotoga maritima, Pyrococcus furiosus and Sulfolobus tokodaii have heat resistance and may be utilized advantageously in the present invention.

As isomerase (xylose isomerase) (EC 5.3.1.5) for association, there may be utilized those of which a function of catalyzing the following chemical reaction is identified and a base sequence of the gene DNA or an amino acid sequence of the enzyme is already known:
D-xylose=D-xylulose
Examples of such imsoemrase gene include those derived from Bacillus genus, such as Bacillus subtilis 168 [Nature 390:249-56 (1997)], Lactococcus genus, such as Lactococcus lactis subsp. lactis IL1403 [Genome Res 11:731-53 (2001)], and Thermotoga genus, such as Thermotoga maritima MSB8 [Nature 399:323-9 (1999)]. In particular, an enzyme derived from Thermotoga maritima has heat resistance, and may be utilized advantageously in the present invention.

In the associated protein to be employed in the enzyme electrode of the present invention, even in the case that the functional unit of enzyme activity is not a single-chain polypeptide (monomer), the objects of the present invention may be accomplished by adopted any of the following structures.

(1) In a case where the functional unit of any of the enzymes is a homooligomer: Let it be assumed that the structure of functional polypeptide is represented by αₙ [n being an integer of n>1], and that polypeptide in the polypeptide association site is represented by β. In order to construct such enzyme as an association element of the associated protein, a polypeptide (a) constituting the enzyme having the homooligomer as the functional unit is co-expressed, together with the fused polypeptide (α::β) in a host cell. In this manner, there can be obtained a fusion protein of a polypeptide structure (αₙ::β). Two polypeptides to be co-expressed (α::β and α) may be encoded on a same plasmid or on mutually different plasmids. However, in case of utilizing different plasmids, it is necessary that both plasmids are free from incompatibility. In this system, there may be generated, in addition to the desired fusion protein (α::β), undesired proteins of peptide structures such as αₙ and αₙ₋ₓ(αₙ::β)ₓ [x being an integer of n+1>x>1]. These may be separated, for example by differences in the molecular weight, by a gel filtration or an ultrafiltration. Also the fused polypeptide α::β may be expressed in a state where a tag for purification is further fused. Also a fusion protein of a structure αₙ₋ₓ(αₙ::β)ₓ [x being an integer of n+1>x>1] may also be used, if the enzyme activity is retained, as an association element of the associated protein to be used in the enzyme electrode of the present invention.

(2) In a case where the functional unit of any of the enzymes is a heterooligomer: Let it be assumed that the functional polypeptide structure is represented by αₙα′ [wherein α′ represents polypeptide chains collectively other than α, and n is an integer of n>0], that polypeptide in the polypeptide association site is represented by β. In order to construct such enzyme as an association element of the associated protein, polypeptides (α and α′) constituting the enzyme having the heterooligomer as the functional unit are co-expressed, together with the fused polypeptide (α::β), in a host cell. In this manner, there can be obtained a fusion protein of a polypeptide structure (αₙα′::β). The polypeptide chains to be co-expressed (α::β, α and α′) may be encoded on a same plasmid or on mutually different plasmids. However, in case of utilizing different plasmids, it is necessary that both plasmids are free from incompatibility. In this system, there may be generated, in addition to the desired fusion protein (αₙα′::β), undesired proteins of peptide structures such as αₙ₋ₓα′ (α::β)ₓ [x being an integer of n+1>x>0], and αₙα′. These may be separated, for example by differences in the molecular weight, by a gel filtration or an ultrafiltration. Also a fusion protein of a structure αₙ₋ₓα′ (α::β)ₓ [x being an integer of n+1>x>0] may also be used, if the enzyme activity is retained, in the enzyme electrode of the present invention. In order to suppress generation of such fusion protein, it is preferable to select, among the constituent units of the heterooligomer, one of a least number within the functional structure, namely one with n=1, as α. When a constituent unit of n=1 as α, the polypeptide chains to be co-expressed may be limited to α::β and α′. Also the fused polypeptide α::β may be expressed in a state where a tag for purification is further fused.

Also in the cases of (1) and (2) above, it is possible to adopt an in vitro cell-less protein synthesizing system instead of the in-cell co-expression system. In such case, the fusion protein of the desired polypeptide structure may be formed by synthesizing respective constituent polypeptides by the in vitro protein synthesizing system and then mixing these.

In the following, there will be explained a producing method for the enzyme protein for association, utilizing a recombinant DNA technology.

A recombinant vector enabling expression of an enzyme protein for association in various hosts may be constructed by inserting a DNA, encoding the amino acid sequence of the enzyme protein for association, in a downstream side of a promoter of an appropriate expression vector. It is possible, for those skilled in the art, to construct a protein expressing vector, which constructs a functional associated protein in an arbitrary host cell, according to a normal process in this technical field. The promoter may be suitably selected from those already known, or may be prepared anew. It is also possible, by a modification by an ordinary technology (for example by exchanging promoter), to construct an expression vector, capable of producing, at a high level, a protein constituting the associated protein.

A host cell, to be employed for a transformation by the vector expressing a protein constituting the associated protein, may be a procaryotic cell such as E. coli, or an eucaryotic cell such as yeast, or may also be a cell of higher living organisms commonly utilized. Examples of the host cell include microorganisms [procaryotic living organisms (bacteria such as E. coli or bacillus subtilis), or eucaryotic living organisms (such as yeast)], animal cells and cultured vegetable cells. As the bacteria, procaryotic bacteria or yeast is preferred. As the procaryotic bacteria, strains belonging to Escherichia genus (such as E. coli) are particularly preferred. As the yeast, those belonging to Saccharomyces genus (such as S. cerevisiae) or those belong to Candida genus (such as C. Boidinii) are preferred. Examples of the animal cells include mouse L929 cells and Chinese hamster ovary (CHO) cells.

An expression vector suitable for using bacteria, particularly E. coli as the host cell is already known, and examples thereof include those having a common promoter such as a lac promoter or a tac promoter. As an expression vector for expressing an enzyme protein for association in yeast, there are preferred those containing a promoter such as GAL promoter or AOD promoter. Also as an expression vector for expressing an enzyme protein for association in a mammal, there can be used one having a promoter such as SV40 promoter. However, in consideration of ease in operation and in availability, the host cell is preferably a procaryotic host, particularly E. coli. The procaryotic host-vector system is described in many references and is already known in such technical field, and will be briefly explained below (for example, cf. Molecular Cloning: A LABORATORY MANUAL, Cold Spring Harbor Laboratory Press). In order to express DNA encoding the enzyme protein for association by E. coli, such DNA is inserted in the downstream side of a promoter of a plasmid suitable for a transformation utilizing E. coli. Examples to be explained later describe an embodiment of expression utilizing E. coli. In another embodiment, the enzyme protein for association can be expressed in various hosts by treating DNA, encoding the enzyme protein for association, with a suitable enzyme to obtain a DNA fragment encoding the associated protein, and incorporating it in a suitable vector. Examples of the enzyme to be used in such treatment include a restriction enzyme, alkali phosphatase, polynucleotide kinase, DNA ligase and DNA polymerase.

The method of transformation of the host cell by an expression vector for an enzyme protein for association is already known, and it may be executed by a method described in Molecular Cloning : A LABOLATORY MANUAL, Cold Spring Harbor Laboratory Press. For example, in case of a procaryotic cell host, there may be employed a competent cell preparation method, also in case of an eucaryotic cell host, there may be employed a competent cell preparation method, and in case of a mammal cell, there may be employed a transfection method or an electropolation method. Then the obtained transform is cultured on a suitable culture medium. The culture medium may contain a carbon source (such as glucose, methanol, galactose, or fructose), and an inorganic or organic nitrogen source (such as ammonium sulfate, ammonium chloride, sodium nitrate, peptone or casamino acid). If desired, other nutrition sources (for example inorganic salts (such as sodium chloride or potassium chloride), vitamins (such as vitamin B1), and antibiotics (such as ampicillin, tetracyclin, or kanamycin)) may be added to the culture medium. For culturing a mammal cell, Eagle culture medium is suitable.

The transform is usually cultured within a pH range of from 6.0 to 8.0, preferably at a pH of 7.0, at a temperature of from 25 to 40° C., preferably from 30 to 37° C. for 8 to 48 hours. In the case that the produced enzyme protein for association is present in a culture medium, the cultured substance is separated by filtration or by centrifuging. The purification may be executed, from a recovered supernatant liquid, by ordinary methods utilizing for purification and isolation of a protein, constituting a natural or synthetic protein. For such treatment, there can for example be utilized dialysis, gel filtration, affinity column chromatography utilizing a monoclonal antibody for the protein constituting the associated protein, column chromatography utilizing an appropriate adsorbent, or high-speed liquid chromatography. In the case that the produced enzyme protein for association is present in a periplasma or cytoplasma of the cultured transform, the cells are collected by filtration or centrifuging, and the cell wall and/or cell membrane is cleaved for example by an ultrasonic and/or lysozyme treatment to obtain cell fragments. The protein constituting the associated protein can be purified by mixing an appropriate aqueous solution (such as a buffer) with the cell fragments, followed by an ordinary process.

The protein constituting the associated protein, produced in E. coli, may be regenerated (refolded) by an ordinary method, if necessary.

Particularly in case of preparing an associated enzyme derived from thermophilic bacteria, a simple purification is possible by maintaining the liquid containing the cell fragments at a temperature of 70° C. of higher to coagulate the protein derived from the host bacteria, and by separating the coagulated substance for example by a centrifuging. It may also be converted into a fold having an activity.

Also depending on the application, the enzyme protein for association and the associated protein need not necessarily be completely purified but may be any of (1) to (6) below:

(1) live cell: a cell separated from a cultured substance by an ordinary method such as filtration or centrifuging;

(2) dry cell: a cell obtained by freeze drying or vacuum drying of the live cell described in (1);

(3) cell extract: a product obtained by processing the cell as described in (1) or (2) by an ordinary method (such as a self-dissolution in an organic solvent, a crushing by mixing with alumina or sea sand, or an ultrasonic treatment);

(4) enzyme solution: a product obtained by a purification or a partial purification of the cell extracted as described in (3) by an ordinary method;

(5) purified enzyme: a product not containing an impurity, obtained by further purifying the enzyme solution described in (4);

(6) fragment having enzyme activity: a peptide fragment obtained by an appropriate fragmentation of the purified enzyme as described in (5).

The enzyme protein for association to be employed in the enzyme electrode of the present invention, for example obtained by a biosynthesis in a cell of recombinant E. coli, is often obtained as a holoenzyme bonded with a prosthetic group such as a flavin compound, a metal atom (such as Fe, Cu or Mo) or a heme. It is possible to increase the recovery rate of holoenzyme by adding such prosthetic group in advance in the culture medium at the biosynthesis. On the other hand, in case of obtaining a peptide of the protein constituting the associated protein, in in vitro for example by a cell-less protein synthesizing device, an apoenzyme not bonded with the prosthetic group is obtained. In such case, the obtained apoenzyme may be subjected to a process of maintaining in a buffer containing the prosthetic group, thereby re-constructing the holoenzyme.

An ordinary genetic engineering process may be applied to produce a protein in which introduced is a variation such as a substitution, a deletion, an insertion, a transfer or an addition of one or plural (several) amino acids in the amino acid sequence of the enzyme protein for association. Examples of the method for such variation/transformation/modification include those described in The Japanese Biochemical Society ed., “Continued Biochemical Experiments 1, Gene research II”, p. 105 (Susumu Hirose), Tokyo Kagaku Dojin (1986); The Japanese Biochemical Society ed., “New Biochemical Experiments 2, Nucleic acid III (recombinant DNA technology)”, p. 233 (Susumu Hirose), Tokyo Kagaku Dojin(1992); R. Wu, L. Grossman, ed., “Methods in Enzymology”, Vol. 154, p. 350 & p. 367, Academic Press, New York (1987); R. Wu, L. Grossman, ed., “Methods in Enzymology”, Vol. 100, p. 457 & p. 468, Academic Press, New York (1983); J. A. Wells et al., Gene, 34: 315, 1985; T. Grundstroem et al., Nucleic Acids Res., 13: 3305, 1985; J. Taylor et al., Nucleic Acids Res., 13: 8765, 1985; R. Wu ed., “Methods in Enzymology”, Vol. 155, p. 568, Academic Press, New York(1987); and A. R. Oliphant et al., Gene, 44: 177, 1986. Examples include a position-designated variation introducing method (position-designated specific variation introducing method) utilizing for example a synthetic oligonucleotide, a Kunkel method, a dNTP [αS] (Eckstein) method, and an area-designated variation introducing method.

It is also possible, based on the gene base sequence of the enzyme protein for association obtained by the aforementioned methods, to modify the amino acid residue of such enzyme protein by a chemical method. It is furthermore possible to modify or partially decompose the enzyme protein for association obtained by the aforementioned methods with an enzyme such as a peptidase, thereby obtaining a derivative thereof. For such peptidase, there may be employed for example pepsin, chymotripsin, papain, bromelain, endopeptidase or exopeptidase.

Also the enzyme protein for association may be expressed as a fusion protein in which a purification tag or a transitional signal sequence is fused. A protein, constituting the associated protein, in which a purification tag or a transitional signal sequence is fused, may be produced by a fused production method commonly utilized in the genetic engineering, and, in the enzyme protein for association in which a purification tag is fused, such purification tag may be utilized for a purification by affinity chromatography or as a immobilization tag for forming the enzyme immobilizing layer described above.

Thus, the enzyme protein for association may be different in one or more amino acid residues, in identity, from the natural amino acid residues, or different in the positions of one or more amino acid residues from those of the natural amino acid residues. Also the enzyme protein for association may be a deleted analog in which one or more amino acid residues, specific to the natural protein, are deleted. A number of such deleted amino acid residues may be within a range of from 1 to 80, preferably from 1 to 60, more preferably from 1 to 40, further preferably from 1 to 20, and particularly preferably from 1 to 10. Also the enzyme protein for association may be a substituted analog in which one or more amino acid residues, specific to the natural protein, are substituted. A number of such substituted amino acid residues may be within a range of from 1 to 80, preferably from 1 to 60, more preferably from 1 to 40, further preferably from 1 to 20, and particularly preferably from 1 to 10. Also the enzyme protein for association may be an added analog in which one or more amino acid residues are added to those specific to the natural protein. A number of such added amino acid residues may be within a range of from 1 to 80, preferably from 1 to 60, more preferably from 1 to 40, further preferably from 1 to 20, and particularly preferably from 1 to 10. This is because the enzyme proteins for association may include those having a primary structural conformation substantially equivalent to that of the natural protein, or a part thereof, as long as domain structures featuring the natural protein of the enzyme are respectively retained. Also this is because the enzyme proteins for association may include those having a biological activity, substantially same as that of the natural protein. Therefore, the aforementioned variations are all usable as the associated protein in the enzyme electrode of the present invention.

The variation protein as the enzyme protein for association may be, for example, those having a high homology with the amino acid sequences indicated by sequence number in sequence table of 15, 16, 27, 28, 36, 44, 61, 62, 73, 74, 82, 90, 98, 106, 114, 115, 121, 122, 128, 129, 135, 136, 150, 151, 152, 157, 158 or 159, and maintaining the desired enzyme activity. Examples of such homologous amino acid sequence include those preferably having a homology of 70% or higher, more preferably 80% or higher and particularly preferably 90% or higher.

In the enzyme, “Substantially equivalent” to the natural protein means that is substantially same in the activities of the protein, such as catalytic activity, physiological activity and biological activity. A substitution, a deletion or an insertion of amino acids often does not cause a significant change in the physiological or chemical properties of polypeptide. In such case, the polypeptide subjected to such substitution, deletion or insertion is considered substantially same as the polypeptide not subjected to such substitution, deletion or insertion. A substitute, substantially same as an amino acid in an amino acid sequence, may be selected from other amino acids of a class to which such amino acid belongs. Examples of a non-polar (hydrophobic) amino acid include alanine, phenylalanine, leucine, isoleucine, valine, proline, triptophane, and methionine, and examples of polar (neutral) amino acid include glycine, serine, threonine, cysteine, tyrosine, asparagine, and glutamine. Examples of an amino acid having a positive charge (basic amino acid) include alginine, lysine and hystidine, and those of an amino acid having a negative charge (acidic amino acid) include aspartic acid and glutamic acid.

(Electron Transporting Mediator)

The electron transfer mediator in the enzyme electrode of the present invention is a substance included in the electrode system, in order to promote an electron donating-accepting reaction between the enzyme and the conductive base member, and to improve a measuring sensitivity and a current density. The electron transfer mediator to be employed in the enzyme electrode of the present invention is not particularly restricted as long as the properties above are satisfied. Examples thereof include a metal complex formed by a center metal and a ligand thereof, an ionized substance thereof, a cetallocene, phenazine methosulfate, 1-methoxy-phenazine methosulfate, a quinone and a derivative of these substances.

Examples of the metal complex compound include those containing, as a center metal, at least one selected from Os, Fe, Ru, and Co. Examples of the ligand to the center metal include pyrrole, pyrazole, imidazole, 1,2,3- or 1,2,4-triazole and derivatives thereof.

Specific examples of the metal complex and the ionized substance thereof include a bipyridine complex formed by a metal such as osmium, ruthenium, cobalt or nickel, and bipyridine, and a metal complex ion such a ferricyan ion, an octacyanotungstate ion, and an octacyanomolybdenate ion.

Examples of metallocene include ferrocene and ferrocene derivatives such as 1,1′-dimethylferrocene, ferrocenecarboxylic acid or ferrocene carboxyaldehyde.

Such electron transfer mediator may be used in a combination of two or more kinds, as long as the effects of the present invention are not hindered.

The electron transfer mediator is contained, in the enzyme electrode of the present invention, in an amount of from 0.5 to 10 wt %, preferably from 1 to 5 wt %, of all the constituent components. All the constituent components mean the components contained in the enzyme immobilizing layer on the conductive base member.

(Constitution of Electrode System)

An enzyme electrode device (such as a biosensor, a fuel cell, or an electrochemical reaction device) applicable to various purpose can be prepared by connecting, to the enzyme electrode of the invention, a wiring for electron exchange. Such device can be constituted by utilizing the aforementioned plate-shaped (or film-shaped or layer-shaped) enzyme electrode in a single layer or with a plurality thereof. In case of utilizing plural layers, they may be laminated in such a manner that a top surface and a rear surface thereof are mutually opposed. Also in case of utilizing plural layers, the enzyme electrodes may have uniform characteristics or may be combined with different characteristics. For example, as in a fuel cell to be explained later, anodes and cathodes may be disposed alternately. Such device can adapt to a voltage or an output required, by changing the number of layers of the electrodes from a single layer to plural layers. The enzyme employed as the catalyst for the enzyme electrode has a higher substrate selectivity, in comparison with a precious metal catalyst (for example platinum), commonly employed in the electrochemical field. Consequently, there is not required a mechanism for separating reaction substances in an electrode and in the other electrode, and the device can therefore be simplified.

(Sensor)

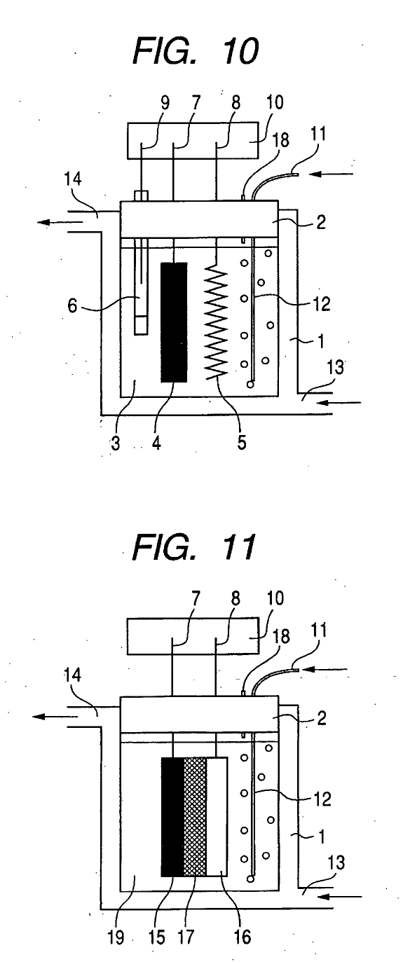

A sensor, constituting a preferable embodiment of the present invention, has a structure utilizing the enzyme electrode of the present invention, as a detection site for detecting a substance. In a representative structure, an enzyme electrode is used as a reaction electrode in a set with a reference electrode and a counter electrode, and a current is detected by the enzyme electrode (by the function of the enzyme immobilized on the electrode). The detection of presence/absence or an amount of current allows to detect presence/absence or an amount of a substance in a liquid with which the electrode is in contact. A specific example of the sensor has a structure as shown in FIG. 10.

The sensor shown in FIG. 10 includes a reaction electrode 4, a platinum line counter protein 5, and a silver chloride reference protein 6, and lead wires 7, 8 and 9 connected respectively to these electrodes are connected to a potentiostat 10. Such sensor is positioned in a storing area of a sample solution 3, in a water-jacketed cell 1 which can be sealed by a cover 2. A substrate in the electrolyte can be detected by applying a potential to the reaction electrode and measuring a constant current. In a case that a measurement under an inert gas atmosphere is necessary, an inert gas such as nitrogen is introduced from a gas blowing aperture 11 at the external end of a gas tube 12. Also the temperature can be controlled by a supply of a temperature controlling liquid, utilizing a temperature controlling water inlet 13 and a temperature controlling water outlet 14.

(Fuel Cell)

A fuel cell, constituting a preferable embodiment of the present invention, is characterized in employing the enzyme electrode of the present invention in at least either of an anode and a cathode. Also in this case, the enzyme electrode may be employed, in a plate shape or a layer shape, in a single layer or in a laminated structure of two or more layers. Also in case of the laminate structure, the anodes and the cathodes may be arranged in a predetermined arrangement in the direction of lamination. A representative structure includes a reaction tank capable of storing an electrolyte liquid containing a fuel substance, and an anode and a cathode positioned with a predetermined gap in the reaction tank, wherein an enzyme electrode of the present invention is utilized in at least either of the anode and the cathode. The fuel cell may be constructed in a type in which the electrolyte is replenished or recycled, or in a type in which the electrolyte is not replenished nor recycled. The fuel cell is not restricted in a fuel type, a structure or a function, as long as the enzyme electrode is usable. As the enzyme employed as the catalyst for the enzyme electrode has a higher substrate selectivity, there is not required a mechanism for separating reaction substances in an electrode and in the other electrode, and the device can therefore be simplified. The fuel cell, owing to the high catalytic function specific to the enzyme employed as the catalyst of the electrode reaction, can achieve oxidation/reduction of the substance with a low overvoltage, thereby providing a high drive voltage, and can provide a long service life and a high output by a high stability and a high current density.

An example of the fuel cell is shown in FIG. 11. The cell of the fuel cell is constructed approximately same as the substrate measuring device in the sensor shown in FIG. 10, and similar members are represented by same reference numerals. In place for the sensor shown in FIG. 10, there is employed an electrode unit formed by laminating an anode 15 and a cathode 16 across a porous polypropylene film 17, and is operated as a fuel cell by introducing oxygen gas into the cell through a tube 12.

(Electrochemical Reaction Device)

An electrochemical reaction device, constituting a preferable embodiment of the present invention, can achieve a high substrate selectivity and a high catalytic ability, specific to the enzyme employed as the catalyst for the electrode reaction. In addition, it can achieve a quantitative property of the reaction, characteristics of an electrochemical reaction. As a result, it can be controlled quantitatively with a high selectivity and a high efficiency, and also can provide a long service life and a high output, owing to a high stability and a high current density based on the enzyme electrode utilizing a carrier and plural mediators. Also in this case, the enzyme electrode may be employed, in a plate shape or a layer shape, in a single layer or in a laminated structure of two or more layers. In a representative structure, a pair of electrodes, and a reference electrode provided if necessary, are positioned in a reaction tank capable of storing a reaction liquid, and a current is supplied between the pair of electrodes to induce an electrochemical reaction in a substance in the reaction liquid, thereby obtaining a reaction product or a decomposition product desired. In such case, in at least either of the pair of electrodes, the enzyme electrode of the present invention may be employed. The structure of device relating to a type of reaction liquid and a reacting condition are not particularly restricted as long as the enzyme electrode of the present invention is applicable. For example the device may be utilized for obtaining a reaction product by an oxidation/reduction reaction or obtaining a decomposition product.

### EXAMPLES

In the following, the present invention will be clarified further by examples, but the present invention is not limited to such examples.

In the sensor, fuel cell and electrochemical reaction device, utilizing an enzyme electrode, in Examples and Comparative Examples, utilize an device of the following structure, except that the enzyme electrode is changed.

(Sensor)

A common structure of a sensor is illustrated in FIG. 10. An enzyme electrode is used as a reaction electrode 4, thereby constructing a sensor for a measurement by the enzyme electrode. A counter electrode 5 is formed by a platinum wire, and a reference electrode 6 is formed by a silver/silver chloride electrode. These electrodes measure for example a current, through a potentiostat 10. A sample solution 3 is a solution (for example a buffer), containing a compound to be measured at a predetermined concentration. A measuring temperature is maintained at 37° C. by a thermostatic circulating tank. A quantitative measurement of the compound to be measured is executed in the manner. A potential of 300 V is applied to the reaction electrode 4, with respect to the reference electrode 6. In this state, the object compound in the sample solution 3 reacts with the enzyme electrode to generate a current by an electron movement through the reaction electrode 4. Such current is measured.

(Fuel Cell)

A common structure for a fuel cell is schematically illustrated in FIG. 11. In this device, an enzyme electrode is utilized as an anode electrode 15. A cathode electrode 16 formed by a platinum plate of 0.5 cm². An electrolyte solution 3 is formed by a buffer, containing for example a compound capable of reacting the enzyme electrode thereby generating a current. The anode electrode 15 and the cathode electrode 16 are positioned across a porous polypropylene film (thickness: 20 μm) 17, and positioned in the electrolyte solution 3 (10 mL) in a water-jacketed cell 1 having a cover 2. A measuring temperature is maintained at 37° C. by a thermostatic circulating tank. Leads are connected to a potentiostat (model 2000, manufactured by Toho Giken Co.) 10, and voltage-current characteristics were measured by varying the voltage from −1.2 V to 0.1 V.

(Electrochemical Reaction Device)

The device shown in FIG. 10 is used as an electrochemical reaction device. In this case, there is constructed a three-electrode cell, employing an enzyme electrode as the reaction electrode 4, a silver chloride/silver electrode as the reference electrode 6 and a platinum wire as the counter electrode 5. A buffer electrolyte liquid containing a raw material is used as the sample solution 3, and the temperature thereof is maintained at 37° C. by a thermostatic circulating tank. A potential of 0.3 VvsAg/AgCl is applied for 100 minutes in the water-jacketed cell 1 and under a nitrogen atmosphere, and a product is quantitatively measured by a high-speed liquid chromatography.

(Enzyme Preparation Method)

An enzyme was obtained from a transform by a method 1 or 2 below.

(Method 1)

A transform is pre-cultured overnight in an LB medium in which ampicillin and streptomycin are added as antibiotics. Then 0.2 mL of the medium are added to 100 mL of LB-Amp culture medium, which is vibration cultured for 4 hours at 30° C. and 170 rpm. Then IPTG is added (end concentration: 1 mM), and the culture is continued for 4 to 12 hours at 37° C. The IPTG-induced transform is collected (8000×g, 2 minutes, 4° C.), and re-suspended in PBS of a 1/10 amount at 4° C. The bacteria are crushed by freezing, thawing and sonication, and centrifuged (8000×g, 10 minutes, 4° C.) to eliminate solids. After confirmation by SDS-PAGE of the presence of the desired expressed protein in the supernatant, the protein fused with His tag, thus expressed, is purified by a nickel chelate column.

(Method 2)

An enzyme is obtained in the same manner as in the method 1, except for employing ampicillin only as the antibiotic in the LB medium for pre-culture. Example Nos. 14, 15, 16, 22, 23, 24, 30, 31, 32, 38, 39, 40, 46, 47, 48, 54, 55, 56, 59 and 60 are intentionally missing.

### (Example 1) Preparation of Associated Protein (His-busGDH-Fos and ppuDp-Jun) [SEQ ID NO:15, 16 of Glucose Dehydrogenase (busGDH) Derived from Bacillus subtilis and Diaphorase (ppuDp) Derived from Pseudomonas putida

A genome DNA is prepared from Bacillus subtilis [ATCC 27370] by an ordinary method. A PCR is executed utilizing this genome DNA as a template and employing synthetic oligo DNAs SEQ ID NO:1 having a sequence recognized by BamHI and SEQ ID NO:2 having a sequence recognized by HindIII as primers to obtain an amplified DNA product of 805 bp.

The amplified DNA product is digestion cleaved by restriction enzymes BamHI and HindIII, and is inserted into a same restriction enzyme site of pETDuet-1 (manufactured by Novagen) to obtain a His-busGDH expressing vector pETDuet-busGDH.

Then, genome DNA is prepared from Pseudomonas putida KT2440 [ATCC 47054] by an ordinary method. A PCR is executed utilizing this genome DNA as a template and employing synthetic oligo DNAs SEQ ID NO:3 having a sequence recognized by NdeI and SEQ ID NO:4 having a sequence recognized by XhoI as primers to obtain an amplified DNA product of 729 bp.

The amplified DNA product is digestion cleaved by restriction enzymes NdeI and XhoI, and is inserted into a same restriction enzyme site of pETDuet-busGDH to obtain a vector pETDuet-busGDH-ppuDp, co-expressing His-busGDH and ppuDp.

Then, 5′-terminal phosphated synthetic oligoDNAs (SEQ ID NO:5 and 6) are mixed in equal amounts in a TE buffer, and annealed by heating followed by standing to cool.

This DNA fragment (double strand) is inserted in a recognition site for restriction enzymes HindIII and AflII of pETDuet-busGDH/ppuDp, thereby obtaining a co-expression vector pETDuet-busGDH-Fos/ppuDp of a fusion protein, in which an association site sequence SEQ ID NO:168-Fos (SEQ ID NO:7) is fused to a C-terminal side of His-busGDH, and ppuDp.

Then, 5′-terminal phosphated synthetic oligoDNAs (SEQ ID NO:8 and 9) are mixed in equal amounts in a TE buffer, and annealed by heating followed by standing to cool.

This DNA fragment (double strand) is inserted in a recognition site for restriction enzymes XhoI and AvrII of pETDuet-busGDH-Fos/ppuDp, thereby obtaining a co-expression vector pETDuet-busGDH-Fos/ppuDp-Jun (SEQ ID NO:11) of a fusion protein, in which an association site sequence SEQ ID NO:168-Fos (SEQ ID NO:7) is fused to a C-terminal side of His-busGDH, and a fusion protein in which an association site sequence SEQ ID NO:168-Jun (SEQ ID NO:10) is fused at a C-terminal side of ppuDp.

Then a PCR is executed utilizing the genome DNA of Bacillus subtilis [ATCC 27370] as a template and employing a synthetic oligoDNAs SEQ ID NO:12 having a sequence recognized by NcoI and SEQ ID NO:2 having a sequence recognized by HindIII as primers to obtain an amplified DNA product of 805 bp.

The amplified DNA product is digestion cleaved by restriction enzymes NcoI and HindIII, and is inserted into a same restriction enzyme site of pCDFDuet-1 (manufactured by Novagen) to obtain a busGDH expression vector pCDFDuet-busGDH.

Then, a PCR is executed utilizing the genome DNA of Pseudomonas putida KT2440 [ATCC 47054] as a template and employing synthetic oligoDNAs SEQ ID NO:3 having a sequence recognized by NdeI and SEQ ID NO:13 having a sequence recognized by XhoI as primers to obtain an amplified DNA product of 729 bp.

The amplified DNA product is digestion cleaved by restriction enzymes NdeI and XhoI, and is inserted into a same restriction enzyme site of pCDFDuet-busGDH to obtain a co-expression vector pCDFDuet-busGDH-ppuDp (SEQ ID NO:14) of busGDH and ppuDp.

Expression vectors pETDuet-busGDH-Fos/ppuDp-Jun and pCDFDuet-busGDH-ppuDp are transformed into E. coli BL21(DE3) by an ordinary method. The transform can be selected as a resistant strain to antibiotics ampicillin and streptomycin. Transform was used for preparing an enzyme by the method 1.

### (Comparative Example 1) Preparation of busGDH and ppuDp as Reference

A genome DNA is prepared from Bacillus subtilis [ATCC 27370] by an ordinary method. A PCR is executed utilizing this genome DNA as a template and employing synthetic oligo DNAs SEQ ID NO:17 having a sequence recognized by NdeI and SEQ ID NO:18 having a sequence recognized by XhoI as primers to obtain an amplified DNA -product of about 800 bp.

The amplified DNA product is digestion cleaved by restriction enzymes NdeI and XhoI, and is inserted into a same restriction enzyme site of pET-21a(+) (manufactured by Novagen) to obtain a busGDH-His expression vector pET21-busGDH.

Then, a genome DNA is prepared from Pseudomonas putida KT2440 [ATCC 47054] by an ordinary method. A PCR is executed utilizing this genome DNA as a template and employing oligo DNAs SEQ ID NO:3 and 4 as primers to obtain an amplified DNA product of 729 bp.

The amplified DNA product is digestion cleaved by restriction enzymes NdeI and XhoI, and is inserted into a same restriction enzyme site of pET-21a(+) (manufactured by Novagen) to obtain a ppuDp-His expression vector pET21-ppuDp.

Expression vectors pET21-busGDH and pET21-ppuDp are individually transformed into E. coli BL21(DE3) by an ordinary method. The transform can be selected as a resistant strain to antibiotic ampicillin.

These transforms were individually used to prepare enzymes by the method 2.

### (Example 2) Glucose Sensor

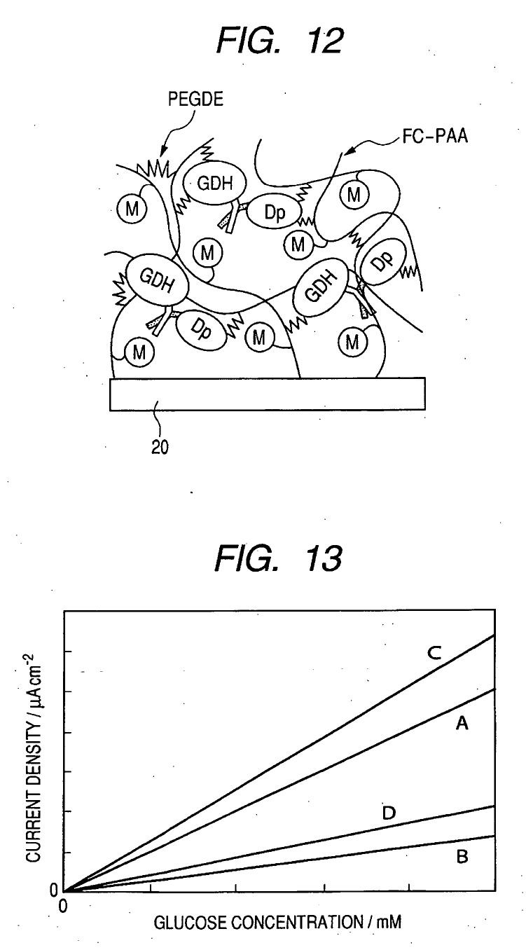

A structure of the enzyme electrode is schematically shown in FIG. 12. A conductive base member 20 is formed by glassy carbon of a diameter of 3 mm. On the conductive base member 20, an associated protein (His-busGDH/ppuDp) of glucose dehydrogenase (busGDH) derived from Bacillus subtilis in Example 1 and diaphorase (ppuDp) derived from Pseudomonas putida, and a ferrocene-bonded polyallylamine (Fc-PAA) are immobilized by crosslinking by poly(ethylene glycol) diglycigyl ether (PEGDE). Components immobilized on the enzyme electrode have the following formulation:

His-busGDH-Fos/ppuDp-Jun (busGDH: 0.3 units, ppuDp: 0.6 units)

Fc-PAA: 16 μg

PEGDE: 10 μg

The enzyme electrode is prepared by mixing the aqueous solutions of the components above on the electrode, and standing and drying at the room temperature for 2 hours or longer.

This enzyme electrode part is used as the reaction electrode 4 in FIG. 10. Also the sample solution 3 is formed by a 0.1M PIPES-NaOH aqueous buffer solution (pH 7.5) containing glucose of a predetermined concentration and NAD of 1 mM. Under an application of a predetermined potential to the reaction electrode 4, glucose in the sample solution 3 is oxidized, in the presence of glucose dehydrogenase, to gluconolactone, and NAD is reduced to NADH in this reaction. Then, in the presence of diaphorase associated with glucose dehydrogenase, NADH is oxidized to NAD, and in this reaction, ferrocene serving as the electron transfer mediator is oxidized to generate a ferricinium ion. As the reaction electrode 4 has a predetermined potential with respect to the reference electrode 6, the ferricinium ion receives an electron from the reaction electrode 4 and is reduced to ferrocene. A change in the reduction current corresponding to the glucose concentration in the sample solution can be measured by measuring a current caused by an electron transfer at the reaction electrode 4.

An example of the result of measurement is shown in FIG. 13. FIG. 13 shows a relationship between a variation in the reduction current, measured at the reaction electrode 4 and a glucose concentration (line A).

### (Comparative Example 2) Glucose Sensor

An enzyme electrode is prepared in the same manner as in Example 2, except that a glucose dehydrogenase (bus GDH) derived from Bacillus subtilis, diaphorase (ppuDp) derived from Pseudomonas putida and ferrocene-bonded polyallylamine (Fc-PAA) are immobilized by crosslinking with PEGDE:

busGDH: 0.3 units

ppuDp: 0.6 units

Fc-PAA: 16 μg

PEGDE: 10 μg

This enzyme electrode is used to construct a glucose sensor as in Example 2, and a change in the reduction current corresponding to the glucose concentration in the sample solution can be measured by measuring a current caused by an electron transfer at the reaction electrode 4.

An example of measured result in Comparative Example 2 is shown by B in FIG. 13. The proportion coefficient is smaller than in Example 2, represented by A.

Based on Example 2 and Comparative Example 2, it is identified that the glucose sensor of Example 2, in comparison with that of Comparative Example 2, has a higher sensitivity to the glucose concentration and is capable of quantitative determination of glucose of a lower concentration. This is assumed because, in the enzyme electrode of Example 2, despite of a fact that the amounts of glucose dehydrogenase and diaphorase immobilized on the enzyme electrode are same, glucose dehydrogenase and diaphorase are retained in a physical proximity as both enzymes are associated, whereby the electron exchange between the both enzymes via NAD/NADH is executed more promptly.

### (Example 3) Glucose Fuel Cell

An enzyme electrode is prepared in the same manner as in Example 2, except for employing glassy carbon of 0.5 cm² as the conductive base member. The obtained enzyme electrode has a structured shown in FIG. 12.

A fuel cell is prepared by employing this enzyme electrode as an anode electrode shown in FIG. 11. The electrolyte solution 3 is a 50 mM phosphate buffer (pH 7.5) containing 100 mM of sodium chloride, 5 mM of glucose, and 1 mM of nicotinamide adenine dinucleotide. A predetermined voltage is applied between the anode electrode 15 and the cathode electrode 16 and voltage-current characteristics are measured to obtain the following results:

shortcircuit current density: 988 μA/cm²

maximum electric power: 101 μW/cm²

### (Comparative Example 3) Glucose Fuel Cell

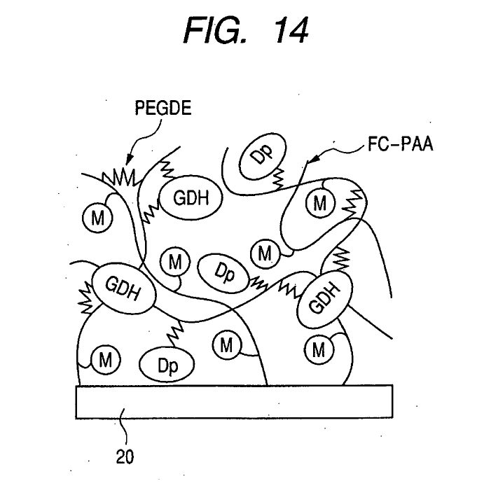

An enzyme electrode of a structure of FIG. 14 and a fuel cell of a structure of FIG. 11 are prepared in the same manner as in Example 3, except for employing the enzymes of Example 1, and voltage-current characteristics are measured to obtain the following results:

shortcircuit current density: 297 μA/cm²

maximum electric power: 32 μW/cm²

Based on Example 3 and Comparative Example 3, it is identified that the glucose fuel cell of Example 3, in comparison with that of Comparative Example 3, has a higher current density and is capable of providing a larger current. This is assumed because, in the fuel cell of Example 3, despite of a fact that the amounts of glucose dehydrogenase and diaphorase immobilized on the enzyme electrode are same, glucose dehydrogenase and diaphorase are retained in a physical proximity as both enzymes are associated, whereby the electron exchange between the both enzymes via NAD/NADH is executed more promptly.

### (Example 4) Glucose Electrochemical Reaction Device

The enzyme electrode prepared in Example 3 was used to prepare an electrochemical reaction device shown in FIG. 10. A 50 mM phosphate buffer (pH 7.5) containing 100 mM of sodium chloride, 5 mM of glucose, and 1 mM of nicotinamide adenine dinucleotide is used as the sample solution 3, then a potential of 0.3 VvsAg/AgCl is applied for 100 minutes under a nitrogen atmosphere, and a product is quantitatively measured by a high-speed liquid chromatography. Gluconolactone is detected from the reaction electrolyte solution, and a reaction charge amount and an amount of generated gluconolactone show a high correlation, indicating that the reaction proceeds quantitatively.

### (Comparative Example 4) Glucose Electrochemical Reaction Device

An electrochemical reaction device was prepared in the same manner as in Example 4, except for employing the enzyme electrode prepared in Comparative Example 3, and was used for producing gluconolactone, utilizing glucose as a substrate. Gluconolactone is detected from the reaction electrolyte solution, and a reaction charge amount and an amount of generated gluconolactone show a high correlation, indicating that the reaction proceeds quantitatively. It is however found that the reaction charge per unit time is smaller than in Example 4.

Based on Example 4 and Comparative Example 4, it is identified that the glucose electrochemical reaction device of Example 4, in comparison with that of Comparative Example 4, has a larger reaction charge per unit time and is capable of converting glucose into gluconolactone more efficiently. This is assumed because, in the electrochemical reaction device of Example 4, despite of a fact that the amounts of glucose dehydrogenase and diaphorase immobilized on the enzyme electrode are same, glucose dehydrogenase and diaphorase are retained in a physical proximity as both enzymes are associated, whereby the electron exchange between the both enzymes via NAD/NADH is executed more promptly.

### (Example 5) Preparation of Associated Protein (His-pfuGDH-Fos and phoDp-Jun) [SEQ ID NOS. 27, 28] of Glucose Dehydrogenase (pfuGDH) Derived from Pyrococcus furiosus and Diaphorase (phoDp) Derived from Pyrococcus horikoshii

A genome DNA is prepared from Pyrococcus furiosus [ATCC 43587] by an ordinary method. A PCR is executed utilizing this genome DNA as a template and employing synthetic oligoDNAs SEQ ID NO:19 having a sequence recognized by BamHI and SEQ ID NO:20 having a sequence recognized by HindIII as primers to obtain an amplified DNA product of 799 bp.

The amplified DNA product is digestion cleaved by restriction enzymes BamHI and HindIII, and is inserted into a same restriction enzyme site of pETDuet-1 (manufactured by Novagen) to obtain a His-pfuGDH expression vector pETDuet-pfuGDH.

Then, a genome DNA is prepared from Pyrococcus horikoshii KT2440 [ATCC 700860] by an ordinary method. A PCR is executed utilizing this genome DNA as a template and employing synthetic oligoDNAs SEQ ID NO:21 having a sequence recognized by NdeI and SEQ ID NO:22 having a sequence recognized by XhoI as primers to obtain an amplified DNA product of 1344 bp.

The amplified DNA product is digestion cleaved by restriction enzymes NdeI and XhoI, and is inserted into a same restriction enzyme site of pETDuet-pfuGDH to obtain a co-expression vector pETDuet-pfuGDH-phoDp, co-expressing His-pfuGDH and phoDp.

Then, as in Example 1,5′-terminal phosphated synthetic oligoDNAs (SEQ ID NO:5 and 6) are used to prepare a DNA fragment. This DNA fragment is inserted in a recognition site for restriction enzymes HindIII and AflII of pETDuet-pfuGDH-phoDp. As a result, there is obtained a co-expression vector pETDuet-pfuGDH-Fos/phoDp of a fusion protein, in which an association site sequence SEQ ID NO:168-Fos (SEQ ID NO:7) is fused to a C-terminal side of His-pfuGDH, and phoDp.

Then, as in Example 1,5′-terminal phosphated synthetic oligoDNAs (SEQ ID NO:8 and 9) are used to prepare a DNA fragment. This DNA fragment is inserted in a recognition site for restriction enzymes XhoI and AvrII of pETDuet-pfuGDH-Fos/ppuDp, thereby obtaining an expression vector pETDuet-pfuGDH-Fos/phoDp-Jun (SEQ ID NO:23) of a fusion protein, formed by a fusion protein in which an association site sequence SEQ ID NO:168-Fos (SEQ ID NO:7) is fused to a C-terminal side of His-pfuGDH, and an association site sequence SEQ ID NO:168-Jun (SEQ ID NO:10) fused at a C-terminal side of phoDp.

Then, a PCR is executed utilizing the genome DNA of Pyrococcus furiosus [ATCC 43587] as a template and employing synthetic oligoDNAs SEQ ID NO:24 having a sequence recognized by NcoI and SEQ ID NO:20 having a sequence recognized by HindIII as primers to obtain an amplified DNA product of 797 bp.

The amplified DNA product is digestion cleaved by restriction enzymes NcoI and HindIII, and is inserted into a same restriction enzyme site of pCDFDuet-1 (manufactured by Novagen) to obtain a pfuGDH expression vector pCDFDuet-pfuGDH.

Then, a PCR is executed utilizing the genome DNA of Pyrococcus horikoshii KT2440 [ATCC 700860] as a template and employing synthetic oligoDNAs SEQ ID NO:21 having a sequence recognized by NdeI and SEQ ID NO:25 having a sequence recognized by XhoI as primers to obtain an amplified DNA product of 1344 bp.

The amplified DNA product is digestion cleaved by restriction enzymes NdeI and XhoI, and is inserted into a same restriction enzyme site of pCDFDuet-pfuGDH to obtain a co-expression vector pCDFDuet-pfuGDH-phoDp (SEQ ID NO:26) of pfuGDH and phoDp.

Expression vectors pETDuet-pfuGDH-Fos/phoDp-Jun and pCDFDuet-pfuGDH-phoDp are transformed into E. coli BL21(DE3) by an ordinary method. The transform can be selected as a resistant strain to antibiotics ampicillin and streptomycin. The transforms were used to prepare enzymes by the method 1.

### (Comparative Example 5) Preparation of pfuGDH and phoDp as Reference

A genome DNA is prepared from Pyrococcus furiosus [ATCC 43587] by an ordinary method. A PCR is executed utilizing this genome DNA as a template and employing synthetic oligo DNAs SEQ ID NO:29 having a sequence recognized by NdeI and SEQ ID NO:30 having a sequence recognized by XhoI as primers to obtain an amplified DNA product of about 800 bp.

The amplified DNA product is digestion cleaved by restriction enzymes NdeI and XhoI, and is inserted into a same restriction enzyme site of pET-21a(+) (manufactured by Novagen) to obtain a pfuGDH-His expression vector pET21-pfuGDH.

Then, a genome DNA is prepared from Pyrococcus horikoshii KT2440 [ATCC 700860] by an ordinary method. A PCR is executed utilizing this genome DNA as a template and employing oligo DNAs SEQ ID NO:21 and 22 as primers to obtain an amplified DNA product of 1344 bp.

The amplified DNA product is digestion cleaved by restriction enzymes NdeI and XhoI, and is inserted into a same restriction enzyme site of pET-21a(+) (manufactured by Novagen) to obtain a phoDp-His expression vector pET21-phoDp.

Expression vectors pET21-pfuGDH and pET21-phoDp are individually transformed into E. coli BL21(DE3) by an ordinary method. The transform can be selected as a resistant strain to antibiotic ampicillin.

These transforms were used to prepare enzymes by the method 2.

### (Example 6) Glucose Sensor

An enzyme electrode of a structure shown in FIG. 12 is prepared in the same manner as in Example 2, except for employing the enzymes prepared in Example 5. The components immobilized on the enzyme electrode has the following composition:

His-pfuGDH-Fos/phoDp-Jun (pfuGDH: 0.3 units, phoDp: 0.6 units)

Fc-PAA: 16 μg

PEGDE: 10 μg

Then a glucose sensor is constructed in the same manner as in Example 2, except for employing this enzyme electrode, and is used for measuring a reduction current corresponding to the glucose concentration in the sample solution. A result of measurement in Example 6 is shown as C in FIG. 13 (having a gradient larger than A).

### (Comparative Example 6) Glucose Sensor

An enzyme electrode of a structure shown in FIG. 14 is prepared in the same manner as in Example 6, except for employing the enzymes prepared in Comparative Example 5. The components immobilized on the enzyme electrode has the following composition:

pfuGDH:0.3 units

phoDp: 0.6 units

Fc-PAA: 16 μg

PEGDE: 10 μg

Then a glucose sensor of a structure of FIG. 10 is constructed in the same manner as in Example 6, except for employing this enzyme electrode, and is used for measuring a reduction current corresponding to the glucose concentration in the sample solution.

A result of measurement in Comparative Example 6 is shown as D in FIG. 13. The proportional coefficient was smaller than in the case of Example 6, represented by C. Based on Example 6 and Comparative Example 6, it is identified that the glucose sensor of Example 6, in comparison with that of Comparative Example 6, has a higher sensitivity to the glucose concentration and is capable of quantitative determination of glucose of a lower concentration. This is assumed because, in the enzyme electrode of Example 6, despite of a fact that the amounts of glucose dehydrogenase and diaphorase immobilized on the enzyme electrode are same, glucose dehydrogenase and diaphorase are retained in a physical proximity as both enzymes are associated, whereby the electron exchange between the both enzymes via NAD/NADH is executed more promptly.

### (Example 7) Glucose Fuel Cell

An enzyme electrode of a structure shown in FIG. 12 is prepared in the same manner as in Example 3, except for employing the enzymes prepared in Comparative Example 5. The components immobilized on the enzyme electrode has the following composition:

His-pfuGDH-Fos/phoDp-Jun (pfuGDH: 0.3 units, phoDp: 0.6 units)

Fc-PAA: 16 μg

PEGDE: 10 μg

A fuel cell of a structure of FIG. 10 is prepared in the same manner as in Example 3, except for employing this enzyme electrode, and voltage-current characteristics are measured to obtain the following results:

shortcircuit current density: 1321 μA/cm²

maximum electric power: 126 μW/cm²

### (Comparative Example 7) Glucose Fuel Cell

An enzyme electrode of a structure shown in FIG. 14 is prepared in the same manner as in Example 7, except for employing the enzymes prepared in Comparative Example 5. The components immobilized on the enzyme electrode has the following composition:

pfuGDH: 0.3 units

phoDp: 0.6 units

Fc-PAA: 16 μg

PEGDE: 10 μg

A fuel cell of a structure are prepared in the same manner as in Example 7, except for employing this enzyme electrode, and voltage-current characteristics are measured to obtain the following results:

shortcircuit current density: 423 μA/cm²

maximum electric power: 41 μW/cm²

Based on Example 7 and Comparative Example 7, it is identified that the glucose fuel cell of Example 7, in comparison with that of Comparative Example 7, has a higher current density and is capable of providing a larger current. This is assumed because, in the fuel cell of Example 7, despite of a fact that the amounts of glucose dehydrogenase and diaphorase immobilized on the enzyme electrode are same, glucose dehydrogenase and diaphorase are retained in a physical proximity as both enzymes are associated, whereby the electron exchange between the both enzymes via NAD/NADH is executed more promptly.

### (Example 8) Glucose Electrochemical Reaction Device

An electrochemical reaction device of a structure shown in FIG. 10 was constructed in the same manner as in Example 4, except for employing the enzyme electrode prepared in Comparative Example 7, and was subjected to an electrochemical reaction. Gluconolactone is detected from the reaction electrolyte solution, and a reaction charge amount and an amount of generated gluconolactone show a high correlation, indicating that the reaction proceeds quantitatively.

### (Comparative Example 8) Glucose Electrochemical Reaction Device

An electrochemical reaction device of FIG. 10 is prepared in the same manner as in Example 8, except for employing the enzyme electrode prepared in Comparative Example 7, and was used for an electrochemical reaction. Gluconolactone is detected from the reaction electrolyte solution, and a reaction charge amount and an amount of generated gluconolactone show a high correlation, indicating that the reaction proceeds quantitatively. It is however found that the reaction charge per unit time is smaller than in Example 8.

Based on Example 8 and Comparative Example 8, it is identified that the glucose electrochemical reaction device of Example 8, in comparison with that of Comparative Example 8, has a larger reaction charge per unit time and is capable of converting glucose into gluconolactone more efficiently. This is assumed because, in the electrochemical reaction device of Example 8, despite of a fact that the amounts of glucose dehydrogenase and diaphorase immobilized on the enzyme electrode are same, glucose dehydrogenase and diaphorase are retained in a physical proximity as both enzymes are associated, whereby the electron exchange between the both enzymes via NAD/NADH is executed more promptly.

### (Example 9) Preparation of Associated Protein (His-sceADH-Fos and ppuDp-Jun) [SEQ ID NOS. 36, 16] of Alcohol Dehydrogenase (sceADH) Derived from Saccharomyces cerevisiae and Diaphorase (ppuDp) Derived from Pseudomonas putida

A genome DNA is prepared from Saccharomyces cerevisiae [ATCC 47058] by an ordinary method. A PCR is executed utilizing this genome DNA as a template and employing synthetic oligoDNAs SEQ ID NO:31 having a sequence recognized by BamHI and SEQ ID NO:32 having a sequence recognized by HindIII as primers to obtain an amplified DNA product of 1075 bp.

The amplified DNA product is digestion cleaved by restriction enzymes BamHI and HindIII, and is inserted into a same restriction enzyme site of pETDuet-1 (manufactured by Novagen) to obtain a His-sceADH expression vector pETDuet-sceADH.

Then, a genome DNA is prepared from Pseudomonas putida KT2440 [ATCC 47054] by an ordinary method. A PCR is executed utilizing this genome DNA as a template and employing synthetic oligoDNAs SEQ ID NOS. 3 and 4 as primers to obtain an amplified DNA product of 729 bp.

The amplified DNA product is digestion cleaved by restriction enzymes NdeI and XhoI, and is inserted into a same restriction enzyme site of pETDuet-sceADH to obtain a co-expression vector pETDuet-sceADH-ppuDp, co-expressing His-sceADH and ppuDp.

Then, as in Example 1,5′-terminal phosphated synthetic oligoDNAs (SEQ ID NO:5 and 6) are used to prepare a DNA fragment. This DNA fragment is inserted in a recognition site for restriction enzymes HindIII and AflII of pETDuet-sceADH-ppuDp. As a result, there is obtained an expression vector pETDuet-sceADH-Fos/ppuDp of a fusion protein, in which an association site sequence SEQ ID NO:168-Fos (SEQ ID NO:7) is fused to a C-terminal side of His-sceADH, and ppuDp.

Then, as in Example 1,5′-terminal phosphated synthetic oligoDNAs (SEQ ID NO:8 and 9) are used to prepare a DNA fragment. This DNA fragment is inserted in a recognition site for restriction enzymes XhoI and AvrII of pETDuet-sceADH-Fos/ppuDp, thereby obtaining an expression vector pETDuet-sceADH-Fos/ppuDp-Jun (SEQ ID NO:33) of a fusion protein, formed by a fusion protein in which an association site sequence SEQ ID NO:168-Fos (SEQ ID NO:7) is fused to a C-terminal side of His-sceADH, and an association site sequence SEQ ID NO:168-Jun (SEQ ID NO:10) fused at a C-terminal side of ppuDp.

Then, a PCR is executed utilizing the genome DNA of Saccharomyces cerevisiae [ATCC 47058] as a template and employing synthetic oligoDNAs SEQ ID NO:34 having a sequence recognized by NcoI and SEQ ID NO:32 having a sequence recognized by HindIII as primers to obtain an amplified DNA product of 1073 bp. The amplified DNA product is digestion cleaved by restriction enzymes NcoI and HindIII, and is inserted into a same restriction enzyme site of pCDFDuet-1 (manufactured by Novagen) to obtain a sceADH expression vector pCDFDuet-sceADH.

Then, a PCR is executed utilizing the genome DNA of Pseudomonas putida KT2440 [ATCC 47054] as a template and employing synthetic oligoDNAs SEQ ID NO:3 and SEQ ID NO:13 as primers to obtain an amplified DNA product of 729 bp. The amplified DNA product is digestion cleaved by restriction enzymes NdeI and XhoI, and is inserted into a same restriction enzyme site of pCDFDuet-sceADH to obtain a co-expression vector pCDFDuet-sceADH-ppuDp (SEQ ID NO:35) of sceADH and ppuDp.

Expression vectors pETDuet-sceADH-Fos/ppuDp-Jun and pCDFDuet-sceADH-ppuDp are transformed into E. coli BL21(DE3) by an ordinary method. The transform can be selected as a resistant strain to antibiotics ampicillin and streptomycin. The transforms were used to prepare enzymes by the method 1.

### (Comparative Example 9) Preparation of sceADH and ppuDp as Reference

A genome DNA is prepared from Saccharomyces cerevisiae [ATCC 47058] by an ordinary method. A PCR is executed utilizing this genome DNA as a template and employing synthetic oligo DNAs SEQ ID NO:37 having a sequence recognized by NdeI and SEQ ID NO:38 having a sequence recognized by XhoI as primers to obtain an amplified DNA product of about 1074 bp. The amplified DNA product is digestion cleaved by restriction enzymes NdeI and XhoI, and is inserted into a same restriction enzyme site of pET-21a(+) (manufactured by Novagen) to obtain a sceADH-His expression vector pET21-sceADH.

Then, a genome DNA is prepared from Pseudomonas putida KT2440 [ATCC 47054] by an ordinary method. A PCR is executed utilizing this genome DNA as a template and employing oligo DNAs SEQ ID NO:3 and SEQ ID NO:4 as primers to obtain an amplified DNA product of 729 bp. The amplified DNA product is digestion cleaved by restriction enzymes NdeI and XhoI, and is inserted into a same restriction enzyme site of pET-21a(+) (manufactured by Novagen) to obtain a ppuDp-His expression vector pET21-ppuDp.

Expression vectors pET21-sceADH and pET21-ppuDp are individually transformed into E. coli BL21(DE3) by an ordinary method. The transform can be selected as a resistant strain to antibiotic ampicillin.

The transforms were individually used to prepare enzymes by the method 2.

### (Example 10) Alcohol Sensor

A conductive base member 20 is formed by glassy carbon of a diameter of 3 mm. On the conductive base member 20, an associated protein (His-sceADH-Fos/ppuDp-Jun) of alcohol dehydrogenase (sceADH) derived from Saccharomyces cerevisiae in Example 9 and diaphorase (ppuDp) derived from Pseudomonas putida, and a ferrocene-bonded polyallylamine (Fc-PAA) are immobilized by crosslinking by poly(ethylene glycol) diglycigyl ether (PEGDE). Components immobilized on the enzyme electrode have the following formulation:

His-sceADH-Fos/ppuDp-Jun (sceADH: 0.3 units, ppuDp: 0.6 units)

Fc-PAA: 161g

PEGDE: 10 μg

This enzyme electrode is used as the reaction electrode 4 in FIG. 10 to construct an alcohol sensor. Also the sample solution 3 is formed by a 0.1M PIPES-NaOH aqueous buffer solution (pH 7.5) containing alcohol of a predetermined concentration and NAD of 1 mM. A potential of 300 mV is applied to the reaction electrode 4 with respect to the reference electrode 6. In this state, alcohol in the sample solution 3 is oxidized, in the presence of alcohol dehydrogenase, to acetaldehyde, and NAD is reduced to NADH in this reaction. Then, in the presence of diaphorase associated with alcohol dehydrogenase, NADH is oxidized to NAD, and in this reaction, ferrocene serving as the electron transfer mediator is oxidized to generate a ferricinium ion. As the reaction electrode 4 has a potential of 300 mV with respect to the reference electrode 6, the ferricinium ion receives an electron from the reaction electrode 4 and is reduced to ferrocene. A change in the reduction current corresponding to the alcohol concentration in the sample solution can be measured by measuring a current caused by an electron transfer at the reaction electrode 4.

### (Comparative Example 10) Alcohol Sensor

An enzyme electrode is prepared in the same manner as in Example 10, except for employing the enzymes prepared in Comparative Example 9, and a sensor of the structure shown in FIG. 10 is prepared. Components of the enzyme electrode have the following compositions:

sceADH: 0.3 units

ppuDp: 0.6 units

Fc-PAA: 16 μg

PEGDE: 10 μg

This sensor is used to measure a change in the reduction current corresponding to the ethanol as in Example 10. The difference in the measured results of Example 10 and Comparative Example 10 is similar to that between A and B in FIG. 13.

Based on Example 10 and Comparative Example 10, it is identified that the alcohol sensor of Example 10, in comparison with that of Comparative Example 10, has a higher sensitivity to the alcohol concentration and is capable of quantitative determination of glucose of a lower concentration. This is assumed because, in the enzyme electrode of Example 10, despite of a fact that the amounts of alcohol dehydrogenase and diaphorase immobilized on the enzyme electrode are same, alcohol dehydrogenase and diaphorase are retained in a physical proximity as both enzymes are associated, whereby the electron exchange between the both enzymes via NAD/NADH is executed more promptly.

### (Example 11) Alcohol Fuel Cell

A conductive base member 20 is formed by glassy carbon of 0.5 cm². On the conductive base member 20, an associated protein (His-sceADH-Fos::ppuDp-Jun) of alcohol dehydrogenase (sceADH) derived from Saccharomyces cerevisiae in Example 9 and diaphorase (ppuDp) derived from Pseudomonas putida, and a ferrocene-bonded polyallylamine (Fc-PAA) are immobilized by crosslinking by poly(ethylene glycol) diglycigyl ether (PEGDE). Components immobilized on the enzyme electrode have the following formulation:

His-sceADH-Fos::ppuDp (sceADH: 0.3 units, ppuDp: 0.6 units)

Fc-PAA: 16 μg

PEGDE: 10 μg

A fuel cell is prepared by employing this enzyme electrode as an anode electrode shown in FIG. 11. The electrolyte solution 3 is a 50 mM phosphate- buffer (pH 7.5)-containing-100 mM of sodium chloride, 5 mM of alcohol (ethanol), and 1 mM of nicotinamide adenine dinucleotide. A predetermined voltage is applied between the anode electrode 15 and the cathode electrode 16 and voltage-current characteristics are measured to obtain the following results:

shortcircuit current density: 821 μA/cm²

maximum electric power: 80 μW/cm²

### [Comparative Example 11] Alcohol Fuel Cell

An enzyme electrode is prepared in the same manner as in Example 11, except for employing enzyme prepared in Comparative Example 9. Components immobilized on the enzyme electrode have the following formulation:

sceADH: 0.3 units

ppuDp: 0.6 units

Fc-PAA: 16 μg

PEGDE: 10 μg

A fuel cell of a structure of FIG. 11 is prepared in the same manner as in Example 11, except for employing this enzyme electrode, and voltage-current characteristics are measured to obtain the following results:

shortcircuit current density: 246 μA/cm²

maximum electric power: 25 μW/cm²

Based on Example 11 and Comparative Example 11, it is identified that the alcohol fuel cell of Example 11, in comparison with that of Comparative Example 11, has a higher current density and is capable of providing a larger current. This is assumed because, in the fuel cell of Example 11, despite of a fact that the amounts of alcohol dehydrogenase and diaphorase immobilized on the enzyme electrode are same, alcohol dehydrogenase and diaphorase are retained in a physical proximity as both enzymes are associated, whereby the electron exchange between the both enzymes via NAD/NADH is executed more promptly.

### (Example 12) Alcohol Electrochemical Reaction Device

The enzyme electrode prepared in Example 11 was used to prepare an electrochemical reaction device shown in FIG. 10. A 50 mM phosphate buffer (pH 7.5) containing 100 mM of sodium chloride, 5 mM of alcohol, and 1 mM of nicotinamide adenine dinucleotide is used as the sample solution 3, then a predetermined potential is applied , and a product is quantitatively mepredeasured by a high-speed liquid chromatography. Acetaldehyde is detected from the reaction electrolyte solution, and a reaction charge amount and an amount of generated acetaldehyde show a high correlation, indicating that the reaction proceeds quantitatively.

### (Comparative Example 12) Alcohol Electrochemical Reaction Device

An electrochemical reaction device as shown in FIG. 10 was prepared in the same manner as in Example 12, except for employing the enzyme electrode prepared in Comparative Example 11, and was used for executing an electrochemical reaction. Acetaldehyde is detected from the reaction electrolyte solution, and a reaction charge amount and an amount of generated acetaldehyde show a high correlation, indicating that the reaction proceeds quantitatively. It is however found that the reaction charge per unit time is smaller than in Example 12.

Based on Example 12 and Comparative Example 12, it is identified that the alcohol electrochemical reaction device of Example 12, in comparison with that of Comparative Example 12, has a larger reaction charge per unit time and is capable of converting alcohol into acetaldehyde more efficiently. This is assumed because, in the electrochemical reaction device of Example 12, despite of a fact that the amounts of alcohol dehydrogenase and diaphorase immobilized on the enzyme electrode are same, alcohol dehydrogenase and diaphorase are retained in a physical proximity as both enzymes are associated, whereby the electron exchange between the both enzymes via NAD/NADH is executed more promptly.

### (Example 13) Preparation of Associated Protein (His-pfuADH-Fos/phoDp-Jun) [SEQ ID NOS. 44, 28] of Alcohol Dehydrogenase (pfuADH) Derived from Pyrococcus furiosus and Diaphorase (phoDp) Derived from Pyrococcus horikoshii

A genome DNA is prepared from Pyrococcus furiosus [ATCC 43587] by an ordinary method. A PCR is executed utilizing this genome DNA as a template and employing synthetic oligoDNAs SEQ ID NO:39 having a sequence recognized by BamHI and SEQ ID NO:40 having a sequence recognized by HindIII as primers to obtain an amplified DNA product of 1147 bp. The amplified DNA product is digestion cleaved by restriction enzymes BamHI and HindIII, and is inserted into a same restriction enzyme site of pETDuet-1 (manufactured by Novagen) to obtain a His-pfuADH expression vector pETDuet-pfuADH.

Then, a genome DNA is prepared from Pyrococcus horikoshii KT2440 [ATCC 700860] by an ordinary method. A PCR is executed utilizing this genome DNA as a template and employing synthetic oligoDNAs SEQ ID NO:21 and SEQ ID NO:22 as primers to obtain an amplified DNA product of 1344 bp.

The amplified DNA product is digestion cleaved by restriction enzymes NdeI and XhoI, and is inserted into a same restriction enzyme site of pETDuet-pfuADH to obtain a co-expression vector pETDuet-pfuADH-phoDp, co-expressing His-pfuADH and phoDp.

Then, as in Example 1,5′-terminal phosphated synthetic oligoDNAs (SEQ ID NO:5 and 6) are used to prepare a DNA fragment (double strand). This DNA fragment is inserted in a recognition site for restriction enzymes HindIII and AflII of pETDuet-pfuADH-phoDp. As a result, there is obtained a co-expression vector pETDuet-pfuADH-Fos/phoDp of a fusion protein, in which an association site sequence SEQ ID NO:168-Fos (SEQ ID NO:7) is fused to a C-terminal side of His-pfuADH, and phoDp.

Then, as in Example 1, 5′-terminal phosphated synthetic oligoDNAs (SEQ ID NO:8 and 9) are used to prepare a DNA fragment (double strand). This DNA fragment is inserted in a recognition site for restriction enzymes XhoI and AvrII of pETDuet-pfuADH-Fos/ppuDp, thereby obtaining an expression vector pETDuet-pfuADH-Fos/phoDp-Jun (SEQ ID NO:41) of a fusion protein, formed by a fusion protein in which an association site sequence SEQ ID NO:168-Fos (SEQ ID NO:7) is fused to a C-terminal side of His-pfuADH, and an association site sequence SEQ ID NO:168-Jun (SEQ ID NO:10) fused at a C-terminal side of phoDp.

Then, a PCR is executed utilizing the genome DNA of Pyrococcus furiosus [ATCC 43587) as a template and employing synthetic oligoDNAs SEQ ID NO:42 having a sequence recognized by NcoI and SEQ ID NO:40 having a sequence recognized by HindIII as primers to obtain an amplified DNA product of 1145 bp. The amplified DNA product is digestion cleaved by restriction enzymes NcoI and HindIII, and is inserted into a same restriction enzyme site of pCDFDuet-1 (manufactured by Novagen) to obtain a pfuADH expression vector pCDFDuet-pfuADH.

Then, a PCR is executed utilizing the genome DNA of Pyrococcus horikoshii KT2440 [ATCC 700860] as a template and employing synthetic oligoDNAs SEQ ID NO:21 and SEQ ID NO:25 as primers to obtain an amplified DNA product of 1344 bp. The amplified DNA product is digestion cleaved by restriction enzymes NdeI and XhoI, and is inserted into a same restriction enzyme site of pCDFDuet-pfuADH to obtain a co-expression vector pCDFDuet-pfuADH-phoDp (SEQ ID NO:43) of pfuADH and phoDp.

Expression vectors pETDuet-pfuADH::phoDp and pCDFDuet-pfuADH-phoDp are transformed into E. coli BL21(DE3) by an ordinary method. The transform can be selected as a resistant strain to antibiotics ampicillin and streptomycin. The transforms were used to prepare enzymes by the method 1. Enzymes, thus obtained from thermophilic bacteria, may be used for constructing an enzyme electrode and the like.

### (Example 17) Preparation of Associated Protein (His-lplLDH-Dzip1and goxDp-Dzip2) [SEQ ID Nos. 61, 62] of Lactic Acid Dehydrogenase (lplLDH) Derived from Lactobacillus plantarum and Diaphorase (goxDp) Derived from Gluconobacter oxydans

A genome DNA is prepared from Lactobacillus plantarum [ATCC 10241] by an ordinary method. A PCR is executed utilizing this genome DNA as a template and employing synthetic oligo DNAs SEQ ID NO:47 having a sequence recognized by BamHI and SEQ ID NO:48 having a sequence recognized by HindIII as primers to obtain an amplified DNA product of 982 bp. The amplified DNA product is digestion cleaved by restriction enzymes BamHI and HindIII, and is inserted into a same restriction enzyme site of pETDuet-1 (manufactured by Novagen) to obtain a His-lplLDH expressing vector pETDuet-lplLDH.

Then, a genome DNA is prepared from Gluconobacter oxydans [ATCC 621H] by an ordinary method. A PCR is executed utilizing this genome DNA as a template and employing synthetic oligo DNAs SEQ ID NO:49 and SEQ ID NO:50 as primers to obtain an amplified DNA product of 1428 bp. The amplified DNA product is digestion cleaved by restriction enzymes NdeI and XhoI, and is inserted into a same restriction enzyme site of pETDuet-lplLDH to obtain a co-expression vector pETDuet-lplLDH-goxDp, co-expressing His-lplLDH and goxDp.

Then, 5′-terminal phosphated synthetic oligoDNAs (SEQ ID NO:51 and 52) are mixed in equal amounts in a TE buffer, and annealed by heating followed by standing to cool. This DNA fragment is inserted in a recognition site for restriction enzymes HindIII and AflII of pETDuet-lplLDH/goxDp, thereby obtaining a co-expression vector pETDuet-lplLDH-Dzip1/goxDp of a fusion protein, in which an association site sequence SEQ ID NO:169-Dzip1(SEQ ID NO:53) is fused to a C-terminal side of His-lplLDH, and goxDp.

Then, 5′-terminal phosphated synthetic oligoDNAs (SEQ ID NO:54 and 55) are mixed in equal amounts in a TE buffer, and annealed by heating followed by standing to cool. This DNA fragment is inserted in a recognition site for restriction enzymes XhoI and AvrII of pETDuet-lplLDH-goxDp, thereby obtaining a co-expression vector pETDuet-lplLDH-Dzip1/goxDp-Dzip2 (SEQ ID NO:57) of a fusion protein, in which an association site sequence SEQ ID NO:169-Dzip1 (SEQ ID NO:53) is fused to a C-terminal side of His-lplLDH, and a fusion protein in which an association site sequence SEQ ID NO:169-Dzip2 (SEQ ID NO:56) is fused at a C-terminal side of goxDp.

Then a PCR is executed utilizing the genome DNA of Lactobacillus plantarum [ATCC 10241] as a template and employing synthetic oligoDNAs SEQ ID NO:58 having a sequence recognized by NcoI and SEQ ID NO:48 having a sequence recognized by HindIII as primers to obtain an amplified DNA product of 980 bp. The amplified DNA product is digestion cleaved by restriction enzymes NcoI and HindIII, and is inserted into a same restriction enzyme site of pCDFDuet-1 (manufactured by Novagen) to obtain a lplLDH expression vector pCDFDuet-lplLDH.

Then, a PCR is executed utilizing the genome DNA of Gluconobacter oxydans [ATCC 621H] as a template and employing synthetic oligoDNAs SEQ ID NO:49 having a sequence recognized by NdeI and SEQ ID NO:59 having a sequence recognized by XhoI as primers to obtain an amplified DNA product of 1428 bp. The amplified DNA product is digestion cleaved by restriction enzymes NdeI and XhoI, and is inserted into a same restriction enzyme site of pCDFDuet-lplLDH to obtain a co-expression vector pCDFDuet-lplLDH-goxDp (SEQ ID NO:60) of lplLDH and goxDp.

Expression vectors pETDuet-lplLDH-Dzip1/goxDp-Dzip2 and pCDFDuet-lplLDH-goxDp are transformed into E. coli BL21(DE3) by an ordinary method. The transform can be selected as a resistant strain to antibiotics ampicillin and streptomycin. The transforms were used to prepare enzymes by the method 1.

### (Comparative Example 17) Preparation of lplLDH and goxDp as Reference

A genome DNA is prepared from Lactobacillus plantarum [ATCC 10241] by an ordinary method. A PCR is executed utilizing this genome DNA as a template and employing synthetic oligo DNAs SEQ ID NO:63 having a sequence recognized by NdeI and SEQ ID NO:64 having a sequence recognized by XhoI as primers to obtain an amplified DNA product of about 980 bp. The amplified DNA product is digestion cleaved by restriction enzymes NdeI and XhoI, and is inserted into a same restriction enzyme site of pET-21a(+) (manufactured by Novagen) to obtain a lplLDH-His expression vector pET21-lplLDH.

Then, a genome DNA is prepared from Gluconobacter oxydans [ATCC 621H] by an ordinary method. A PCR is executed utilizing this genome DNA as a template and employing oligo DNAs SEQ ID NO:49 having a sequence recognized by NdeI and SEQ ID NO:50 having a sequence recognized by XhoI as primers to obtain an amplified DNA product of about 1428 bp. The amplified DNA product is digestion cleaved by restriction enzymes NdeI and XhoI, and is inserted into a same restriction enzyme site of pET-21a(+) (manufactured by Novagen) to obtain a goxDp-His expression vector pET21-goxDp.

Expression vectors pET21-lplLDH and pET21-goxDp are individually transformed into E. coli BL21(DE3) by an ordinary method. The transform can be selected as a resistant strain to antibiotic ampicillin.

These transforms were individually used to prepare enzymes by the method 2.

### (Example 18) Lactic Acid Sensor

A structure of the enzyme electrode is schematically shown in FIG. 12. A conductive base member 20 is formed by glassy carbon of a diameter of 3 mm. On the conductive base member 20, an associated protein (His-lplLDH-Dzip1/goxDp-Dzip2) of lactic acid dehydrogenase (lplLDH) derived from Lactobacillus plantarum in Example 17 and diaphorase (goxDp) derived from Gluconobacter oxydans, and a ferrocene-bonded polyallylamine (Fc-PAA) are immobilized by crosslinking by poly(ethylene glycol) diglycigyl ether (PEGDE). Components immobilized on the enzyme electrode have the following formulation:

His-lplLDH-Dzip1/goxDp-Dzip2-(lplLDH:-0.3 units, goxDp: 0.6 units)

Fc-PAA: 16 μg

PEGDE: 10 μg

This enzyme electrode part is used as the reaction electrode 4 in FIG. 10 to construct a lactic acid sensor. Also the sample solution 3 is formed by a 0.1M PIPES-NaOH aqueous buffer solution (pH 7.5) containing lactic acid (L-lactic acid) of a predetermined concentration and 1 mM of NAD. Under an application of a potential of 300 mV to the reaction electrode 4 with respect to the reference electrode 6, lactic acid in the sample solution 3 is oxidized, in the presence of lactic acid dehydrogenase, to pyruvic acid, and NAD is reduced to NADH in this reaction. Then, in the presence of diaphorase associated with lactic acid dehydrogenase, NADH is oxidized to NAD, and in this reaction, ferrocene serving as the electron transfer mediator is oxidized to generate a ferricinium ion. As the reaction electrode 4 has a potential of 300 mV with respect to the reference electrode 6, the ferricinium ion receives an electron from the reaction electrode 4 and is reduced to ferrocene. A reduction current corresponding to the lactic acid concentration in the sample solution can be measured by measuring a current caused by an electron transfer at the reaction electrode 4.

### (Comparative Example 18) Lactic Acid Sensor

An enzyme electrode is prepared in the same manner as in Example 18, except for employing the enzymes prepared in Comparative Example 17. Components immobilized on the enzyme electrode have the following compositions:

lplLDH: 0.3 units

goxDp: 0.6 units

Fc-PAA: 16 μg

PEGDE: 10 μg

The enzyme electrode is used as the reaction electrode 4 in FIG. 10 for constructing a lactic acid sensor, and is used to measure a change in the reduction current corresponding to the lactic acid concentration as in Example 18.

The difference in the measured results of Example 18 and Comparative Example 18 is similar to that between A and B in FIG. 13.

### (Example 19) Lactic Acid Fuel Cell

A conductive base member 20 is formed by glassy carbon of 0.5 cm². On the conductive base member 20, an associated protein (His-lplLDH-Dzip1/goxDp-Dzip2) of lactic acid dehydrogenase (lplLDH) derived from Lactobacillus plantarum in Example 17 and diaphorase (goxDp) derived from Gluconobacter oxydans, and a ferrocene-bonded polyallylamine (Fc-PAA) are immobilized by crosslinking by poly(ethylene glycol) diglycigyl ether (PEGDE). Components immobilized on the enzyme electrode have the following formulation:

His-lplLDH-Dzip1/goxDp-Dzip2 (lplLDH: 0.3 units, goxDp: 0.6 units)

Fc-PAA: 16 μg

PEGDE: 10 μg

A fuel cell is prepared by employing this enzyme electrode as an anode electrode shown in FIG. 11. The electrolyte solution 3 is a 50 mM phosphate buffer (pH 7.5) containing 100 mM of sodium chloride, 5 mM of lactic acid (L-lactic acid), and 1 mM of nicotinamide adenine dinucleotide. A predetermined voltage is applied between the anode electrode 15 and the cathode electrode 16 and voltage-current characteristics are measured to obtain the following results:

shortcircuit current density: 1331 μA/cm²

maximum electric power: 121 μW/cm²

### (Comparative Example 19) Lactic Acid Fuel Cell

An enzyme electrode was prepared in the same manner as in Example 19, except for employing enzymes prepared in Comparative Example 17. Components immobilized on the enzyme electrode have the following formulation:

lplLDH: 0.3 units

goxDp: 0.6 units

Fc-PAA: 16 μg

PEGDE: 10 μg

A fuel cell of a structure of FIG. 11 was prepared in the same manner as in Example 19, except for employing this enzyme electrode, and voltage-current characteristics were measured to obtain the following results:

shortcircuit current density: 397 μA/cm²

maximum electric power: 38 μW/cm²

Based on Example 19 and Comparative Example 19, it is identified that the lactic acid fuel cell of Example 19, in comparison with that of Comparative Example 19, has a higher current density and is capable of providing a larger current. This is assumed because, in the fuel cell of Example 19, despite of a fact that the amounts of lactic acid dehydrogenase and diaphorase immobilized on the enzyme electrode are same, lactic acid dehydrogenase and diaphorase are retained in a physical proximity as both enzymes are associated, whereby the electron exchange between the both enzymes via NAD/NADH is executed more promptly.

### (Example 20) Lactic Acid Electrochemical Reaction Device

The enzyme electrode prepared in Example 19 was used to prepare an electrochemical reaction device shown in FIG. 10. A 50 mM phosphate buffer (pH 7.5) containing 100 mM of sodium chloride, 5 mM of lactic acid (L-lactic acid), and 1 mM of nicotinamide adenine dinucleotide is used as the sample solution 3, then a potential of 0.3 VvsAg/AgCl is applied for 100 minutes under a nitrogen atmosphere, and a product is quantitatively measured by a high-speed liquid chromatography. Pyruvic acid is detected from the reaction electrolyte solution, and a reaction charge amount and an amount of generated pyruvic acid show a high correlation, indicating that the reaction proceeds quantitatively.

### (Comparative Example 20) Lactic Acid Electrochemical Reaction Device

An electrochemical reaction device shown in FIG. 10 was prepared in the same manner as in Example 20, except for employing the enzyme electrode prepared in Comparative Example 19, and was used for executing an electrochemical reaction. Pyruvic acid is detected from the reaction electrolyte solution, and a reaction charge amount and an amount of generated pyruvic acid show a high correlation, indicating that the reaction proceeds quantitatively. It is however found that the reaction charge per unit time is smaller than in Example 20.

Based on Example 20 and Comparative Example 20, it is identified that the lactic acid electrochemical reaction device of Example 20, in comparison with that of Comparative Example 20, has a larger reaction charge per unit time and is capable of converting lactic acid into pyruvic acid more efficiently. This is assumed because, in the electrochemical reaction device of Example 20, despite of a fact that the amounts of lactic acid dehydrogenase and diaphorase immobilized on the enzyme electrode are same, lactic acid dehydrogenase and diaphorase are retained in a physical proximity as both enzymes are associated, whereby the electron exchange between the both enzymes via NAD/NADH is executed more promptly.

### (Example 21) Preparation of Associated Protein (His-tmaLDH-Dzip1and tmaDp-Dzip2) [SEQ ID NOS. 73, 74] of Lactic Acid Dehydrogenase (tmaLDH) Derived from Thermotoga maritima and Diaphorase (tmaDp) Derived from Thermotoga maritime

A genome DNA is prepared from Thermotoga maritima [ATCC 43589] by an ordinary method. A PCR is executed utilizing this genome DNA as a template and employing synthetic oligoDNAs SEQ ID NO:65 having a sequence recognized by BamHI and SEQ ID NO:66 having a sequence recognized by HindIII as primers to obtain an amplified DNA product of 979 bp. The amplified DNA product is digestion cleaved by restriction enzymes BamHI and HindIII, and is inserted into a same restriction enzyme site of pETDuet-1 (manufactured by Novagen) to obtain a His-tamLDH expression vector pETDuet-tmaLDH.

Then, a genome DNA is prepared from Thermotoga maritima [ATCC 43589] by an ordinary method. A PCR is executed utilizing this genome DNA as a template and employing synthetic oligoDNAs SEQ ID NO:67 having a sequence recognized by NdeI and SEQ ID NO:68 having a sequence recognized by XhoI as primers to obtain an amplified DNA product of 1371 bp. The amplified DNA product is digestion cleaved by restriction enzymes NdeI and XhoI, and is inserted into a same restriction enzyme site of pETDuet-tmaLDH to obtain a co-expression vector pETDuet-tmaLDH-tmaDp, co-expressing His-tmaLDH and tmaDp.

Then, as in Example 15, a DNA fragment (double strand) is obtained from SEQ ID NO:51 and 52. This DNA fragment is inserted in a recognition site for restriction enzymes HindIII and AflII of pETDuet-tmaLDH-tmaDp. As a result, there is obtained a co-expression vector pETDuet- tmaLDH-Dzip1/tmaDp of a fusion protein, in which an association site sequence SEQ ID NO:169-Dzip1(SEQ ID NO:53) is fused to a C-terminal side of His-tmaLDH, and tmaDp.

Then, as in Example 17, 5′-terminal phosphated synthetic oligoDNAs (SEQ ID NO:54 and 55) are used to prepare a DNA fragment (double strand). This DNA fragment is inserted in a recognition site for restriction enzymes XhoI and AvrII of pETDuet-tmaLDH-Dzip1/tmaDp, thereby obtaining a co-expression vector pETDuet-tmaLDH-Dzip1/tmaDp-Dzip2 (SEQ ID NO:69) of a fusion protein, in which an association site sequence SEQ ID NO:169-Dzip1(SEQ ID NO:53) is fused to a C-terminal side of His-tmaLDH, and and a fusion protein in which an association site sequence SEQ ID NO:169-Dzip2 (SEQ ID NO:56) is fused at a C-terminal side of tmaDp.

Then, a PCR is executed utilizing the genome DNA of Thermotoga maritima [ATCC 43589] as a template and employing synthetic oligoDNAs SEQ ID NO:70 having a sequence recognized by NcoI and SEQ ID NO:66 having a sequence recognized by HindIII as primers to obtain an amplified DNA product of 977 bp. The amplified DNA product is digestion cleaved by restriction enzymes NcoI and HindIII, and is inserted into a same restriction enzyme site of pCDFDuet-1 (manufactured by Novagen) to obtain a tmaLDH expression vector pCDFDuet-tmaLDH.

Then, a PCR is executed utilizing the genome DNA of Thermotoga maritima [ATCC 43589] as a template and employing synthetic oligoDNAs SEQ ID NO:67 having a sequence recognized by NdeI and SEQ ID NO:71 having a sequence recognized by XhoI as primers to obtain an amplified DNA product of 1371 bp. The amplified DNA product is digestion cleaved by restriction enzymes NdeI and XhoI, and is inserted into a same restriction enzyme site of pCDFDuet-tmaLDH to obtain a co-expression vector pCDFDuet-tmaLDH-tmaDp (SEQ ID NO:72) of tmaLDH and tmaDp.

Expression vectors pETDuet-tmaLDH-Dzip1/tmaDp-Dzip2 and pCDFDuet-tmaLDH-tmaDp are transformed into E. coli BL21(DE3) by an ordinary method. The transform can be selected as a resistant strain to antibiotics ampicillin and streptomycin. The transforms were used to prepare enzymes by the method 1. Enzymes, thus obtained from thermophilic bacteria, may be used for constructing an enzyme electrode and the like.

### (Example 25) Preparation of Associated Protein (His-ppuMDH-Dzip1and goxDp-Dzip2) [SEQ ID NOS. 82, 62] of Malic Acid Dehydrogenase (ppuMDH) Derived from Pseudomonas putida and Diaphorase (goxDp) Derived from Gluconobacter oxydans

A genome DNA is prepared from Pseudomonas putida [ATCC 47054] by an ordinary method. A PCR is executed utilizing this genome DNA as a template and employing synthetic oligoDNAs SEQ ID NO:77 having a sequence recognized by BamHI and SEQ ID NO:78 having a sequence recognized by HindIII as primers to obtain an amplified DNA product of 1288 bp. The amplified DNA product is digestion cleaved by restriction enzymes BamHI and HindIII, and is inserted into a same restriction enzyme site of pETDuet-1 (manufactured by Novagen) to obtain a His-ppuMDH expression vector pETDuet-ppuMDH.

Then, a genome DNA is prepared from Gluconobacter oxydans [ATCC 621H] by an ordinary method. A PCR is executed utilizing this genome DNA as a template and employing synthetic oligoDNAs SEQ ID NO:49 and SEQ ID NO:50 as primers to obtain an amplified DNA product of 1428 bp. The amplified DNA product is digestion cleaved by restriction enzymes NdeI and XhoI, and is inserted into a same restriction enzyme site of pETDuet-ppuMDH to obtain a co-expression vector pETDuet-ppuMDH-goxDp, co-expressing His-ppuMDH and goxDp.

Then, as in Example 17, 5′-terminal phosphated synthetic oligoDNAs (SEQ ID NO:51 and 52) are used to prepare a DNA fragment (double strand). This DNA fragment is inserted in a recognition site for restriction enzymes HindIII and AflII of pETDuet-ppuMDH-goxDp. As a result, there is obtained a co-expression vector pETDuet-ppuMDH-Dzip1/goxDp of a fusion protein, in which an association site sequence SEQ ID NO:169-Dzip1(SEQ ID NO:53) is fused to a C-terminal side of His-ppuMDH, and goxDp.

Then, as in Example 17, 5′-terminal phosphated synthetic oligoDNAs (SEQ ID NO:54 and 55) are used to prepare a DNA fragment (double strand) This DNA fragment is inserted in a recognition site for restriction enzymes XhoI and AvrII of pETDuet-ppuMDH-Dzip1/goxDp, thereby obtaining a co-expression vector pETDuet-ppuMDH-Dzip1/goxDp-Dzip2 (SEQ ID NO:79) of a fusion protein, in which an association site sequence SEQ ID NO:169-Dzip1(SEQ ID NO:53) is fused to a C-terminal side of His-ppuMDH, and a fusion protein in which an association site sequence SEQ ID NO:169-Dzip2 (SEQ ID NO:56) is fused at a C-terminal side of goxDp.

Then, a PCR is executed utilizing the genome DNA of Pseudomonas putida [ATCC 47054] as a template and employing synthetic oligoDNAs SEQ ID NO:80 having a sequence recognized by NcoI and SEQ ID NO:78 having a sequence recognized by HindIII as primers to obtain an amplified DNA product of 1286 bp. The amplified DNA product is digestion cleaved by restriction enzymes NcoI and HindIII, and is inserted into a same restriction enzyme site of pCDFDuet-1 (manufactured by Novagen) to obtain a ppuMDH expression vector pCDFDuet-ppuMDH.

Then, a PCR is executed utilizing the genome DNA of Gluconobacter oxydans [ATCC 621H] as a template and employing synthetic oligoDNAs SEQ ID NO:49 and SEQ ID NO:59 as primers to obtain an amplified DNA product of 1428 bp. The amplified DNA product is digestion cleaved by restriction enzymes NdeI and XhoI, and is inserted into a same restriction enzyme site of pCDFDuet-ppuMDH to obtain a co-expression vector pCDFDuet-ppuMDH-goxDp (SEQ ID NO:81) of ppuMDH and goxDp.

Expression vectors pETDuet-ppuMDH-Dzip1/goxDp-Dzip2 and pCDFDuet-ppuMDH-goxDp are transformed into E. coli BL21(DE3) by an ordinary method. The transform can be selected as a resistant strain to antibiotics ampicillin and streptomycin. The transforms were used to prepare enzymes by the method 1.

### [Comparative Example 25] Preparation of ppuMDH and goxDp as Reference

A genome DNA is prepared from Pseudomonas putida [ATCC 47054] by an ordinary method. A PCR is executed utilizing this genome DNA as a template and employing synthetic oligo DNAs SEQ ID NO:83 having a sequence recognized by NdeI and SEQ ID NO:84 having a sequence recognized by XhoI as primers to obtain an amplified DNA product of about 1290 bp. The amplified DNA product is digestion cleaved by restriction enzymes NdeI and XhoI, and is inserted into a same restriction enzyme site of pET-21a(+) (manufactured by Novagen) to obtain a ppuMDH-His expression vector pET21-ppuMDH.

Then, a genome DNA is prepared from Gluconobacter oxydans [ATCC 621H] by an ordinary method. A PCR is executed utilizing this genome DNA as a template and employing oligo DNAs SEQ ID NO:49 and SEQ ID NO:50 as primers to obtain an amplified DNA product of about 1420 bp. The amplified DNA product is digestion cleaved by restriction enzymes NdeI and XhoI, and is inserted into a same restriction enzyme site of pET-21a(+) (manufactured by Novagen) to obtain a goxDp-His expression vector pET21-goxDp.

Expression vectors pET21-ppuMDH and pET21-goxDp are individually transformed into E. coli BL21(DE3) by an ordinary method. The transform can be selected as a resistant strain to antibiotic ampicillin.

These transforms were individually used to prepare enzymes by the method 2.

### (Example 26) Malic Acid Sensor

A structure of the enzyme electrode is schematically shown in FIG. 4. A conductive base member 20 is formed by glassy carbon of a diameter of 3 mm. On the conductive base member 20, an associated protein (His-ppuMDH-Dzip1/goxDp-Dzip2) of malic acid dehydrogenase (ppuMDH) derived from Pseudomonas putida in Example 25 and diaphorase (goxDp) derived from Gluconobacter oxydans, and a ferrocene-bonded polyallylamine (Fc-PAA) are immobilized by crosslinking by poly(ethylene glycol) diglycigyl ether (PEGDE). Components immobilized on the enzyme electrode have the following formulation:

His-ppuMDH-Dzip1/goxDp-Dzip2 (ppuMDH: 0.3 units, goxDp: 0.6 units)

Fc-PAA: 16 μg

PEGDE: 10 μg

This enzyme electrode is used as the reaction electrode 4 in FIG. 10 to construct a malic acid sensor. Also the sample solution 3 is formed by a 0.1M PIPES-NaOH aqueous buffer solution (pH 7.5) containing malic acid of a predetermined concentration and 1 mM of NAD. Under an application of a potential of 300 mV to the reaction electrode 4 with respect to the reference electrode 6, malic acid in the sample solution 3 is oxidized, in the presence of malic acid dehydrogenase, to pyruvic acid, and NAD is reduced to NADH in this reaction. Then, in the presence of diaphorase associated with malic acid dehydrogenase, NADH is oxidized to NAD, and in this reaction, ferrocene serving as the electron transfer mediator is oxidized to generate a ferricinium ion. As the reaction electrode 4 has a potential of 300 mV with respect to the reference electrode 6, the ferricinium ion receives an electron from the reaction electrode 4 and is reduced to ferrocene. A reduction current corresponding to the malic acid concentration in the sample solution can be measured by measuring a current caused by an electron transfer at the reaction electrode 4.

### (Comparative Example 26) Malic Acid Sensor

An enzyme electrode is prepared in the same manner as in Example 26, except for employing the enzymes prepared in Comparative Example 25. Components immobilized on the enzyme electrode have the following compositions:

ppuMDH: 0.3 units

goxDp: 0.6 units

Fc-PAA: 16 μg

PEGDE: 10 μg

The enzyme electrode is used as the reaction electrode 4 in FIG. 10 for constructing a malic acid sensor, and is used to measure a change in the reduction current corresponding to the malic acid concentration as in Example 26. A difference between Example 26 and Comparative Example 26 is similar to the difference between A and B in FIG. 13.

### (Example 27) Malic Acid Fuel Cell

FIG. 4 shows the structure of the enzyme electrode. A conductive base member 20 is formed by glassy carbon of 0.5 cm². On the conductive base member 20, an associated protein (His-ppuMDH-Dzip1/goxDp-Dzip2) of malic acid dehydrogenase (ppuMDH) derived from Pseudomonas putida in Example 25 and diaphorase (goxDp) derived from Gluconobacter oxydans, and a ferrocene-bonded polyallylamine (Fc-PAA) are immobilized by crosslinking by poly(ethylene glycol) diglycigyl ether (PEGDE). Components immobilized on the enzyme electrode have the following formulation:

His-ppuMDH-Dzip1/goxDp-Dzip2 (ppuMDH: 0.3 units, goxDp: 0.6 units)

Fc-PAA: 16 μg

PEGDE: 10 μg

A fuel cell is prepared by employing this enzyme electrode as an anode electrode shown in FIG. 11. The electrolyte solution 3 is a 50 mM phosphate buffer (pH 7.5)-containing 100 mM of sodium chloride, 5 mM of malic acid, and 1 mM of nicotinamide adenine dinucleotide. A predetermined voltage is applied between the anode electrode 15 and the cathode electrode 16 and voltage-current characteristics are measured to obtain the following results:

shortcircuit current density: 1026 μA/cm²

maximum electric power: 99 μW/cm²

### (Comparative Example 27) Malic Acid Fuel Cell

An enzyme electrode was prepared in the same manner as in Example 27, except for employing enzymes prepared in Comparative Example 25. Components immobilized on the enzyme electrode have the following formulation:

ppuMDH: 0.3 units

goxDp: 0.6 units

Fc-PAA: 16 μg

PEGDE: 10 μg

A fuel cell of a structure of FIG. 11 was prepared with this enzyme electrode, in the same manner as in Example 27, except for employing, as the electrolyte solution 3, a 50 mM phosphate buffer (pH 7.5) containing 100 mM of sodium chloride, 5 mM of malic acid, and 1 mM of nicotinamide adenine dinucleotide, and voltage-current characteristics were measured to obtain the following results:

shortcircuit current density: 298 μA/cm²

maximum electric power: 31 μW/cm²

Based on Example 27 and Comparative Example 27, it is identified that the malic acid, fuel cell of Example 27, in comparison with that of Comparative Example 27, has a higher current density and is capable of providing a larger current. This is assumed because, in the fuel cell of Example 27, despite of a fact that the amounts of malic acid dehydrogenase and diaphorase immobilized on the enzyme electrode are same, malic acid dehydrogenase and diaphorase are retained in a physical proximity as both enzymes are associated, whereby the electron exchange between the both enzymes via NAD/NADH is executed more promptly.

### (Example 28) Malic Acid Electrochemical Reaction Device

The enzyme electrode prepared in Example 27 was used to prepare an electrochemical reaction device shown in FIG. 10. A 50 mM phosphate buffer (pH 7.5) containing 100 mM of sodium chloride, 5 mM of malic acid, and 1 mM of nicotinamide adenine dinucleotide is used as the sample solution 3, then a potential of 0.3 VvsAg/AgCl is applied for 100 minutes under a nitrogen atmosphere, and a product is quantitatively measured by a high-speed liquid chromatography. Pyruvic acid is detected from the reaction electrolyte solution, and a reaction charge amount and an amount of generated pyruvic acid show a high correlation, indicating that the reaction proceeds quantitatively.

### (Comparative Example 28) Malic Acid Electrochemical Reaction Device

An electrochemical reaction device was prepared in the same manner as in Example 28, except for employing the enzyme electrode prepared in Comparative Example 27, and was used for executing an electrochemical reaction. Pyruvic acid is detected from the reaction electrolyte solution, and a reaction charge amount and an amount of generated pyruvic acid show a high correlation, indicating that the reaction proceeds quantitatively. It is however found that the reaction charge per unit time is smaller than in Example 28.

Based on Example 28 and Comparative Example 28, it is identified that the malic acid electrochemical reaction device of Example 28, in comparison with that of Comparative Example 28, has a larger reaction charge per unit time and is capable of converting malic acid into pyruvic acid more efficiently. This is assumed because, in the electrochemical reaction device of Example 28, despite of a fact that the amounts of malic acid dehydrogenase and diaphorase immobilized on the enzyme electrode are same, malic acid dehydrogenase and diaphorase are retained in a physical proximity as both enzymes are associated, whereby the electron exchange between the both enzymes via NAD/NADH is executed more promptly.

### (Example 29) Preparation of Associated Protein (His-pfuMDH-Dzip1and tmaDp-Dzip2) [SEQ ID NOS. 90, 74] of Malic Acid Dehydrogenase (pfuMDH) Derived from Pyrococcus furiosus and Diaphorase (tmaDp) derived from Thermotoga maritime

A genome DNA is prepared from Pyrococcus furiosus [ATCC 43587] by an ordinary method. A PCR is executed utilizing this genome DNA as a template and employing synthetic oligoDNAs SEQ ID NO:85 having a sequence recognized by BamHI and SEQ ID NO:86 having a sequence recognized by HindIII as primers to obtain an amplified DNA product of 1327 bp. The amplified DNA product is digestion cleaved by restriction enzymes BamHI and HindIII, and is inserted into a same restriction enzyme site of pETDuet-1 (manufactured by Novagen) to obtain a His-pfuMDH expression vector pETDuet-pfuMDH.

Then, a genome DNA is prepared from Thermotoga maritima [ATCC 43589] by an ordinary method. A PCR is executed utilizing this genome DNA as a template and employing synthetic oligoDNAs SEQ ID NO:67 having a sequence recognized by NdeI and SEQ ID NO:68 having a sequence recognized by XhoI as primers to obtain an amplified DNA product of 1371 bp. The amplified DNA product is digestion cleaved by restriction enzymes NdeI and XhoI, and is inserted into a same restriction enzyme site of pETDuet-pfuMDH to obtain a co-expression vector pETDuet-pfuMDH-tmaDp, co-expressing His-pfuMDH and tmaDp.

Then, as in Example 17, 5′-terminal phosphated synthetic oligoDNAs (SEQ ID NO:51 and 52) are used to prepare a DNA fragment (double strand) This DNA fragment is inserted in a recognition site for restriction enzymes HindIII and AflII of pETDuet-pfuMDH-tmaDp. As a result, there is obtained a co-expression vector pETDuet-pfuMDH-Dzip1/tmaDp of a fusion protein, in which an association site sequence SEQ ID NO:169-Dzip1(SEQ ID NO:53) is fused to a C-terminal side of His-pfuMDH, and tmaDp.

Then, as in Example 17, 5′-terminal phosphated synthetic oligoDNAs (SEQ ID NO:54 and 55) are used to prepare a DNA fragment (double strand) This DNA fragment is inserted in a recognition site for restriction enzymes XhoI and AvrII of pETDuet-pfuMDH-Dzip1/tmaDp, thereby obtaining a co-expression vector pETDuet-pfuMDH-Dzip1/tmaDp-Dzip2 (SEQ ID NO:87) of a fusion protein, in which an association site sequence SEQ ID NO:169-Dzip1(SEQ ID NO:53) is fused to a C-terminal side of His-pfuMDH, and a fusion protein in which an association site sequence SEQ ID NO:169-Dzip2 (SEQ ID NO:56) is fused at a C-terminal side of tmaDp.

Then, a PCR is executed utilizing the genome DNA of Pyrococcus furiosus (ATCC 43587] as a template and employing synthetic oligoDNAs SEQ ID NO:88 having a sequence recognized by NcoI and SEQ ID NO:86 having a sequence recognized by HindIII as primers to obtain an amplified DNA product of 1325 bp. The amplified DNA product is digestion cleaved by restriction enzymes NcoI and HindIII, and is inserted into a same restriction enzyme site of pCDFDuet-1 (manufactured by Novagen) to obtain a pfuMDH expression vector pCDFDuet-pfuMDH.

Then, a PCR is executed utilizing the genome DNA of Thermotoga maritima [ATCC 43589] as a template and employing synthetic oligoDNAs SEQ ID NO:67 having a sequence recognized by NdeI and SEQ ID NO:71 having a sequence recognized by XhoI as primers to obtain an amplified DNA product of 1371 bp. The amplified DNA product is digestion cleaved by restriction enzymes NdeI and XhoI, and is inserted into a same restriction enzyme site of pCDFDuet-pfuMDH to obtain a co-expression vector pCDFDuet-pfuMDH-tmaDp (SEQ ID NO:89) of pfuMDH and tmaDp.

Expression vectors pETDuet-pfuMDH-Dzip1/tmaDp-Dzip2 and pCDFDuet-pfuMDH-tmaDp are transformed into E. coli BL21(DE3) by an ordinary method. The transform can be selected as a resistant strain to antibiotics ampicillin and streptomycin. The transforms were used to prepare enzymes by the method 1. Enzymes, thus obtained from thermophilic bacteria, may be used for constructing an enzyme electrode and the like.

### (Example 33) Preparation of associated protein (His-bmaEDH-Dzip1and goxDp-Dzip2) [SEQ ID NOS. 98, 62] of Glutamic Acid Dehydrogenase (bmaEDH) Derived from Burkholderia mallei and Diaphorase (goxDp) Derived from Gluconobacter oxydans

A genome DNA is prepared from Burkholderia mallei [ATCC 23344] by an ordinary method. A PCR is executed utilizing this genome DNA as a template and employing synthetic oligoDNAs SEQ ID NO:93 having a sequence recognized by BamHI and SEQ ID NO:94 having a sequence recognized by HindIII as primers to obtain an amplified DNA product of 1324 bp. The amplified DNA product is digestion cleaved by restriction enzymes BamHI and HindIII, and is inserted into a same restriction enzyme site of pETDuet-1 (manufactured by Novagen) to obtain a His-bmaEDH expression vector pETDuet-bmaEDH.

Then, a genome DNA is prepared from Gluconobacter oxydans [ATCC 621H] by an ordinary method. A PCR is executed utilizing this genome DNA as a template and employing synthetic oligoDNAs SEQ ID NO:49 and SEQ ID NO:50 as primers to obtain an amplified DNA product of 1428 bp. The amplified DNA product is digestion cleaved by restriction enzymes NdeI and XhoI, and is inserted into a same restriction enzyme site of pETDuet-bmaEDH to obtain a co-expression vector pETDuet-bmaEDH-goxDp, co-expressing His-bmaEDH and goxDp.

Then, as in Example 17, 5′-terminal phosphated synthetic oligoDNAs (SEQ ID NO:51 and 52) are used to prepare a DNA fragment (double strand). This DNA-fragment is inserted in a recognition site for restriction enzymes HindIII and AflII of pETDuet-bmaEDH-goxDp. As a result, there is obtained a co-expression vector pETDuet-bmaEDH-Dzip1/goxDp of a fusion protein, in which an association site sequence SEQ ID NO:169-Dzip1(SEQ ID NO:53) is fused to a C-terminal side of His-bmaEDH, and goxDp.

Then, as in Example 17, 5′-terminal phosphated synthetic oligoDNAs (SEQ ID NO:54 and 55) are used to prepare a DNA fragment (double strand). This DNA fragment is inserted in a recognition site for restriction enzymes XhoI and AvrII of pETDuet-bmaEDH-Dzip1/goxDp, thereby obtaining a co-expression vector pETDuet-bmaEDH-Dzip1/goxDp-Dzip2 (SEQ ID NO:95) of a fusion protein, in which an association site sequence SEQ ID NO:169-Dzip1(SEQ ID NO:53) is fused to a C-terminal side of His-bmaEDH, and a fusion protein in which an association site sequence SEQ ID NO:169-Dzip2 (SEQ ID NO:56) is fused at a C-terminal side of goxDp.

Then, a PCR is executed utilizing the genome DNA of Burkholderia mallei [ATCC 23344] as a template and employing synthetic oligoDNAs SEQ ID NO:96 having a sequence recognized by NcoI and SEQ ID NO:94 having a sequence recognized by HindIII as primers to obtain an amplified DNA product of 1322 bp. The amplified DNA product is digestion cleaved by restriction enzymes NcoI and HindIII, and is inserted into a same restriction enzyme site of pCDFDuet-1 (manufactured by Novagen) to obtain a bmaEDH expression vector pCDFDuet-bmaEDH.

Then, a PCR is executed utilizing the genome DNA of Gluconobacter oxydans [ATCC 621H] as a template and employing synthetic oligoDNAs SEQ ID NO:49 and SEQ ID NO:59 as primers to obtain an amplified DNA product of 1428 bp. The amplified DNA product is digestion cleaved by restriction enzymes NdeI and XhoI, and is inserted into a same restriction enzyme site of pCDFDuet-bmaEDH to obtain a co-expression vector pCDFDuet-bmaEDH-goxDp (SEQ ID NO:97) of bmaEDH and goxDp.

Expression vectors pETDuet-bmaEDH-Dzip1/goxDp-Dzip2 and pCDFDuet-bmaEDH-goxDp are transformed into E. coli BL21(DE3) by an ordinary method. The transform can be selected as a resistant strain to antibiotics ampicillin and streptomycin. The transforms were used to prepare enzymes by the method 1.

### (Comparative Example 33) Preparation of bmaEDH and goxDp as Reference

A genome DNA is prepared from Burkholderia mallei [ATCC 23344] by an ordinary method. A PCR is executed utilizing this genome DNA as a template and employing synthetic oligo DNAs SEQ ID NO:99 having a sequence recognized by NdeI and SEQ ID NO:100 having a sequence recognized by XhoI as primers to obtain an amplified DNA product of about 1320 bp. The amplified DNA product is digestion cleaved by restriction enzymes NdeI and XhoI, and is inserted into a same restriction enzyme site of pET-21a(+) (manufactured by Novagen) to obtain a bmaEDH-His expression vector pET21-bmaEDH.

Then, a genome DNA is prepared from Gluconobacter oxydans [ATCC 621H] by an ordinary method. A PCR is executed utilizing this genome DNA as a template and employing oligo DNAs SEQ ID NO:49 and SEQ ID NO:50 as primers to obtain an amplified DNA product of about 1420 bp. The amplified DNA product is digestion cleaved by restriction enzymes NdeI and XhoI, and is inserted into a same restriction enzyme site of pET-21a(+) (manufactured by Novagen) to obtain a goxDp-His expression vector pET21-goxDp.

Expression vectors pET21-bmaEDH and pET21-goxDp are individually transformed into E. coli BL21(DE3) by an ordinary method. The transform can be selected as a resistant strain to antibiotic ampicillin.

These transforms were individually used to prepare enzymes by the method 2.

### (Example 34) Glutamic Acid Sensor

A conductive base member 20 is formed by glassy carbon of a diameter of 3 mm. On the conductive base member 20, an associated protein (His-bmaEDH-Dzip1/goxDp-Dzip2) of glutamic acid dehydrogenase (bmaEDH) derived from Burkholderia mallei in Example 33 and diaphorase (goxDp) derived from Gluconobacter oxydans, and a ferrocene-bonded polyallylamine (Fc-PAA) are immobilized- by- cross-linking by poly(ethylene glycol) diglycigyl ether (PEGDE). Components immobilized on the enzyme electrode have the following formulation:

His-bmaEDH-Dzip1/goxDp-Dzip2 (bmaEDH: 0.3 units, goxDp: 0.6 units)

Fc-PAA: 16 μg

PEGDE: 10 μg

This enzyme electrode is used as the reaction electrode 4 in FIG. 10 to construct a glutamic acid sensor. Also the sample solution 3 is formed by a 0.1M PIPES-NaOH aqueous buffer solution (pH 7.5) containing glutamic acid of a predetermined concentration and 1 mM of NAD. Under an application of a potential of 300 mV to the reaction electrode 4 with respect to the reference electrode 6, glutamic acid in the sample solution 3 is oxidized, in the presence of glutamic acid dehydrogenase, to 2-oxoglutaric acid, and NAD is reduced to NADH in this reaction. Then, in the presence of diaphorase associated with glutamic acid dehydrogenase, NADH is oxidized to NAD, and in this reaction, ferrocene serving as the electron transfer mediator is oxidized to generate a ferricinium ion. As the reaction electrode 4 has a potential of 300 mV with respect to the reference electrode 6, the ferricinium ion receives an electron from the reaction electrode 4 and is reduced to ferrocene. A reduction current corresponding to the glutamic acid concentration in the sample solution can be measured by measuring a current caused by an electron transfer at the reaction electrode 4.

### (Comparative Example 34) Glutamic Acid Sensor

An enzyme electrode is prepared in the same manner as in Example 34, except for employing the enzymes prepared in Comparative Example 33. Components immobilized on the enzyme electrode have the following compositions:

bmaEDH: 0.3 units

goxDp: 0.6 units

Fc-PAA: 16 μg

PEGDE: 10 μg

The enzyme electrode is used as the reaction electrode 4 in FIG. 10 for constructing a glutamic acid sensor, and is used to measure a change in the reduction current corresponding to the glutamic acid concentration in the sample solution as in Example 34. A difference between Example 34 and Comparative Example 34 is similar to the difference between A and B in FIG. 13.

### (Example 35) Glutamic Acid Fuel Cell

FIG. 5 shows the structure of the enzyme electrode. A conductive base member 20 is formed by glassy carbon of 0.5 cm². On the conductive base member 20, an associated protein (His-bmaEDH-Dzip1/goxDp-Dzip2) of glutamic acid dehydrogenase (bmaEDH) derived from Burkholderia mallei in Example 33 and diaphorase (goxDp) derived from Gluconobacter oxydans, and a ferrocene-bonded polyallylamine (Fc-PAA) are immobilized by crosslinking by poly(ethylene glycol) diglycigyl ether (PEGDE). Components immobilized on the enzyme electrode have the following formulation:

His-bmaEDH-Dzip1/goxDp-Dzip2 (bmaEDH: 0.3 units, goxDp: 0.6 units)

Fc-PAA: 16 μg

PEGDE: 10 μg

A fuel cell is prepared by employing this enzyme electrode as an anode electrode shown in FIG. 11. The electrolyte solution 3 is a 50 mM phosphate buffer (pH 7.5) containing 100 mM of sodium chloride, 5 mM of glutamic acid, and 1 mM of nicotinamide adenine dinucleotide. A predetermined voltage is applied between the anode electrode 15 and the cathode electrode 16 and voltage-current characteristics are measured to obtain the following results:

shortcircuit current density: 1226 μA/cm²

maximum electric power: 106 μW/cm²

### (Comparative Example 35) Glutamic Acid Fuel Cell

An enzyme electrode was prepared in the same manner as in Example 35, except for employing enzymes prepared in Comparative Example 33. Components immobilized on the enzyme electrode have the following formulation:

bmaEDH: 0.3 units

goxDp: 0.6 units

Fc-PAA: 16 μg

PEGDE: 10 μg

A fuel cell was prepared with this enzyme electrode as an anode in FIG. 11, and voltage-current characteristics were measured in the same manner as in Example 35 to obtain the following results:

shortcircuit current density: 387 μA/cm²

maximum electric power: 34 μW/cm²

Based on Example 35 and Comparative Example 35, it is identified that the glutamic acid fuel cell of Example 35, in comparison with that of Comparative Example 35, has a higher current density and is capable of providing a larger current. This is assumed because, in the fuel cell of Example 35, despite of a fact that the amounts of glutamic acid dehydrogenase and diaphorase immobilized on the enzyme electrode are same, glutamic acid dehydrogenase and diaphorase are retained in a physical proximity as both enzymes are associated,. whereby the electron exchange between the both enzymes via NAD/NADH is executed more promptly.

### (Example 36) Glutamic Acid Electrochemical Reaction Device

The enzyme electrode prepared in Example 35 was used to prepare an electrochemical reaction device shown in FIG. 10. A 50 mM phosphate buffer (pH 7.5) containing 100 mM of sodium chloride, 5 mM of glutamic acid, and 1 mM of nicotinamide adenine dinucleotide is used as the sample solution 3, then a potential of 0.3 VvsAg/AgCl is applied for 100 minutes under a nitrogen atmosphere, and a product is quantitatively measured by a high-speed liquid chromatography. 2-oxoglutaric acid is detected from the reaction electrolyte solution, and a reaction charge amount and an amount of generated 2-oxoglutaric acid show a high correlation, indicating that the reaction proceeds quantitatively.

### (Comparative Example 36) Glutamic Acid Electrochemical Reaction Device

An electrochemical reaction device was prepared in the same manner as in Example 36, except for employing the enzyme electrode prepared in Comparative Example 35, and was used for executing an electrochemical reaction. 2-oxoglutaric acid is detected from the reaction electrolyte solution, and a reaction charge amount and an amount of generated 2-oxoglutaric acid show a high correlation, indicating that the reaction proceeds quantitatively. It is however found that the reaction charge per unit time is smaller than in Example 36.

Based on Example 36 and Comparative Example 36, it is identified that the glutamic acid electrochemical reaction device of Example 36, in comparison with that of Comparative Example 36, has a larger reaction charge per unit time and is capable of converting glutamic acid into 2-oxoglutaric acid more efficiently. This is assumed because, in the electrochemical reaction device of Example 36, despite of a fact that the amounts of glutamic acid dehydrogenase and diaphorase immobilized on the enzyme electrode are same, glutamic acid dehydrogenase and diaphorase are retained in a physical proximity as both enzymes are associated, whereby the electron exchange between the both enzymes via NAD/NADH is executed more promptly.

### (Example 37) Preparation of Associated Protein (His-pfuEDH-Dzip1and tmaDp-Dzip2) [SEQ ID NOS. 106, 74] of Glutamic Acid Dehydrogenase (pfuEDH) Derived from Pyrococcus furiosus and Diaphorase (tmaDp) Derived from Thermotoga maritime

A genome DNA is prepared from Pyrococcus furiosus [ATCC 43587] by an ordinary method. A PCR is executed utilizing this genome DNA as a template and employing synthetic oligoDNAs SEQ ID NO:101 having a sequence recognized by BamHI and SEQ ID NO:102 having a sequence recognized by HindIII as primers to obtain an amplified DNA product of 1282 bp. The amplified DNA product is digestion cleaved by restriction enzymes BamHI and HindIII, and is inserted into a same restriction enzyme site of pETDuet-1 (manufactured by Novagen) to obtain a His-pfuEDH expression vector pETDuet-pfuEDH.

Then, a genome DNA is prepared from Thermotoga maritima [ATCC 43589] by an ordinary method. A PCR is executed utilizing this genome DNA as a template and employing synthetic oligoDNAs SEQ ID NO:67 and SEQ ID NO:68 as primers to obtain an amplified DNA product of 1371 bp. The amplified DNA product is digestion cleaved by restriction enzymes NdeI and XhoI, and is inserted into a same restriction enzyme site of pETDuet-pfuEDH to obtain a co-expression vector pETDuet-pfuEDH-tmaDp, co-expressing His-pfuEDH and tmaDp.

Then, as in Example 17, 5′-terminal phosphated synthetic oligoDNAs (SEQ ID NO:51 and 52) are used to prepare a DNA fragment (double strand) This DNA fragment is inserted in a recognition site for restriction enzymes HindIII and AflII of pETDuet-pfuEDH-tmaDp. As a result, there is obtained a co-expression vector pETDuet-pfuEDH-Dzip1/tmaDp of a fusion protein, in which an association site sequence SEQ ID NO:169-Dzip1(SEQ ID NO:53) is fused to a C-terminal side of His-pfuEDH, and tmaDp.

Then, as in Example 17, 5′-terminal phosphated synthetic oligoDNAs (SEQ ID NO:54 and 55) are used to prepare a DNA fragment (double strand) This DNA fragment is inserted in a recognition site for restriction enzymes XhoI and AvrII of pETDuet-pfuEDH-Dzip1/tmaDp, thereby obtaining a co-expression vector pETDuet-pfuEDH-Dzip1/tmaDp-Dzip2 (SEQ ID NO:103) of a fusion protein, in which an association site sequence SEQ ID NO:169-Dzip1(SEQ ID NO:53) is fused to a C-terminal side of His-pfuEDH, and a fusion protein in which an association site sequence SEQ ID NO:169-Dzip2 (SEQ ID NO:56) is fused at a C-terminal side of tmaDp.

Then, a PCR is executed utilizing the genome DNA of Pyrococcus furiosus [ATCC 43587] as a template and employing synthetic oligoDNAs SEQ ID NO:104 having a sequence recognized by NcoI and SEQ ID NO:102 having a sequence recognized by HindIII as primers to obtain an amplified DNA product of 1280 bp. The amplified DNA product is digestion cleaved by restriction enzymes NcoI and HindIII, and is inserted into a same restriction enzyme site of pCDFDuet-1 (manufactured by Novagen) to obtain a pfuEDH expression vector pCDFDuet-pfuEDH.

Then, a PCR is executed utilizing the genome DNA of Thermotoga maritima [ATCC 43589] as a template and employing synthetic oligoDNAs SEQ ID NO:67 and SEQ ID NO:71 as primers to obtain an amplified DNA product of 1371 bp. The amplified DNA product is digestion cleaved by restriction enzymes NdeI and XhoI, and is inserted into a same restriction enzyme site of pCDFDuet-pfuEDH to obtain a co-expression vector pCDFDuet-pfuEDH-tmaDp (SEQ ID NO:105) of pfuEDH and tmaDp.

Expression vectors pETDuet-pfuEDH-Dzip1/tmaDp-Dzip2 and pCDFDuet-pfuEDH-tmaDp are transformed into E. coli BL21(DE3) by an ordinary method. The transform can be selected as a resistant strain to antibiotics ampicillin and streptomycin. The transforms were used to prepare enzymes by the method 1. Enzymes, thus obtained from thermophilic bacteria, may be used for constructing an enzyme electrode and the like.

### (Example 41) Preparation of Associated Protein (His-sceADH-Dzip1and goxALDH-Dzip2) [SEQ ID NOS. 114, 115] of Alcohol Dehydrogenase (sceADH) Derived from Saccharomyces cerevisiae and Aldehyde Dehydrogenase (goxALDH) Derived from Gluconobacter oxydans

A genome DNA is prepared from Saccharomyces cerevisiae [ATCC 47058] by an ordinary method. A PCR is executed utilizing this genome DNA as a template and employing synthetic oligoDNAs SEQ ID NO:31 having a sequence recognized by BamHI and SEQ ID NO:32 having a sequence recognized by HindIII as primers to obtain an amplified DNA product of 1075 bp. The amplified DNA product is digestion cleaved by restriction enzymes BamHI and HindIII, and is inserted into a same restriction enzyme site of pETDuet-1 (manufactured by Novagen) to obtain a His-sceADH expression vector pETDuet-sceADH.

Then, a genome DNA is prepared from Gluconobacter oxydans [ATCC 621H] by an ordinary method. A PCR is executed utilizing this genome DNA as a template and employing synthetic oligoDNAs SEQ ID NO:109 having a sequence recognized by NdeI and SEQ ID NO:110 having a sequence recognized by XhoI as primers to obtain an amplified DNA product of 1560 bp. The amplified DNA product is digestion cleaved by restriction enzymes NdeI and XhoI, and is inserted into a same restriction enzyme site of pETDuet-sceADH to obtain a co-expression vector pETDuet-sceADH-goxALDH, co-expressing His-sceADH and goxALDH.

Then, as in Example 17, 5′-terminal phosphated synthetic oligoDNAs (SEQ ID NO:51 and 52) are used to prepare a DNA fragment (double strand). This DNA fragment is inserted in a recognition site for restriction enzymes HindIII and AflII of pETDuet-sceADH-goxALDH, thereby obtaining a co-expression vector pETDuet-sceADH-Dzip1/goxALDH of a fusion protein, in which an association site sequence SEQ ID NO:169-Dzip1(SEQ ID NO:53) is fused to a C-terminal side of His-sceADH, and goxALDH.

Then, as in Example 17, 5′-terminal phosphated synthetic oligoDNAs (SEQ ID NO:54 and 55) are used to prepare a DNA fragment (double strand) This DNA fragment is inserted in a recognition site for restriction enzymes XhoI and AvrII of pETDuet-sceADH-Dzip1/goxALDH, thereby obtaining a co-expression vector pETDuet-sceADH-Dzip1/goxALDH-Dzip2 (SEQ ID NO:111) of a fusion protein, in which an association site sequence SEQ ID NO:169-Dzip1(SEQ ID NO:53) is fused to a C-terminal side of His-sceADH, and a fusion protein in which an association site sequence SEQ ID NO:169-Dzip2 (SEQ ID NO:56) is fused at a C-terminal side of goxALDH.

Then, a PCR is executed utilizing the genome DNA of Saccharomyces cerevisiae [ATCC 47058] as a template and employing synthetic oligoDNAs SEQ ID NO:34 and SEQ ID NO:32 as primers to obtain an amplified DNA product of 1073 bp. The amplified DNA product is digestion cleaved by restriction enzymes NcoI and HindIII, and is inserted into a same restriction enzyme site of pCDFDuet-1 (manufactured by Novagen) to obtain a sceADH expression vector pCDFDuet-sceADH.

Then, a PCR is executed utilizing the genome DNA of Gluconobacter oxydans [ATCC 621H] as a template and employing synthetic oligoDNAs SEQ ID NO:109 having a sequence recognized by NdeI and SEQ ID NO:112 having a sequence recognized by XhoI as primers to obtain an amplified DNA product of 1560 bp. The amplified DNA product is digestion cleaved by restriction enzymes NdeI and XhoI, and is inserted into a same restriction enzyme site of pCDFDuet-sceADH to obtain a co-expression vector pCDFDuet-sceADH-goxALDH (SEQ ID NO:113) of sceADH and goxALDH.

Expression vectors pETDuet-sceADH-Dzip1/goxALDH-Dzip2 and pCDFDuet-sceADH-goxALDH are transformed into E. coli BL21(DE3) by an ordinary method. The transform can be selected as a resistant strain to antibiotics ampicillin and streptomycin. The transforms were used to prepare enzymes by the method 1.

Then, a genome DNA is prepared from Pseudomonas putida KT2440 [ATCC 47054] by an ordinary method. A PCR is executed utilizing this genome DNA as a template and employing synthetic oligoDNAs SEQ ID NO:3 and SEQ ID NO:4 as primers to obtain an amplified DNA product of 729 bp. The amplified DNA product is digestion cleaved by restriction enzymes NdeI and XhoI, and is inserted into a same restriction enzyme site of pET-21a(+) (manufactured by Novagen) to obtain a ppuDp-His expression vector pET21-ppuDp.

Expression vector pET21-ppuDp is transformed into E. coli BL21(DE3) by an ordinary method. The transform can be selected as a resistant strain to antibiotics ampicillin. The transform was used to prepare an enzyme by the method 1.

### (Comparative Example 41) Preparation of sceADH and goxALDH as Reference

A genome DNA is prepared from Saccharomyces cerevisiae (ATCC 47058] by an ordinary method. A PCR is executed utilizing this genome DNA as a template and employing synthetic oligo DNAs SEQ ID NO:37 and SEQ ID NO:38 as primers to obtain an amplified DNA product of about 1074 bp. The amplified DNA product is digestion cleaved by restriction enzymes NdeI and XhoI, and is inserted into a same restriction enzyme site of pET-21a(+) (manufactured by Novagen) to obtain a sceADH-His expression vector pET21-sceADH.

Then, a genome DNA is prepared from Gluconobacter oxydans [ATCC 621H] by an ordinary method. A PCR is executed utilizing this genome DNA as a template and employing oligo DNAs SEQ ID NO:109 having a sequence recognized by NdeI and SEQ ID NO:110 having a sequence recognized by XhoI as primers to obtain an amplified DNA product of 1557 bp. The amplified DNA product is digestion cleaved by restriction enzymes NdeI and XhoI, and is inserted into a same restriction enzyme site of pET-21a(+) (manufactured by Novagen) to obtain a goxALDH-His expression vector pET21-goxALDH.

Then, a genome DNA is prepared from Pseudomonas putida KT2440 [ATCC 47054] by an ordinary method. A PCR is executed utilizing this genome DNA as a template and employing oligo DNAs SEQ ID NO:3 and SEQ ID NO:4 as primers to obtain an amplified DNA product of 729 bp. The amplified DNA product is digestion cleaved by restriction enzymes NdeI and XhoI, and is inserted into a same restriction enzyme site of pET-21a(+) (manufactured by Novagen) to obtain a ppuDp-His expression vector pET21-ppuDp.

Expression vectors pET21-sceADH, pET21-goxALDH and pET21-ppuDp are individually transformed into E. coli BL21(DE3) by an ordinary method. The transform can be selected as a resistant strain to antibiotic ampicillin.

These transforms were individually used for preparing enzymes by the method 2.

### (Example 42) Alcohol Sensor

A conductive base member 20 is formed by glassy carbon of a diameter of 3 mm. On the conductive base member 20, an associated protein (His-sceADH-Dzip1/goxALDH-Dzip2) of alcohol dehydrogenase (sceADH) derived from Saccharomyces cerevisiae in Example 41 and aldehyde dehydrogenase (goxALDH) derived from Gluconobacter oxydans, a diaphorase (ppuDp) derived from Pseudomonas putida, and a ferrocene-bonded polyallylamine (Fc-PAA) are immobilized by crosslinking by poly(ethylene glycol) diglycigyl ether (PEGDE). Components immobilized on the enzyme electrode have the following formulation:

His-sceADH-Dzip1/goxALDH-Dzip2 (sceADH: 0.3 units, goxALDH: 0.6 units)

ppuDp: 0.6 units

Fc-PAA: 16 μg

PEGDE: 10 μg

This enzyme electrode is used as the reaction electrode 4 in FIG. 10 to construct an alcohol sensor. Also the sample solution 3 is formed by a 0.1M PIPES-NaOH aqueous buffer solution (pH 7.5) containing alcohol (ethanol) of a predetermined concentration and 1 mM of NAD. Under an application of a potential of 300 mV to the reaction electrode 4 with respect to the reference electrode 6, alcohol in the sample solution 3 is oxidized, in the presence of alcohol dehydrogenase, to acetaldehyde, and NAD is reduced to NADH in this reaction. Then, in the presence of aldehyde dehydrogenase associated with alcohol dehydrogenase, acetaldehyde is oxidized to a carboxylic acid, and NAD is simultaneously reduced to NADH. NADH, generated by the enzyme reactions of alcohol dehydrogenase and aldehyde dehydrogenase, is oxidized in the presence of diaphorase to NAD, and in this reaction, ferrocene serving as the electron transfer mediator is oxidized to generate a ferricinium ion. As the reaction electrode 4 has a potential of 300 mV with respect to the reference electrode 6, the ferricinium ion receives an electron from the reaction electrode 4 and is reduced to ferrocene. A reduction current corresponding to the alcohol concentration in the sample solution can be measured by measuring a current caused by an electron transfer at the reaction electrode 4.

### (Comparative Example 42) Alcohol Sensor

An enzyme electrode is prepared in the same manner as in Example 42, except for employing the enzymes prepared in Comparative Example 41. Components immobilized on the enzyme electrode have the following compositions:

sceADH: 0.3 units

goxALDH: 0.6 units

ppuDp: 0.6 units

Fc-PAA: 16 μg

PEGDE: 10 μg

The enzyme electrode is used as the reaction electrode 4 in FIG. 10 for constructing an alcohol sensor, and is used to measure a change in the reduction current corresponding to the alcohol concentration in the sample solution as in Example 42. A difference between Example 42 and Comparative Example 42 is similar to the difference in FIG. 13.

### (Example 43) Alcohol Fuel Cell

FIG. 6 shows the structure of the enzyme electrode. A conductive base member 20 is formed by glassy carbon of 0.5 cm². On the conductive base member 20, an associated protein (His-sceADH-Dzip1/goxALDH-Dzip2) of alcohol dehydrogenase (sceADH) derived from Saccharomyces cerevisiae in Example 41 and aldehyde dehydrogenase (goxALDH) derived from Gluconobacter oxydans, diaphorase (ppuDp) derived from Pseudomonas putida, and a ferrocene-bonded polyallylamine (Fc-PAA) are immobilized by crosslinking by poly(ethylene glycol) diglycigyl ether (PEGDE). Components immobilized on the enzyme electrode have the following formulation:

His-sceADH-Dzip1/goxALDH-Dzip2 (sceADH: 0.3 units, goxALDH: 0.6 units

ppuDp: 0.6 units

Fc-PAA: 16 μg

PEGDE: 10 μg

A fuel cell is prepared by employing this enzyme electrode as an anode electrode shown in FIG. 11. The electrolyte solution 3 is a 50 mM phosphate buffer (pH 7.5) containing 100 mM of sodium chloride, 5 mM of alcohol (ethanol), and 1 mM of nicotinamide adenine dinucleotide. A predetermined voltage is applied between the anode electrode 15 and the cathode electrode 16 and voltage-current characteristics are measured to obtain the following results:

shortcircuit current density: 1621 μA/cm²

maximum electric power: 173 μW/cm²

### (Comparative Example 43) Alcohol Fuel Cell

An enzyme electrode was prepared in the same manner as in Example 43, except for employing enzymes prepared in Comparative Example 41. Components immobilized on the enzyme electrode have the following formulation:

sceADH: 0.3 units

goxALDH: 0.6 units

ppuDp: 0.6 units

Fc-PAA: 16 μg

PEGDE: 10 μg

A fuel cell was prepared with this enzyme electrode as an anode in FIG. 11, and voltage-current characteristics were measured in the same manner as in Example 41 to obtain the following results:

shortcircuit current density: 503 μA/cm²

maximum electric power: 51 μW/cm²

Based on Example 43 and Comparative Example 43, it is identified that the alcohol fuel cell of Example 43, in comparison with that of Comparative Example 43, has a higher current density and is capable of providing a larger current. This is assumed because, in the fuel cell of Example 43, despite of a fact that the amounts of alcohol dehydrogenase, aldehyde dehydrogenase and diaphorase immobilized on the enzyme electrode are same, alcohol dehydrogenase and aldehyde dehydrogenase are retained in a physical proximity as both enzymes are associated, whereby NADH amount generated in unit time is made larger.

### (Example 44) Alcohol Electrochemical Reaction Device

The enzyme electrode prepared in Example 43 was used to prepare an electrochemical reaction device shown in FIG. 10. A 50 mM phosphate buffer (pH 7.5) containing 100 mM of sodium chloride, 5 mM of alcohol, and 1 mM of nicotinamide adenine dinucleotide is used as the sample solution 3, then a potential of 0.3 VvsAg/AgCl is applied for 100 minutes under a nitrogen atmosphere, and a product is quantitatively measured by a high-speed liquid chromatography. A carboxylic acid (acetic acid) is detected from the reaction electrolyte solution, and a reaction charge amount and an amount of generated carboxylic acid (acetic acid) show a high correlation, indicating that the reaction proceeds quantitatively.

### (Comparative Example 44) Alcohol Electrochemical Reaction Device

An electrochemical reaction device was prepared in the same manner as in Example 44, except for employing the enzyme electrode prepared in Comparative Example 43, and was used for executing an electrochemical reaction. A carboxylic acid (acetic acid) is detected from the reaction electrolyte solution, and a reaction charge amount and an amount of generated carboxylic acid (acetic acid) show a high correlation, indicating that the reaction proceeds quantitatively. It is however found that the reaction charge per unit time is smaller than in Example 44.

Based on Example 44 and Comparative Example 44, it is identified that the alcohol electrochemical reaction device of Example 44, in comparison with that of Comparative Example 44, has a larger reaction charge per unit time and is capable of converting alcohol into carboxylic acid more efficiently. This is assumed because, in the electrochemical reaction device of Example 44, despite of a fact that the amounts of alcohol dehydrogenase, aldehyde dehydrogenase and diaphorase immobilized on the enzyme electrode are same, alcohol dehydrogenase and aldehyde dehydrogenase are retained in a physical proximity as both enzymes are associated, whereby an amount of NADH generated per unit time is made larger.

### (Example 45) Preparation of Associated Protein (His-pfuADH-Dzip1/tthALDH-Dzip2) [SEQ ID NOS. 121, 122] of Alcohol Dehydrogenase (pfuADH) derived from Pyrococcus furiosus and Aldehyde Dehydrogenase (tthALDH) Derived from Thermus thermophilus

A genome DNA is prepared from Pyrococcus furiosus [ATCC 43587] by an ordinary method. A PCR is executed utilizing this genome DNA as a template and employing synthetic oligoDNAs SEQ ID NO:39 and SEQ ID NO:40 as primers to obtain an amplified DNA product of 1147 bp.

The amplified DNA product is digestion cleaved by restriction enzymes BamHI and HindIII, and is inserted into a same restriction enzyme site of pETDuet-1 (manufactured by Novagen) to obtain a His-pfuADH expression vector pETDuet-pfuADH.

Then, a genome DNA is prepared from Thermus thermophilus [ATCC BAA-163] by an ordinary method. A PCR is executed utilizing this genome DNA as a template and employing synthetic oligoDNAs SEQ ID NO:116 having a sequence recognized by NdeI and SEQ ID NO:117 having a sequence recognized by XhoI as primers to obtain an amplified DNA product of 1614 bp.

The amplified DNA product is digestion cleaved by restriction enzymes NdeI and XhoI, and is inserted into a same restriction enzyme site of pETDuet-pfuADH to obtain a co-expression vector pETDuet-pfuADH-tthALDH, co-expressing His-pfuADH and tthALDH.

Then, as in Example 17, 5′-terminal phosphated synthetic oligoDNAs (SEQ ID NO:51 and 52) are used to prepare a DNA fragment (double strand). This DNA fragment is inserted in a recognition site for restriction enzymes HindIII and AflII of pETDuet-pfuADH-tthALDH. As a result, there is obtained a co-expression vector pETDuet-pfuADH-Dzip1/tthALDH of a fusion protein, in which an association site sequence SEQ ID NO:169-Dzip1(SEQ ID NO:53) is fused to a C-terminal side of His-pfuADH, and tthALDH.

Then, as in Example 17, 5′-terminal phosphated synthetic oligoDNAs (SEQ ID NO:54 and 55) are used to prepare a DNA fragment (double strand). This DNA fragment is inserted in a recognition site for restriction enzymes XhoI and AvrII of pETDuet-pfuADH-Dzip1/tthALDH, thereby obtaining a co-expression vector pETDuet-pfuADH-Dzip1/tthALDH-Dzip2 (SEQ ID NO:118) of a fusion protein, in which an association site sequence SEQ ID NO:169-Dzip1(SEQ ID NO:53) is fused to a C-terminal side of His-pfuADH, and a fusion protein in which an association site sequence SEQ ID NO:169-Dzip2 (SEQ ID NO:56) is fused at a C-terminal side of tthALDH.

Then, a PCR is executed utilizing the genome DNA of Pyrococcus furiosus [ATCC 43587) as a template and employing synthetic oligoDNAs SEQ ID NO:42 and SEQ ID NO:40 as primers to obtain an amplified DNA product of 1145 bp.

The amplified DNA product is digestion cleaved by restriction enzymes NcoI and HindIII, and is inserted into a same restriction enzyme site of pCDFDuet-1 (manufactured by Novagen) to obtain a pfuADH expression vector pCDFDuet-pfuADH.

Then, a PCR is executed utilizing the genome DNA of Thermus thermophilus [ATCC BAA-163] as a template and employing synthetic oligoDNAs SEQ ID NO:116 having a sequence recognized by NdeI and SEQ ID NO:119 having a sequence recognized by XhoI as primers to obtain an amplified DNA product of 1614 bp.

The amplified DNA product is digestion cleaved by restriction enzymes NdeI and XhoI, and is inserted into a same restriction enzyme site of pCDFDuet-pfuADH to obtain a co-expression vector pCDFDuet-pfuADH-tthALDH (SEQ ID NO:120) of pfuADH and tthALDH.

Expression vectors pETDuet-pfuADH-Dzip1/tthALDH-Dzip2 and pCDFDuet-pfuADH-tthALDH are transformed into E. coli BL21(DE3) by an ordinary method. The transform can be selected as a resistant strain to antibiotics ampicillin and streptomycin. The transforms were used to prepare enzymes by the method 1.

Then, a genome DNA is prepared from Pyrococcus horikoshii KT2440 [ATCC 700860] by an ordinary method. A PCR is executed utilizing this genome DNA as a template and employing synthetic oligoDNAs SEQ ID NO:21 and SEQ ID NO:22 as primers to obtain an amplified DNA product of 1344 bp.

The amplified DNA product is digestion cleaved by restriction enzymes NdeI and XhoI, and is inserted into a same restriction enzyme site of pET-21a(+) (manufactured by Novagen) to obtain a phoDp-His expression vector pET21-phoDp.

The expression vector pET21-phoDp is transformed into E. coli BL21(DE3) by an ordinary method. The transform can be selected as a resistant strain to antibiotic ampicillin. The transform was used to prepare an enzyme by the method 1. Enzyme, thus obtained, may be used for constructing an enzyme electrode.

### (Example 49) Preparation of Associated Protein (His-busGDH-Dzip1/ecoISO-Dzip2) [SEQ ID NOS. 128, 129] of Glucose Dehydrogenase (busGDH) Derived from Bacillus subtilis and Xylose Isomerase (ecoISO) Derived from Escherichia coli

A genome DNA is prepared from Bacillus subtilis [ATCC 27370] by an ordinary method. A PCR is executed utilizing this genome DNA as a template and employing synthetic oligoDNAs SEQ ID NO:1 and SEQ ID NO:2 as primers to obtain an amplified DNA product of 805 bp. The amplified DNA product is digestion cleaved by restriction enzymes BamHI and HindIII, and is inserted into a same restriction enzyme site of pETDuet-1 (manufactured by Novagen) to obtain a His-busGDH expression vector pETDuet-busGDH.

Then, a genome DNA is prepared from Escherichia coli [ATCC 29425] by an ordinary method. A PCR is executed utilizing this genome DNA as a template and employing synthetic oligoDNAs SEQ ID NO:123 having a sequence recognized by NdeI and SEQ ID NO:124 having a sequence recognized by XhoI as primers to obtain an amplified DNA product of 1344 bp.

The amplified DNA product is digestion cleaved by restriction enzymes NdeI and XhoI, and is inserted into a same restriction enzyme site of pETDuet-busGDH to obtain a co-expression vector pETDuet-busGDH-ecoISO, co-expressing His-busGDH and ecoISO.

Then, as in Example 17, 5′-terminal phosphated synthetic oligoDNAs (SEQ ID NO:51 and 52) are used to prepare a DNA fragment (double strand). This DNA fragment is inserted in a recognition site for restriction enzymes HindIII and AflII of pETDuet-busGDH-ecoISO. As a result, there is obtained a co-expression vector pETDuet-busGDH-Dzip1/ecoISO of a fusion protein, in which an association site sequence SEQ ID NO:169-Dzip1(SEQ ID NO:53) is fused to a C-terminal side of His-busGDH, and ecoISO.

Then, as in Example 17, 5′-terminal phosphated synthetic oligoDNAs (SEQ ID NO:54 and 55) are used to prepare a DNA fragment (double strand). This DNA fragment is inserted in a recognition site for restriction enzymes XhoI and AvrII of pETDuet-busGDH-Dzip1/ecoISO, thereby obtaining a co-expression vector pETDuet-busGDH-Dzip1/ecoISO-Dzip2 (SEQ ID NO:125) of a fusion protein, in which an association site sequence SEQ ID NO:169-Dzip1(SEQ ID NO:53) is fused to a C-terminal side of His-busGDH, and a fusion protein in which an association site sequence SEQ ID NO:169-Dzip2 (SEQ ID NO:56) is fused at a C-terminal side of ecoISO.

Then, a PCR is executed utilizing the genome DNA of Bacillus subtilis [ATCC 27370] as a template and employing synthetic oligoDNAs SEQ ID NO:12 and SEQ ID NO:2 as primers to obtain an amplified DNA product of 805 bp.

The amplified DNA product is digestion cleaved by restriction enzymes NcoI and HindIII, and is inserted into a same restriction enzyme site of pCDFDuet-1 (manufactured by Novagen) to obtain a busGDH expression vector pCDFDuet-busGDH.

Then, a PCR is executed utilizing the genome DNA of Escherichia coli [ATCC 29425] as a template and employing synthetic oligoDNAs SEQ ID NO:123 having a sequence recognized by NdeI and SEQ ID NO:XhoI having a sequence recognized by as primers to obtain an amplified DNA product of 1344 bp.

The amplified DNA product is digestion cleaved by restriction enzymes NdeI and XhoI, and is inserted into a same restriction enzyme site of pCDFDuet-busGDH to obtain a co-expression vector pCDFDuet-busGDH-ecoISO (SEQ ID NO:127) of busGDH and ecoISO.

Expression vectors pETDuet-busGDH::ecoISO and pCDFDuet-busGDH-ecoISO are transformed into E. coli BL21(DE3) by an ordinary method. The transform can be selected as a resistant strain to antibiotics ampicillin and streptomycin. The transforms were used to prepare enzymes by the method 1.

Then, a genome DNA is prepared from Pseudomonas putida KT2440 [ATCC 47054] by an ordinary method. A PCR is executed utilizing this- genome DNA as a template and employing synthetic oligoDNAs SEQ ID NO:3 and SEQ ID NO:4 as primers to obtain an amplified DNA product of 729 bp.

The amplified DNA product is digestion cleaved by restriction enzymes NdeI and XhoI, and is inserted into a same restriction enzyme site of pET-21a(+) (manufactured by Novagen) to obtain a ppuDp-His expression vector pET21-ppuDp.

The expression vector pET21-ppuDp is transformed into E. coli BL21(DE3) by an ordinary method. The transform can be selected as a resistant strain to antibiotic ampicillin. The transform was used to prepare an enzyme by the method 1.

### (Comparative Example 49) Preparation of busGDH and ecoISO as Reference

A genome DNA is prepared from Bacillus subtilis [ATCC 27370] by an ordinary method. A PCR is executed utilizing this genome DNA as a template and employing synthetic oligo DNAs SEQ ID NO:17 and SEQ ID NO:18 as primers to obtain an amplified DNA product of about 800 bp. The amplified DNA product is digestion cleaved by restriction enzymes NdeI and XhoI, and is inserted into a same restriction enzyme site of pET-21a(+) (manufactured by Novagen) to obtain a busGDH-His expression vector pET21-busGDH.

Then, a genome DNA is prepared from Escherichia coli (ATCC 29425] by an ordinary method. A PCR is executed utilizing this genome DNA as a template and employing synthetic oligo DNAs SEQ ID NO:123 having a sequence recognized by NdeI and SEQ ID NO:124 having a sequence recognized by XhoI as primers to obtain an amplified DNA product of 1341 bp.

The amplified DNA product is digestion cleaved by restriction enzymes NdeI and XhoI, and is inserted into a same restriction enzyme site of pET-21a(+) (manufactured by Novagen) to obtain a ecoISO-His expression vector pET21-ecoISO.

Then, a genome DNA is prepared from Pseudomonas putida KT2440 [ATCC 47054] by an ordinary method. A PCR is executed utilizing this genome DNA as a template and employing synthetic oligo DNAs SEQ ID NO:3 and SEQ ID-NO:4 as primers to obtain an amplified DNA product of 729 bp. The amplified DNA product is digestion cleaved by restriction enzymes NdeI and XhoI, and is inserted into a same restriction enzyme site of pET-21a(+) (manufactured by Novagen) to obtain a ppuDp-His expression vector pET21-ppuDp.

Expression vectors pET21-busGDH pET21-ecoISO and pET21-ppuDp are individually transformed into E. coli BL21(DE3) by an ordinary method. The transform can be selected as a resistant strain to antibiotic ampicillin.

These transforms were individually used for preparing enzymes by the method 2.

### (Example 50) Fructose Sensor

A structure of the enzyme electrode is shown in FIG. 7. A conductive base member 20 is formed by glassy carbon of a diameter of 3 mm. On the conductive base member 20, an associated protein (His-busGDH-Dzip1/ecoISO-Dzip2) of glucose dehydrogenase (busGDH) derived from Bacillus subtilis in Example 49 and xylose isomerase (ecoISO) derived from Escherichia coli, a diaphorase (ppuDp) derived from Pseudomonas putida, and a ferrocene-bonded polyallylamine (Fc-PAA) are immobilized by crosslinking by PEGDE. Components immobilized on the enzyme electrode have the following formulation:

His-busGDH-Dzip1/ecoISO-Dzip2 (busGDH: 0.3 units, ecoISO: 0.6 units)

ppuDp: 0.6 units

Fc-PAA: 16 μg

PEGDE: 10 μg

This enzyme electrode is used as the reaction electrode 4 in FIG. 10 to construct a fructose sensor. Also the sample solution 3 is formed by a 0.1M PIPES-NaOH aqueous buffer solution (pH 7.5) containing fructose of a predetermined concentration and 1 mM of NAD. Under an application of a potential of 300 mV to the reaction electrode 4 with respect to the reference electrode 6, fructose in the sample solution 3 is converted, in the presence of xylose isomerase, to glucose, which is then oxidized to gluocolactone in the presence of glucose dehydrogenase associated with xylose isomerase, while NAD is reduced to NADH in this reaction. Then, NADH is oxidized in the presence of diaphorase to NAD, and in this reaction, ferrocene serving as the electron transfer mediator is oxidized to generate a ferricinium ion. As the reaction electrode 4 has a potential of 300 mV with respect to the reference electrode 6, the ferricinium ion receives an electron from the reaction electrode 4 and is reduced to ferrocene. A fructose concentration in the sample solution can be measured by measuring a current caused by an electron transfer at the reaction electrode 4.

### (Comparative Example 50) Fructose sensor

An enzyme electrode is prepared in the same manner as in Example 50, except for employing the enzymes prepared in Comparative Example 49. Components immobilized on the enzyme electrode have the following compositions:

busGDH: 0.3 units

ecoISO: 0.6 units

ppuDp: 0.6 units

Fc-PAA: 16 μg

PEGDE: 10 μg

The enzyme electrode is used as the reaction electrode 4 in FIG. 10 for constructing a fructose sensor, and is used to measure a change in the reduction current corresponding to the fructose concentration as in Example 50. A difference between Example 50 and Comparative Example 50 is similar to the difference between A and B in FIG. 13.

### (Example 51) Fructose Fuel Cell

FIG. 7 shows the structure of the enzyme electrode. A conductive base member 20 is formed by glassy carbon of 0.5 cm². On the conductive base member 20, an associated protein (His-busGDH-Dzip1/ecoISO-Dzip2) of glucose dehydrogenase (busGDH) derived from Bacillus subtilis in Example 49 and xylose isomerase (ecoISO) derived from Escherichia coli, diaphorase (ppuDp) derived from Pseudomonas putida, and a ferrocene-bonded polyallylamine (Fc-PAA) are immobilized by crosslinking by PEGDE. Components immobilized on the enzyme electrode have the following formulation:

His-busGDH-Dzip1/ecoISO-Dzip2 (busGDH: 0.3 units, ecoISO: 0.6 units)

ppuDp: 0.6 units

Fc-PAA: 16 μg

PEGDE: 10 μg

A fuel cell is prepared by employing this enzyme electrode as an anode electrode shown in FIG. 11. The electrolyte solution 3 is a 50 mM phosphate buffer (pH 7.5) containing 100 mM of sodium chloride, 5 mM of fructose, and 1 mM of nicotinamide adenine dinucleotide. A predetermined voltage is applied between the anode electrode 15 and the cathode electrode 16 and voltage-current characteristics are measured to obtain the following results:

shortcircuit current density: 1106 μA/cm²

maximum electric power: 101 μW/cm²

### (Comparative Example 51) Fructose Fuel Cell

An enzyme electrode was prepared in the same manner as in Example 51, except for employing enzymes prepared in Comparative Example 49. Components immobilized on the enzyme electrode have the following formulation:

busGDH: 0.3 units

ecoISO: 0.6 units

ppuDp: 0.6 units

Fc-PAA: 16 μg

PEGDE: 10 μg

A fuel cell was prepared in the same manner as in Example 51, except for employing this enzyme electrode, and voltage-current characteristics were measured to obtain the following results:

shortcircuit current density: 310 μA/cm²

maximum electric power: 31 μW/cm²

Based on Example 51 and Comparative Example 51, it is identified that the fructose fuel cell of Example 51, in comparison with that of Comparative Example 51, has a higher current density and is capable of providing a larger current. This is assumed because, in the fuel cell of Example 51, despite of a fact that the amounts of glucose dehydrogenase, xylose isomerase and diaphorase immobilized on the enzyme electrode are same, glucose dehydrogenase and xylose isomerase are retained in a physical proximity as both enzymes are associated, whereby a conversion/oxidation reaction from fructose to gluconolactone via- glucose is executed promptly to generate NADH in a larger amount per unit time.

### (Example 52) Fructose Electrochemical Reaction Device

The enzyme electrode prepared in Example 51 was used to prepare an electrochemical reaction device shown in FIG. 10. A 50 mM phosphate buffer (pH 7.5) containing 100 mM of sodium chloride, 5 mM of fructose, and 1 mM of nicotinamide adenine dinucleotide is used as the sample solution 3, then a potential of 0.3 VvsAg/AgCl is applied for 100 minutes under a nitrogen atmosphere, and a product is quantitatively measured by a high-speed liquid chromatography. Gluconolactone is detected from the reaction electrolyte solution, and a reaction charge amount and an amount of generated gluconolactone show a high correlation, indicating that the reaction proceeds quantitatively.

### (Comparative Example 52) Fructose Electrochemical Reaction Device

An electrochemical reaction device was prepared in the same manner as in Example 52, except for employing the enzyme electrode prepared in Comparative Example 51, and was used for executing an electrochemical reaction. Gluconolactone is detected from the reaction electrolyte solution, and a reaction charge amount and an amount of generated gluconolace show a high correlation, indicating that the reaction proceeds quantitatively. It is however found that the reaction charge per unit time is smaller than in Example 52.

Based on Example 52 and Comparative Example 52, it is identified that the fructose electrochemical reaction device of Example 52, in comparison with that of Comparative Example 52, has a larger reaction charge per unit time and is capable of converting glucose into gluconolactone more efficiently. This is assumed because, in the electrochemical reaction device of Example 52, despite of a fact that the amounts of glucose dehydrogenase, xylose isomerase and diaphorase immobilized on the enzyme electrode are same, glucose dehydrogenase and xylose isomerase are retained in a physical proximity as both enzymes are associated, whereby the conversion/oxidation reaction from fructose to gluconolactone via glucose is executed more promptly and an amount of NADH generated per unit time is made larger.

### (Example 53) Preparation of Associated Protein (His-pfuGDH-Dzip1and tmaISO-Dzip2) [SEQ ID NOS. 135, 136] of Glucose Dehydrogenase (pfuGDH) Derived from Pyrococcus furiosus and Xylose Isomerase (tmaISO) Derived from Thermotoga maritime

A genome DNA is prepared from Pyrococcus furiosus [ATCC 43587] by an ordinary method. A PCR is executed utilizing this genome DNA as a template and employing synthetic oligoDNAs SEQ ID NO:19 and SEQ ID NO:20 as primers to obtain an amplified DNA product of 799 bp. The amplified DNA product is digestion cleaved by restriction enzymes BamHI and HindIII, and is inserted into a same restriction enzyme site of pETDuet-1 (manufactured by Novagen) to obtain a His-pfuGDH expression vector pETDuet-pfuGDH.

Then, a genome DNA is prepared from Thermotoga maritima [ATCC 43589] by an ordinary method. A PCR is executed utilizing this genome DNA as a template and employing synthetic oligoDNAs SEQ ID NO:130 having a sequence recognized by NdeI and SEQ ID NO:131 having a sequence recognized by XhoI as primers to obtain an amplified DNA product of 1356 bp.

The amplified DNA product is digestion cleaved by restriction enzymes NdeI and XhoI, and is inserted into a same restriction enzyme site of pETDuet-pfuGDH to obtain a co-expression vector pETDuet-pfuGDH-tmaISO, co-expressing His-pfuGDH and tmaISO.

Then, as in Example 17, 5′-terminal phosphated synthetic oligoDNAs (SEQ ID NO:51 and 52) are used to prepare a DNA fragment (double strand). This DNA fragment is inserted in a recognition site for restriction enzymes HindIII and AflII of pETDuet-pfuGDH-tmaISO. As a result, there is obtained a co-expression vector pETDuet-pfuGDH-Dzip1/tmaISO of a fusion protein, in which an association site sequence SEQ ID NO:169-Dzip1(SEQ ID NO:53) is fused to a C-terminal side of His-pfuGDH, and tmaISO.

Then, as in Example 17, 5′-terminal phosphated synthetic oligoDNAs (SEQ ID NO:54 and 55) are used to prepare a DNA fragment. This DNA fragment is inserted in a recognition site for restriction enzymes XhoI and AvrII of pETDuet-pfuGDH-Dzip1/tmaISO, thereby obtaining a co-expression vector pETDuet-pfuGDH-Dzip1/tmaISO-Dzip2 (SEQ ID NO:132) of a fusion protein, in, which an association site sequence SEQ ID NO:169-Dzip1(SEQ ID NO:53) is fused to a C-terminal side of His-pfuGDH, and a fusion protein in which an association site sequence SEQ ID NO:169-Dzip2 (SEQ ID NO:56) is fused at a C-terminal side of tmaISO.

Then, a PCR is executed utilizing the genome DNA of Pyrococcus furiosus [ATCC 43587] Pyrococcus furiosus [ATCC 43587] as a template and employing synthetic oligoDNAs SEQ ID NO:24 and SEQ ID NO:20 as primers to obtain an amplified DNA product of 797 bp. The amplified DNA product is digestion cleaved by restriction enzymes NcoI and HindIII, and is inserted into a same restriction enzyme site of pCDFDuet-1 (manufactured by Novagen) to obtain a pfuGDH expression vector pCDFDuet-pfuGDH.

Then, a PCR is executed utilizing the genome DNA of Thermotoga maritima [ATCC 43589] as a template and employing synthetic oligoDNAs SEQ ID NO:130 having a sequence recognized by NdeI and SEQ ID NO:133 having a sequence recognized by XhoI as primers to obtain an amplified DNA product of 1356 bp.

The amplified DNA product is digestion cleaved by restriction enzymes NdeI and XhoI, and is inserted into a same restriction enzyme site of pCDFDuet-pfuGDH to obtain a co-expression vector pCDFDuet-pfuGDH-tmaISO (SEQ ID NO:134) of pfuGDH and tmaISO.

Expression vectors pETDuet-pfuGDH::tmaISO and pCDFDuet-pfuGDH-tmaISO are transformed into E. coli BL21(DE3) by an ordinary method. The transform can be selected as a resistant strain to antibiotics ampicillin and streptomycin. The transforms were used to prepare enzymes by the method 1.

Then, a genome DNA is prepared from Pyrococcus horikoshii KT2440 [ATCC 700860] by an ordinary method. A PCR is executed utilizing this genome DNA as a template and employing synthetic oligoDNAs SEQ ID NO:21 and SEQ ID NO:22 as primers to obtain an amplified DNA product of 1344 bp. The amplified DNA product is digestion cleaved by restriction enzymes NdeI and XhoI, and is inserted into a same restriction enzyme site of pET-21a(+) (manufactured by Novagen) to obtain a phoDp-His expression vector pET21-phoDp.

The expression vector pET21-phoDp is transformed into E. coli BL21(DE3) by an ordinary method. The transform can be selected as a resistant strain to antibiotic ampicillin. The transform was used to prepare an enzyme by the method 1. The enzyme thus obtained may be used for constructing an enzyme electrode and the like.

### (Example 57) Preparation of Associated Protein (His-sceADH-Dzip3, goxALDH-Dzip4/ppuDp-Dzip4 and ppuDp-Dzip5) [SEQ ID NOS. 150, 151 and 152] of Alcohol Dehydrogenase (sceADH) Derived from Saccharomyces cerevisiae, Aldehyde Dehydrogenase (goxALDH) Derived from Gluconobacter oxydans and diaphorase (ppuDp) derived from Pseudomonas putida

A genome DNA is prepared from Saccharomyces cerevisiae [ATCC 47058] by an ordinary method. A PCR is executed utilizing this genome DNA as a template and employing synthetic oligoDNAs SEQ ID NO:31 and SEQ ID NO:32 as primers to obtain an amplified DNA product of 1075 bp.

The amplified DNA product is digestion cleaved by restriction enzymes BamHI and HindIII, and is inserted into a same restriction enzyme site of pETDuet-1 (manufactured by Novagen) to obtain a His-sceADH expression vector pETDuet-sceADH.

Then, a genome DNA is prepared from Gluconobacter oxydans [ATCC 621H] by an ordinary method. A PCR is executed utilizing this genome DNA as a template and employing synthetic oligoDNAs SEQ ID NO:109 having a sequence recognized by NdeI and SEQ ID NO:110 having a sequence recognized by XhoI as primers to obtain an amplified DNA product of 1560 bp.

The amplified DNA product is digestion cleaved by restriction enzymes NdeI and XhoI, and is inserted into a same restriction enzyme site of pETDuet-sceADH to obtain a co-expression vector pETDuet-sceADH-goxALDH, co-expressing His-sceADH and goxALDH.

Then, 5′-terminal phosphated synthetic oligoDNAs SEQ ID NO:137 and 138 are mixed in equal amounts in a TE buffer, and annealed by heating followed by standing to cool:

(SEQ ID No. 137)

(SEQ ID No. 138)

This DNA fragment is inserted in a recognition site for restriction enzymes HindIII and AflII of pETDuet-sceADH-goxALDH, thereby obtaining a co-expression vector pETDuet-sceADH-Dzip3/goxALDH of a fusion protein, in which an association site sequence SEQ ID NO:169-Dzip3 (SEQ ID NO:139) is fused to a C-terminal side of His-sceADH, and goxALDH.

Then, 5′-terminal phosphated synthetic oligoDNAs (SEQ ID NO:140 and 141) are mixed in equal amounts in a TE buffer, and annealed by heating followed by standing to cool.

This DNA fragment is inserted in a recognition site for restriction enzymes XhoI and AvrII of pETDuet-sceADH-Dzip3/goxALDH, thereby obtaining a co-expression vector pETDuet-sceADH-Dzip3/goxALDH-Dzip4 (SEQ ID NO:143) of a fusion protein, in which an association site sequence SEQ ID NO:169-Dzip3 (SEQ ID NO:139) is fused to a C-terminal side of His-sceADH, and a fusion protein in which an association site sequence SEQ ID NO:169-Dzip4 (SEQ ID NO:142) is fused at a C-terminal side of goxALDH.

Then a PCR is executed utilizing the genome DNA of Saccharomyces cerevisiae [ATCC 47058] as a template and employing synthetic oligoDNAs SEQ ID NO:34 and SEQ ID NO:32 as primers to obtain an amplified DNA product of 1073 bp.

The amplified DNA product is digestion cleaved by restriction enzymes NcoI and HindIII, and is inserted into a same restriction enzyme site of pCDFDuet-1 (manufactured by Novagen) to obtain a sceADH expression vector pCDFDuet-sceADH.

Then, a PCR is executed utilizing the genome DNA of Gluconobacter oxydans [ATCC 621H] as a template and employing synthetic oligoDNAs SEQ ID NO:109 having a sequence recognized by NdeI and SEQ -ID NO:112 having a sequence recognized by XhoI as primers to obtain an amplified DNA product of 1560 bp.

The amplified DNA product is digestion cleaved by restriction enzymes NdeI and XhoI, and is inserted into a same restriction enzyme site of pCDFDuet-sceADH to obtain a co-expression vector pCDFDuet-sceADH-goxALDH (SEQ ID NO:113) of sceADH and goxALDH.

Then a genome DNA is prepared from Pseudomonas putida KT2440 [ATCC 47054] by an ordinary method. A PCR is executed utilizing this genome DNA as a template and employing synthetic oligoDNAs SEQ ID NO:144 having a sequence recognized by NcoI and SEQ ID NO:145 having a sequence recognized by NcoI as primers to obtain an amplified DNA product of 725 bp.

The amplified DNA product is digestion cleaved by restriction enzymes NcoI and HindIII, and is inserted into a same restriction enzyme site of pCOLADuet-1 (manufactured by Novagen) to obtain a ppuDp expression vector pCOLADuet-ppuDp.

Then, a PCR is executed utilizing the genome DNA of Pseudomonas putida KT2440 as a template and employing synthetic oligoDNAs SEQ ID NO:3 and SEQ ID NO:13 as primers to obtain an amplified DNA product of 729 bp. The amplified DNA product is digestion cleaved by restriction enzymes NdeI and XhoI, and is inserted into a same restriction enzyme site of pCOLADuet-ppuDp to obtain a ppuDp co-expression vector pCOLADuet-ppuDp/ppuDp.

Then, 5′-terminal phosphated synthetic oligoDNAs SEQ ID NO:146 and 147 are mixed in equal amounts in a TE buffer, and annealed by heating followed by standing to cool.

This DNA fragment is inserted in a recognition site for restriction enzymes HindIII and AflII of pCOLADuet-ppuDp/ppuDp, thereby obtaining a co-expression vector pCOLADuet-ppuDp-Dzip5/ppuDp (SEQ ID NO:149) of a fusion protein, in which an association site sequence SEQ ID NO:169-Dzip5 (SEQ ID NO:148) is fused to a C-terminal side of ppuDp, and ppuDp.

Expression vectors pETDuet-sceADH-Dzip4/goxALDH-Dzip5, pCDFDuet-sceADH-goxALDH and pCOLADuet-ppuDp-Dzip5/ppuDp are transformed into E. coli BL21(DE3) by an ordinary method. The transform can be selected as a resistant strain to antibiotics ampicillin, streptomycin and kanamycin. The transforms were used to prepare enzymes by the method 1.

### (Example 58) Alcohol Sensor

Structure of an enzyme electrode is shown in FIG. 8. A conductive base member 20 is formed by glassy carbon of a diameter of 3 mm. On the conductive base member 20, an associated protein (His-sceADH-Dzip3/goxALDH-Dzip4/ppuDp-Dzip5) of alcohol dehydrogenase (sceADH) derived from Saccharomyces cerevisiae in Example 57, aldehyde dehydrogenase (goxALDH) derived from Gluconobacter oxydans, and diaphorase (ppuDp) derived from Pseudomonas putida, and a ferrocene-bonded polyallylamine (Fc-PAA) are immobilized by crosslinking by PEGDE. Components immobilized on the enzyme electrode have the following formulation:

His-sceADH-Dzip3/goxALDH-Dzip4/ppuDp-Dzip5 (sceADH: 0.3 units, goxALDH: 0.6 units, and ppuDp: 0.6 units)

Fc-PAA: 16 μg

PEGDE: 10 μg

This enzyme electrode is used as the reaction electrode 4 in FIG. 10 to construct an alcohol sensor. Also the sample solution 3 is formed by a 0.1M PIPES-NaOH aqueous buffer solution (pH 7.5) containing alcohol (ethanol) of a predetermined concentration and 1 mM of NAD. Under an application of a potential of 300 mV to the reaction electrode 4 with respect to the reference electrode 6, alcohol in the sample solution 3 is oxidized, in the presence of alcohol dehydrogenase, to acetaldehyde, and NAD is reduced to NADH in this reaction. Then, in the presence of aldehyde dehydrogenase associated with alcohol dehydrogenase, acetaldehyde is oxidized to a carboxylic acid, and NAD is simultaneously reduced to NADH. NADH, generated by the enzyme reactions of alcohol dehydrogenase and aldehyde dehydrogenase, is oxidized in the presence of diaphorase associated with both enzymes to NAD, and in this reaction, ferrocene serving as the electron transfer mediator is oxidized to generate a ferricinium ion. As the reaction electrode 4 has a potential of 300 mV with respect to the reference electrode 6, the ferricinium ion receives an electron from the reaction electrode 4 and is reduced to ferrocene. A reduction current corresponding to the alcohol concentration in the sample solution can be measured by measuring a current caused by an electron transfer at the reaction electrode 4.

Based on Example 58 and Comparative Example 42, it is identified that the alcohol sensor of Example 58, in comparison with that of Comparative Example 42, has a higher sensitivity to the alcohol concentration and is capable of quantitative determination of alcohol of a lower concentration. This is assumed because, in the enzyme electrode of Example 58, despite of a fact that the amounts of alcohol dehydrogenase, aldehyde dehydrogenase and diaphorase immobilized on the enzyme electrode are same, alcohol dehydrogenase, aldehyde dehydrogenase and diaphorase are retained in a physical proximity as these enzymes are associated, whereby an amount of NADH generated per unit time is made larger.

The enzymes of Example 58 may also be utilized for constructing an alcohol fuel cell and the like.

### (Example 61) Preparation of Associated Protein (His-pfuADH-Dzip3, tthALDH-Dzip4 and phoDp-Dzip5) [SEQ ID NOS. 157, 158 and 159] of Alcohol Dehydrogenase (pfuADH) derived from Pyrococcus furiosus, Aldehyde Dehydrogenase (tthALDH) Derived from Thermus thermophilus and Diaphorase (phoDp) Derived from Pyrococcus horikoshii

A genome DNA is prepared from Pyrococcus furiosus [ATCC 43587] by an ordinary method. A PCR is executed utilizing this genome DNA as a template and employing synthetic oligoDNAs SEQ ID NO:39 and SEQ ID NO:40 as primers to obtain an amplified DNA product of 1147 bp.

The amplified DNA product is digestion cleaved by restriction enzymes BamHI and HindIII, and is inserted into a same restriction enzyme site of pETDuet-1 (manufactured by Novagen) to obtain a His-pfuADH expression vector pETDuet-pfuADH.

Then, a genome DNA is prepared from Thermus thermophilus [ATCC BAA-163] by an ordinary method. A PCR is executed utilizing this genome DNA as a template and employing synthetic oligoDNAs SEQ ID NO:117 having a sequence recognized by NdeI and SEQ ID NO:117 having a sequence recognized by XhoI as primers to obtain an amplified DNA product of 1614 bp.

The amplified DNA product is digestion cleaved by restriction enzymes NdeI and XhoI, and is inserted into a same restriction enzyme site of pETDuet-pfuADH to obtain a co-expression vector pETDuet-pfuADH-tthALDH, co-expressing His-pfuADH and tthALDH.

Then, 5′-terminal phosphated synthetic oligoDNAs (SEQ ID NO:137 and 138) are mixed in equal amounts in a TE buffer, and annealed by heating followed by standing to cool:

(SEQ ID No. 137)

(SEQ ID No. 138)

This DNA fragment is inserted in a recognition site. for restriction enzymes HindIII and AflII of pETDuet-pfuADH-tthALDH, thereby obtaining a co-expression vector pETDuet-pfuADH-Dzip3/tthALDH of a fusion protein, in which an association site sequence SEQ ID NO:169-Dzip3 (SEQ ID NO:139) is fused to a C-terminal side of His-pfuADH, and tthALDH.

Then, 5′-terminal phosphated synthetic oligoDNAs (SEQ ID NO:140 and 141) are mixed in equal amounts in a TE buffer, and annealed by heating followed by standing to cool.

This DNA fragment is inserted in a recognition site for restriction enzymes XhoI and AvrII of pETDuet-pfuADH-Dzip3/tthALDH, thereby obtaining a co-expression vector pETDuet-pfuADH-Dzip3/tthALDH-Dzip4 (SEQ ID NO:153) of a fusion protein, in which an association site sequence SEQ ID NO:169-Dzip3 (SEQ ID NO:139) is fused to a C-terminal side of His-pfuADH, and a fusion protein in which an association site sequence SEQ ID NO:169-Dzip4 (SEQ ID NO:142) is fused at a C-terminal side of tthALDH.

Then a PCR is executed utilizing the genome DNA of Pyrococcus furiosus (ATCC 43587] as a template and employing synthetic oligoDNAs SEQ ID NO:42 and SEQ ID NO:40 as primers to obtain an amplified DNA product of 1145 bp.

The amplified DNA product is digestion cleaved by restriction enzymes NcoI and HindIII, and is inserted into a same restriction enzyme site of pCDFDuet-1 (manufactured by Novagen) to obtain a pfuADH expression vector pCDFDuet-pfuADH.

Then, a PCR is executed utilizing the genome DNA of Thermus thermophilus [ATCC BAA-163] as a template and employing synthetic oligoDNAs SEQ ID NO:116 having a sequence recognized by NdeI and SEQ ID NO:119 having a sequence recognized by XhoI as primers to obtain an amplified DNA product of 1614 bp.

The amplified DNA product is digestion cleaved by restriction enzymes NdeI and XhoI, and is inserted into a same restriction enzyme site of pCDFDuet-pfuADH to obtain a co-expression vector pCDFDuet-pfuADH-tthALDH (SEQ ID NO:120) of pfuADH and tthALDH.

Then a genome DNA is prepared from Pyrococcus horikoshii [ATCC 700860] by an ordinary method. A PCR is executed utilizing this genome DNA as a template and employing synthetic oligoDNAs SEQ ID NO:154 having a sequence recognized by NcoI and SEQ ID NO:155 having a sequence recognized by HindIII as primers to obtain an amplified DNA product of 1344 bp.

The amplified DNA product is digestion cleaved by restriction enzymes NcoI and HindIII, and is inserted into a same restriction enzyme site of pCOLADuet-1 (manufactured by Novagen) to obtain a phoDp expression vector pCOLADuet-phoDp.

Then, a PCR is executed utilizing the genome DNA of Pyrococcus horikoshii as a template and employing synthetic oligoDNAs SEQ ID NO:21 and SEQ ID NO:25 as primers to obtain an amplified DNA product of 1344 bp. The amplified DNA product is digestion cleaved by restriction enzymes NdeI and XhoI, and is inserted into a same restriction enzyme site of pCOLADuet-phoDp to obtain a phoDp co-expression vector pCOLADuet-phoDp/phoDp.

Then, 5′-terminal phosphated synthetic oligoDNAs SEQ ID NO:146 and 147 are mixed in equal amounts in a TE buffer, and annealed by heating followed by standing to cool.

This DNA fragment is inserted in a recognition site for restriction enzymes HindIII and AflII of pCOLADuet-phoDp/phoDp, thereby obtaining a co-expression vector pCOLADuet-phoDp-Dzip5/phoDp (SEQ ID NO:156) of a fusion protein, in which an association site sequence SEQ ID NO:169-Dzip5 (SEQ ID NO:148) is fused to a C-terminal side of phoDp, and phoDp.

Expression vectors pETDuet-pfuADH-Dzip3/tthALDH-Dzip4, pCDFDuet-pfuADH-tthALDH and pCOLADuet-phoDp-Dzip5/phoDp are transformed into E. coli BL21(DE3) by an ordinary method. The transform can be selected as a resistant strain to antibiotics ampicillin, streptomycin and kanamycin.

The transforms were used to prepare enzymes by the method 1. The enzymes thus obtained may be used for constructing an enzyme electrode or a fuel cell.

In the present invention, explained by Examples and Comparative Examples thereof, an associated protein of enzymes derived from thermophilic bacteria shows, in comparison with an associated protein of enzymes derived from normal-temperature bacteria, smaller loss in the detection sensitivity even after a prolonged time, thus being superior in durability. Also in case of constructing a fuel cell with such enzymes, those derived from thermophilic bacteria are liable to show smaller output loss after the lapse of time.

While the present invention has been described with reference to exemplary embodiments, it is to be understood that the invention is not limited to the disclosed exemplary embodiments. The scope of the claims is to be accorded the broadest interpretation so as to encompass all such modification and equivalent structures and functions.

This application claims the benefit of Japanese Patent Application No. 2005-359172, filed Dec. 13, 2005, which is hereby incorporated by reference in its entirety.

What is claimed is:

1. An enzyme electrode comprising an electroconductive base member and an enzyme, wherein the enzyme is formed by an associated protein in which two or more different enzyme proteins are associated.

2. The enzyme electrode according to claim 1, wherein the enzyme is immobilized on the electroconductive base member, and the enzyme and the electroconductive base member are electrically connected via an electron transfer mediator.

3. The enzyme electrode according to claim 1, wherein the enzyme is formed by an associated protein which is formed by a first enzyme catalyzing a chemical reaction for generating a first reaction product from a first reaction substrate and a second enzyme catalyzing a chemical reaction for generating a second reaction product from a second reaction substrate, and
at least a chemical substance in the first reaction product is same as at least a chemical substance in the second reaction substrate.

4. The enzyme electrode according to claim 3, wherein the first enzyme is a dehydrogenase, and the second enzyme is a diaphorase.

5. The enzyme electrode according to claim 3, wherein the dehydrogenase is at least one selected from a class of glucose dehydrogenase, alcohol dehydrogenase, aldehyde dehydrogenase, lactic acid dehydrogenase, malic acid dehydrogenase and glutamic acid dehydrogenase.

6. The enzyme electrode according to claim 3, wherein the first enzyme is alcohol dehydrogenase, the second enzyme is aldehyde dehydrogenase, and diaphorase is supported in an enzyme immobilizing layer.

7. The enzyme electrode according to claim 3, wherein the first enzyme is isomerase, the second enzyme is glucose dehydrogenase, and diaphorase is supported in an enzyme immobilizing layer.

8. The enzyme electrode according to claim 1, wherein at least one of plural enzyme proteins constituting the associated protein is derived from thermophilic bacteria.

9. A sensor comprising the enzyme electrode according to claim 1 as a detecting part for detecting a substance.

10. A fuel cell comprising the enzyme electrode according to claim 1 as an anode or a cathode.

11. An electrochemical reaction device comprising the enzyme electrode according to claim 1 as a reaction electrode.
